SEQUENCE LISTING (5 sequences)

SEQ ID NO: 1 - protein, 132 aa, Artificial Sequence
  recombinant IL-2
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - DNA, 402 bp, Artificial Sequence
  recombinant IL-2
atgccaacctcctcctctaccaagaaaactcaactgcaactggaacacctgctgctggatctgcaaatgattctgaacggcattaacaactacaagaacccgaaactgacccgtatgctgaccttcaaattctatatgccgaagaaagctaccgaactgaaacacctgcaatgcctggaagaggagctgaaaccgctggaggaggttctgaacctggctcagagcaagaactttcatctgcgtccacgtgacctgatttccaacatcaacgttatcgttctggaactgaaaggtagcgaaaccactttcatgtgcgagtacgctgacgaaaccgctaccatcgttgaatttctgaaccgctggatcaccttctctcagtccattatctctactctgacctaa

SEQ ID NO: 3 - protein, 133 aa, Artificial Sequence [sequence omitted]

SEQ ID NO: 4 - DNA, 32 bp, Artificial Sequence
  forward primer
aatcatatggcacctacttcaagttctacaaa

SEQ ID NO: 5 - DNA, 27 bp, Artificial Sequence
  reverse primer
aatttatcaagttagtgttgagatgat

(19) United States
(12) Patent Application Publication
(10) Pub. No.: US 2022/0401561 A1
(43) Pub. Date: Dec. 22, 2022

(54) PROTEIN-MACROMOLECULE CONJUGATES AND METHODS OF USE THEREOF

(71) Applicant: Beijing Xuanyi PharmaSciences Co., Ltd., Beijing (CN)

(72) Inventors: Yuntao SONG, Palo Alto, CA (US); Hui LI, Santa Clara, CA (US); Haiping ZHOU, Beijing (CN); Chuan LIAO, Beijing (CN)

(21) Appl. No.: 17/764,475

(22) PCT Filed: Sep. 30, 2020
(86) PCT No.: PCT/US2020/053572

§ 371 Date: Mar. 28, 2022

Related U.S. Application Data
(60) Provisional application No. 62/908,435, filed on Sep. 30, 2019.

(51) Int. Cl. A61K 47/54; A61K 47/60; A61K 47/34; A61P 35/00

(52) U.S. Cl. CPC A61K 47/545; A61K 47/60; A61K 47/34; A61P 35/00

(57) ABSTRACT

The present disclosure provides protein-macromolecule conjugates, releasable linkers, and macromolecules, as defined herein. The disclosed conjugates provide unique properties that ae based at least upon the properties of linker and number of linker-Macromolecule moieties. Also provided herein are a method of synthesis and use of conjugates in treating diseases and disorders.

### CROSS-REFERENCE TO RELATED APPLICATIONS

This application claims the benefit of and priority to U.S. Provisional Application No. 62/908,435 filed Sep. 30, 2019, which is hereby incorporated by reference in its entirety.

### SEQUENCE LISTING

This application is being filed electronically via EFS-Web and includes an electronically submitted sequence listing in .txt format. The .txt file contains a sequence listing entitled “CSPL_008_01WO_SeqList_ST25.txt” created on Sep. 29, 2020 and having a size of ˜3.71 kilobytes. The sequence listing contained in this .txt file is part of the specification and is incorporated herein by reference in its entirety.

### FIELD OF THE INVENTION

The present disclosure relates to the methodology for preparing protein-macromolecule conjugates, through utilization of bifunctional linkers. In addition, the present disclosure relates to novel conjugates that are designed for pharmacokinetic control in delivering proteins with biological function. In particular, the disclosure relates to protein-macromolecule conjugates having desired rates of protein release. More specifically, the disclosure relates to conjugates having an IL-2 moiety (i.e. a moiety having at least some activity similar to human IL-2) and macromolecules with one or more linkers. In addition, the present disclosure relates to conjugates compositions, methods for preparing conjugates, methods of administering a conjugate, and method of using the conjugates in the field of cancer therapy.

### BACKGROUND OF THE INVENTION

Many drugs suffer from unfavorable pharmacokinetic parameters that limit their effectiveness. Rapid clearance of such drugs from physiological compartments, either via metabolism or excretion, results in short lifetimes and reduced exposure to targets. For example, therapeutic agonists based on natural proteins are attractive immune modulators that can help mount an effective durable anti-tumor response; however, they are not ideal pharmaceutical agents due to poor pharmacokinetics (PK), poor tolerability, and pleiotropic activity that may be exacerbated by frequent dose administration.

The cytokine interleukin-2 (IL-2) is an endogenous agonist of the IL-2 pathway and is a well-described stimulator of CD8⁺ T cell (CD8 T) and NK cells. A high-dose IL-2 regimen administered every eight hours in a hospital setting using an IL-2 variant known as ‘aldesleukin’ was approved in the 1990s by the United States Food and Drug Administration for the treatment of metastatic melanoma and renal cell carcinoma, providing up to 25% durable responses. High doses of IL-2 are needed to activate CD8 T cells and NK cells, which tend to express the low-affinity IL-2 receptor beta gamma subunits (IL-2Rβγ). Compounding the need for high doses of IL-2 is the poor PK profile of this protein. High-dose aldesleukin is not broadly used because of severe toxicities associated with over-activation of the immune system. In addition of these toxicities, IL-2 also stimulates proliferation and activation of regulatory T cells (Tregs). These cells constitutively express the high-affinity heterotrimeric IL-2 receptor alpha beta gamma subunits (IL-2Rαβγ). Treg activation may exacerbate immune suppression, potentially compromising the intended anti-tumor response.

Polymeric prodrugs and polymer-drug conjugates can improve effectiveness of drugs for therapeutic applications. Polymer conjugated drugs generally exhibit prolonged half-life, higher stability, water solubility, lower immunogenicity and antigenicity and specific targeting to tissues or cells. Polymers are used as carriers in polymeric prodrugs/macromolecular prodrugs for the delivery of drugs, proteins, targeting moieties, and imaging agents. Polymeric prodrugs can be regarded as drug delivery systems that exhibit their therapeutic activities by means of releasing smaller therapeutic drug molecules from a polymer chain molecule for a prolonged period of time, which results in enhanced pharmacokinetic behavior by increasing the half-life, bioavailability, and hence prolonged pharmacological action.

In the attempt to address the toxicity concerns and poor PK properties of IL-2, certain conjugates of IL-2 have been suggested. See, for example, U.S. Pat. Nos. 4,766,106, 5,206,344, 5,089,261, 4,902,502, 9,861,705 and WO 2019/028419.

In addition to extending plasma half-life and reducing immunogenicity, PEGylation provides an opportunity to control the selectivity of protein binding. As an example, NKTR-214, a PEGylated IL-2 clinical candidate, displays reduced binding to the TL-2 receptor α-subunit (IL-2Rα) owing to site-specific PEGylation with releasable linkers at the lysine residues of the IL-2-IL-2Rα interface. Binding to the IL-2 receptor J-subunit (IL-2Rβ) is minimally impacted. Consequently, NKTR-214 affords increased proliferation of CD8+ tumor-killing memory effector T cells, reduced proliferation of immunosuppressive regulatory T cells and enhanced antitumor efficacy relative to IL-2 in preclinical evaluation. See, for example, U.S. Pat. No. 9,861,705, Clin. Cancer Res. 22, 680-690 (2016); PLOS ONE 12, e0179431 (2017).

Choice of linker chemistry is important in the design of polymer-drug conjugate therapeutics, as it confers spatiotemporal control over the cleavage and subsequent release of active agents. Without sufficient linker stability, a conjugated drug can exhibit premature release, annulling the advantages of its macromolecular carrier. However, in the case of an inactive polymeric prodrug, insufficient drug release may result in sub-therapeutic drug levels and, consequently, suboptimal therapeutic efficacy. Therefore, a sustained drug release profile that affords prolonged therapeutic efficacy is highly desirable.

Some prodrug molecules release active drugs under physiological conditions by virtue of pH-dependent beta elimination. This approach utilizes a spontaneous, first-order rate of cleavage of the drug from the PEG carriers that is initiated when the conjugate is exposed to physiological pH. Their cleavage rates are predetermined by the acidity of a C—H bond on the linker; the acidity is in turn controlled by electron-withdrawing groups attached to the ionizable C—H. See, for example, U.S. Pat. Nos. 6,504,005, 8,680,315, and WO 2004/089279.

Despite its widespread use, a considerable limitation of PEG and its subsequent utility in therapeutics is its non-biodegradability. At present, approved PEGylated protein therapeutics employ PEGs of ≤40 kDa molecular mass, close to the glomerular filtration threshold of approximately 50 kDa. Although increased molecular mass generally affords extended circulation time, concerns regarding the accumulation of non-biodegradable PEG limit the optimization of polymer molecular mass and the resultant pharmacokinetics.

### SUMMARY OF THE INVENTION

Described herein is the general design of protein-[macromolecule]z conjugates with multiple linkers. The unique linkers of the present disclosure enable the construction of drug conjugates having predictable, tunable release kinetics. In addition, the molecular mass of each macromolecule can be controlled under the desirable mass for renal clearance, which in some embodiments is less than 40-50 kDa. By increasing the number of macromolecules (z) on the protein, the total molecular mass of the conjugates can be increased and, subsequently, the circulation time of the conjugates can be extended. Besides using tunable electron withdrawing groups on the releasable linker, the release rate of the active protein can be further controlled and optimized by changing the number of macromolecules (z) on the protein.

Generally, conjugation of multiple macromolecules to one protein is difficult and not efficient. We envisioned a general approach to conjugation of a protein with multiple bifunctional linkers, then reaction of the linkers with macromolecules to provide protein-[macromolecule]z conjugates. This technique provides the advantage of minimized steric hindrance and therefore improves reaction efficiency. Moreover, the synthetic and purification steps are simplified and less costly. Therefore this technique provides a considerable advantage for the large-scale production and manufacture of polymer-protein therapeutics.

The present disclosure describes this general strategy for providing protein-[macromolecule]z conjugates having releasable linkers of predictable and controllable release rate. These conjugates bearing controllable release rate can provide a valuable therapeutic tool for the treatment of disease. In some embodiments, the present disclosure describes protein-[macromolecule]z conjugates having non-releasable linkers and releasable linkers. Embodiments of the present disclosure are therefore directed to methodology for preparing such conjugates, compositions comprising the conjugates and methods of use thereof, which are novel and completely unsuggested by the art.

Accordingly, in one or more embodiments of the disclosure, the present disclosure relates to conjugation methods for preparing conjugates having a protein with relevant biological functions and multiple macromolecules connecting with linkers. In some embodiments, the conjugation methods involve the functionalization of a protein with bifunctional linkers, followed conjugation to a macromolecule. In some embodiments, the protein includes, but is not limited to, cytokines, chemokines, antibodies, and peptides. In some embodiments, the macromolecule includes, but is not limited to, water-soluble polymers, PEG, lipid, polysialic acid, albumin, and Fc.

The present disclosure also relates to novel bifunctional releasable linkers and compositions thereof, utilization of novel bifunctional releasable linkers in therapeutic applications, and methods for preparing. Among the advantages of the disclosed technology is the ability to efficiently functionalize proteins with a plurality of bifunctional releasable linkers provided herein. Conjugation to macromolecules can then be utilized to improve the pharmacokinetic properties of the highly functionalized protein.

In one or more embodiments of the disclosure, a conjugate is provided, the conjugate comprising a residue of an IL-2 moiety covalently attached to one or more water-soluble polymers through releasable linkers.

In one or more embodiments of the disclosure, a conjugate is provided, the conjugate comprising a residue of an IL-2 moiety covalently attached to one or more water-soluble polymers through non-releasable linkers.

In one or more embodiments of the disclosure, a conjugate is provided, the conjugate comprising a residue of an IL-2 moiety covalently attached to one or more water-soluble polymers through non-releasable and releasable linkers.

In one or more embodiments of the disclosure, a method for delivering a conjugate is provided, the method comprising the step of intravenously or subcutaneously administering to a patient a composition comprised of a conjugate of a residue of an IL-2 and water-soluble polymers.

In one or more embodiments of the disclosure, a method for delivering a conjugate is provided, the method comprising the steps of administering to a cancer patient: (a) a composition comprising a conjugate of a residue of an IL-2 and one or more water-soluble polymers; and (b) an effective amount of an anti-CTLA-4 antibody or an effective amount of an anti-PD-1/PD-L1 antibody. In some embodiments, an effective amount of an anti-CTLA-4 antibody is an amount that inhibits a CTLA-4 pathway. In some embodiments, an effective amount of an anti-PD-1/PD-L1 antibody is an amount that inhibits a PD-1/PD-L1 pathway. By way of clarity, with regard to the sequence of steps in accordance with this method, unless otherwise indicated, the method is not limited to the sequence of steps and step (a) can be performed before, after or simultaneously with, performing step (b).

The present disclosure provides protein-macromolecule conjugates, releasable linkers, and macromolecules, as defined herein. The disclosed conjugates provide unique properties that are based at least upon the properties of linker and number of linker-Macromolecule moieties. Also provided herein are unique method of synthesis and use of conjugates in treating diseases and disorders.

Additional embodiments of the disclosure are set forth in the following description and claims.

### BRIEF DESCRIPTION OF THE DRAWINGS

FIG. 1 shows the nucleotide and amino acid sequences of rIL-2 (SEQ ID NOs:1-3).

FIG. 2 shows IL-2-(N3)z distributions determined for Example 14, Example 16, Example 18 and Example 22 by LC-MS.

FIG. 3 shows SDS-PAGE (Tris Acetate) analysis of click-PEGylation product rIL-2-(PEG)_{z} for Example 15, example 17, Example 19 and Example 22.

FIG. 4A to FIG. 4E show dose-response curves comparing CTLL-2 cell proliferation assay of IL-2, unreleased conjugates and released conjugates from Example 15 (4A), Example 17 (4B), Example 19 (4C), Example 22 (4D) and Example 27 (4E). The Y-axis is labeled A₄₅₀-A₆₃₀.

FIG. 5, FIG. 6, FIG. 7, FIG. 8 and FIG. 9 show tumor growth inhibition following the administration of rIL-2 and rIL-2-polymer conjugates at different administration schemes.

### DETAILED DESCRIPTION OF THE DISCLOSURE

### Definitions

In describing and claiming one or more embodiments of the present disclosure, the following terminology will be used in accordance with the definitions described below.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

The term “compound(s) of the present disclosure” or “compound(s) of the present disclosure” refers to compounds of formulae disclosed herein or any subgenera thereof, or a pharmaceutically acceptable salt, stereoisomer, solvate or hydrate thereof, as disclosed herein. In certain embodiments, intermediates are contemplated as compounds of the present disclosure.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)-for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof. In some embodiments, “stereoisomer”, as used herein, refers to an enantiomer, a mixture of enantiomers, a diastereomer, or a mixture of two or more diastereomers.

“Enantiomers” refer to two stereoisomers of a compound which are non-superimposable mirror images of one another. A mixture of such isomers can be called an enantiomeric mixture.

A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. The disclosure includes all stereoisomers of the compounds described herein.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may be separated under high resolution analytical procedures such as electrophoresis and chromatography.

The term “regioisomer” is art-recognized and refers to compounds having the same molecular formula but differing in the degree of atomic connectivity. Thus, a “regioselective process” is one in which the formation of a specific regioisomer is preferred over others, for example, the reaction significantly increases the yield of a specific regioisomer. As used herein, “regioisomer” can refer to a single regioisoimer or a mixture of two or more regiosiomers.

A “tautomer” refers to a proton shift from one atom of a molecule to another atom of the same molecule. The present disclosure includes tautomers of any said compounds.

The terms “pharmaceutical combination,” “therapeutic combination” or “combination” as used herein, refers to a single dosage form comprising at least two therapeutically active agents, or separate dosage forms comprising at least two therapeutically active agents together or separately for use in combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 software naming program (CambridgeSoft). For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

The term “composition” or “formulation” denotes one or more substance in a physical form, such as solid, liquid, gas, or a mixture thereof. One example of composition is a pharmaceutical composition, i.e., a composition related to, prepared for, or used in medical treatment.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfo aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

As used herein, “solvate” means a complex formed by solvation (the combination of solvent molecules with molecules or ions of the active agent of the present disclosure), or an aggregate that consists of a solute ion or molecule (the active agent of the present disclosure) with one or more solvent molecules. In the present disclosure, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present disclosure. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The terms “excipient”, “carrier”, and “vehicle” are used interchangeably throughout this application and denote a substance with which a compound of the present disclosure is administered.

“Therapeutically effective amount” means the amount of a compound or a therapeutically active agent that, when administered to a patient for treating a disease or other undesirable medical condition, is sufficient to have a beneficial effect with respect to that disease or condition. The therapeutically effective amount will vary depending on the type of the selected compound or a therapeutically active agent, the disease or condition and its severity, and the age, weight, etc. of the patient to be treated. Determining the therapeutically effective amount of a given compound or a therapeutically active agent is within the ordinary skill of the art and requires no more than routine experimentation.

“Treating” or “treatment” as used herein covers the treatment of the disease or condition of interest in a mammal, preferably a human, having the disease or condition of interest, and includes: preventing the disease or condition from occurring in a mammal, in particular, when such mammal is predisposed to the condition but has not yet been diagnosed as having it; inhibiting the disease or condition, i.e., arresting its development; relieving the disease or condition, i.e., causing regression of the disease or condition; or relieving the symptoms resulting from the disease or condition, i.e., relieving pain without addressing the underlying disease or condition.

As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

The present disclosure is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

As used herein, a “subject” can be a human, non-human primate, mammal, rat, mouse, cow, horse, pig, sheep, goat, dog, cat and the like. The terms “subject” and “patient” are used interchangeably herein in reference, e.g., to a mammalian subject, such as a human subject.

The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

“PEG”, “polyethylene glycol” and “poly(ethylene glycol)” as used herein, are interchangeable and encompass any nonpeptidic water-soluble poly(ethylene oxide). Typically, PEGs for use in accordance with the disclosure comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced, e.g., during a synthetic transformation. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀alkoxy group, and still more preferably a C₁₋₅alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃O(CH₂CH₂O)ₙ— and CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin. The end-capping group may also include a targeting moiety, such that the polymer—as well as anything, e.g., an IL-2 moiety, attached thereto—can preferentially localize in an area of interest.

“Non-naturally occurring” with respect to a polymer as described herein, means a polymer that in its entirety is not found in nature. A non-naturally occurring polymer may, however, contain one or more monomers or segments of monomers that are naturally occurring, so long as the overall polymer structure is not found in nature.

The term “water soluble” as in a “water-soluble polymer” polymer is any polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the disclosure are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

The terms “active,” “reactive” or “activated” when used in conjunction with a particular functional group, refers to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

As used herein, the term “electron altering group” is meant to include any atom or functional group that modifies the electron density of the moiety to which it is attached. Electron altering groups include electron donating groups, which donate electron density (e.g., amine, hydroxy, alkoxyl, alkyl) and electron withdrawing groups (e.g., nitro, cyano, trifluoromethyl) which withdraw electron density.

The terms “spacer moiety,” “linkage” and “linker” are used herein to refer to a bond or an atom or a collection of atoms optionally used to link interconnecting moieties such as a terminus of a macromolecule segment and a protein or an electrophile or nucleophile of a protein. The spacer moiety may be hydrolytically stable or may include a physiologically hydrolyzable or enzymatically degradable linkage. Unless the context clearly dictates otherwise, a spacer moiety optionally exists between any two elements of a compound (e.g., the provided conjugates comprising a residue of protein and macromolecule can be attached directly or indirectly through a spacer moiety).

Suitable spacers of the present disclosure, include spacers comprising a linker that can include one or more of carbon atoms, nitrogen atoms, sulfur atoms, phosphorus atoms, oxygen atoms, and combinations thereof. A suitable spacer moiety may comprise an amide, secondary amine, carbamate, thioether, phosphate, phosphorothioate, disulfide group and/or click chemistry product groups. Non-limiting examples of specific spacer moieties include those selected from the group consisting of —O—, —S—, —S—S—, —C(O)—, —C(O)—NH—, —NH—C(O)—NH—, —O—C(O)—NH—, —OP(O)(OH)—, —OP(S)(OH)—, —C(S)—, —CH₂—, —CH₂—CH₂—, —CH₂—CH₂—CH₂—, —CH₂—CH₂—CH₂—CH₂—, —CH₂—CH₂—CH₂—CH₂—CH₂—, O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]₁—(OCH₂CH₂)ₘ—, bivalent cycloalkyl group, bivalent aryl, —O—, —S—, a divalent amino acid residue, —N(R³)—, and combinations of two or more of any of the foregoing, wherein R³ is H or an organic radical selected from the groups consisting of substituted or unsubstituted alkyl, substituted or unsubstituted alkenyl, substituted or unsubstituted alkynyl, substituted or unsubstituted aryl, (1) is zero to six, and (m) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes.

The term “bifunctional linker” refers to a linker, as defined above, having two reactive atoms or functional groups. In certain embodiments, the two reactive groups are orthogonal functional groups with different modes of reactivity, so that each functional group is capable is reacting independently of the other and in a particular sequence, if so desired. As would be understood by one of skill in the art, the bifunctional linkers disclosed herein can be used to carry out site-specific reactions to assemble protein-macromolecule conjugates.

“Acyl” refers to —C(═O)-alkyl radical.

“Amino” refers to the —NH₂ radical.

“Cyano” refers to the —CN radical.

“Halo” “halide” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Sulfhydryl” and “mercapto” refers to —SH radical.

Hydrogen is H or D.

“Alkyl” or “alkyl group” refers to a fully saturated, straight (linear) or branched hydrocarbon chain radical having from one to twenty carbon atoms, and which is attached to the rest of the molecule by a single bond. Alkyls comprising any number of carbon atoms from 1 to 20 are included. An alkyl comprising up to 20 carbon atoms is a C₁-C₂₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted. The term “lower alkyl” refers to a C₁-C₆ alkyl, which can be linear or branched, for example including branched C₃-C₆ alkyl. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl as well as cycloalkylene-containing alkyl.

“Alkylene”, “-alkyl-” or “alkylene chain” refers to a fully saturated, straight or branched divalent hydrocarbon chain radical, and having from one to twenty carbon atoms. Non-limiting examples of C₁-C₂₀ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twenty carbon atoms, and having one or more carbon-carbon double bonds. Each alkenyl group is attached to the rest of the molecule by a single bond. Alkenyl group comprising any number of carbon atoms from 2 to 20 are included. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twenty carbon atoms, and having one or more carbon-carbon double bonds. Non-limiting examples of C₂-C₂₀ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twenty carbon atoms, and having one or more carbon-carbon triple bonds. Each alkynyl group is attached to the rest of the molecule by a single bond. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twenty carbon atoms, and having one or more carbon-carbon triple bonds. Non-limiting examples of C₂-C₂₀ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” or “—O-alkyl” refers to a radical of the formula —ORₐ where Rₐ is an alkyl, alkenyl or alknyl radical as defined above containing one to twenty carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

“Aryl” refers to a hydrocarbon ring system radical comprising hydrogen, 6 to 18 carbon atoms and at least one aromatic ring. For purposes of this disclosure, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Aralkyl”, “arylalkyl” or “-alkylaryl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

“Alkoxy” refers to an —OR group, wherein R is alkyl or substituted alkyl, preferably C₁₋₆ alkyl (e.g., methoxy, ethoxy, propyloxy, and so forth).

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to to about 12 carbon atoms, more preferably 3 to about 8 carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, bicyclo[3.1.0]hexane, octahydropentalene, bicyclo[1.1.1]pentane, cubane, and the like. Unless otherwise stated specifically in the specification, a cycloalkyl group can be optionally substituted. “Cycloalkylene” refers to a cycloalkyl group that is inserted into an alkyl chain by bonding of the chain at any two carbons in the cyclic ring system.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” or “-alkylcycloalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one, two, three, four, five, six or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one, two, three, four, five, six or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one, two, three, four, five, six or more halo radicals, as defined above, e.g., 1-fluoropropynyl, 1-fluorobutynyl, and the like. Unless stated otherwise specifically in the specification, a haloalkenyl group can be optionally substituted.

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; nitro; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para).

“Noninterfering substituents” are those groups that, when present in a molecule, are typically nonreactive with other functional groups contained within the molecule. Non-limiting examples include halogen (F, Br, Cl, I), alkyl (e.g., methyl, ethyl, propyl, isopropyl, butyl, isobutyl, s-butyl, pentyl, neopentyl, hexyl, isoamyl, and the like), haloalkyl (e.g., CF₃, CHF₂, CH₂F, and the like), cycloalkyl (cyclopropyl, cyclobutyl, cyclopentyl, cyclohexyl, and the like), alkoxy (—OR), haloalkoxy (e.g., —OCF₃, —OCHF₂, —OCH₂F, and the like), amino (e.g., —N(H)alkyl, —N(alkyl)₂, —NH(cycloalkyl), —NH(aryl), and the like), amido (e.g, —NH(COR), sulfonyl (e.g., —SO₂R), acyl (e.g., —C(O)R, cyano, nitro, phenyl, and heteroaryl (e.g., oxazolyl, thiazolyl, imidazolyl, pyridyl, pyrimidinyl, and the like), wherein R is independently H, alkyl, alkyoxy, amino, or aryl (e.g., phenyl).

“Heterocyclyl,” “heterocyclic ring” or “heterocycle” refers to a stable 3- to 20-membered non-aromatic ring radical which consists of two to twelve carbon atoms and from one to six heteroatoms preferably selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted. In some embodiments, “substituted heterocycle” is a heterocycle having one or more side chains formed from noninterfering substituents.

The term “hydroxyalkyl” or “hydroxylalkyl” refers to an alkyl group that is substituted with one or more hydroxyl (—OH) groups. In certain embodiments, a hydroxyalkyl group is substituted with one, two, three, four, five or more —OH groups. A hydroxyalkyl group may optionally be substituted with one or more additional substituents as described herein.

The term “hydrocarbyl” refers to a monovalent hydrocarbon radical, whether aliphatic, partially or fully unsaturated, acyclic, cyclic or aromatic, or any combination of the preceding. In certain embodiments, a hydrocarbyl group has 1 to 40 or more, 1 to 30 or more, 1 to 20 or more, or 1 to 10 or more, carbon atoms. The term “hydrocarbylene” refers to a divalent hydrocarbyl group. A hydrocarbyl or hydrocarbylene group may optionally be substituted with one or more substituents as described herein.

The term “heterohydrocarbyl” refers to a hydrocarbyl group in which one or more of the carbon atoms are each independently replaced by a heteroatom selected from oxygen, sulfur, nitrogen and phosphorus. In certain embodiments, a heterohydrocarbyl group has 1 to 40 or more, 1 to 30 or more, 1 to 20 or more, or 1 to 10 or more, carbon atoms, and 1 to 10 or more, or 1 to 5 or more, heteroatoms. The term “heterohydrocarbylene” refers to a divalent hydrocarbyl group. Examples of heterohydrocarbyl and heterohydrocarbylene groups include without limitation ethylene glycol and polyethylene glycol moieties, such as (—CH₂CH₂O—)ₙH (a monovalent heterohydrocarbyl group) and (—CH₂CH₂O—)ₙ (a divalent heterohydrocarbylene group) where n is an integer from 1 to 12 or more, and propylene glycol and polypropylene glycol moieties, such as (—CH₂CH₂CH₂O—)ₙH and (—CH₂CH(CH₃)O—)ₙH (monovalent heterohydrocarbyl groups) and (—CH₂CH₂CH₂O—)ₙ and (—CH₂CH(CH₃)O—)ₙ (divalent heterohydrocarbylene groups) where n is an integer from 1 to 12 or more. A heterohydrocarbyl or heterohydrocarbylene group may optionally be substituted with one or more substituents as described herein.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” or “-alkylheterocyclyl” refers to a radical of the formula —R_{b}—R_{c} where R_{b} is an alkylene, alkenylene, or alkynylene chain as defined above and R_{c} is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms preferably selected from the group consisting of nitrogen, oxygen and sulfur, and at least one aromatic ring. For purposes of this disclosure, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted. In some embodiments, “substituted heteroaryl” is heteroaryl having one or more noninterfering groups as substituents.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “-alkylheteroaryl” refers to a radical of the formula —R_{b}—R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms with a list provided herein. If no substituent list is included, substituents can be, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with halide, cyano, nitro, hydroxyl, sulfhydryl, amino, —OR_{g}, —SR_{g}, —NRₕRᵢ, alkyl, alkenyl, alkynyl, haloalkyl, hydroxyalkyl, aminoalkyl,-alkylcycloalkyl,-alkylheterocyclyl, -alkylaryl, -alkylheteroaryl, cycloalkyl, heterocyclyl, aryl, heteroaryl, —C(═O)R_{g}, —C(═NRⱼ)R_{g}, —S(═O)R_{g}, —S(═O)₂R, —S(═O)₂ORₖ, —C(═O)ORₖ, —OC(═O)R_{g}, —C(═O)NRₕRᵢ, —NR_{g}C(═O)R_{g}, —S(═O)₂NRₕRᵢ, —NR_{g}S(═O)₂R_{g}, —OC(═O)OR_{g}, —OC(═O)NRₕRᵢ, —NR_{g}C(═O)OR_{g}, —NR_{g}C(═O)NRₕRᵢ, —NR_{g}C(═NRⱼ)NRₕRᵢ, —P(═O)(R_{g})₂, —P(═O)(ORₖ)R_{g}, —P(═O)(ORₖ)₂, —OP(═O)(R_{g})₂, —OP(═O)(ORₖ)R_{g}, and —OP(═O)(ORₖ)₂, wherein: each occurrence of R_{g} is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; each occurrence of Rₕ and Rᵢ is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, or R_{b} and Rᵢ, together with the nitrogen atom to which they are attached, form a heterocyclic or heteroaryl ring; each occurrence of Rⱼ independently is hydrogen, —OR_{g}, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; and each occurrence of Rₖ independently is hydrogen, W, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, wherein each occurrence of W independently is H⁺, Li⁺, Na⁺, K⁺, Cs⁺, Mg⁺², Ca⁺², or —⁺N(R_{g})₂RₕRᵢ.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

An “organic radical” as used herein shall include alkyl, substituted alkyl, alkenyl, substituted alkenyl, alkynyl, substituted alkynyl, aryl, and substituted aryl.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “ [symbol] ” infers that when R³ is “XY”, the point of attachment bond is the same bond as the bond by which R³ is depicted as being bonded to CH₃.

“Fused” refers to any ring structure described herein which is fused to an existing ring structure in the compounds of the disclosure. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Electrophile” and “electrophilic group” refer to an ion or atom or collection of atoms, which may be ionic, having an electrophilic center, i.e., a center that is electron seeking, capable of reacting with a nucleophile.

“Nucleophile” and “nucleophilic group” refers to an ion or atom or collection of atoms that may be ionic, having a nucleophilic center, i.e., a center that is seeking an electrophilic center or with an electrophile.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carbamate, carboxylate ester, phosphate ester, anhydrides, acetals, ketals, acyloxyalkyl ether, imines, orthoesters, peptides and oligonucleotides.

A “releasable linker” refers to a linker that connects protein with macromolecules. Either through hydrolysis, enzymatic processes, catalytic processes or otherwise, the macromolecule is released, thereby resulting in the unconjugated protein moiety. In certain embodiments, the releasable linker releases the macromolecule by the aforementioned processes that take place in vivo.

An “enzymatically degradable linkage” means a linkage that is subject to degradation by one or more enzymes.

A “hydrolytically stable” linkage or bond refers to a chemical bond, typically a covalent bond, which is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), carbon-sulfur bonds, ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Pharmaceutically acceptable excipient or carrier” refers to an excipient that may optionally be included in the compositions of the disclosure and that causes no significant adverse toxicological effects to the patient. “Pharmacologically effective amount,” “physiologically effective amount,” and “therapeutically effective amount” are used interchangeably herein to mean the amount of a protein-macromolecule conjugate that is needed to provide a desired level of the conjugate (or corresponding unconjugated protein) in the bloodstream or in the target tissue. The precise amount will depend upon numerous factors, e.g., the particular protein, the components and physical characteristics of the therapeutic composition, intended patient population, individual patient considerations, and the like, and can readily be determined by one skilled in the art, based upon the information provided herein.

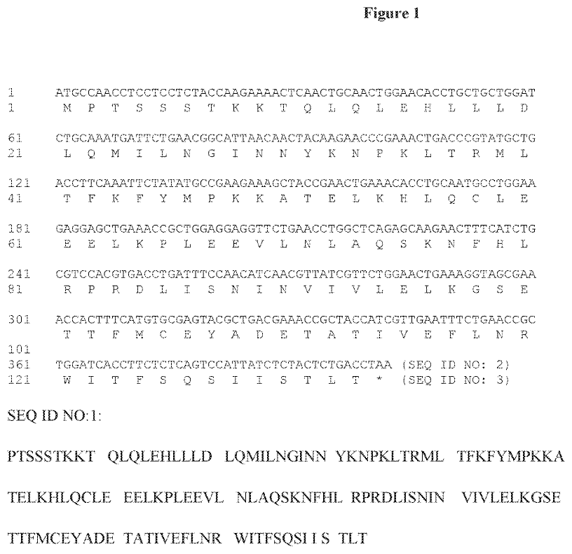

The term “IL-2 moiety,” as used herein, refers to a moiety having human IL-2 activity. The IL-2 moiety will also have at least one electrophilic group or nucleophilic group suitable for reaction with a polymeric reagent. In addition, the term “IL-2 moiety” encompasses both the IL-2 moiety prior to conjugation as well as the IL-2 moiety residue following conjugation. As will be explained in further detail below, one of ordinary skill in the art can determine whether any given moiety has IL-2 activity. Proteins comprising an amino acid sequence corresponding to the sequence in FIG. 1 is an IL-2 moiety, as well as any protein or polypeptide substantially homologous thereto. As used herein, the term “IL-2 moiety” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties.

The term “substantially homologous” means that a particular subject sequence, for example, a mutant sequence, varies from a reference sequence by one or more substitutions, deletions, or additions, the net effect of which does not result in an adverse functional dissimilarity between the reference and subject sequences. For purposes of the present disclosure, sequences having greater than 80 percent (more preferably greater than 85 percent, still more preferably greater than 90 percent, with greater than 95 percent being most preferred) homology, equivalent biological activity (although not necessarily equivalent strength of biological activity), and equivalent expression characteristics are considered substantially homologous. For purposes of determining homology, truncation of the mature sequence should be disregarded.

The term “fragment” means any protein or polypeptide having the amino acid sequence of a portion or fragment of an IL-2 moiety, and which has the biological activity of IL-2. Fragments include proteins or polypeptides produced by proteolytic degradation of an IL-2 moiety as well as proteins or polypeptides produced by chemical synthesis by methods routine in the art.

The term “patient,” refers to a living organism suffering from or prone to a condition that can be prevented or treated by administration of an active agent (e.g., conjugate), and includes both humans and animals.

“Optional” or “optionally” means that the subsequently described circumstance may or may not occur, so that the description includes instances where the circumstance occurs and instances where it does not.

“Substantially” means nearly totally or completely, for instance, satisfying one or more of the following: greater than 50%, 51% or greater, 75% or greater, 80% or greater, 90% or greater, and 95% or greater of the condition.

Amino acid residues in peptides are abbreviated as follows: Phenylalanine is Phe or F; Leucine is Leu or L; Isoleucine is lie or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The present disclosure includes all pharmaceutically acceptable isotopically labeled compounds of the disclosure wherein one or more atoms are replaced by atoms having the same atomic number, but an atomic mass or mass number different from the atomic mass or mass number usually found in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵, ¹⁷O and ¹⁸O, phosphorus, such as ³²P, and sulfur, such as ³⁵S.

Certain isotopically-labeled compounds of the disclosure, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²K may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the disclosure can generally be prepared by conventional techniques known to those skilled in the art.

The phrase “an enantiomer, a mixture of enantiomers, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, a tautomer, a mixture of two or more tautomers, a regioisomer, a mixture of two or more regioisomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, a tautomer, a mixture of two or more tautomers, a regioisomer, a mixture of two or more regioisomers, or an isotopic variant of the compound referenced therein.”

### Methods of Preparation

The present disclosure provides the method for preparing protein-[macromolecule]z conjugates for controlling the delivery rate of therapeutic protein agents when administered to patients requiring treatment with the therapeutic agents. The conjugates prepared through the methods of the disclosure provide a means of delivery therapeutic agents over a sustained period of time, controlled by the releasable rate of the linkers and number of the macromolecules.

In one aspect, the disclosure is directed to the methods for preparing Protein-Macromolecule conjugates using the scheme (I):

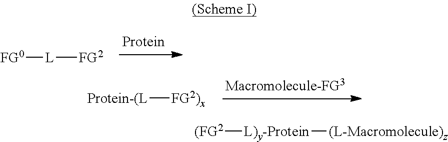

wherein x is an integer from 1-25;

y is an integer from 0-24;

z is an integer from 1-25;

x=y+z;

L is a linker;

FG⁰ is a functional group capable of reacting with a nucleophilic group of an active protein agent to form a linkage, including a carbamate linkage, a thiol bridge and the like;

FG² is a functional group capable of reacting with FG³ through click chemistry, including but not limited to azide, alkynyl, and cycloalkynyl groups (e.g., dibenzocyclooctyne (DBCO));

FG³ is a functional group capable of reacting with FG² through click chemistry, including but not limited to azide, alkynyl, and cycloalkynyl (e.g., dibenzocyclooctyne (DBCO)) groups;

Protein is a chemokine, a chemokine antagonist, a cytokine, a cytokine antagonist, an antibody, or a therapeutic peptide;

The cytokine includes GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, or TNF-β.

In certain embodiments, the cytokine is IL-2.

In certain embodiments, the IL-2 comprises about 80%, 85%, 90%, 95%, 96%, 97%, 98%, or 99% sequence identity to SEQ ID NO:1.

The chemokine includes MCP-1, MCP-2, MCP-3, MCP-24, MCP-5, CXCL76, I-309 (CCL1), BCA1 (CXCL13), MIG, SDF-1/PBSF, IP-10, I-TAC, MIP-1α, MIP-1p, RANTES, eotaxin-1, eotaxin-2, GCP-2, Gro-α, Gro-β, Gro-γ, LARC (CCL20), ELC (CCL19), SLC (CCL21), ENA-78, PBP, TECK(CCL25), CTACK (CCL27), MEC, XCL1, XCL2, HCC-1, HCC-2, HCC-3, or HCC-4.

The antibody targets one or more of angiopoietin 2, AXL, ACVR2B, angiopoietin 3, activin receptor-like kinase 1, amyloid A protein, β-amyloid, AOC3, BAFF, BAFF-R, B7-H3, BCMAC, A-125 (imitation), C5, CA-125, CCL11 (eotaxin-1), CEA, CSF1R, CD2, CD3, CD4, CD6, CD15, CD19, CD20, CD22, CD23, CD25, CD28, CD30, CD33, CD37, CD38, CD40, CD41, CD44, CD51, CD52, CD54, CD56, CD70, CD74, CD97B, CD125, D134, CD147, CD152, CD154, CD279, CD221, C242 antigen, CD276, CD278, CD319, Clostridium difficile, claudin 18 isoform 2, CSF1R, CEACAM5, CSF2, carbonic anhydrase 9, CLDN18.2, cardiac myosin, CCR4, CGRP, coagulation factor III, c-Met, CTLA-4, DPP4, DR5, DLL3, DLL4, dabigatran, EpCAM, ebolavirus glycoprotein, endoglin, episialin, EPHA3, c-Met, FGFR2, fibrin II beta chain, FGF 23, folate receptor 1, GMCSF, GD2 ganglioside, GDF-8, GCGR, gelatinase B, glypican 3, GPNMB, GMCSF receptor α-chain, kallikrein, KIR2D, ICAM-1, ICOS, IGF1, IGF2, IGF-1 receptor, IL-1α, IL-1β, IL-2, IL-4Rα, IL-5, IL-6, IL-6 R, IL-9, IL-12, IL-13, ILI7A, ILI7F, IL-20, IL-22, IL-23, IL-31, IFN-α, IFN-β, IFN-γ, integrin α/β, interferon α/β receptor, Influenza A hemagglutinin, ILGF2, HER1, HER2, HER3, HHGFR, HGF, HLA-DR, hepatitis B surface antigen, HNGF, Hsp90, HGFR, L-selectin, Lewis-Y antigen, LYPD3, LOXL2, LIV-1, MUC1, MCP-1, MSLN, mesothelin, MIF, MCAM, NCA-90, NCA-90Notch 1, nectin-4, PCDP1, PD-L1, PD-1, PCSK9, PTK7, PCDC1, phosphatidylserine, RANKL, RTN4, Rhesus factor, ROR1, SLAMF7, Staphylococcus aureus alpha toxin, Staphylococcus aureus bi-component leucocidin, SOST, selectin P, SLITRK6, SDC1, TFPI, TRAIL-R2, tumor antigen CTAA16.88, TNF-α, TWEAK receptor, TNFRSF8, TYRP1, tau protein, TAG-72, TSLP, TRAIL-R1, TRAIL-R2, TGF-β, TAG-72, TRAP, TIGIT, tenascin C, OX-40, VEGF-A, VWF, VEGFR1, or VEGFR2.

Peptides include but are not limited to: glucagon-like peptide 1 (GLP-1), exendin-2, exendin-3, exendin-4, atrial natriuretic factor (ANF), ghrellin, vasopressin, growth hormone, growth hormone-releasing hormone (GHRH), RC-3095, somatostatin, bombesin, PCK-3145, Phe-His-Ser-Cys-Asn (PHSCN), IGF1, B-type natriuretic peptide, peptide YY (PYY), interferons, thrombospondin, angiopoietin, calcitonin, gonadotropin-releasing hormone, hirudin, glucagon, anti-TNF-alpha, fibroblast growth factor, granulocyte colony stimulating factor, obinepitide, pituitary thyroid hormone (PTH), leuprolide, sermorelin, pramorelin, nesiritide, rotigaptide, cilengitide, MBP-8298, AL-108, enfuvirtide, thymalfasin, daptamycin, HLFI-II, Lactoferrin, Delmitide, glutathione, T-cell epitope PRI, Protease-3 peptides 1-11, B-cell epitope P3, lutenizing hormone-releasing hormone (LHRH), substance P, neurokinin A, neurokinin B, CCK-8, enkephalins, including leucine enkephalin and methionine enkephalin, dermaseptin, [des-Ala20, G1n34]-dermaseptin, surfactant-associated antimicrobial anionic peptide, Apidaecin IA; Apidaecin IB; OV-2; 1025, Acetyl-Adhesin Peptide (1025-1044) amide; Theroma-cin (49-63); Pexiganan (MSI-78); Indolicidin; Apelin-15 (63-77); CFPlO (71-85); Lethal Factor (LF) Inhibitor Anthrax related; Bactenecin; Hepatitis Virus C NS3 Protease Inhibitor 2; Hepatitis Virus C NS3 Protease Inhibitor 3; Hepatitis Virus NS3 Protease Inhibitor 4; NS4A-NS4B Hepatitis Virus C (NS3 Protease Inhibitor I); HIV-1, HIV-2 Protease Substrate; Anti-FM Peptide; Bak-BH3; Bax BH3 peptide (55-74) (wild type); Bid BH3-r8; CTT (Gelatinase Inhibitor); E75 (Her-2/neu) (369-377); GRP78 Binding Chimeric. Peptide Motif; p53(17-26); EGFR2/KDR Antagonist; Colivelin AGA-(C8R) HNGI 7 (Humanin derivative); Activity-Dependent Neurotrophic Factor (ADNF); Beta-Secretase Inhibitor I; Beta-Secretase Inhibitor 2; ch[beta]-Amyloid (30-16); Humanun (HN) sHNG, [Glyl4]-HN, [Glyl 4]-Humanin; Angiotensin Converting Enzyme Inhibitor (BPP); Renin Inhibitor III; Annexin I (ANXA-I; Ac2-12); Anti-Inflammatory Peptide I; Anti-Inflammatory Peptide 2; Anti-Inflammatory Apelin 12; [D-Phel2, Leul4]-Bombesin; Antennapedia Peptide (acid) (penetratin); Antennepedia Leader Peptide (CT); Mastoparan; [Thr28, Nle31]-Cholecystokinin (25-33) sulfated; Nociceptin (1-13) (amide); Fibrinolysis Inhibiting Factor; Gamma-Fibrinogen (377-395); Xenin; Obestatin (human); [Hisl, Lys6]-GHRp (GHRP-6); [Ala5, [beta]-Ala8]-NeurokininA (4-10); Neuromedin B; Neuromedin C; Neuromedin N; Activity-Dependent Neurotrophic Factor (ADNF-14); Acetalin I (Opioid Receptor Antagonist I); Acetalin 2 (Opioid Receptor Antagonist 2); Acetalin 3 (Opioid Receptor Antagonist 3); ACTH (1-39) (human); ACTH (7-38) (human); Sauvagine; Adipokinetic Hormone (Locusta Migratoria); Myristoylated ADP-Ribosylation Factor 6, myr-ARF6 (2-13); PAMP (1-20) (Proadrenomedullin (1-20) human); AGRp (25-51); Amylin (8-37) (human); Angiotensin I (human); Angiotensin II (human); Apstatin (Aminopeptidase P Inhibitor); Brevinin-I; Magainin I; RL-37; LL-37 (Antimicrobial Peptide) (human); Cecropin A; Antioxidant peptide A; Antioxidant peptide B; L-Camosine; BcI 9-2; NPVF; NeuropeptideAF (hNPAF) (Human); Bax BH3 peptide (55-74); bFGF Inhibitory Peptide; bFGF inhibitory Pep tide II; Bradykinin; [Des-Argl OJ-HOE 140; Caspase I Inhibitor II; Caspase I Inhibitor VIII; Smac N7 Protein (MEKI Derived Peptide Inhibitor I; hBD-1 ([beta]-Defensin-1) (human); hBD-3 ([beta]-Defensin-3) (human); hBD-4 ([beta]-Defensin-4) (human); HNP-I (Defensin Human Neutrophil Peptide I); HNP-2 (Defensin Human neutrophil Peptide-2 Dynorphin A (1-17)); Endomorphin-I; [beta]-Endorphin (human porcine); Endothelin 2 (human); Fibrinogen Binding Inhibitor Peptide; Cyclo(-GRGDSP); TP508 (Thrombin-derived Peptide); Galanin (human); GIP (human); Gastrin Releasing Peptide (human); Gastrin-1 (human); Ghrelin (human); PDGF-BB peptide; [D-Lys3]-GHRP-6; HCV Core Protein (1-20); a3B1 Integrin Peptide Fragment (325) (amide); Laminin Pentapeptide (amide) Mel-anotropin-Potentiating Factor (MPF); VA-[beta]-MSH, Lipo-tropin-Y (Proopiomelanocortin-derived); Atrial Natriuretic Peptide (1-28) (human); Vasonatrin Peptide (1-27); [Ala5, B-Ala8]-Neurokinin A (4-10); Neuromedin L (NKA); Ac-(Leu28, 31)-Neuropeptide Y (24-26); Alytesin; Brain Neuropeptide II; [D-tyrll]-Neurotensin; IKKy NEMO Binding Domain (NBD) Inhibitory Peptide; PTD-p50 (NLS) Inhibitory Peptide; OrexinA (bovine, human, mouse, rat); Orexin B (human); Aquaporin-2(254-267) (human Pancreastatin)(37-52); Pancreatic Polypeptide (human); Neuropeptide; Peptide YY (3-36) (human); Hydroxymethyl-Phytochelatin 2; PACAP (I-27) (amide, human, bovine, rat); Prolactin Releasing Peptide (1-31) (human); Salusin-alpha; Salusin-beta; Saposin C22; Secretin (human); L-Selectin; Endokinin A/B; Endokinin C (Human); Endokinin D (Human); Thrombin Receptor (42-48) Agonist (human); LSKL (Inhibitor of Thrombospondin); Thyrotropin Releasing Hormone (TRH); P55-TNFR Fragment; Urotensin II (human); VIP (human, porcine, rat); VIP Antagonist; Helodermin; Exenatide; ZPIO (AVEOOIOO); Pramlinitide; AC162352 (PYY)(3-36); PYY; Obinepitide; Glucagon; GRP; Ghrelin (GHRP6); Leuprolide; Histrelin; Oxytocin; Atosiban (RWJ22164); Sermorelin; Nesiritide; bivalirudin (Hirulog); Icatibant; Aviptadin; Rotigaptide (ZP123, GAP486); Cilengitide (EMD-121924, RGD Peptides); A1buBNP; BN-054; Angiotensin II; MBP-8298; Peptide Leucine Arginine; Ziconotide; AL-208; AL-108; Carbeticon; Tripeptide; SAL; Coliven; Humanin; ADNF-14; VIP (Vasoactive Intestinal Peptide); Thymalfasin; Bacitracin; Gramidicin; Pexiganan (MSI-78); P1 13; PAC-113; SCV-07; HLF1-I1 (Lactoferrin); DAPTA; TRI-1144; Tritrpticin; Anti-flammin 2; Gattex (Teduglutide, ALX-0600); Stimuvax (L-BLP25); Chrysalin (TP508); Melanonan II; Spantide II; Ceruletide; Sincalide; Pentagastin; Secretin; Endostatin peptide; E-selectin; HER2; IL-6; IL-8; IL-10; PDGF; Thrombospondin; uPA (I); uPA (2); VEGF; VEGF (2); Pentapeptide-3; XXLRR; Beta-Amyloid Fibrillogenesis; Endomorphin-2; TIP 39 (Tuberoinfundibular Neuropeptide); PACAP (1-38) (amide, human, bovine, rat); TGFB activating peptide; Insulin sensitizing factor (ISF402); Transforming Growth Factor BI Peptide (TGF-B1); Caerulein Releasing Factor; IELLQAR (8-branchMAPS); Tigapotide PK3145; Goserelin; Abarelix; Cetrorelix; Ganirelix; Degarelix (Triptorelin); Barusiban (FE 200440); Pralmorelin; Octreotide; Eptifibatide; Netamiftide (INN-00835); Daptamycin; Spantide II; Delmitide (RDP-58); AL-209; Enfuvirtide; IDR-I; Hexapeptide-6; Insulin-A chain; Lanreotide; Hexa[rho]eptide-3; Insulin B-chain; Glargine-A chain; Glargine-B chain; Insulin-LisPro B-chain analog; Insulin-Aspart B-chain analog; Insulin-Glulisine B chain analog; Insulin-Determir B chain analog; Somatostatin Tumor Inhibiting Analog; Pancreastatin (37-52); Vasoactive Intestinal Peptide fragment (KKYL-NH2); and Dynorphin A. Examples of proteins suitable for use in the disclosure include but are not limited to: immunotoxin SSlP, adenosine deaminase, argininase, and others.

The macromolecule can be a water-soluble polymer, a lipid, a protein or a polypeptide. In some embodiments, the macromolecule comprises a fatty acid comprising from about 6 to about 26 carbon atoms, a polymer selected from the group consisting of 2-methacryloyl-oxyethyl phosphoyl cholins, poly(acrylic acids), poly(acrylates), poly(acrylamides), poly(N-acryloylmorpholine), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethylene glycols), poly(ethylene oxides), poly(ethyl phosphates), poly(ethyloxazolines), poly(glycolic acids), poly(α-hydroxy acid), poly(hydroxyethyl acrylates), poly(hydroxyethyloxazolines), poly(hydroxymethacrylates), poly(hydroxyalkylmethacrylamides), poly(hydroxyalkylmethacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(oxyethylated polyol), poly(olefinic alcohol), polyphosphazene, poly(propylene glycols), poly(saccharide), poly(siloxanes), poly(urethanes), poly(vinyl alcohols), poly(vinyl amines), poly(vinylmethylethers), poly(vinylpyrrolidones), silicones, amylose, celluloses, carbomethyl celluloses, hydroxypropyl methylcelluloses, chitins, chitosans, dextrans, dextrins, gelatins, hyaluronic acids (HA) and derivatives, functionalized hyaluronic acids, mannans, pectins, heparin, heparan sulfate (HS), rhamnogalacturonans, starches, hydroxyalkyl starches, hydroxyethyl starches (HES), polysialic acid (PSA) and other carbohydrate-based polymers, xylans, and copolymers.

The macromolecule can also be a protein or polypeptide selected from the group consisting of albumin, transferrin, transthyretin, immunoglobulin, a XTEN peptide, a glycine-rich homoamino acid polymer (HAP), a PAS polypeptide, an elastin-like polypeptide (ELP), a CTP peptide, or a gelatin-like protein (GLK) polymer.

In certain embodiments, linker L is the residue of a releasable linker (RL).

In certain embodiments, x or z is 2 or more. In certain embodiments, x or z is 3 or more. In certain embodiments, x or z is 4 or more. In certain embodiments, x or z is 5 or more. In certain embodiments, x or z is 6 or more. In certain embodiments, x or z is more than 6.

In certain embodiments, the methods of preparation described herein relate to a first step involving conjugation of a protein with multiple bifunctional linkers. It is expected that due to the small size of the linkers, the conjugation process is more efficient and higher instances of conjugation can be achieved, compared to the conjugation of a protein with macromolecules directly. Also described herein, the second step of the disclosed methods can involve click chemistry designed to connect the linkers with macromolecules with high efficiency. Without being bound by any particular theory, it is believed that this method provides the advantage of minimized steric hindrance, which can therefore improve reaction efficiency. Moreover, the synthetic and purification steps are simplified and less costly, therefore this method provides a considerable advantage for the large-scale production and manufacture of polymer-protein therapeutics.

### Bifunctional Releasable Linkers

The conjugates of the present disclosure can be derived from bifunctional releasable linkers.

In some aspects, the present disclosure is directed to the bifunctional releasable linkers of the formula (I):

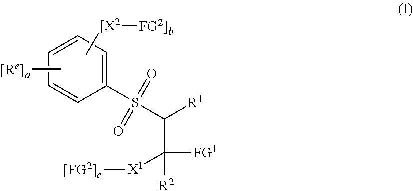

or a stereoisomer, tautomer or mixtures thereof, or isotopic variant thereof;

wherein:

X¹ is a first spacer moiety;

X² is a second spacer moiety;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;

a is an integer from 0 to 4;

b is an integer from 1 to 3;

c is an integer from 0 to 1;

FG¹ is a functional group capable of reacting with an amino group of an active agent to form a releasable linkage, such as a carbamate linkage; and

FG² is a functional group capable of reacting through click chemistry, independently including but not limited to azide, alkynyl, and cycloalkynyl (e.g., dibenzocyclooctyne (DBCO)) groups.

In some embodiments of formula (I), R¹ and R² are each independently a C₁₋₅ alkyl, a substituted C₁₋₅ alkyl, a C₂₋₆ alkenyl, a substituted C₂₋₆ alkenyl, a C₂₋₆ alkynyl, a substituted C₂₋₆ alkynyl, a phenyl, or a substituted phenyl. In certain embodiments, R¹ and R² are each independently a C₁₋₅ alkyl or a substituted C₁₋₅ alkyl.

In some embodiments of formula (I), R^{e} is nitro, cyano, halogen, —CONH(C₁₋₅ alkyl) or —CONH(phenyl), substituted —CONH(C₁₋₅ alkyl) or —CONH(phenyl), —SO₂NH(C₁₋₅ alkyl) or —SO₂NH(phenyl), substituted —SO₂NH(C₁₋₅ alkyl) or —SO₂NH(phenyl), —SO₂(C₁₋₅ alkyl) or —SO₂(phenyl), substituted —SO₂(C₁₋₅ alkyl) or —SO₂(phenyl), C₁₋₅ alkoxy, substituted C₁₋₅ alkoxy, C₁₋₅ alkyl or C₃₋₆ cycloalkyl, substituted C₁₋₅ alkyl or C₃₋₆ cycloalkyl, phenyl or 5- to 6-membered heteroaryl, or substituted phenyl or 5- to 6-membered heteroaryl.

In some embodiments of formula (I), a is an integer from 0 to 3. In some embodiments, a is an integer from 0 to 2. In some embodiments, a is 0. In some embodiments, a is 1. In some embodiments, a is 2. In some embodiments, a is 3. In some embodiments, a is 4.

In some embodiments of formula (I), b is an integer from 1 or 2. In some embodiments, b is 1. In some embodiments, b is 2. In some embodiments, b is 3.

In some embodiments of formula (I), In some embodiments, c is 0. In some embodiments, c is 1.

In some embodiments of formula (I), X¹ and X² are each independently selected from the spacer moieties described herein. In some embodiments, X¹ and X² are the same spacer moiety. In some embodiments, X¹ and X² are different spacer moieties.

Within formula (I), bifunctional releasable linkers having the more defined structures are provided:

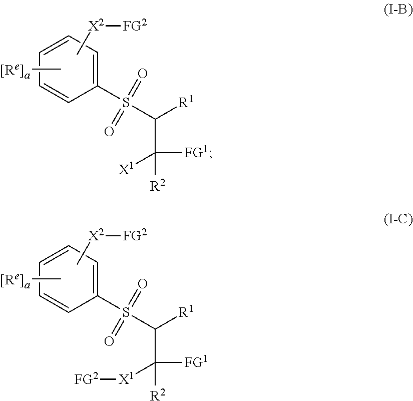

wherein each of X¹ is a first spacer moiety; X² is a second spacer moiety; R¹, R², [R^{e}]ₐ, FG¹ and FG² are as previously defined.

In certain embodiments of formula (I), (I-B), or (I-C), a is an integer from 0 to 2; R¹ and R² are each independently H, Me, or Et; and R^{e} is nitro, cyano, halogen, —CF₃, —CONHMe, —SO₂NHMe, —OMe, —NHMe, —NHAc, —NHSO₂Me, or —OCF₃.

In certain embodiments of formula (I), (I-B), or (I-C), the bifunctional releasable linker has following structure:

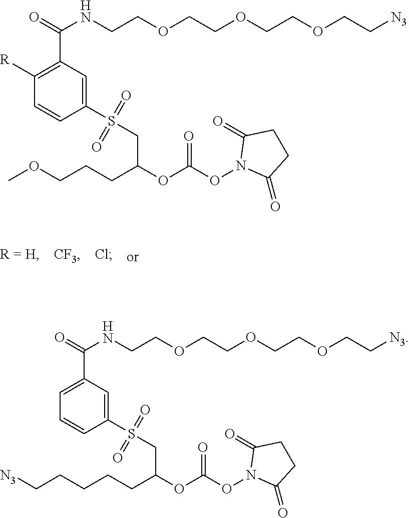

In another aspect, the present disclosure is directed to bifunctional releasable linkers of the formula (XVIII):

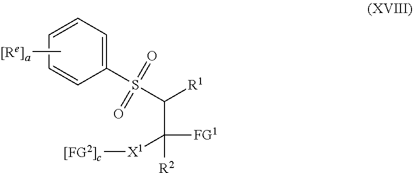

or a stereoisomer, tautomer or mixtures thereof, or isotopic variant thereof;

wherein:

X¹ is a spacer moiety;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;
a is an integer from 0 to 4;
c is 2;

FG¹ is a functional group capable of reacting with an amino group of an active agent to form a releasable linkage; and

FG² is a functional group capable of reacting through click chemistry.

In certain embodiments of formula (XVIII), a is an integer from 0 to 2; R¹ and R² are each independently hydrogen, Me, or Et; and R^{e} is nitro, cyano, halogen, —CF₃, —CONHMe, —SO₂NHMe, —OMe, —NHMe, —NHAc, —NHSO₂Me, or —OCF₃.

In certain embodiments of formula (XVIII), the bifunctional releasable linker has one of the following structures:

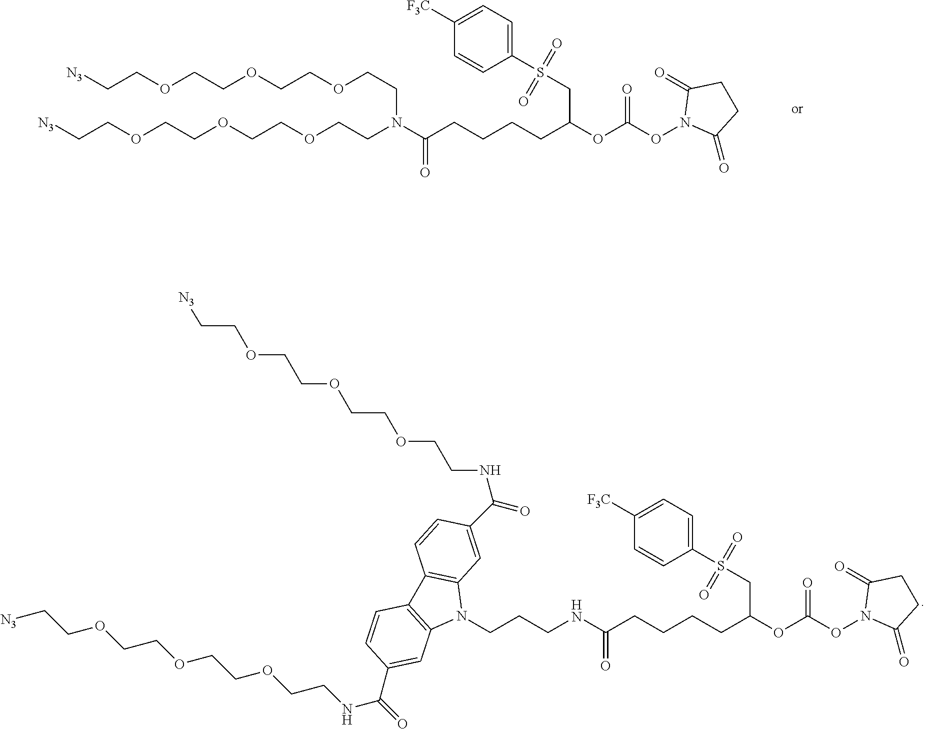 [SMILES: [N-]=[N+]=NCCOCCOCCOCCN(CCOCCOCCOCCN=[N+]=[N-])C(=O)CCCCC(CS(=O)(=O)c1ccc(C(F)(F)F)cc1)OC(=O)ON1C(=O)CCC1=O ; [N-]=[N+]=NCCOCCOCCOCCNC(=O)c1ccc2c3ccc(C(=O)NCCOCCOCCOCCN=[N+]=[N-])cc3n(CCCNC(=O)CCCCC(CS(=O)(=O)c3ccc(C(F)(F)F)cc3)OC(=O)ON3C(=O)CCC3=O)c2c1]

In another aspect, the present disclosure is directed to a bifunctional releasable linker of the formula (II):

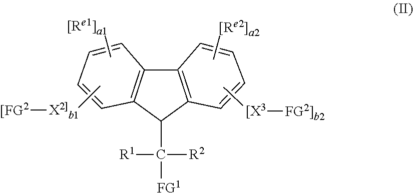

or a stereoisomer, tautomer or mixtures thereof, or isotopic variant thereof;
wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

R^{e1}, when present, is a first electron altering group;

R^{e2}, when present, is a second electron altering group;

X², when present, is a second spacer moiety;

X³, when present, is a third spacer moiety;

FG¹ is a functional group capable of reacting with an amino group of an active agent to form a releasable linkage, such as a carbamate linkage; and

FG² is a functional group capable of reacting through click chemistry, independently including but not limited to azide, alkynyl, and cycloalkynyl (e.g., dibenzocyclooctyne (DBCO)) groups.

In some embodiments of formula (II), R¹ and R² are each independently a C₁₋₅ alkyl, a substituted C₁₋₅ alkyl, a C₂₄ alkenyl, a substituted C₂₄ alkenyl, a C₂₋₆ alkynyl, a substituted C₂₋₆ alkynyl, a phenyl, or a substituted phenyl. In certain embodiments, R¹ and R² are each independently a C₁₋₅ alkyl or a substituted C₁₋₅ alkyl.

In some embodiments of formula (II), R^{e1} and R^{e2} are each independently nitro, cyano, halogen, haloalkyl (e.g., —CF₃, —CHF₂, —CH₂F, —CH₂F), —OC₁₋₅ alkyl, —O-haloalkyl (e.g., —OCF₃, —OCHF₂, —OCH₂F, —OCH₂F), —NH(C₁₋₅ alkyl), —NHCO(C₁₋₅ alkyl), —NHSO₂(C₁₋₅ alkyl), —CONH(C₁₋₅ alkyl), or —SO₂NH(C₁₋₅ alkyl). In certain embodiments, R^{e1} and R^{e2} are each independently nitro, cyano, halogen, —CF₃, —CONHMe, —SO₂NHMe, —OMe, —NHMe, —NHAc, —NHSO₂Me, or —OCF₃.

In some embodiments of formula (II), X² and X³ are each independently selected from the spacer moieties described herein. In some embodiments, X² and X³ are the same spacer moiety. In some embodiments, X² and X³ are different spacer moieties.

In certain embodiments of formula (II), a1 and a2 are each independently an integer from 0 to 2; R¹ and R² are each independently H, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Further exemplary bifunctional linkers fall within the following formula (II-A) or (II-B):

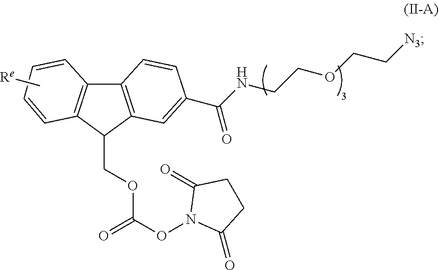

wherein R^{e} is hydrogen or an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl. In certain embodiments, R^{e} is hydrogen or fluoro.

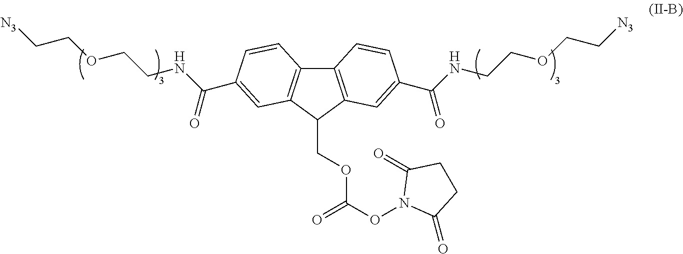

These releasable linkage-providing reagents can be prepared in accordance with the procedures set forth in US20060293499A1.

In another aspect, the present disclosure is directed to bifunctional releasable linkers of formula (UI):

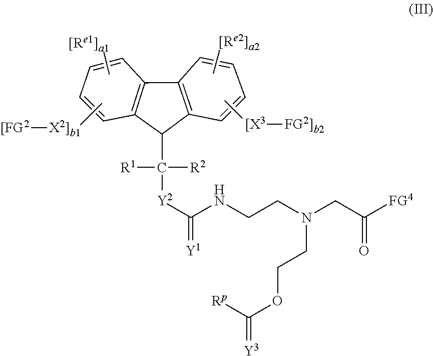

or a stereoisomer, tautomer or mixtures thereof, or isotopic variant thereof;
wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

R^{e1}, when present, is a first electron altering group;

R², when present, is a second electron altering group;

R^{p} is a hydrogen, alkyl, substituted alkyl, alkenyl, substituted alkenyl, alkynyl, substituted alkynyl, aryl, or substituted aryl;

X², when present, is a spacer moiety;

X³, when present, is a spacer moiety;

Y¹ is O or S;

Y² is O or S;

Y³ is O or S;

FG² is a functional group capable of reacting through click chemistry, independently including but not limited to azide, alkynyl, and cycloalkynyl (e.g., dibenzocyclooctyne (DBCO)) groups; and

FG⁴ is a functional group capable of reacting with an amino group of an active agent to form an amide linkage.

In some embodiments of formula (III), R¹, R² and R^{p} are each independently a C₁₋₅ alkyl, a substituted C₁₋₅ alkyl, a C₂₋₆ alkenyl, a substituted C₂₋₆ alkenyl, a C₂₋₆ alkynyl, a substituted C₂₋₆ alkynyl, a phenyl, or a substituted phenyl. In certain embodiments, R¹ and R² are each independently a C₁₋₅ alkyl or a substituted C₁₋₅ alkyl.

In some embodiments of formula (III), R^{e1} and R^{e2} are each independently nitro, cyano, halogen, haloalkyl (e.g., —CF₃, —CHF₂, —CH₂F, —CH₂F), —OC₁₋₅ alkyl, —O-haloalkyl (e.g., —OCF₃, —OCHF₂, —OCH₂F, —OCH₂F), —NH(C₁₋₅ alkyl), —NHCO(C₁₋₅ alkyl), —NHSO₂(C₁₋₅ alkyl), —CONH(C₁₋₅ alkyl), or —SO₂NH(C₁₋₅ alkyl). In certain embodiments, R^{e1} and R² are each independently nitro, cyano, halogen, —CF₃, —CONHMe, —SO₂NHMe, —OMe, —NHMe, —NHAc, —NHSO₂Me, or —OCF₃.

In some embodiments of formula (III), X² and X³ are each independently selected from the spacer moieties described herein. In some embodiments, X² and X³ are the same spacer moiety. In some embodiments, X² and X³ are different spacer moieties.

In certain embodiments of formula (III), a1 and a2 are each independently an integer from 0 to 2; R¹ and R² are each independently H, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Exemplary bifunctional releasable linkers fall within the following formula (III-A):

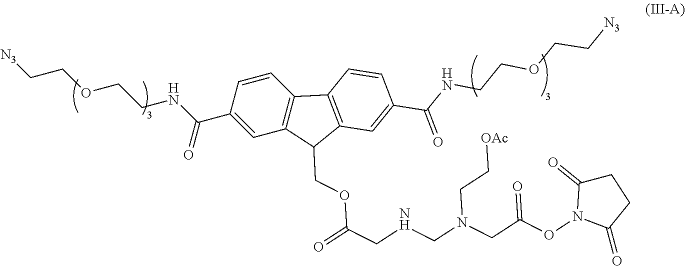

In another aspect, the present disclosure is directed to bifunctional releasable linkers of formula (IV):

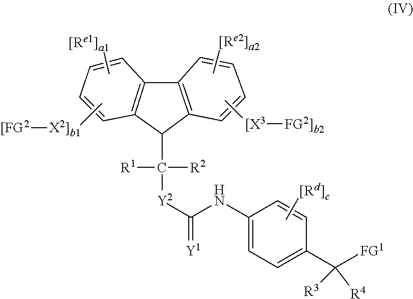

or a stereoisomer, tautomer or mixtures thereof, or isotopic variant thereof;
wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R³ is a hydrogen, alkyl, substituted alkyl, alkenyl, substituted alkenyl, alkynyl, substituted alkynyl, aryl, or substituted aryl;

R⁴ is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

c is an integer from 0 to 4;

R^{e1}, when present, is a first electron altering group;

R^{e2}, when present, is a second electron altering group;

R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, or substituted aryl or heteroaryl;

X², when present, is a spacer moiety;

X³, when present, is a spacer moiety;

Y¹ is O or S;

Y² is O or S;

FG¹ is a functional group capable of reacting with an amino group of an active agent to form a releasable linkage, such as a carbamate linkage; and

FG² is a functional group capable of reacting through click chemistry, independently including but not limited to azide, alkynyl, and cycloalkynyl (e.g., dibenzocyclooctyne (DBCO)) groups.

In some embodiments of formula (IV), R¹, R², R³ and R⁴ are each independently a C₁₋₅ alkyl, a substituted C₁₋₅ alkyl, a C₂₋₆ alkenyl, a substituted C₂₋₆ alkenyl, a C₂₋₆alkynyl, a substituted C₂₋₆ alkynyl, a phenyl, or a substituted phenyl. In certain embodiments, R¹, R², R³ and R⁴ are each independently a C₁₋₅ alkyl or a substituted C₁₋₅ alkyl.

In some embodiments of formula (IV), R^{e1} and R^{e2} are each independently nitro, cyano, halogen, haloalkyl (e.g., —CF₃, —CHF₂, —CH₂F, —CH₂F), —OC₁₋₅ alkyl, —O-haloalkyl (e.g., —OCF₃, —OCHF₂, —OCH₂F, —OCH₂F), —NH(C₁₋₅ alkyl), —NHCO(C₁₋₅ alkyl), —NHSO₂(C₁₋₅ alkyl), —CONH(C₁₋₅ alkyl), or —SO₂NH(C₁₋₅ alkyl). In certain embodiments, R^{e1} and R² are each independently nitro, cyano, halogen, —CF₃, —CONHMe, —SO₂NHMe, —OMe, —NHMe, —NHAc, —NHSO₂Me, or —OCF₃.

In some embodiments of formula (IV), R^{d} is nitro, cyano, halogen, —CONH(C₁₋₅ alkyl) or —CONH(phenyl), substituted —CONH(C₁₋₅ alkyl) or —CONH(phenyl), —SO₂NH(C₁₋₅ alkyl) or —SO₂NH(phenyl), substituted —SO₂NH(C₁₋₅ alkyl) or —SO₂NH(phenyl), —SO₂(C₁₋₅ alkyl) or —SO₂(phenyl), substituted —SO₂(C₁₋₅ alkyl) or —SO₂(phenyl), C₁₋₅ alkoxy, substituted C₁₋₅ alkoxy, C₁₋₅ alkyl or C₃₋₆ cycloalkyl, substituted C₁₋₅ alkyl or C₃₋₆ cycloalkyl, phenyl or 5- to 6-membered heteroaryl, or substituted phenyl or 5- to 6-membered heteroaryl.

In some embodiments of formula (IV), X² and X³ are each independently selected from the spacer moieties described herein. In some embodiments, X² and X³ are the same spacer moiety. In some embodiments, X² and X³ are different spacer moieties.

The advantage of using releasable linkers, such as those of formula (III) and formula (IV), is the potential of improving the stability that affords sustained drug release and ultimately provide prolonged therapeutic efficacy. Therefore, the linkers of the present disclosure provide advantages for the stability and storages of polymer-protein therapeutics over those of the prior art.

Polymeric Reagents with Releasable Linkers

The present disclosure is also directed to conjugates that can be derived from polymeric reagents with releasable linkers.

In some aspects, the disclosure is directed to the polymeric reagent with releasable linkers of the formula (V):

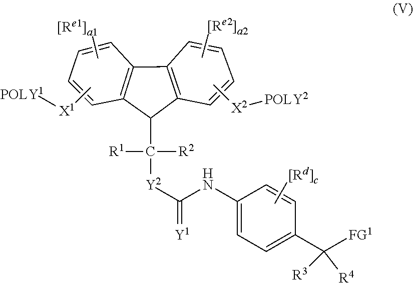

or a stereoisomer, tautomer or mixtures thereof, or isotopic variant thereof;
wherein:

POLY¹ is a first water-soluble polymer;

POLY² is a second water-soluble polymer;

X¹ is a first spacer moiety;

X² is a second spacer moiety;

Y¹ is O or S;

Y² is O or S;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R³ is a hydrogen, alkyl, substituted alkyl, alkenyl, substituted alkenyl, alkynyl, substituted alkynyl, aryl, or substituted aryl;

R⁴ is a hydrogen, alkyl, substituted alkyl, alkenyl, substituted alkenyl, alkynyl, substituted alkynyl, aryl, or substituted aryl;

a1 is an integer from 0 to 3;

a2 is an integer from 0 to 3;

c is an integer from 0 to 4;

R^{e1}, when present, is a first electron altering group;

R^{e2}, when present, is a second electron altering group;

R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl; and

FG¹ is a functional group capable of reacting with an amino group of an active agent to form a releasable linkage, such as a carbamate linkage.

In some embodiments, R¹, R², R³, R⁴, R^{e1}, R^{e2} and R^{d} are defined as above in formula (IV).

In some embodiments of formula (V), R^{e1} and R^{e2} are the same electron altering group. In some embodiments, R^{e1} and R² are the different electron altering groups.

In some embodiments of formula (V), POLY¹ and POLY² are each independently selected from the water soluble polymers described herein. In some embodiments, POLY¹ and POLY² are the same water-soluble polymer. In some embodiments, POLY¹ and POLY² are different water-soluble polymers.

In some embodiments of formula (V), X¹ and X² are each independently selected from the spacer moieties described herein. In some embodiments, X¹ and X² are the same spacer moiety. In some embodiments, X¹ and X² are different spacer moieties. Exemplary polymeric reagents fall within the following formula (V-A):

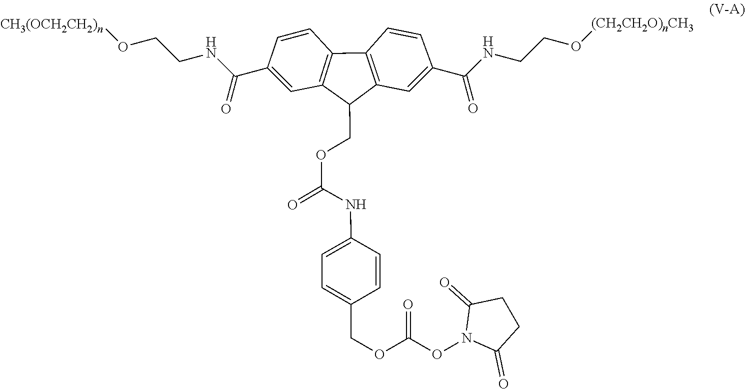

wherein n is independently an integer from 4 to 1500, e.g., 4, 25, 50, 75, 100, 200, 300, 400, 500, 600, 700, 800, 900, 1000, 1100, 1200, 1300, 1400, 1500, including all ranges and values therebetween.

Other polymeric reagents with two releasable linkages encompass the following formula (VI):

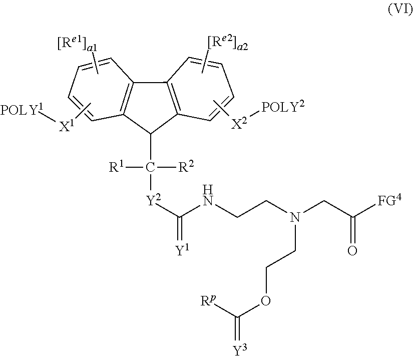

or a stereoisomer, tautomer or mixtures thereof, or isotopic variant thereof;
wherein:

POLY¹ is a first water-soluble polymer;

POLY² is a second water-soluble polymer;

X¹ is a first spacer moiety;

X² is a second spacer moiety;

Y¹ is O or S;

Y² is O or S;

Y³ is O or S;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 is an integer from 0-3;

a2 is an integer from 0-3;

R^{e1}, when present, is a first electron altering group;

R^{e2}, when present, is a second electron altering group;

R^{p} is a hydrogen, alkyl, substituted alkyl, alkenyl, substituted alkenyl, alkynyl, substituted alkynyl, aryl, or substituted aryl; and

FG⁴ is a functional group capable of reacting with an amino group of an active agent to form a releasable linkage, such as an amide linkage.

In some embodiments of formula (VI), R¹, R², and R^{p} are each independently a C₁₋₅ alkyl, a substituted C₁₋₅ alkyl, a C₂₄ alkenyl, a substituted C₂₄ alkenyl, a C₂₋₆ alkynyl, a substituted C₂₋₆ alkynyl, a phenyl, or a substituted phenyl. In certain embodiments, R¹, R², R³ and R⁴ are each independently a C₁₋₅ alkyl or a substituted C₁₋₅ alkyl.

In some embodiments of formula (VI), R¹ and R² are each independently a nitro, cyano, halogen, —CONH(C₁₋₅ alkyl) or —CONH(phenyl), substituted —CONH(C₁₋₅ alkyl) or —CONH(phenyl), —SO₂NH(C₁₋₅ alkyl) or —SO₂NH(phenyl), substituted —SO₂NH(C₁₋₅ alkyl) or —SO₂NH(phenyl), —SO₂(C₁₋₅ alkyl) or —SO₂(phenyl), substituted —SO₂(C₁₋₅ alkyl) or —SO₂(phenyl), C₁₋₅ alkoxy, substituted C₁₋₅ alkoxy, C₁₋₅ alkyl or C₃₄ cycloalkyl, substituted C₁₋₅ alkyl or C₃₄ cycloalkyl, phenyl or 5- to 6-membered heteroaryl, or substituted phenyl or 5- to 6-membered heteroaryl.

In some embodiments of formula (VI), POLY¹ and POLY² are each independently selected from the water soluble polymers described herein. In some embodiments, POLY¹ and POLY² are the same water-soluble polymer. In some embodiments, POLY¹ and POLY² are different water-soluble polymers.

In some embodiments of formula (VI), X¹ and X² are each independently selected from the spacer moieties described herein. In some embodiments, X¹ and X² are the same spacer moiety. In some embodiments, X¹ and X² are different spacer moieties.

Exemplary polymeric reagents fall within the following formula (VI-A):

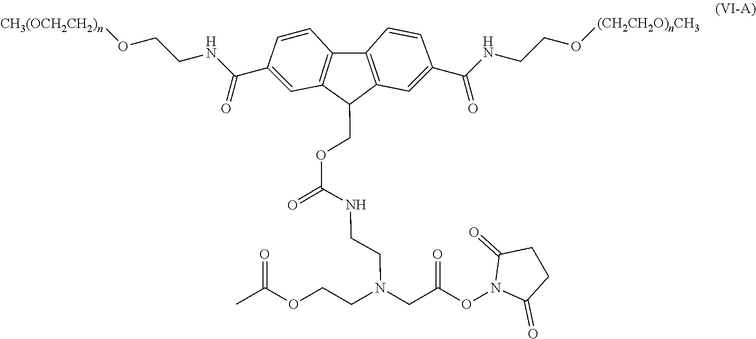

wherein n is independently an integer from 4 to 1500, e.g., 4, 25, 50, 75, 100, 200, 300, 400, 500, 600, 700, 800, 900, 1000, 1100, 1200, 1300, 1400, 1500, including all ranges and values therebetween.

### Protein-Linker Conugates

In some embodiments, the present disclosure provides a conjugate, the conjugate comprising a residue of a protein covalently attached with one or more linkers, wherein the conjugate comprises a structure according to formula (XIX):

Protein-(L)z (XIX)

or a stereoisomer, regioisomer, tautomer or mixtures thereof, or isotopic variant thereof; or a pharmaceutically acceptable salt, solvate, hydrate, or prodrug thereof;
wherein:

z is an integer from 1 to 25;

L is a linker; and

Protein is a chemokine, a chemokine antagonist, a cytokine, a cytokine antagonist, an antibody, or a therapeutic peptide.

The conjugates described herein are the product of the step one synthesis from Scheme (I). In certain embodiments, the linker is a non-releasable linker. In certain embodiments, the linker is a releasable linker. In some embodiments, the releasable linker is a derivative of the bifunctional releasable linker (e.g., a linker of formula (I), formula (II), formula (III) or formula (IV)) disclosed herein.

In certain embodiments, the linker is covalently attached to an amine group of a residue within the protein. In certain embodiments, the residue is lysine. In certain embodiments, a composition is provided comprising mixtures of conjugates comprising different numbers of linkers attached to a protein.

Exemplary conjugates formed using bifunctional releasable linkage-providing reagents conjugated with a protein include those of the formula (VII):

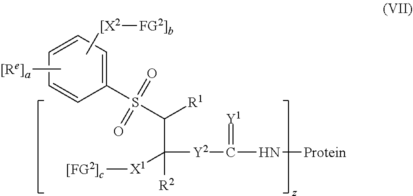

wherein:

X¹ is a first spacer moiety;

X², when present, is a second spacer moiety;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;

a is an integer from 0 to 5;

b is an integer from 0 to 3;

c is an integer from 0 to 2;

z is an integer from 1 to 25;

Y¹ is O or S;

Y² is O or S;

FG² is a functional group capable of reacting through click chemistry, independently including but not limited to azide, alkynyl, and cycloalkynyl (e.g., dibenzocyclooctyne (DBCO)) groups;
—NH— is an amine group of a residue within the protein; and
Protein is a chemokine, a chemokine antagonist, a cytokine, a cytokine antagonist, an antibody, or a therapeutic peptide.

In some embodiments, R¹, R², and R^{e} are as defined above in formula (I).

In some embodiments of formula (VII), a is an integer from 0 to 4. In some embodiments, a is an integer from 0 to 3. In some embodiments, a is an integer from 0 to 2. In some embodiments, a is 0. In some embodiments, a is 1. In some embodiments, a is 2. In some embodiments, a is 3. In some embodiments, a is 4. In some embodiments, a is 5.

In some embodiments of formula (VII), b is an integer from 0 to 2. In some embodiments, b is 0. In some embodiments, b is 1. In some embodiments, b is 2. In some embodiments, b is 3.

In some embodiments of formula (VII), c is 0 or 1. In some embodiments, c is 0. In some embodiments, c is 1. In some embodiments, c is 2.

In some embodiments of formula (VII), z is an integer from 1 to 20. In some embodiments, z is an integer from 1 to 15. In some embodiments, z is an integer from 1 to 10. In some embodiments, z is an integer from 1 to 8. In some embodiments, z is an integer from 1 to 5.

Those of ordinary skill will recognize that the values and ranges for a, b, c, and z described herein can be combined in any manner to provide a conjugate of the present disclosure. For example, in some embodiments, a is an integer from 0 to 2, b is 0 or 1, c is 0 or 1, and z is an integer from 1 to 25. In some embodiments, a is 1, b is 1, c is 1, and Z is an integer from 1 to 25. In some embodiments, a is 1, b is 0, c is 1, and z is an integer from 1 to 25. In some embodiments, a is 1, b is 1, c is 0, and z is an integer from 1 to 25. These and numerous other combinations are contemplated in the present disclosure. In some embodiments, X¹ and X² are each independently selected from the spacer moieties described herein. In some embodiments, X¹ and X² are the same spacer moiety. In some embodiments, X¹ and X² are different spacer moieties.

Within formula (VII), conjugates having the more defined structure are contemplated as formula (VII-A), (VII-B), (VII-C), or (II-D):

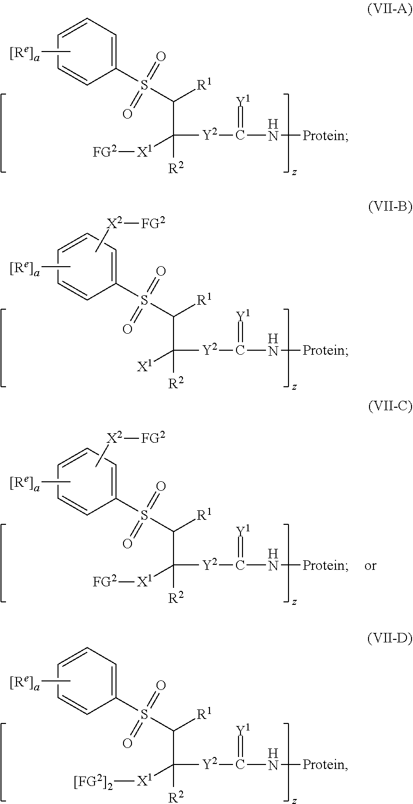

wherein X¹ is a first spacer moiety; X² is a second spacer moiety; R¹, R², R^{e}, a, z, Y¹, Y², FG² and protein are as defined above in formula (VII).

In certain embodiments of formula (VII), (VII-A), (VII-B), (VII-C), or (VII-D), a is an integer from 0 to 2; R¹ and R² are each independently hydrogen, Me, or Et; and R^{e} is nitro, cyano, halogen, —CF₃, —CONHMe, —SO₂NHMe, —OMe, —NHMe, —NHAc, —NHSO₂Me, or —OCF₃.

Further exemplary conjugates have the following structure (VII-A1):

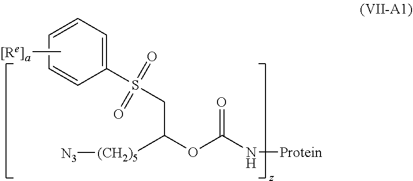

wherein, R is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl; z is an integer from 1-25; “—NH—” represents one or more linkers individually attached to a protein moiety. In certain embodiments, wherein a is an integer from 1 to 2; and R^{e} is 4-F, 4-Cl, 4CF₃, 2,4-difluoro, or 2-CF₃-4-F substitution.

Further exemplary conjugates have the following structures:

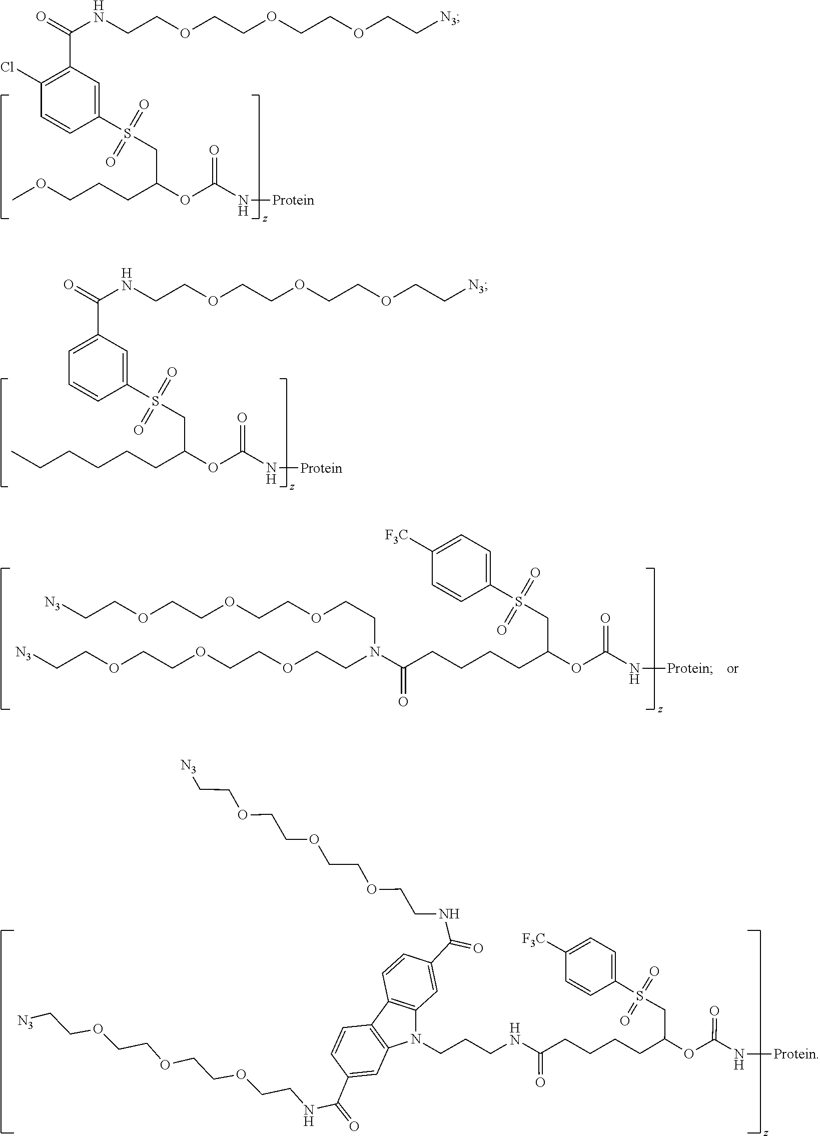

Other exemplary conjugates formed using bifunctional releasable linkage-providing reagents include those of the following formula (VIII):

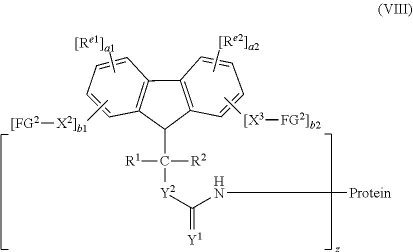

wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

z is an integer from 1 to 25;

R^{e1}, when present, is a first electron altering group;

R^{e2}, when present, is a second electron altering group;

X², when present, is a spacer moiety;

X³, when present, is a spacer moiety;

Y¹ is O or S;

Y² is O or S;

FG² is a functional group capable of reacting through click chemistry, independently including but not limited to azide, alkynyl, and cycloalkynyl (e.g., dibenzocyclooctyne (DBCO)) groups;

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{e1}, and R^{e2} are as defined above in formula (VI).

In certain embodiments of formula (VIII), a1 and a2 are each independently an integer from 0 to 2; R¹ and R² are each independently hydrogen, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Within formula (VIII), conjugates having the more defined structure are contemplated as formula (VII-A):

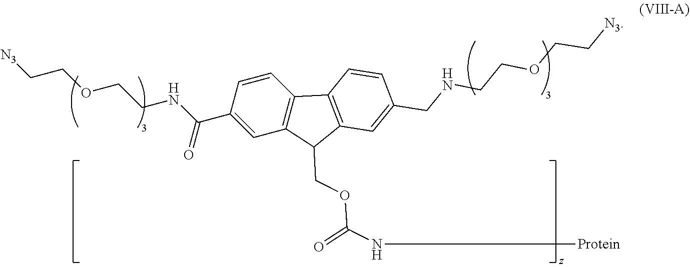

Other exemplary conjugates formed using two releasable linkage-providing reagents include those of the following formula (IX):

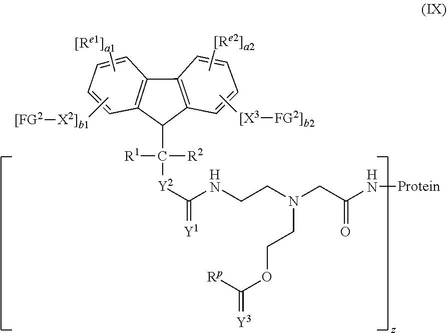

wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

z is an integer from 1 to 25;

R^{e1}, when present, is a first electron altering group;

R^{e2}, when present, is a second electron altering group;

R^{p} is a hydrogen, alkyl, substituted alkyl, alkenyl, substituted alkenyl, alkynyl, substituted alkynyl, aryl, or substituted aryl;

X², when present, is a spacer moiety;

X³, when present, is a spacer moiety;

Y¹ is O or S;

Y² is O or S;

Y³ is O or S;

FG² is a functional group capable of reacting through click chemistry, independently including but not limited to azide, alkynyl, and cycloalkynyl (e.g., dibenzocyclooctyne (DBCO)) groups;

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{p}, R^{e1}, and R² are as defined above in formula (VI).

In certain embodiments of formula (IX), wherein a1 and a2 are each independently an integer from 0 to 2; R¹ and R² are each independently hydrogen, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Within formula (IX), conjugates having the more defined structure are as following formula (IX-A):

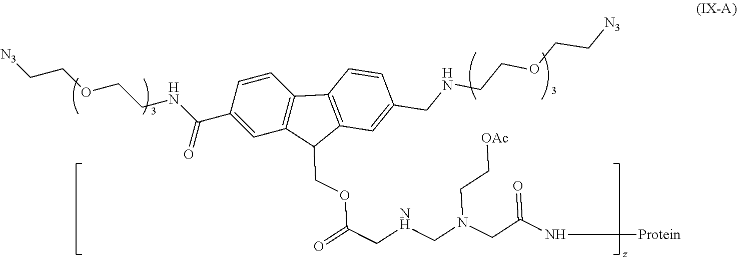

Other exemplary conjugates formed using two releasable linkage-providing reagents include those of the following formula (X):

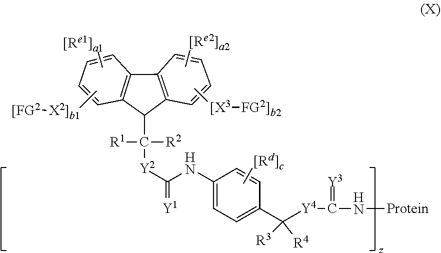

wherein:

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R³ is a hydrogen, alkyl, substituted alkyl, alkenyl, substituted alkenyl, alkynyl, substituted alkynyl, aryl, or substituted aryl;

R⁴ is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

c is an integer from 0 to 4;

z is an integer from 1 to 25;

R^{e1}, when present, is a first electron altering group;

R^{e2}, when present, is a second electron altering group;

R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;

X², when present, is a spacer moiety;

X³, when present, is a spacer moiety;

Y¹ is O or S;

Y² is O or S;

Y³ is O or S;

Y⁴ is O or S;

FG² is a functional group capable of reacting through click chemistry, independently including but not limited to azide, alkynyl, and cycloalkynyl (e.g., dibenzocyclooctyne (DBCO)) groups;

—NH— is an amine group of a residue within the protein; and

Protein is a chemokine, a chemokine antagonist, a cytokine, a cytokine antagonist, an antibody, or a therapeutic peptide.

In some embodiments, R¹, R², R³, R⁴, R^{d}, R^{e1}, and R^{e2} are as defined above in formula (IV).

In certain embodiments of the formulas disclosed herein, z is an integer from 1 to 22, 1 to 20, 1 to 18, 1 to 15, 1 to 12, 1 to 10, 1 to 8, 1 to 5, or 1 to 3, wherein z represents the number of releasable linkers conjugated to the protein.

Protein-Macromolecule Conjugates

In one or more embodiments of the disclosure, a protein-macromolecule conjugates is provided, the conjugate comprising a protein, at least one linker, and at least one water-soluble polymer, wherein the protein is covalently attached to each of the water-soluble polymer via a linker, wherein the macromolecule is straight or branched water-soluble polymer. In certain embodiments, the at least one linker is two or more linkers. In certain embodiments, the two or more linkers comprise at least one non-releasable linker. In certain embodiments, the two or more linkers comprise at least one releasable linker. In certain embodiments, the two or more linkers comprise at least one non-releasable linker and one releasable linker. In certain embodiments, the two or more linkers comprise at least one non-releasable linker and from one to eight releasable linkers.

In certain embodiments, the at least one linker is the non-releasable linker. In certain embodiments, the at least one linker is the releasable linker. In certain embodiments, each of the linker is the releasable linker. In certain embodiments, one or more macromolecules are covalently attached to the protein via one or more linkers. In certain embodiments, eight or more macromolecules are covalently attached to the protein via eight or more linkers.

In certain embodiments, the macromolecule is covalently attached to an amine group of a residue within the protein via a linker. In certain embodiments, the residue is lysine. In certain embodiments, the conjugates are a mixtures of conjugates comprising different numbers of macromolecules attached to the protein.

In various embodiments, the macromolecule is a water-soluble polymer, a lipid, a protein or a polypeptide. It can include any of the following: a fatty acid comprises from about 6 to about 26 carbon atoms, one of the polymers selected from the group consisting of 2-methacryloyl-oxyethyl phosphoyl cholins, poly(acrylic acids), poly(acrylates), poly(acrylamides), poly(N-acryloylmorpholine), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethylene glycols), poly(ethylene oxides), poly(ethyl phosphates), poly(ethyloxazolines), poly(glycolic acids), poly(α-hydroxy acid), poly(hydroxyethyl acrylates), poly(hydroxyethyloxazolines), poly(hydroxymethacrylates), poly(hydroxyalkylmethacrylamides), poly(hydroxyalkylmethacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(oxyethylated polyol), poly(olefinic alcohol), polyphosphazene, poly(propylene glycols), poly(saccharide), poly(siloxanes), poly(urethanes), poly(vinyl alcohols), poly(vinyl amines), poly(vinylmethylethers), poly(vinylpyrrolidones), silicones, amylose, celluloses, carbomethyl celluloses, hydroxypropyl methylcelluloses, chitins, chitosans, dextrans, dextrins, gelatins, hyaluronic acids (HA) and derivatives, functionalized hyaluronic acids, mannans, pectins, heparin, heparan sulfate (HS), rhamnogalacturonans, starches, hydroxyalkyl starches, hydroxyethyl starches (HES), polysialic acid (PSA) and other carbohydrate-based polymers, xylans, and copolymers, of albumin, transferrin, transthyretin, immunoglobulin, a XTEN peptide, a glycine-rich homoamino acid polymer (HAP), a PAS polypeptide, an elastin-like polypeptide (ELP), a CTP peptide, or a gelatin-like protein (GLK) polymer.

In certain embodiments, the macromolecule is water-soluble polymer. In certain embodiments, the water-soluble polymer is a polymer of poly(ethylene glycol). In certain embodiments, the poly(ethylene glycol) is terminally capped with an end-capping moiety selected from the group consisting of hydroxy, alkoxy, substituted alkoxy, alkenoxy, substituted alkenoxy, alkynoxy, substituted alkynoxy, aryloxy and substituted aryloxy.

With respect to the water-soluble polymer, the water-soluble polymer is nontoxic, non-naturally occurring and biocompatible. With respect to biocompatibility, a substance is considered biocompatible if the beneficial effects associated with use of the substance alone or with another substance (e.g., an active agent such as an IL-2 moiety) in connection with living tissues (e.g., administration to a patient) outweighs any deleterious effects as evaluated by a clinician, e.g., a physician. With respect to non-immunogenicity, a substance is considered non-immunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the nonpeptidic water-soluble polymer is biocompatible and non-immunogenic.

Further, the polymer is typically characterized as having from 2 to about 300 termini. Examples of such polymers include, but are not limited to, poly(alkylene glycols) such as polyethylene glycol (“PEG”), poly(propylene glycol) (“PPG”), copolymers of ethylene glycol and propylene glycol and the like, poly(oxyethylated polyol), poly(olefmic alcohol), polyvinylpyrrolidone), poly(hydroxyalkylmethacrylamide), poly(hydroxyalkylmethacrylate), polysaccharides), poly(a-hydroxy acid), poly(vinyl alcohol), polyphosphazene, polyoxazolines (“POZ”) (which are described in WO 2008/106186), poly(N-aciyloylmorpholine), and combinations of any of the foregoing.

The water-soluble polymer is not limited to a particular structure and can be linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

Activated PEG and other activated water-soluble polymers (i.e., polymeric reagents) are activated with a suitable activating group appropriate for coupling to a desired site on the protein. Thus, a polymeric reagent will possess a reactive group for reaction with the protein moiety. Representative polymeric reagents and methods for conjugating these polymers to an active moiety are known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182. Exemplary activating groups suitable for coupling to an protein moiety include hydroxyl, maleimide, ester, acetal, ketal, amine, carboxyl, aldehyde, aldehyde hydrate, ketone, vinyl ketone, thione, thiol, vinyl sulfone, hydrazine, among others.

Typically, the weight-average molecular weight of the water-soluble polymer in the conjugate is from about 100 Daltons to about 150,000 Daltons. Exemplary ranges, however, include weight-average molecular weights in the range of from about 500 Daltons to less than 20,000 Daltons, in a range of from about 20,000 Daltons to less than 85,000 Daltons, in a range of from about 85,000 Daltons to about 100,000 Daltons, in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. For any given water-soluble polymer, PEGs having a molecular weight in one or more of these ranges are preferred.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 16,000 Daltons, about 18,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used.

When used as the polymer, PEGs will typically comprise a number of (OCH₂CH₂) monomers [or (CH₂CH₂O) monomers, depending on how the PEG is defined], As used throughout the description, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

One particularly preferred polymer for use in the disclosure is an end-capped polymer, that is, a polymer having at least one terminus capped with a relatively inert group, such as a lower C₁₋₆ alkoxy group, although a hydroxyl group can also be used. When the polymer is PEG, for example, it is preferred to use a methoxy-PEG (commonly referred to as mPEG), which is a linear form of PEG wherein one terminus of the polymer is a methoxy (—OCH₃) group, while the other terminus is a hydroxyl or other functional group that can be optionally chemically modified.

In one form useful in one or more embodiments of the present disclosure, free or unbound PEG is a linear polymer terminated at each end with hydroxyl groups:

HO—CH₂CH₂O—(CH₂CH₂O)ₙ—CH₂CH₂—OH,

wherein (n) typically ranges from zero to about 4,000.

The above polymer, alpha-, omega-dihydroxylpoly(ethylene glycol), can be represented in brief form as HO-PEG-OH where it is understood that the -PEG- symbol can represent the following structural unit:

—CH₂CH₂O—(CH₂CH₂O)ₙ—CH₂CH₂—,

wherein (n) is as defined as above.

Another type of PEG useful in one or more embodiments of the present disclosure is methoxy-PEG-OH, or mPEG-OH in brief, in which one terminus is the relatively inert methoxy group, while the other terminus is a hydroxyl group. The structure of mPEG-OH is given below.

CH₃O—CH₂CH₂O—(CH₂CH₂O)ₙ—CH₂CH₂—OH

wherein (n) is as described above.

Another type of PEG useful in one or more embodiments of the present disclosure is methoxy-PEG-NH₂, or mPEG-NH₂ in brief, in which one terminus is the relatively inert methoxy group, while the other terminus is an amino group. The structure of mPEG-NH₂ is given below.

CH₃O—CH₂CH₂O—(CH₂CH₂O)ₙ—CH₂CH₂—NH₂

wherein (n) is as described above.

Another type of PEG useful in one or more embodiments of the present disclosure is methoxy-PEG-CO₂H, or mPEG-CO₂H in brief, in which one terminus is the relatively inert methoxy group, while the other terminus is a carboxylic acid group. The structure of mPEG-CO₂H is given below.

CH₃O—CH₂CH₂O—(CH₂CH₂O)ₙ—CH₂CH₂—CO₂H

wherein (n) is as described above.

Another type of PEG useful in one or more embodiments of the present disclosure is methoxy-PEG-Ns, or mPEG-N3 in brief, in which one terminus is the relatively inert methoxy group, while the other terminus is an azide group. The structure of mPEG-N3 is given below.

CH₃O—CH₂CH₂O—(CH₂CH₂O)ₙ—CH₂CH₂—N₃

wherein (n) is as described above.

Another type of PEG useful in one or more embodiments of the present disclosure is methoxy-PEG-DBCO, or mPEG-DBCO in brief, in which one terminus is the relatively inert methoxy group, while the other terminus is a dibenzocyclooctyne (DBCO) group. One example of the structure of mPEG-DBCO is given below.

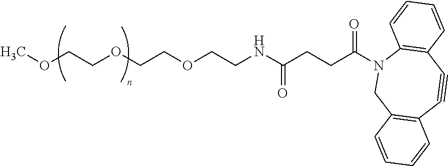

wherein (n) is as described above.

Multi-armed or branched PEG molecules, such as those described in U.S. Pat. No. 5,932,462, can also be used as the PEG polymer. For example, PEG can have the structure:

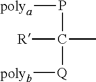

wherein:
polyₐ and poly_{b} are PEG backbones (either the same or different), such as methoxy poly(ethylene glycol); R′ is a nonreactive moiety, such as H, methyl or a PEG backbone; and P and Q are nonreactive linkages.

In addition, the PEG can comprise a forked PEG. An example of a forked PEG is represented by the following structure:

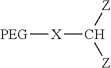

wherein: X is a spacer moiety of one or more atoms and each Z is an activated terminal group linked to CH by a chain of atoms of defined length. International Patent Application Publication WO 99/45964 discloses various forked PEG structures capable of use in one or more embodiments of the present disclosure. The chain of atoms linking the Z functional groups to the branching carbon atom serve as a tethering group and may comprise, for example, alkyl chains, ether chains, ester chains, amide chains and combinations thereof.

The PEG polymer may comprise a pendant PEG molecule having reactive groups, such as carboxyl, covalently attached along the length of the PEG rather than at the end of the PEG chain. The pendant reactive groups can be attached to the PEG directly or through a spacer moiety, such as an alkylene group.

Some hydrolytically degradable linkages, useful as a degradable linkage within a polymer backbone and/or as a degradable linkage to a protein moiety, include: ester linkages, carbonate linkages; imine linkages resulting, for example, from reaction of an amine and an aldehyde (see, e.g., Ouchi et al. (1997) Polymer Preprints 38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

Such optional features of the conjugate, i.e., the introduction of one or more degradable linkages into the polymer chain or to the protein moiety, may provide for additional control over the final desired pharmacological properties of the conjugate upon administration. For example, a large and relatively inert conjugate (i.e., having one or more high molecular weight PEG chains attached thereto, for example, one or more PEG chains having a molecular weight greater than about 10,000, wherein the conjugate possesses essentially no bioactivity) may be administered, which is released to generate a bioactive conjugate possessing a portion of the original PEG chain. In this way, the properties of the conjugate can be more effectively tailored to balance the bioactivity of the conjugate over time.

The water-soluble polymer associated with the conjugate can be “releasable.” That is, the water-soluble polymer releases (either through hydrolysis, enzymatic processes, catalytic processes or otherwise), thereby resulting in the unconjugated protein moiety. In some instances, releasable polymers detach from the protein moiety in vivo without leaving any fragment of the water-soluble polymer. In other instances, releasable polymers detach from the protein moiety in vivo leaving a relatively small fragment (e.g., a succinate tag) from the water-soluble polymer. An exemplary cleavable polymer includes one that attaches to the protein moiety via a carbamate linkage.

Those of ordinary skill in the art will recognize that the foregoing discussion concerning water-soluble polymer is by no means exhaustive and is merely illustrative, and that all polymeric materials having the qualities described above are contemplated. As used herein, the term “polymeric reagent” generally refers to an entire molecule, which can comprise a water-soluble polymer segment and a functional group.

As described above, a conjugate of the present disclosure can comprise multiple water-soluble polymers covalently attached to a protein moiety. In some embodiments, the multiple water-soluble polymers covalently attached to a protein moiety are the same. In some embodiments, at least one of the multiple water-soluble polymers covalently attached to a protein moiety is different. Typically, for any given conjugate, there will be one or more water-soluble polymers covalently attached to one or more moieties having protein activity. In some instances, the conjugate may have 1, 2, 3, 4, 5, 6, 7, 8 or more water-soluble polymers individually attached to a protein moiety. Any given water-soluble polymer may be covalently attached to an amino acid of the protein moiety, or when the protein moiety is (for example) a glycoprotein, to a carbohydrate of the protein moiety. Attachment to a carbohydrate may be carried out, e.g., using metabolic functionalization employing sialic acid-azide chemistry [Luchansky et al. (2004) Biochemistry 43(38): 12358-123661 or other suitable approaches such as the use of glycidol to facilitate the introduction of aldehyde groups [Heldt et al. (2007) European Journal of Organic Chemistry 32:5429-5433].

The particular linkage within the protein moiety and the polymer depends on a number of factors. Such factors include, for example, the particular linkage chemistry employed, the particular protein moiety, the available functional groups within the protein moiety (either for attachment to a linker, polymer or conversion to a suitable attachment site), the presence of additional reactive functional groups within the protein moiety, and the like.

The conjugates of the disclosure can beprodrugs, meaning that the linkage between the polymer and the protein moiety is releasable to allow release of the parent moiety. Apart from the releasable linkers described in this disclosure, other exemplary releasable linkages can include carboxylate ester, phosphate ester, thiol ester, anhydrides, acetals, ketals, acyloxyalkyl ether, imines, orthoesters, peptides and oligonucleotides. Such linkages can be readily prepared by appropriate modification of either the protein moiety (e.g., the carboxyl group C terminus of the protein, or a side chain hydroxyl group of an amino acid such as serine or threonine contained within the protein, or a similar functionality within the carbohydrate) and/or the polymeric reagent using coupling methods commonly employed in the art. Most preferred, however, are releasable linkages that are readily formed by reaction of a suitably activated polymer with a non-modified functional group contained within the protein moiety.

Alternatively, a hydrolytically stable linkage, such as an amide, urethane (also known as carbamate), amine, thioether (also known as sulfide), or urea (also known as carbamide) linkage can also be employed as the linkage for coupling the protein moiety. A preferred hydrolytically stable linkage is an amide. In one approach, a water-soluble polymer bearing an activated ester can be reacted with an amine group on the protein moiety to thereby result in an amide linkage. Another preferred hydrolytically stable linkage is a thiol bridge.

The conjugates (as opposed to an unconjugated protein moiety) may or may not possess a measurable degree of protein activity. That is to say, a polymer-protein conjugate in accordance with the disclosure will possess anywhere from about 0.1% to about 100%, including about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 55%, or about 100%, of the bioactivity of the unmodified parent protein moiety. In some instances, the polymer-protein conjugates may have greater than 100% bioactivity of the unmodified parent protein moiety. Preferably, conjugates possessing little or no protein activity contain a hydrolyzable linkage connecting the polymer to the protein, so that regardless of the lack (or relatively lack) of activity in the conjugate, the active parent molecule (or a derivative thereof) is released upon aqueous-induced cleavage of the hydrolyzable linkage. Such activity may be determined using a suitable in-vivo or in-vitro model, depending upon the known activity of the particular protein.

For conjugates possessing a hydrolytically stable linkage that couples the protein to the polymer, the conjugate will typically possess a measurable degree of bioactivity. For instance, such conjugates are typically characterized as having a bioactivity satisfying one or more of the following percentages relative to that of the unconjugated protein: at least about 2%, at least about 5%, at least about 10%, at least about 15%, at least about 25%, at least about 30%, at least about 40%, at least about 50%, at least about 60%, at least about 80%, at least about 85%, at least about 90%, at least about 95%, at least about 97%, at least about 100%, and more than 105% (when measured in a suitable model, such as those well known in the art). Preferably, conjugates having a hydrolytically stable linkage (e.g., an amide linkage, a thiol bridge) will possess at least some degree of the bioactivity of the unmodified parent protein.

The attachment between the protein and the water-soluble polymer via a linker can be direct, wherein no intervening atoms are located between the linker and the polymer, or indirect, wherein one or more atoms are located between the linkage and the polymer. With respect to the indirect attachment, a “spacer moiety” can serve as a linker between the residue of the linkages and the water-soluble polymer. The one or more atoms making up the spacer moiety can include one or more of carbon atoms, nitrogen atoms, sulfur atoms, oxygen atoms, and combinations thereof. The spacer moiety can comprise an amide, secondary amine, carbamate, thioether, disulfide group and/or click chemistry product groups. Non-limiting examples of specific spacer moieties include those selected from the group consisting of —O—, —S—, —S—S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —CH₂—CH₂—CH₂—CH₂—CH₂—, O—CH₂—, —CH₂—O—, —O—CH₂—CH₂—, —CH₂—O—CH₂—, —CH₂—CH₂—O—, —O-CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]₁-(OCH₂CH₂)ₘ—, divalent cycloalkyl group, —O—, —S—, an amino acid, —N(R³)—, and combinations of two or more of any of the foregoing, wherein R³ is H or an organic radical selected from the groups consisting of alkyl, substituted alkyl, alkenyl, substituted alkenyl, alkynyl, substituted alkynyl, aryl and substituted aryl, (1) is zero to six, and (m) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment.

General protein-macromolecule conjugate comprises a structure according to formula (XX):

Protein-(L-Macromolecule)_{z} (XX)

or a stereoisomer, regioisomer, tautomer or mixtures thereof, or isotopic variant thereof; or a pharmaceutically acceptable salt, solvate, hydrate, or prodrug thereof;
wherein:

z is an integer from 1 to 25;

L is a linker;

Protein is a chemokine, a chemokine antagonist, a cytokine, a cytokine antagonist, an antibody, or a therapeutic peptide; and

macromolecule is a water-soluble polymer, a lipid, a protein or a polypeptide.

In some embodiments, the linker L is a linker of the present disclosure. In some embodiments, L is one or more non-releasable linkers and/or one or more releasable linkers. In some embodiments, the one or more releasable linkers is derived from a bifunctional releasable linker of the present disclosure (e.g., a linker of formula (I), formula (II), formula (III) or formula (IV)) and/or a polymeric reagent with releasable linker (e.g., formula (V) or formula (VI)).

In some embodiments, z is an integer from 1 to 20. In some embodiments, z is an integer from 1 to 15. In some embodiments, z is an integer from 1 to 10. In some embodiments, z is an integer from 1 to 8. In some embodiments, z is an integer from 1 to 5.

In some embodiments, when z is 2 or more, each L-Macromolecule attached to the protein is the same. In some embodiments, when z is 2 or more, at least one L-Macromolecule attached to the protein is different. In some embodiments, when z is 2 or more, each L-Macromolecule attached to the protein is different.

Exemplary protein-macromolecule conjugates of formula XX are encompassed within the following structure:

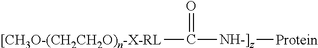

wherein:

n is an integer from 2 to 4000;

X is a spacer moiety;

RL a releasable linker;

z is an integer from 1 to 25;

—NH— is an amine group of a residue within the protein; and

Protein is a chemokine, a chemokine antagonist, a cytokine, a cytokine antagonist, an antibody, or a therapeutic peptide.

In some embodiments, RL is a releasable linker of the present disclosure. In some embodiments, the releasable linker is derived from a bifunctional releasable linker (e.g., a linker of formula (I), formula (II), formula (III) or formula (IV)) or polymeric reagent with releasable linker (e.g., formula (V) or formula (VI)) disclosed herein.

In another aspect, exemplary protein-macromolecule conjugates of formula XX are encompassed within the following structure:

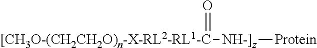

wherein:

n is an integer from 2 to 4000;

X is a spacer moiety;

RL¹ is a first releasable linker;

RL² is a second releasable linker;

z is an integer from 1 to 25;

—NH— is an amine group of a residue within the protein; and

Protein is a chemokine, a chemokine antagonist, a cytokine, a cytokine antagonist, an antibody, or a therapeutic peptide.

Exemplary conjugates of the disclosure wherein the water-soluble polymer is in a branched form include those wherein the water-soluble polymer is encompassed within the following structure:

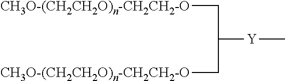

wherein Y═O and NH; each (n) is independently an integer having a value of from 2 to 4000, e.g., 2, 4, 6, 8, 10, 20, 30, 40, 50, 60, 70, 80, 90, 100, 200, 300, 400, 500, 600, 700, 800, 900, 1000, 1500, 2000, 2500, 3000, 3500, or 4000, including all values and ranges therebetween.

Exemplary conjugates of the disclosure wherein the water-soluble polymer is in a branched form include those wherein the water-soluble polymer is encompassed within the following structure:

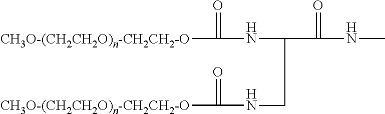

wherein each (n) is independently an integer having a value of from 2 to 4000, e.g., 2, 4, 6, 8, 10, 20, 30, 40, 50, 60, 70, 80, 90, 100, 200, 300, 400, 500, 600, 700, 800, 900, 1000, 1500, 2000, 2500, 3000, 3500, or 4000, including all values and ranges therebetween.

Exemplary protein-macromolecule conjugates formed using two releasable linkage-providing polymeric reagents include those of the following formula (XI):

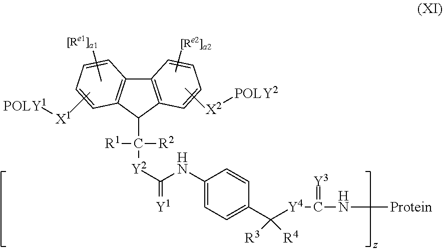

wherein:

POLY¹ is a first water-soluble polymer;

POLY² is a second water-soluble polymer;

X¹ is a first spacer moiety;

X² is a second spacer moiety;

Y¹ is O or S;

Y² is O or S;

Y³ is O or S;

Y⁴ is O or S;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R³ is a hydrogen, alkyl, substituted alkyl, alkenyl, substituted alkenyl, alkynyl, substituted alkynyl, aryl, or substituted aryl;

R⁴ is a hydrogen, alkyl, substituted alkyl, alkenyl, substituted alkenyl, alkynyl, substituted alkynyl, aryl, or substituted aryl;

a1 is an integer from 0 to 3;

a2 is an integer from 0 to 3;

c is an integer from 0 to 4;

z is an integer from 1 to 25;

R^{e1}, when present, is a first electron altering group;

R^{e2}, when present, is a second electron altering group;

R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;

—NH— is an amine group of a residue within the protein; and

Protein is a chemokine, a chemokine antagonist, a cytokine, a cytokine antagonist, an antibody, or a therapeutic peptide.

In some embodiments, R¹, R², R³, R⁴, R^{e1}, R^{e2}, and R^{d} are as defined above in formula (IV).

In some embodiments of formula (XI), POLY¹ and POLY² are each independently selected from the water soluble polymers described herein. In some embodiments, POLY¹ and POLY² are the same water-soluble polymer. In some embodiments, POLY¹ and POLY² are different water-soluble polymers.

In some embodiments of formula (XI), X¹ and X² are each independently selected from the spacer moieties described herein. In some embodiments, X¹ and X² are the same spacer moiety. In some embodiments, X¹ and X² are different spacer moieties.

Exemplary conjugates have the following structure (XU-A):

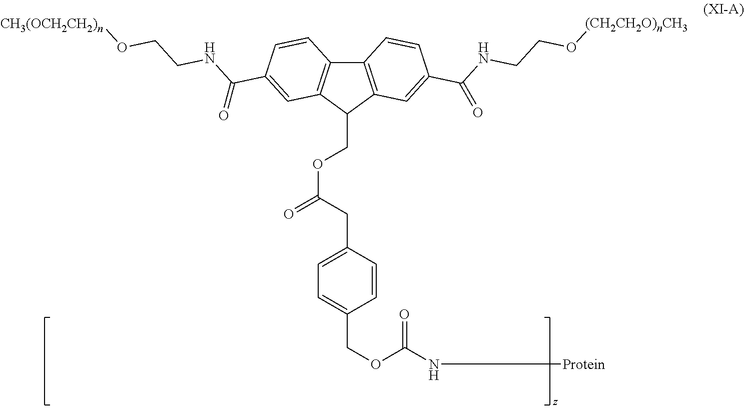

wherein n is independently an integer from 4 to 1500 and z is an integer from 1 to 25.

Other exemplary conjugates formed using two releasable linkage-providing polymeric reagents include those of the following formula (XII):

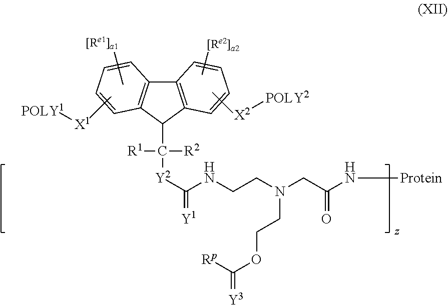

wherein:

POLY¹ is a first water-soluble polymer;

POLY² is a second water-soluble polymer;

X¹ is a first spacer moiety;

X² is a second spacer moiety;

Y¹ is O or S;

Y² is O or S;

Y³ is O or S;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 is an integer from 0-3;

a2 is an integer from 0-3;

z is an integer from 1 to 25;

R^{e1}, when present, is a first electron altering group;

R^{e2}, when present, is a second electron altering group;

R^{p} is a hydrogen, alkyl, substituted alkyl, alkenyl, substituted alkenyl, alkynyl, substituted alkynyl, aryl, or substituted aryl; and

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{e1}, R^{e2}, and R^{p} are as defined above in formula (VI).

In some embodiments of formula (XII), POLY¹ and POLY² are each independently selected from the water soluble polymers described herein. In some embodiments, POLY¹ and POLY² are the same water-soluble polymer. In some embodiments, POLY¹ and POLY² are different water-soluble polymers.

In some embodiments of formula (XII), X¹ and X² are each independently selected from the spacer moieties described herein. In some embodiments, X¹ and X² are the same spacer moiety. In some embodiments, X¹ and X² are different spacer moieties.

Exemplary conjugates have the following structure (XII-A):

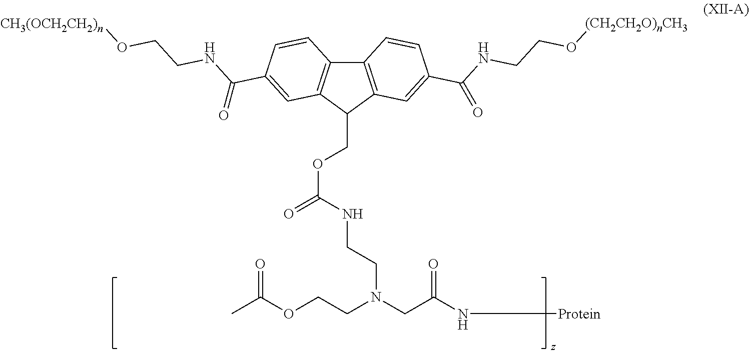

wherein n is independently an integer from 4 to 1500 and z is an integer from 1 to 25.

Exemplary conjugates formed using click chemistry with suitable polymeric reagents include those of the following formula (XIII):

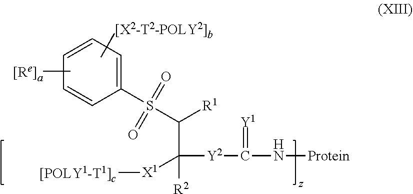

wherein:

POLY¹ is a first straight or branched water-soluble polymer;

POLY² is a second straight or branched water-soluble polymer;

X¹ is a first spacer moiety or —X-FG²;

X², when present, is a second spacer moiety;

T¹ is a first triazole functional group;

T² is a second triazole functional group;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl; and —X-FG²;
wherein:
X is a spacer moiety; and

FG² is a functional group capable of reacting through click chemistry, independently including but not limited to azide, alkynyl, and cycloalkynyl (e.g., dibenzocyclooctyne (DBCO)) groups.

a is an integer from 0 to 5;

b is an integer from 0 to 3;

c is an integer from 0 to 2;

z is an integer from 1 to 25;

Y¹ is O or S;

Y² is O or S; and

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{e}, a, b, c, and z are as defined above in formula (I).

In some embodiments of formula (XIII), POLY¹ and POLY² are each independently selected from the water soluble polymers described herein. In some embodiments, POLY¹ and POLY² are the same water-soluble polymer. In some embodiments, POLY¹ and POLY² are different water-soluble polymers.

In some embodiments of formula (XIII), X¹ and X² are each independently selected from the spacer moieties described herein. In some embodiments, X¹ and X² are the same spacer moiety. In some embodiments, X¹ and X² are different spacer moieties.

Within formula (XIII), conjugates having the more defined structure are contemplated as formula (XIII-A), (XII-B), (XII-C), or (XIII-D):

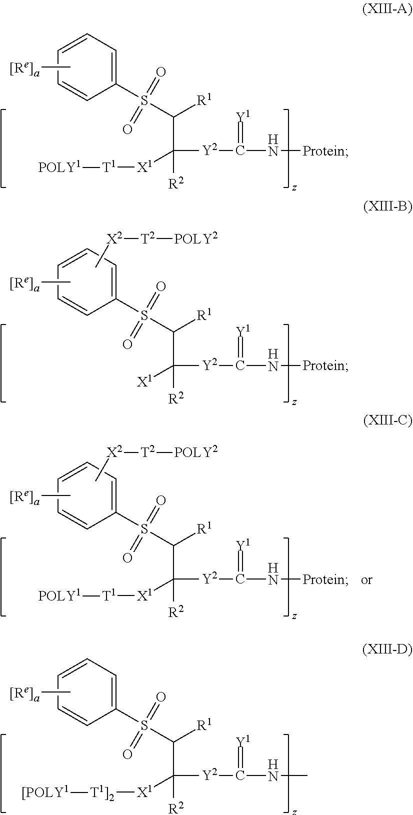

wherein each of X¹ is a first spacer moiety; X² is a second spacer moiety; POLY¹, POLY², T¹, T², R¹, R², R^{e}, a, z, Y¹, Y², and protein are as previously defined

In certain embodiments of formula (XIII), (XIII-A), (XIII-B), (XIII-C), or (XIII-D), a is an integer from 0 to 2; R¹ and R² are each independently hydrogen, Me, or Et; and R^{e} is nitro, cyano, halogen, —CF₃, —CONHMe, —SO₂NHMe, —OMe, —NHMe, —NHAc, —NHSO₂Me, or —OCF₃.

Further exemplary conjugates have the following structure (XIII-A1):

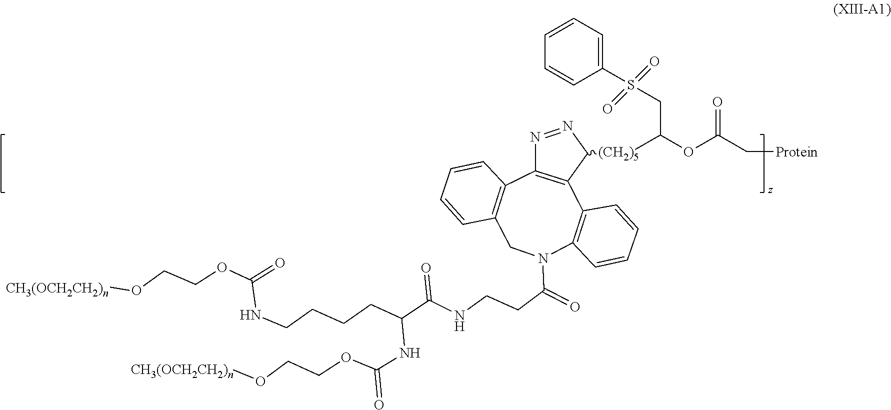

wherein, R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl; n is independently an integer from 4 to 1500; z is an integer from 1 to 25; and “—NH—” is an amine group of a residue within the protein and represents one or more polymers individually attached to the protein. In certain embodiments, a is an integer from 1 to 2; R^{e} is 4-F, 4-C₁, 4-CF₃, 2,4-difluoro, or 2-CF₃-4-F substitution.

Further exemplary conjugates have the following structure as (XIII-B1), (XIII-C1), (XIII-D1), or (XII-D2):

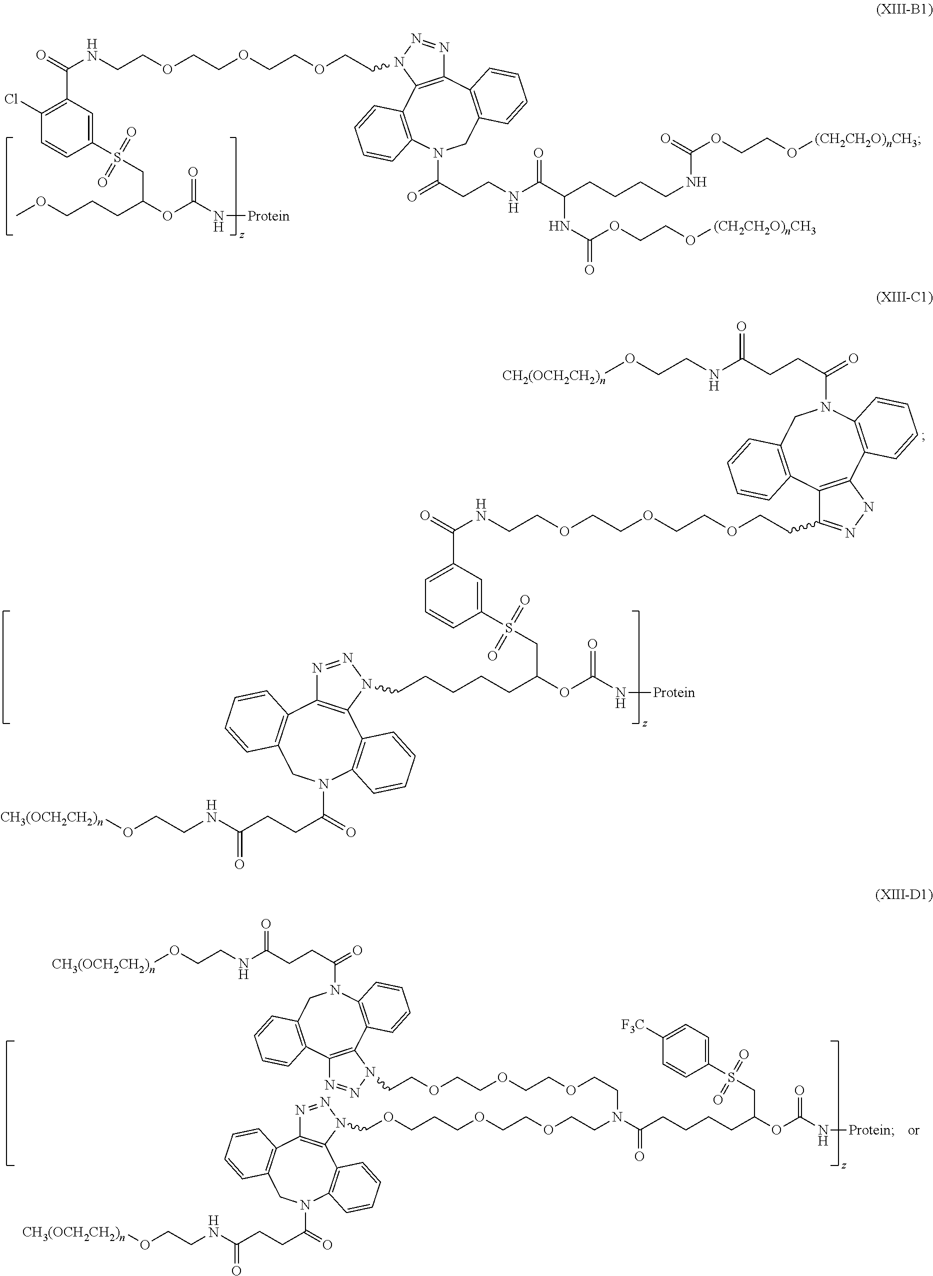

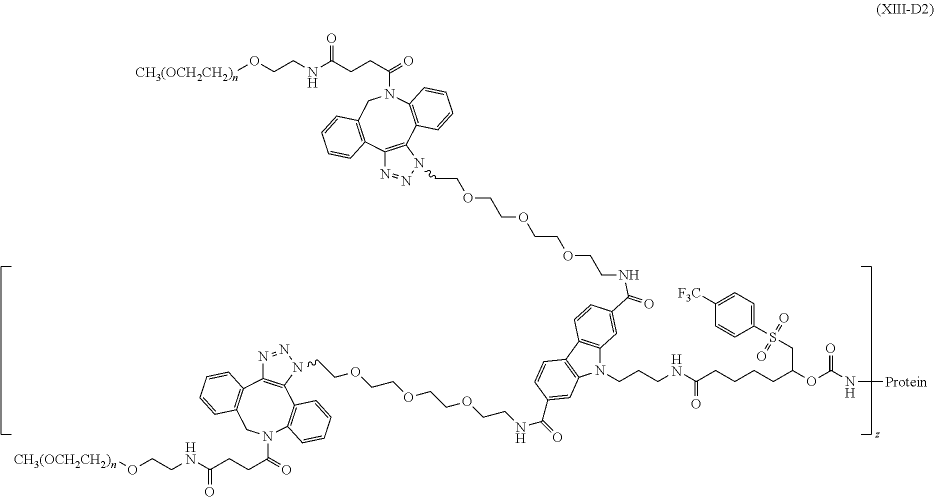

wherein:

n is independently an integer from 4 to 1500;

z is an integer from 1 to 25; and

—NH— is an amine group of a residue within the protein; and

Protein is a chemokine, a chemokine antagonist, a cytokine, a cytokine antagonist, an antibody, or a therapeutic peptide.

Other exemplary conjugates formed using click chemistry with suitable polymeric reagents include those of the following formula (XIV):

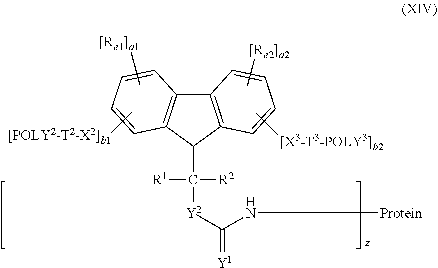

wherein:

POLY² is a straight or branched water-soluble polymer;

POLY³ is a straight or branched water-soluble polymer;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

z is an integer from 1 to 25;

R^{e1}, when present, is a first electron altering group;

R^{e2}, when present, is a second electron altering group; or —X-FG²;
wherein
X is a spacer moiety; and
FG² is a functional group capable of reacting through click chemistry, independently including but not limited to azide, alkynyl, and cycloalkynyl (e.g., dibenzocyclooctyne (DBCO)) groups.

X², when present, is a spacer moiety;

X³, when present, is a spacer moiety;

T² is a triazole functional group;

T³ is a triazole functional group;

Y¹ is O or S;

Y² is O or S; and

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{e1}, and R^{e2} are as defined above in formula (VI).

In some embodiments of formula (XIV), POLY² and POLY³ are each independently selected from the water soluble polymers described herein. In some embodiments, POLY² and POLY³ are the same water-soluble polymer. In some embodiments, POLY² and POLY³ are different water-soluble polymers.

In some embodiments of formula (XIV), X² and X³ are each independently selected from the spacer moieties described herein. In some embodiments, X² and X³ are the same spacer moiety. In some embodiments, X² and X³ are different spacer moieties.

In certain embodiments of formula (XIV), a1 and a2 are each independently an integer from 0 to 2; R¹ and R² are each independently hydrogen, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Within formula (XIV), conjugates having the more defined structure are contemplated as formula (XIV-A):

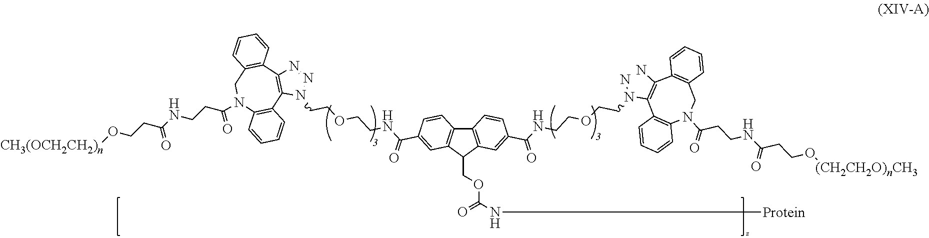

wherein n is independently an integer from 4 to 1500; z is an integer from 1 to 25; and —NH— is an amine group of a residue within the protein.

Other exemplary conjugates formed using click chemistry with suitable polymeric reagents include those of the following formula (XV):

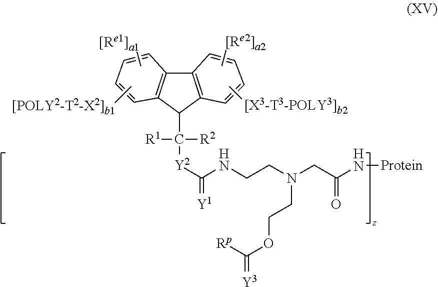

wherein:

POLY² is a straight or branched water-soluble polymer;

POLY³ is a straight or branched water-soluble polymer;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

z is an integer from 1 to 25;

R^{e1}, when present, is a first electron altering group;

R^{e2}, when present, is a second electron altering group; or —X-FG²;
wherein
X is a spacer moiety; and
FG² is a functional group capable of reacting through click chemistry, independently including but not limited to azide, alkynyl, and cycloalkynyl (e.g., dibenzocyclooctyne (DBCO)) groups.

R^{p} is a hydrogen, alkyl, substituted alkyl, alkenyl, substituted alkenyl, alkynyl, substituted alkynyl, aryl, or substituted aryl;

X², when present, is a spacer moiety;

X³, when present, is a spacer moiety;

T² is a triazole functional group;

T³ is a triazole functional group;

Y¹ is O or S;

Y² is O or S;

Y³ is O or S; and

—NH— is an amine group of a residue within the protein; and

Protein is a chemokine, a chemokine antagonist, a cytokine, a cytokine antagonist, an antibody, or a therapeutic peptide.

In some embodiments, R¹, R², R^{p}, R^{e1}, and R² are as defined above in formula (VI).

In some embodiments of formula (XV), POLY² and POLY³ are each independently selected from the water soluble polymers described herein. In some embodiments, POLY² and POLY³ are the same water-soluble polymer. In some embodiments, POLY² and POLY³ are different water-soluble polymers.

In some embodiments of formula (XV), X² and X³ are each independently selected from the spacer moieties described herein. In some embodiments, X² and X³ are the same spacer moiety. In some embodiments, X² and X³ are different spacer moieties.

In certain embodiments of formula (XV), a1 and a2 are each independently an integer from 0 to 2; R¹ and R² are each independently hydrogen, Me, or Et; and R^{e1} and R^{e2} are each independently nitro, cyano, halogen, —CF₃, —CONHMe, —SO₂NHMe, —OMe, —NHMe, —NHAc, —NHSO₂Me, or —OCF₃.

Within formula (XV), conjugates having the more defined structure are as following formula (XV-A):

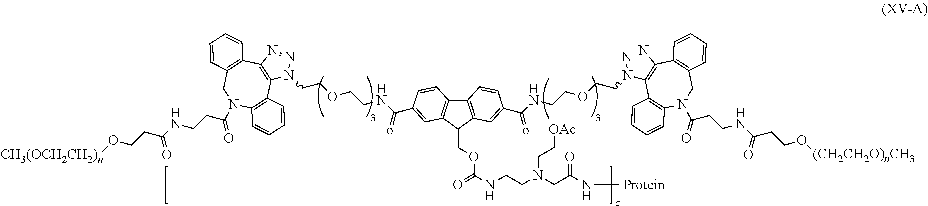

wherein n is independently an integer from 4 to 1500; z is an integer from 1 to 25 and —NH— is an amine group of a residue within the protein.

Other exemplary conjugates formed using click chemistry with suitable polymeric reagents include those of the following formula (XVI):

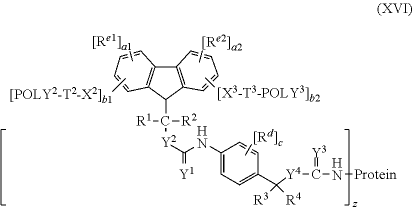

wherein:

POLY² is a straight or branched water-soluble polymer;

POLY³ is a straight or branched water-soluble polymer;

R¹ is a hydrogen, alkyl, substituted alkyl, alkenyl, substituted alkenyl, alkynyl, substituted alkynyl, aryl, or substituted aryl;

R² is a hydrogen, alkyl, substituted alkyl, alkenyl, substituted alkenyl, alkynyl, substituted alkynyl, aryl, or substituted aryl;

R³ is a hydrogen, alkyl, substituted alkyl, alkenyl, substituted alkenyl, alkynyl, substituted alkynyl, aryl, or substituted aryl;

R⁴ is a hydrogen, alkyl, substituted alkyl, alkenyl, substituted alkenyl, alkynyl, substituted alkynyl, aryl, or substituted aryl;

a1 and a2 are each independently an integer from 0 to 4;

b1 is 1;

b2 is an integer from 0 to 1;

c is an integer from 0 to 4;

z is an integer from 1 to 25;

R^{e1}, when present, is a first electron altering group;

R^{e2}, when present, is a second electron altering group; or —X-FG²;
wherein
X is a spacer moiety; and
FG² is a functional group capable of reacting through click chemistry, independently including but not limited to azide, alkynyl, and cycloalkynyl (e.g., dibenzocyclooctyne (DBCO)) groups.

R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;

X², when present, is a spacer moiety;

X³, when present, is a spacer moiety;

T² is a triazole functional group;

T³ is a triazole functional group;

Y¹ is O or S;

Y² is O or S;

Y³ is O or S;

Y⁴ is O or S; and

—NH— is an amine group of a residue within the protein; and

Protein is a chemokine, a chemokine antagonist, a cytokine, a cytokine antagonist, an antibody, or a therapeutic peptide.

In some embodiments, R¹, R², R³, R⁴, R^{d}, R^{e1}, and R^{e2} are as defined above in formula (IV).

In some embodiments of formula (XVI), POLY² and POLY³ are each independently selected from the water soluble polymers described herein. In some embodiments, POLY² and POLY³ are the same water-soluble polymer. In some embodiments, POLY² and POLY³ are different water-soluble polymers.

In some embodiments of formula (XVI), X² and X³ are each independently selected from the spacer moieties described herein. In some embodiments, X² and X³ are the same spacer moiety. In some embodiments, X² and X³ are different spacer moieties. In some embodiments, the protein is a cytokine. The cytokine includes GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, or TNF-β. In certain embodiments, the cytokine is IL-2. In certain embodiment, the IL-2 comprises about 80%, 85%, 90%, 95%, 96%, 97%, 98%, or 99% sequence identity to SEQ ID NO:1.

In some embodiments, the protein is a chemokine. The chemokine includes MCP-1, MCP-2, MCP-3, MCP-24, MCP-5, CXCL76, I-309 (CCL1), BCA1 (CXCL13), MIG, SDF-1/PBSF, IP-10, I-TAC, MIP-1α, MIP-1β, RANTES, eotaxin-1, eotaxin-2, GCP-2, Gro-α, Gro-β, Gro-γ, LARC (CCL20), ELC (CCL19), SLC (CCL21), ENA-78, PBP, TECK(CCL25), CTACK (CCL27), MEC, XCL1, XCL2, HCC-1, HCC-2, HCC-3, or HCC-4.

In some embodiments, the protein is an antibody. The antibody can target one or more of angiopoietin 2, AXL, ACVR2B, angiopoietin 3, activin receptor-like kinase 1, amyloid A protein, β-amyloid, AOC3, BAFF, BAFF-R, B7-H3, BCMAC, A-125 (imitation), C5, CA-125, CCL11 (eotaxin-1), CEA, CSF1R, CD2, CD3, CD4, CD6, CD15, CD19, CD20, CD22, CD23, CD25, CD28, CD30, CD33, CD37, CD38, CD40, CD41, CD44, CD51, CD52, CD54, CD56, CD70, CD74, CD97B, CD125, D134, CD147, CD152, CD154, CD279, CD221, C242 antigen, CD276, CD278, CD319, Clostridium difficile, claudin 18 isoform 2, CSF1R, CEACAM5, CSF2, carbonic anhydrase 9, CLDN18.2, cardiac myosin, CCR4, CGRP, coagulation factor III, c-Met, CTLA-4, DPP4, DR5, DLL3, DLL4, dabigatran, EpCAM, ebolavirus glycoprotein, endoglin, episialin, EPHA3, c-Met, FGFR2, fibrin II beta chain, FGF 23, folate receptor 1, GMCSF, GD2 ganglioside, GDF-8, GCGR, gelatinase B, glypican 3, GPNMB, GMCSF receptor α-chain, kallikrein, KIR2D, ICAM-1, ICOS, IGF1, IGF2, IGF-1 receptor, IL-1α, IL-1β, IL-2, IL-4Rₐ, IL-5, IL-6, IL-6 R, IL-9, IL-12, IL-13, IL17A, IL17F, IL-20, IL-22, IL-23, IL-31, IFN-α, IFN-β, IFN-γ, integrin α4β7, interferon α/β receptor, Influenza A hemagglutinin, ILGF2, HER1, HER2, HER3, HHGFR, HGF, HLA-DR, hepatitis B surface antigen, HNGF, Hsp90, HGFR, L-selectin, Lewis-Y antigen, LYPD3, LOXL2, LIV-1, MUC1, MCP-1, MSLN, mesothelin, MIF, MCAM, NCA-90, NCA-90Notch 1, nectin-4, PCDP1, PD-L1, PD-1, PCSK9, PTK7, PCDC1, phosphatidylserine, RANKL, RTN4, Rhesus factor, ROR1, SLAMF7, Staphylococcus aureus alpha toxin, Staphylococcus aureus bi-component leucocidin, SOST, selectin P, SLITRK6, SDC1, TFPI, TRAIL-R2, tumor antigen CTAA16.88, TNF-α, TWEAK receptor, TNFRSF8, TYRP1, tau protein, TAG-72, TSLP, TRAIL-R1, TRAIL-R2, TGF-β, TAG-72, TRAP, TIGIT, tenascin C, OX-40, VEGF-A, VWF, VEGFR1, or VEGFR2.

In some embodiments, the protein is a therapeutic peptide. Peptides include, but are not limited to: glucagon-like peptide 1 (GLP-1), exendin-2, exendin-3, exendin-4, atrial natriuretic factor (ANF), ghrellin, vasopressin, growth hormone, growth hormone-releasing hormone (GHRH), RC-3095, somatostatin, bombesin, PCK-3145, Phe-His-Ser-Cys-Asn (PHSCN), IGF1, B-type natriuretic peptide, peptide YY (PYY), interferons, thrombospondin, angiopoietin, calcitonin, gonadotropin-releasing hormone, hirudin, glucagon, anti-TNF-alpha, fibroblast growth factor, granulocyte colony stimulating factor, obinepitide, pituitary thyroid hormone (PTH), leuprolide, sermorelin, pramorelin, nesiritide, rotigaptide, cilengitide, MBP-8298, AL-108, enfuvirtide, thymalfasin, daptamycin, HLFI-II, Lactoferrin, Delmitide, glutathione, T-cell epitope PRI, Protease-3 peptides 1-11, B-cell epitope P3, lutenizing hormone-releasing hormone (LHRH), substance P, neurokinin A, neurokinin B, CCK-8, enkephalins, including leucine enkephalin and methionine enkephalin, dermaseptin, [des-Ala20, Gln34]-dermaseptin, surfactant-associated antimicrobial anionic peptide, Apidaecin IA; Apidaecin IB; OV-2; 1025, Acetyl-Adhesin Peptide (1025-1044) amide; Theroma-cin (49-63); Pexiganan (MSI-78); Indolicidin; Apelin-15 (63-77); CFPlO (71-85); Lethal Factor (LF) Inhibitor Anthrax related; Bactenecin; Hepatitis Virus C NS3 Protease Inhibitor 2; Hepatitis Virus C NS3 Protease Inhibitor 3; Hepatitis Virus NS3 Protease Inhibitor 4; NS4A-NS4B Hepatitis Virus C (NS3 Protease Inhibitor I); HIV-1, HIV-2 Protease Substrate; Anti-FM Peptide; Bak-BH3; Bax BH3 peptide (55-74) (wild type); Bid BH3-r8; CTT (Gelatinase Inhibitor); E75 (Her-2/neu) (369-377); GRP78 Binding Chimeric. Peptide Motif; p53(17-26); EGFR2/KDR Antagonist; Colivelin AGA-(C₈R) HNGI 7 (Humanin derivative); Activity-Dependent Neurotrophic Factor (ADNF); Beta-Secretase Inhibitor I; Beta-Secretase Inhibitor 2; ch[beta]-Amyloid (30-16); Humanun (HN) sHNG, [Glyl4]-HN, [Glyl 4]-Humanin; Angiotensin Converting Enzyme Inhibitor (BPP); Renin Inhibitor III; Annexin I (ANXA-I; Ac2-12); Anti-Inflammatory Peptide I; Anti-Inflammatory Peptide 2; Anti-Inflammatory Apelin 12; [D-Phel2, Leul4]-Bombesin; Antennapedia Peptide (acid) (penetratin); Antennepedia Leader Peptide (CT); Mastoparan; [Thr28, Nle31]-Cholecystokinin (25-33) sulfated; Nociceptin (1-13) (amide); Fibrinolysis Inhibiting Factor; Gamma-Fibrinogen (377-395); Xenin; Obestatin (human); [Hisl, Lys6]-GHRP (GHRP-6); [Ala5, [beta]-Ala8]-NeurokininA (4-10); Neuromedin B; Neuromedin C; Neuromedin N; Activity-Dependent Neurotrophic Factor (ADNF-14); Acetalin I (Opioid Receptor Antagonist I); Acetalin 2 (Opioid Receptor Antagonist 2); Acetalin 3 (Opioid Receptor Antagonist 3); ACTH (1-39) (human); ACTH (7-38) (human); Sauvagine; Adipokinetic Hormone (Locusta Migratoria); Myristoylated ADP-Ribosylation Factor 6, myr-ARF6 (2-13); PAMP (1-20) (Proadrenomedullin (1-20) human); AGRP (25-51); Amylin (8-37) (human); Angiotensin I (human); Angiotensin II (human); Apstatin (Aminopeptidase P Inhibitor); Brevinin-I; Magainin I; RL-37; LL-37 (Antimicrobial Peptide) (human); Cecropin A; Antioxidant peptide A; Antioxidant peptide B; L-Camosine; BcI 9-2; NPVF; NeuropeptideAF (hNPAF) (Human); Bax BH3 peptide (55-74); bFGF Inhibitory Peptide; bFGF inhibitory Pep tide II; Bradykinin; [Des-Argl OJ-HOE 140; Caspase I Inhibitor II; Caspase I Inhibitor VIII; Smac N7 Protein (MEKI Derived Peptide Inhibitor I; hBD-1 ([beta]-Defensin-1) (human); hBD-3 ([beta]-Defensin-3) (human); hBD-4 ([beta]-Defensin-4) (human); HNP-I (Defensin Human Neutrophil Peptide I); HNP-2 (Defensin Human neutrophil Peptide-2 Dynorphin A (1-17)); Endomorphin-I; [beta]-Endorphin (human porcine); Endothelin 2 (human); Fibrinogen Binding Inhibitor Peptide; Cyclo(-GRGDSP); TP508 (Thrombin-derived Peptide); Galanin (human); GIP (human); Gastrin Releasing Peptide (human); Gastrin-1 (human); Ghrelin (human); PDGF-BB peptide; [D-Lys3]-GHRP-6; HCV Core Protein (1-20); a3B1 Integrin Peptide Fragment (325) (amide); Laminin Pentapeptide (amide) Mel-anotropin-Potentiating Factor (MPF); VA-[beta]-MSH, Lipo-tropin-Y (Proopiomelanocortin-derived); Atrial Natriuretic Peptide (1-28) (human); Vasonatrin Peptide (1-27); [Ala5, B-Ala8]-Neurokinin A (4-10); Neuromedin L (NKA); Ac-(Leu28, 31)-Neuropeptide Y (24-26); Alytesin; Brain Neuropeptide II; [D-tyrll]-Neurotensin; IKKy NEMO Binding Domain (NBD) Inhibitory Peptide; PTD-p50 (NLS) Inhibitory Peptide; OrexinA (bovine, human, mouse, rat); Orexin B (human); Aquaporin-2(254-267) (human Pancreastatin)(37-52); Pancreatic Polypeptide (human); Neuropeptide; Peptide YY (3-36) (human); Hydroxymethyl-Phytochelatin 2; PACAP (I-27) (amide, human, bovine, rat); Prolactin Releasing Peptide (1-31) (human); Salusin-alpha; Salusin-beta; Saposin C22; Secretin (human); L-Selectin; Endokinin A/B; Endokinin C (Human); Endokinin D (Human); Thrombin Receptor (42-48) Agonist (human); LSKL (Inhibitor of Thrombospondin); Thyrotropin Releasing Hormone (TRH); P55-TNFR Fragment; Urotensin II (human); VIP (human, porcine, rat); VIP Antagonist; Helodermin; Exenatide; ZPlO (AVEOOIOO); Pramlinitide; AC162352 (PYY)(3-36); PYY; Obinepitide; Glucagon; GRP; Ghrelin (GHRP6); Leuprolide; Histrelin; Oxytocin; Atosiban (RWJ22164); Sermorelin; Nesiritide; bivalirudin (Hirulog); Icatibant; Aviptadin; Rotigaptide (ZP123, GAP486); Cilengitide (EMD-121924, RGD Peptides); AlbuBNP; BN-054; Angiotensin II; MBP-8298; Peptide Leucine Arginine; Ziconotide; AL-208; AL-108; Carbeticon; Tripeptide; SAL; Coliven; Humanin; ADNF-14; VIP (Vasoactive Intestinal Peptide); Thymalfasin; Bacitracin; Gramidicin; Pexiganan (MSI-78); P1 13; PAC-113; SCV-07; HLF1-Il (Lactoferrin); DAPTA; TRI-1144; Tritrpticin; Anti-flammin 2; Gattex (Teduglutide, ALX-0600); Stimuvax (L-BLP25); Chrysalin (TP508); Melanonan II; Spantide II; Ceruletide; Sincalide; Pentagastin; Secretin; Endostatin peptide; E-selectin; HER2; IL-6; IL-8; IL-10; PDGF; Thrombospondin; uPA (I); uPA (2); VEGF; VEGF (2); Pentapeptide-3; XXLRR; Beta-Amyloid Fibrillogenesis; Endomorphin-2; TIP 39 (Tuberoinfundibular Neuropeptide); PACAP (1-38) (amide, human, bovine, rat); TGFB activating peptide; Insulin sensitizing factor (ISF402); Transforming Growth Factor BI Peptide (TGF-B1); Caerulein Releasing Factor; IELLQAR (8-branchMAPS); Tigapotide PK3145; Goserelin; Abarelix; Cetrorelix; Ganirelix; Degarelix (Triptorelin); Barusiban (FE 200440); Pralmorelin; Octreotide; Eptifibatide; Netamiftide (INN-00835); Daptamycin; Spantide II; Delmitide (RDP-58); AL-209; Enfuvirtide; IDR-I; Hexapeptide-6; Insulin-A chain; Lanreotide; Hexa[rho]eptide-3; Insulin B-chain; Glargine-A chain; Glargine-B chain; Insulin-LisPro B-chain analog; Insulin-Aspart B-chain analog; Insulin-Glulisine B chain analog; Insulin-Determir B chain analog; Somatostatin Tumor Inhibiting Analog; Pancreastatin (37-52); Vasoactive Intestinal Peptide fragment (KKYL-NH2); and Dynorphin A. Examples of proteins suitable for use in the disclosure include but are not limited to: immunotoxin SS1P, adenosine deaminase, argininase, and others.

### IL-2-Macromolecule Conjugates

Turning to one or more embodiments of the disclosure, a more specific protein-macromolecule conjugate is provided, the conjugate comprising a residue of an IL-2 moiety covalently attached through linkers to multiple water-soluble polymers. The conjugates of the disclosure will have one or more of the following features.

### The IL-2 Moiety

As previously stated, the conjugate generically comprises a residue of an IL-2 moiety covalently attached, through releasable or non-releasable linkers, to one or more water-soluble polymers. As used herein, the term “IL-2 moiety” shall refer to the IL-2 moiety prior to conjugation as well as to the IL-2 moiety following attachment to water-soluble polymers. It will be understood, however, that when the original IL-2 moiety is attached to water-soluble polymers, the IL-2 moiety is slightly altered due to the presence of one or more covalent bonds associated with linkage to the polymer(s). Often, this slightly altered form of the IL-2 moiety attached to another molecule is referred to as a “residue” of the IL-2 moiety.

The IL-2 moiety can be derived from non-recombinant methods and from recombinant methods and the disclosure is not limited in this regard. In addition, the IL-2 moiety can be derived from human sources, animal sources, and plant sources.

Any IL-2 moiety obtained non-recombinant and recombinant approaches can be used as an IL-2 moiety in preparing the conjugates described herein.

Depending on the system used to express proteins having IL-2 activity, the IL-2 moiety can be unglycosylated or glycosylated and either may be used. That is, the IL-2 moiety can be unglycosylated or the IL-2 moiety can be glycosylated. In one or more embodiments of the disclosure, the IL-2 moiety is unglycosylated.

The IL-2 moiety can advantageously be modified to include and/or substitute one or more amino acid residues such as, for example, lysine, cysteine, histidine and/or arginine, in order to provide facile attachment of the polymer to an atom within the side chain of the amino acid. An example of substitution of an IL-2 moiety is described in U.S. Pat. No. 5,206,344. In addition, the IL-2 moiety can be modified to include a non-naturally occurring amino acid residue. An example of substituting non-naturally occurring amino acid residue of an IL-2 moiety is described in WO 2019/028419. Techniques for adding amino acid residues and non-naturally occurring amino acid residues are well known to those of ordinary skill in the art. Reference is made to J. March, Advanced Organic IL-2mistry: Reactions Mechanisms and Structure, 4th Ed. (New York: Wiley-Interscience, 1992).

In addition, the IL-2 moiety can advantageously be modified to include attachment of a functional group (other than through addition of a functional group-containing amino acid residue). For example, the IL-2 moiety can be modified to include a thiol group. In addition, the IL-2 moiety can be modified to include an N-terminal alpha carbon. In addition, the IL-2 moiety can be modified to include one or more carbohydrate moieties. In addition, the IL-2 moiety can be modified to include an aldehyde group. In addition, the IL-2 moiety can be modified to include a ketone group. In certain embodiments of the disclosure, it is preferred that the IL-2 moiety is not modified to include one or more of a thiol group, an N-terminal alpha carbon, carbohydrate, aldehyde group and ketone group.

Exemplary IL-2 moieties are described in the literature and in, for example, U.S. Pat. Nos. 5,116,943, 5,153,310, 5,635,597, 7,101,965 and 7,567,215 and U.S. Patent Application Publication Nos. 2010/0036097 and 2004/0175337. A preferred IL-2 moiety has the amino acid sequence corresponding to FIG. 1.

In some instances, the IL-2 moiety can be in a “monomer” form, wherein a single expression of the corresponding peptide is organized into a discrete unit. In other instances, the IL-2 moiety can be in the form of a “dimer” (e.g., a dimer of recombinant IL-2) wherein two monomer forms of the protein are associated (e.g., by disulfide bonding) to each other. For example, in the context of a dimer of recombinant human IL-2, the dimer may be in the form of two monomers associated to each other by a disulfide bond formed from each monomer's Cys 125 residue.

In addition, precursor forms of IL-2 can be used as the IL-2 moiety. Truncated versions, hybrid variants, and peptide mimetics of any of the foregoing sequences can also serve as the IL-2 moiety. Biologically active fragments, deletion variants, substitution variants or addition variants of any of the foregoing that maintain at least some degree of IL-2 activity can also serve as an IL-2 moiety.

For any given peptide or protein moiety, it is possible to determine whether that moiety has IL-2 activity. Various methods for determining the in vitro IL-2 activity are described in the art. An exemplary approach is the CTLL-2 cell proliferation assay described in the experimental below. An exemplary approach is described in Moreau et al. (1995) Mol. Immunol. 32:1047-1056). Other methodologies known in the art can also be used to assess IL-2 function, including electrometry, spectrophotometry, chromatography, and radiometric methodologies.

More specific exemplary conjugates in accordance with the disclosure will now be described. Typically, such an IL-2 moiety is expected to share (at least in part) a similar amino acid sequence as the sequence provided in FIG. 1. Thus, while reference will be made to specific locations or atoms within the sequence of FIG. 1, such a reference is for convenience only and one having ordinary skill in the art will be able to readily determine the corresponding location or atom in other moieties having IL-2 activity. In particular, the description provided herein for native human IL-2 is often applicable to fragments, deletion variants, substitution variants or addition variants of any of the foregoing.

### Conjugate Assembly

Amino groups on IL-2 moieties provide a point of attachment between the IL-2 moiety and the water-soluble polymer. Using the amino acid sequence provided in FIG. 1, it is evident that there are several lysine residues in each having an 6-amino acid that may be available for conjugation. Further, the N-terminal amine of any protein can also serve as a point of attachment.

There are a number of examples of suitable reagents useful for forming covalent releasable linkages with available amines of an IL-2 moiety. Non-limiting specific examples, along with the corresponding conjugates, are provided in Table 1, below. In the table, the variable “n” represents the number of repeating monomeric units, z is an integer from 1 to 25, and “—NH-IL-2” represents the residue of the IL-2 moiety following conjugation to the polymeric reagents or linkers and forming one or more water-soluble polymers individually attached to an IL-2 moiety, or one or more linkers individually attached to an IL-2 moiety. While each polymeric portion [e.g., (OCH₂CH₂)ₙ or (CH₂CH₂O)ₙ] presented in Table 1 terminates in a “CH₃” group, other groups (such as H and benzyl) can be substituted therefor.

TABLE 1
Examples of Amine-Selective Coupling Reagents and the IL-2 Moiety
Conjugate Formed Therefrom
Coupling Reagent

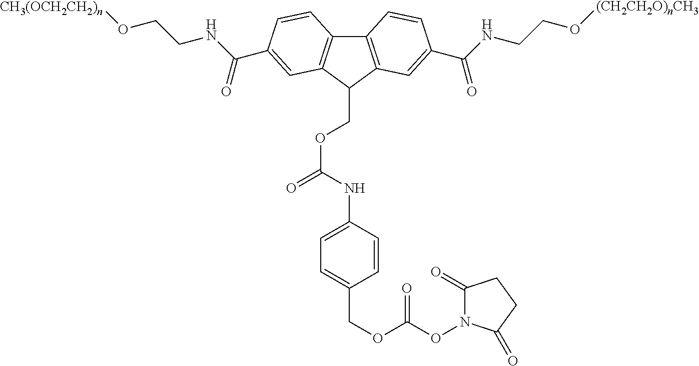

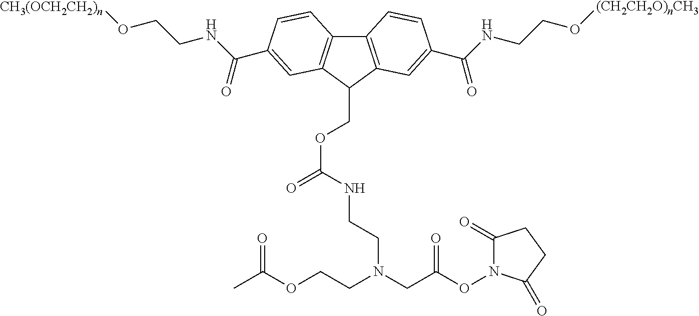

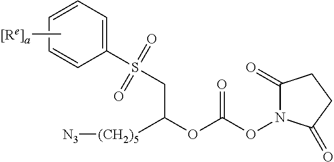

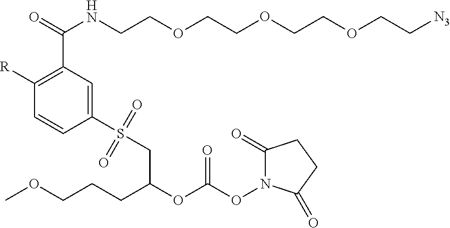

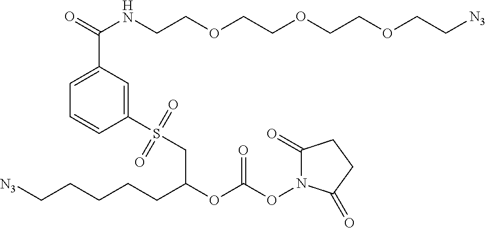 [SMILES: [N-]=[N+]=NCCCCCC(CS(=O)(=O)c1cccc(C(=O)NCCOCCOCCOCCN=[N+]=[N-])c1)OC(=O)ON1C(=O)CCC1=O]

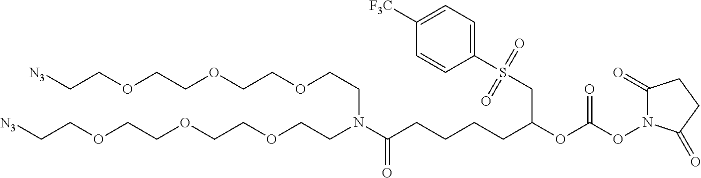 [SMILES: [N-]=[N+]=NCCOCCOCCOCCN(CCOCCOCCOCCN=[N+]=[N-])C(=O)CCCCC(CS(=O)(=O)c1ccc(C(F)(F)F)cc1)OC(=O)ON1C(=O)CCC1=O]

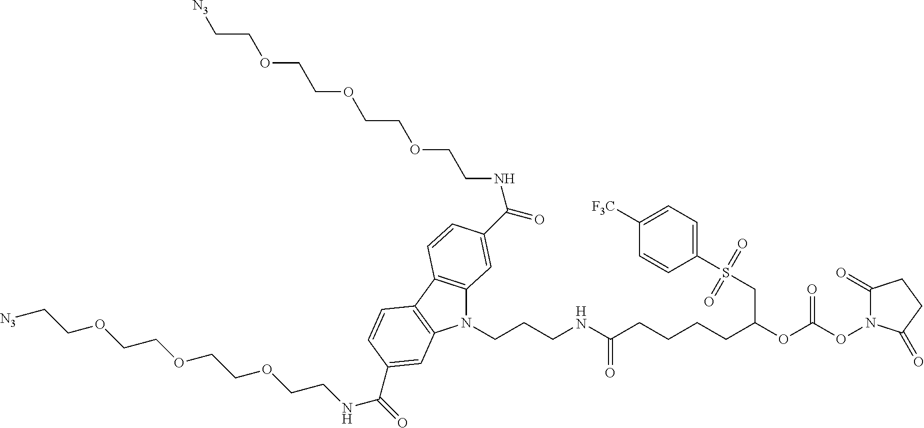 [SMILES: [N-]=[N+]=NCCOCCOCCOCCNC(=O)c1ccc2c3ccc(C(=O)NCCOCCOCCOCCN=[N+]=[N-])cc3n(CCCNC(=O)CCCCC(CS(=O)(=O)c3ccc(C(F)(F)F)cc3)OC(=O)ON3C(=O)CCC3=O)c2c1]

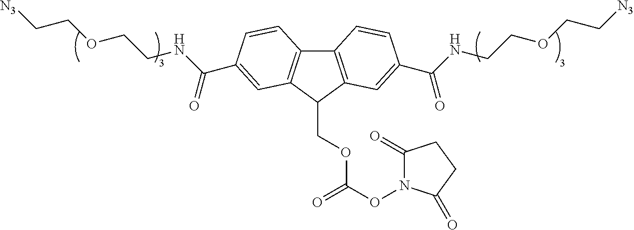

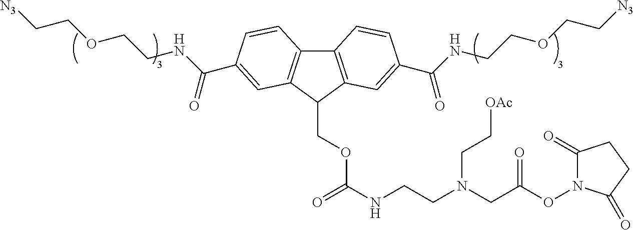

Corresponding Conjugate

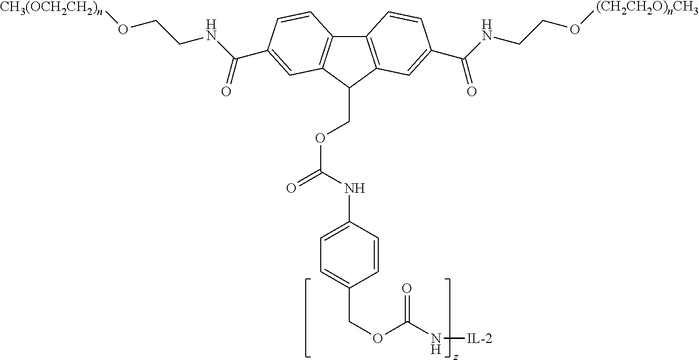

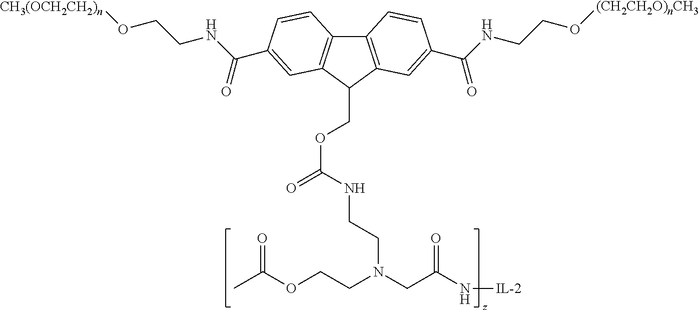

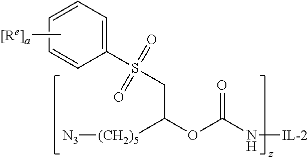

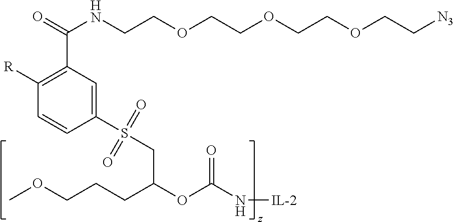

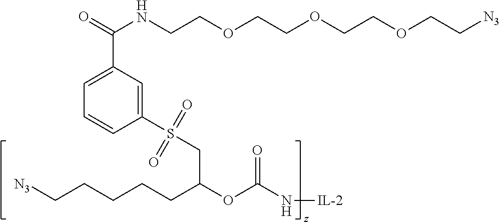

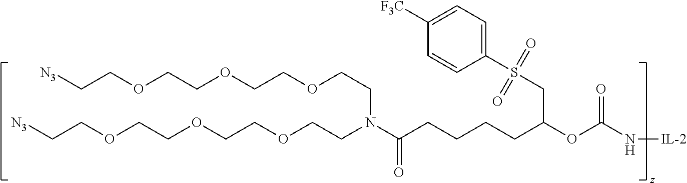

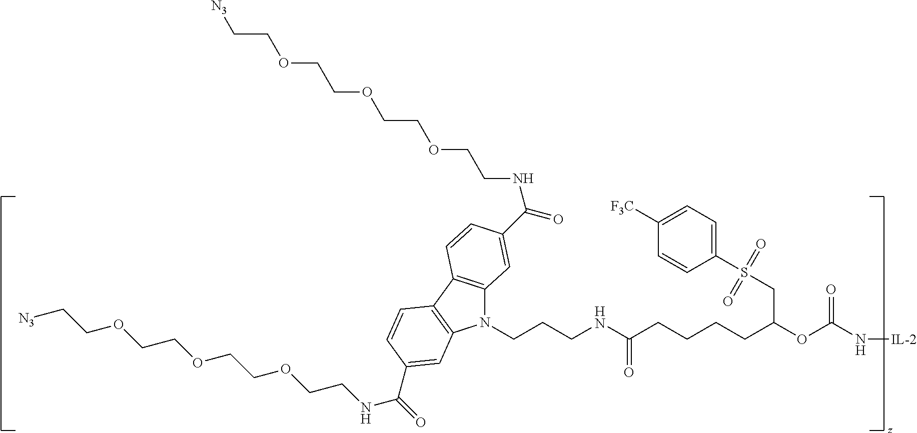

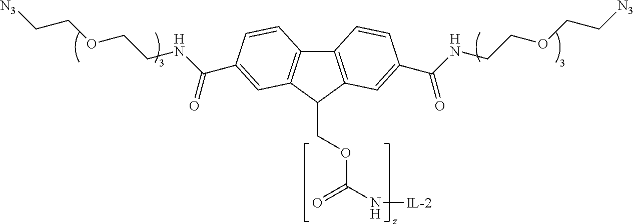

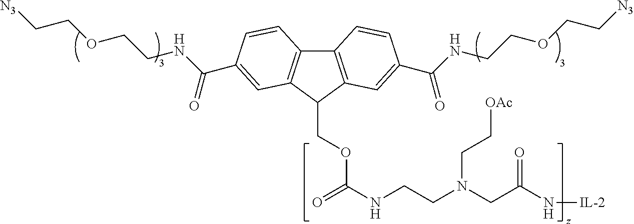

Conjugation of a reagent to an amino group of an IL-2 moiety can be accomplished by a variety of techniques. In one approach, an IL-2 moiety can be conjugated to a coupling reagent functionalized with a succinimidyl derivative (or other activated ester group, wherein approaches similar to those described for these alternative activated ester group-containing reagents can be used). In this approach, the reagent bearing a succinimidyl derivative can be attached to the IL-2 moiety in an aqueous media at a pH of 7 to 9.0, although using different reaction conditions (e.g., a lower pH such as 6 to 7, or different temperatures and/or less than 15° C.) can result in the attachment of the reagent to a different location on the E1-2 moiety.

Since there are multiple amino sites on IL-2, more than one functionalization of IL-2 moiety with the disclosed coupling reagents can be achieved using excess equivalents of the reagents. Very high equivalents of polymeric reagents (eg. 100 eq.) are required to conjugate with multiple amino groups of IL-2 moiety. Utilization of bifunctional linker reagents can achieve high functionalization of IL-2 moiety more efficiently.

The bifunctional linker reagent, in general, can bear a succinimidyl derivative and a reactive group suitable for click chemistry. Conjugation of the bifunctional reagent to amino groups of an IL-2 moiety through NHS coupling can achieve high numbers of functionalization of the IL-2 moiety. Subsequently, click chemistry with suitable polymeric reagents can give highly polymerically derivatized IL-2. Some non-limiting specific examples, along with the corresponding conjugate, are provided in Table 2 below. In the table, the variable (n) represents the number of repeating monomeric units, z is an integer from 1 to 25 and “—NH-IL-2” represents the residue of the IL-2 with one or more water-soluble polymers individually attached. While each polymeric portion [e.g., (OCH₂CH₂)ₙ or (CH₂CH₂O)ₙ] presented in Table 2 terminates in a “CH₃” group, other groups (such as H and benzyl) can be substituted therefor.

TABLE 2
IL-2 Linker Conjugate and the IL-2 Polymeric Conjugate Formed Therefrom
IL-2 Linker Conjugate

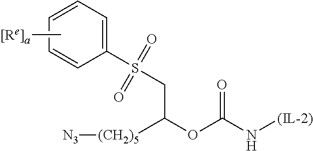

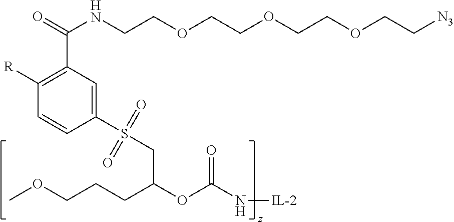

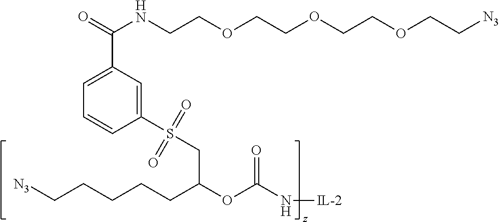

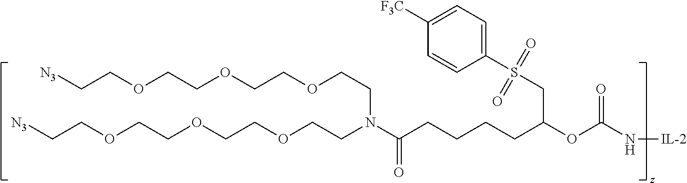

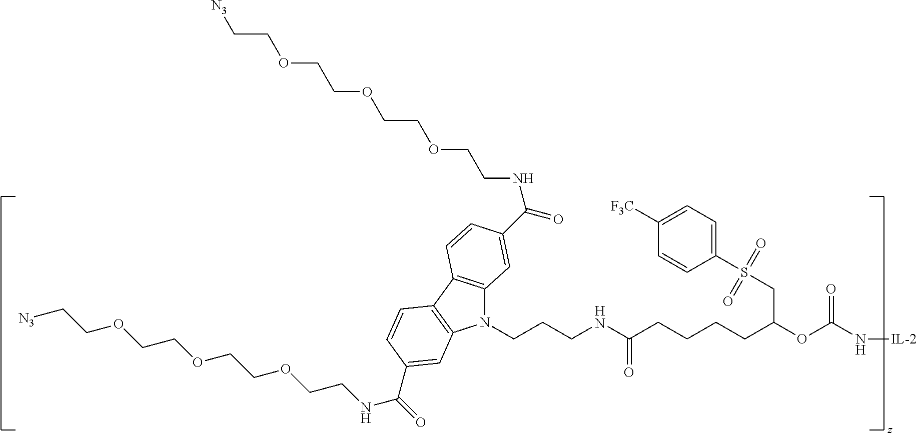

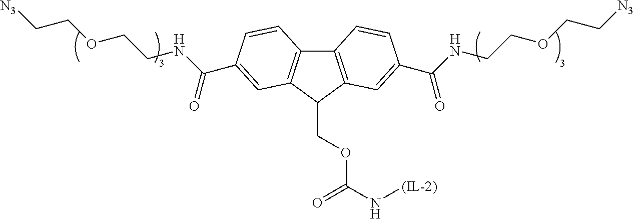

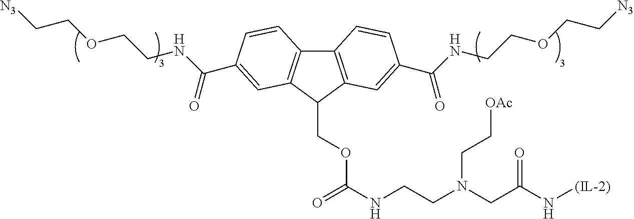

Corresponding Polymeric Conjugate

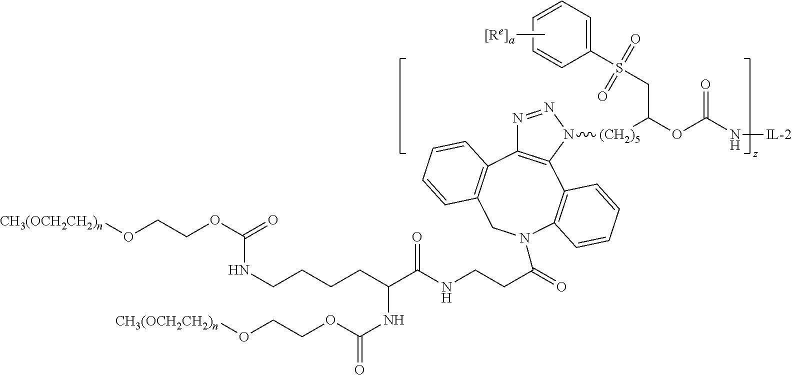

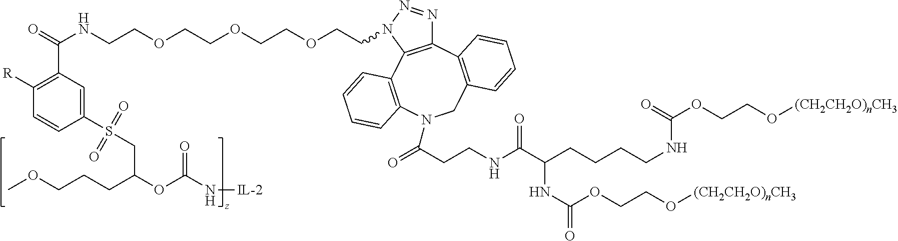

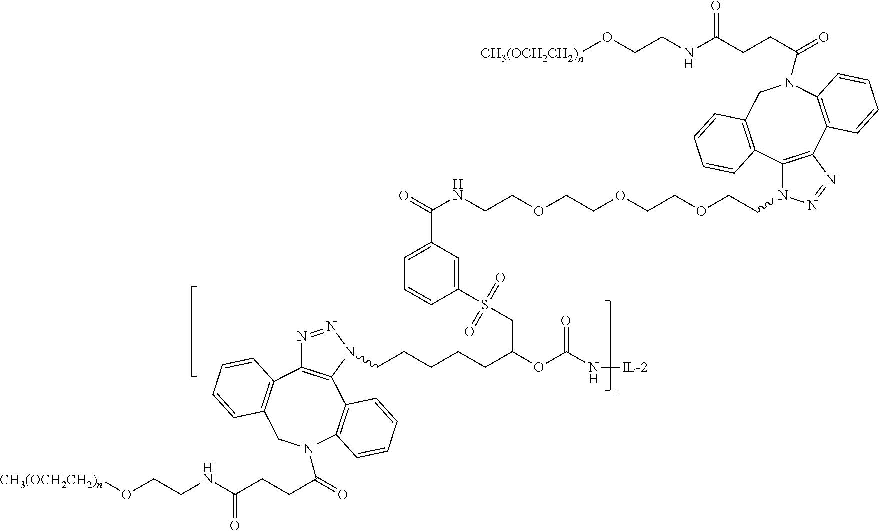

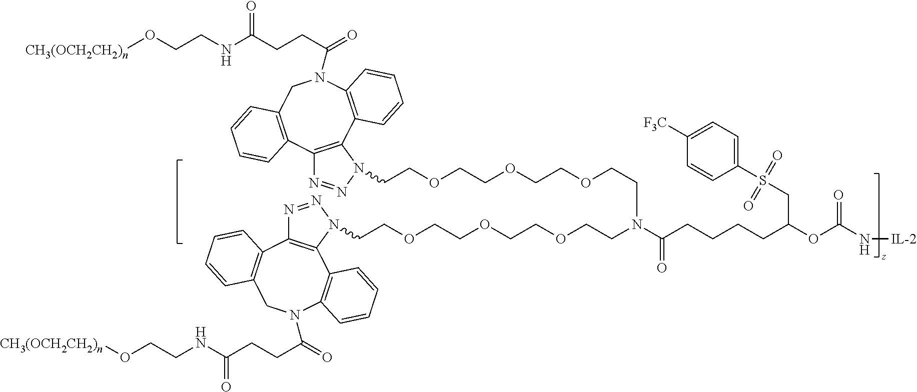

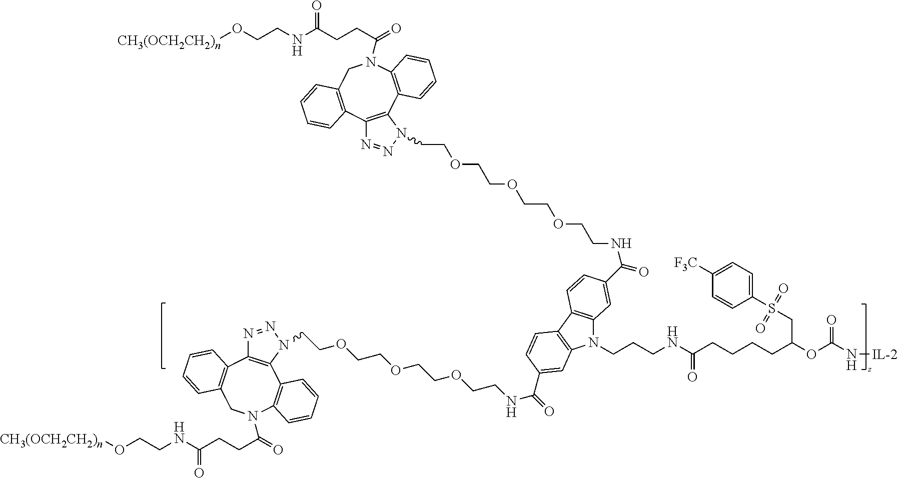

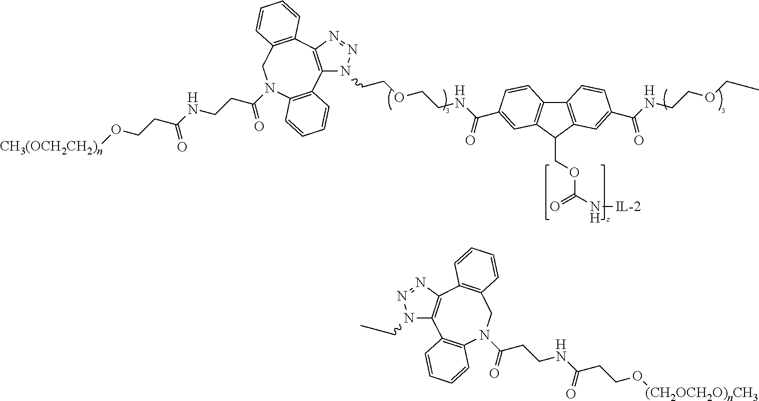

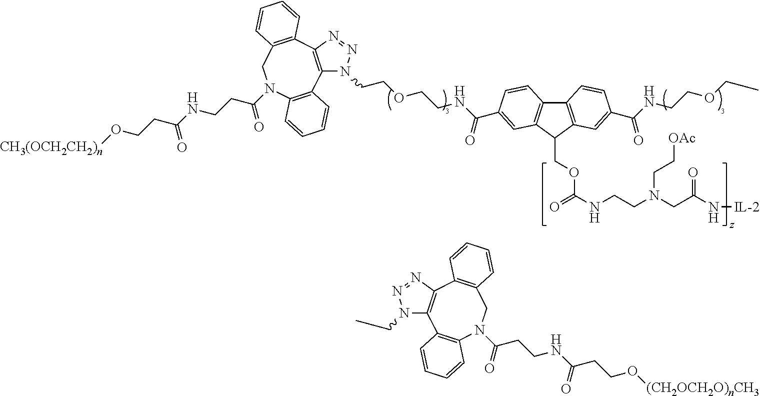

Click chemistry is employed for site-specific PEGylation. The site-specific PEGylation is achieved by incorporation of an azide-containing non-natural amino acid, i.e., a homoazidoalanine into a recombinant protein that allows for site-specific conjugation with an alkyne-PEG molecule.

One major shortcoming of the Cu-catalyzed click reaction is the need for a highly toxic Cu(I) as well as Cu(II). Even in small amounts copper can damage proteins, in particular fluorescent proteins, like GFP. In addition, the presence of reducing agents, ligands and oxygen-free conditions might be required.

A method to achieve site-specific PEGylation with similar efficiency as the Cu-catalyzed click reactions while maintaining protein viability is the introduction of cyclooctynes, where the strain in the eight-membered ring allows the reaction with azides to occur in the absence of catalysts at 4° C. or at room temperature. Dibenzylcyclooctynes, so-called DBCO, belong to this class of reactive cyclooctynes.

DBCO-PEG molecules allow Cu-free PEGylation of an azide-containing protein under mild reaction conditions. Concomitant, the covalent attachment of the PEG molecule to the azide residue is efficient and highly site-specific because of the inherited selectivity of click chemistry.

Click-PEGylation was utilized to convert multiple azide functionalized IL-2 (IL-2-linker conjugates) to multiple PEGylated conjugates (IL-2-polymer conjugates) with high efficiency. When the click reaction occurs between an azide and a non-symmetrical 1,2-disubstituted alkyne, such as DBCO, one of skill in the art would understand that two regioisomeric compounds can be obtained as products. The regioisomers differ in the position of the C—N bond that is formed.

Thiol groups contained within the IL-2 moiety can serve as effective sites of attachment for the water-soluble polymer. There is one solvent accessible disulfide within IL-2 moiety. It typically contributes to the stability of the protein rather than to its structure or its function. As reported in Bioconjugate Chem. 2007, 18, 61-76, mild reduction of an accessible native disulfide bond to liberate the cysteine thiols can be followed by PEGylation with a bis(thiol)-specific reagent. This leads to the bridging of the two cysteine thiols with PEG attached.

A representative conjugate in accordance with the disclosure, using the thiol-bridge PEGylation can include the following formula (XVII):

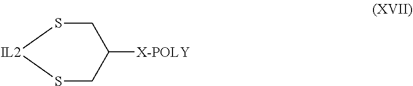

or stereoisomer, a tautomer or mixture thereof, a regioisomeror mixture thereof, or an isotopic variant thereof; or a pharmaceutically acceptable salt, solvate, hydrate, or prodrug thereof; wherein X is a spacer moiety, POLY is a straight or branched water-soluble polymer, and “—S—” is a sulfur group of a residue within the IL-2 moiety. In certain embodiments, the water-soluble polymer is poly(ethylene glycol).

With respect to polymeric reagents, those described here and elsewhere can be purchased from commercial sources or prepared from commercially available starting materials. In addition, methods for preparing the polymeric reagents are described in the literature.

### Click Chemistry

In certain embodiments of the conjugates, linkers, and formula disclosed herein comprise a functional group capable of reacting through click chemistry. As used herein, click chemistry refers to a 1,3-dipolar cycloaddition or [3+2] cycloaddition between an azide and an alkyne to form a 1,2,3-triazole. The terms “1,3-dipolar cycloaddition” and “[3+2] cycloaddition” also encompass “copper-free” 1,3-dipolar cycloadditions between azides and cyclooctynes.

Thus, unless stated otherwise, the description of any triazole compound herein is meant to include regioisomers of a compound, as well as mixtures thereof.

For example, the [3+2] cycloaddition of an azide and alkyne may produce two regioisomeric triazoles as follows:

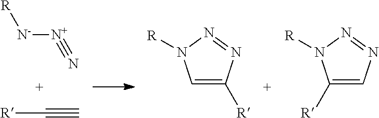

In certain embodiments, the alkyne is a strained cycloalkynyl or heterocycloalkynyl, and the cycloaddition reaction may be performed in the presence or absence of a catalyst. In certain embodiments, for example, the cycloaddition reaction may occur spontaneously by a reaction called strain-promoted azide-alkyne cycloaddition (SPAAC), which is known in the art as “metal-free click chemistry”. In certain embodiments, the strained cycloalkynyl or heterocycloalkynyl is as described herein.

Such catalyst-free [3+2] cycloadditions can be used in methods described herein to form conjugates of the present disclosure. Alkynes can be activated by ring strain such as, by way of example only, eight membered ring structures, appending electron-withdrawing groups to such alkyne rings, or alkynes can be activated by the addition of a Lewis acid such as, Au (I) or Au (III). Alkynes activated by ring strain have been described. For example, the cyclooctynes and difluorocyclooctynes described by Agard et al., J. Am. Chem. Soc, 2004, 126 (46):15046-15047, the dibenzocyclooctynes described by Boon et al., WO2009/067663 A1 (2009), and the aza-dibenzocyclooctynes described by Debets et al., Chem. Comm., 2010, 46:97-99.

In certain embodiments conjugates of the present disclosure can be obtained by reacting a functionalized macromolecule comprising an alkyne group with a functionalized protein comprising an azide group, to form a conjugate, as described herein. In other embodiments the functionalized protein can possess an activated alkyne moiety, and the functionalized macromolecule possesses an azide moiety.

In certain embodiments, the functionalized macromolecule is functionalized PEG. In certain embodiments, the functionalized protein is a functionalized IL-2. In certain embodiments, an azide in a functionalized IL-2 reacts with the alkyne in a functionalized PEG to form a triazole moiety (e.g. via a 1,3-dipolar cycloaddition). In certain embodiments, an azide in a functionalized PEG reacts with the alkyne in a functionalized IL-2 to form a triazole moiety.

In certain embodiments, click chemistry product groups of the present disclosure comprise a triazole group.

In certain embodiments, click chemistry product groups are selected from the group consisting of

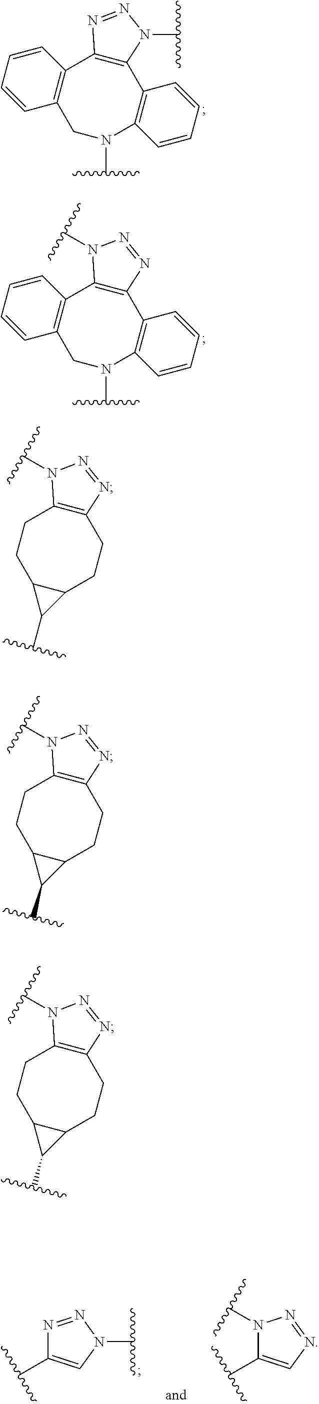

In certain embodiments of the compounds, conjugates, and formula disclosed herein, T is selected from:

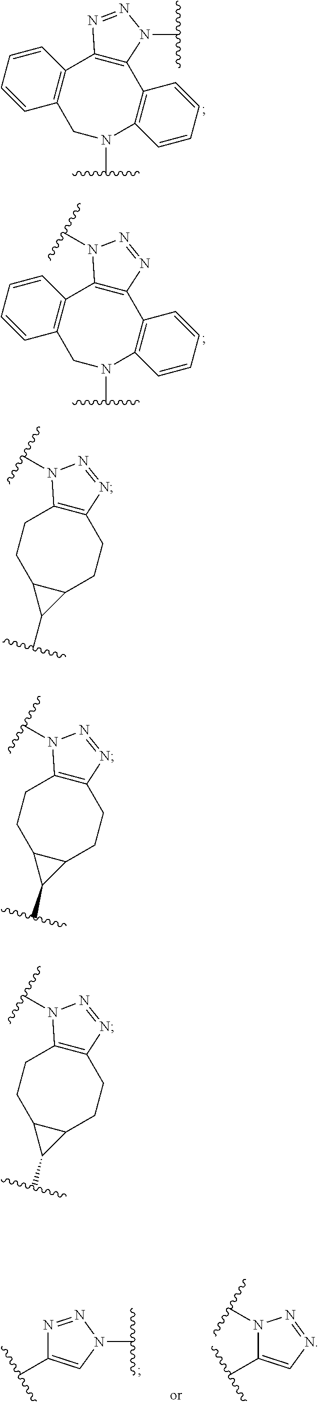

In certain embodiments of the compounds, conjugates, and formulas disclosed herein comprising a triazole functional group (T), the triazole functional group can exist as a mixture of regioisomers resulting in the compounds, or conjugates, to exist as a mixture of regioisomers.

As used herein, the structure of

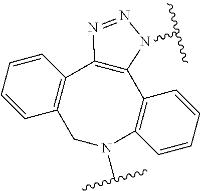

represents the mixture of regioisomers of the following structures:

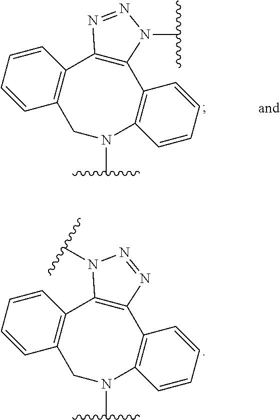 [SMILES: *N1Cc2ccccc2-c2nnn(*)c2-c2ccccc21 ; *N1Cc2ccccc2-c2c(nnn2*)-c2ccccc21]

When a conjugate provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A conjugate provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

The conjugates are typically part of a composition. Generally, the composition comprises a plurality of conjugates. In certain embodiments, each conjugate is comprised of the same protein (i.e., within the entire composition, only one type of protein is found). In addition, the composition can comprise a plurality of conjugates wherein any given conjugate is comprised of a moiety selected from the group consisting of two or more different proteins (i.e., within the entire composition, two or more different proteins are found). In other embodiments, substantially all conjugates in the composition (e.g., 85% or more of the plurality of conjugates in the composition) each comprise the same protein. More specifically, the protein is IL-2.

The composition can comprise a single conjugate species (e.g., a monoPEGylated conjugate, wherein the single polymer is attached at the same location for substantially all conjugates in the composition) or a mixture of conjugate species (e.g., a mixture of monoPEGylated conjugates where attachment of the polymer occurs at different sites and/or a mixture monPEGylated, diPEGylated, triPEGylated and multiple PEGylated conjugates). The compositions can also comprise other conjugates having four, five, six, seven, eight or more polymers attached to any given protein. In addition, the disclosure includes instances wherein the composition comprises a plurality of conjugates, each conjugate comprising one water-soluble polymer covalently attached to one protein, as well as compositions comprising two, three, four, five, six, seven, eight, or more water-soluble polymers covalently attached to one protein. More specifically, the protein is IL-2.

With respect to the conjugates in the composition, the composition will generally satisfy one or more of the following characteristics: at least about 85% of the conjugates in the composition will have from one to ten polymers attached to the protein; at least about 85% of the conjugates in the composition will have from one to nine polymers attached to the protein; at least about 85% of the conjugates in the composition will have from one to eight polymers attached to the protein; at least about 85% of the conjugates in the composition will have from one to seven polymers attached to the protein; at least about 85% of the conjugates in the composition will have from one to six polymers attached to the protein; at least about 85% of the conjugates in the composition will have from one to five polymers attached to the protein; at least about 85% of the conjugates in the composition will have from one to four polymers attached to the protein; at least about 85% of the conjugates in the composition will have from one to three polymers attached to the protein; at least about 85% of the conjugates in the composition will have from one to two polymers attached to the protein; at least about 85% of the conjugates in the composition will have one polymer attached to the protein; at least about 95% of the conjugates in the composition will have from one to ten polymers attached to the protein; at least about 95% of the conjugates in the composition will have from one to nine polymers attached to the protein; at least about 95% of the conjugates in the composition will have from one to eight polymers attached to the protein; at least about 95% of the conjugates in the composition will have from one to seven polymers attached to the protein; at least about 95% of the conjugates in the composition will have from one to six polymers attached to the protein; at least about 95% of the conjugates in the composition will have from one to five polymers attached to the protein; at least about 95% of the conjugates in the composition will have from one to four polymers attached to the protein; at least about 95% of the conjugates in the composition will have from one to three polymers attached to the protein; at least about 95% of the conjugates in the composition will have from one to two polymers attached to the protein; at least about 95% of the conjugates in the composition will have one polymer attached to the protein; at least about 99% of the conjugates in the composition will have from one to ten polymers attached to the protein; at least about 99% of the conjugates in the composition will have from one to nine polymers attached to the protein; at least about 99% of the conjugates in the composition will have from one to eight polymers attached to the protein; at least about 99% of the conjugates in the composition will have from one to seven polymers attached to the protein; at least about 99% of the conjugates in the composition will have from one to six polymers attached to the protein; at least about 99% of the conjugates in the composition will have from one to five polymers attached to the protein; at least about 99% of the conjugates in the composition will have from one to four polymers attached to the protein; at least about 99% of the conjugates in the composition will have from one to three polymers attached to the protein; at least about 99% of the conjugates in the composition will have from one to two polymers attached to the protein; and at least about 99% of the conjugates in the composition will have one polymer attached to the protein. It is understood that a reference to a range of polymers, e.g., “from x to y polymers,” contemplates a number of polymers x to y inclusive (that is, for example, “from one to three polymers” contemplates one polymer, two polymers and three polymers, “from one to two polymers” contemplates one polymer and two polymers, and so forth). More specifically, the protein is IL-2.

Control of the desired number of polymers for any given moiety can be achieved by selecting the proper polymeric reagent, the ratio of polymeric reagent to the protein, temperature, pH conditions, and other aspects of the conjugation reaction. In addition, reduction or elimination of the undesired conjugates can be achieved through purification means.

For example, the polymer-protein moiety conjugates can be purified to obtain/isolate different conjugated species. Specifically, the product mixture can be purified to obtain an average of anywhere from one, two, three, four, five or more PEGs per IL-2 moiety. The strategy for purification of the final conjugate reaction mixture will depend upon a number of factors, including, for example, the molecular weight of the polymeric reagent employed, the particular protein, the desired dosing regimen, and the residual activity and in vivo properties of the individual conjugate(s).

If desired, conjugates having different molecular weights can be isolated using gel filtration chromatography and/or ion exchange chromatography. That is to say, gel filtration chromatography is used to fractionate differently numbered polymer-to-protein moiety ratios (e.g., 1-mer, 2-mer, 3-mer, and so forth, wherein “1-mer” indicates 1 polymer to protein moiety, “2-mer” indicates two polymers to protein moiety, and so on) on the basis of their differing molecular weights (where the difference corresponds essentially to the average molecular weight of the water-soluble polymer portion). For example, in an exemplary reaction where a 15,000 Dalton protein is randomly conjugated to a polymeric reagent having a molecular weight of about 20,000 Daltons, the resulting reaction mixture may contain unmodified protein (having a molecular weight of about 15,000 Daltons), monoPEGylated protein (having a molecular weight of about 35,000 Daltons), diPEGylated protein (having a molecular weight of about 55,000 Daltons), and so forth.

While this approach can be used to separate PEG and other polymer-protein conjugates having different molecular weights, this approach is generally ineffective for separating positional isoforms having different polymer attachment sites within the protein. For example, gel filtration chromatography can be used to separate from each other mixtures of PEG 1-mers, 2-mers, 3-mers, and so forth, although each of the recovered conjugate compositions may contain PEG(s) attached to different reactive groups (e.g., lysine residues) within the protein.

Selection of a particular gel filtration column will depend upon the desired fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin (BSA) as a standard, (iii) iodine testing for PEG content (Sims et al. (1980) Anal. BioIL-2m, 107:60-63), (iv) sodium dodecyl sulfate polyacrylamide gel electrophoresis (SDS PAGE), followed by staining with barium iodide, and (v) high performance liquid chromatography (HPLC).

Separation of positional isoforms is earned out by reverse phase chromatography using a reverse phase-high performance liquid chromatography (R^{p}-HPLC) using a suitable column (e.g., a C18 column or C3 column) or by ion exchange chromatography using an ion exchange column. Either approach can be used to separate polymer-active agent isomers having the same molecular weight (i.e., positional isoforms).

For IL-2-polymer conjugates, the compositions are preferably substantially free of proteins that do not have IL-2 activity. In addition, the compositions preferably are substantially free of all other noncovalently attached water-soluble polymers. In some circumstances, however, the composition can contain a mixture of polymer-IL-2 moiety conjugates and unconjugated IL-2 moiety.

Optionally, the composition of the disclosure further comprises one or more pharmaceutically acceptable carriers or excipients. If desired, the pharmaceutically acceptable excipient can be added to a conjugate to form a composition.

Exemplary excipients include, without limitation, those selected from the group consisting of carbohydrates, inorganic salts, antimicrobial agents, antioxidants, surfactants, buffers, acids, bases, amino acids, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, cyclodextrins, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for one or more embodiments of the present disclosure include benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present disclosure include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88; sorbitan esters; lipids, such as phospholipids such as lecithin and other phosphatidylcholines, phosphatidylethanolamines (although preferably not in liposomal form), fatty acids and fatty esters; steroids, such as cholesterol; and IL-2lating agents, such as EDTA, zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

One or more amino acids can be present as an excipient in the compositions described herein. Exemplary amino acids in this regard include arginine, lysine and glycine.

The amount of the conjugate (i.e., the conjugate formed between the active agent and the polymeric reagent) in the composition will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient, with concentrations less than 30% by weight most preferred.

These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A.H., Handbook of Pharmaceutical Excipients, 3^{rd} Edition, American Pharmaceutical Association, Washington, D.C., 2000.

### Methods of Treatment

The conjugates and compositions thereof may be used to treat any condition that can be remedied or prevented by administration of the conjugate. Those of ordinary skill in the art appreciate which conditions a specific conjugate can effectively treat. For example, the conjugates can be used either alone or in combination with other pharmacotherapy to treat cancers, infectious disease (e.g., viral), and/or autoimmune diseases.

In some embodiments, the present disclosure provides a method of treating cancer in a subject in need thereof, the method comprising, administering to the subject a therapeutically effect amount of a conjugate disclosed herein. In some embodiments, the cancer is a blood cancer. In some embodiments, the blood cancer is multiple myeloma, lymphoma, or leukemia. In some embodiments, the blood cancer is acute myeloid leukemia, non-Hodgkin's lymphoma, cutaneous T-cell lymphoma. In some embodiments, the cancer is a solid tumor cancer. In some embodiments, the solid tumor cancer is renal cell carcinoma, melanoma, breast cancer or bladder cancer. In some embodiments, the melanoma is metastatic melanoma. In some embodiments, the cancer is the cancer that can be treated with IL-2 selected from the group consisting of sarcoma, chordoma, colon cancer, rectal cancer, colorectal cancer, pancreatic cancer, breast cancer, ovarian cancer, prostate cancer, squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, testicular cancer, gastric cancer, non-small cell lung cancer, small cell lung cancer, bladder cancer, renal cell carcinoma, urothelial cancer, epithelial cancer, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, non-Hodgkin's lymphoma, cutaneous T-cell lymphoma, acute myeloid leukemia and leukemias.

In some embodiments, the present disclosure provides a method of an infectious disease in a subject in need thereof, the method comprising, administering to the subject a therapeutically effect amount of a conjugate disclosed herein. In some embodiments, the infectious disease is a viral disease. In some embodiments, the viral disease is human immunodeficiency virus (HIV) or hepatitis C virus (HCV). In some embodiments, the infectious disease is HIV. In some embodiments, the infectious disease is HCV.

In some embodiments, the present disclosure provides a method of an autoimmune disease in a subject in need thereof, the method comprising, administering to the subject a therapeutically effect amount of a conjugate disclosed herein. In some embodiments, the autoimmune disease is rheumatoid arthritis, lupus erythematosus, inflammatory bowel disease (IBD) or atopic dermatitis. In some embodiments, the rheumatoid arthritis is juvenile rheumatoid arthritis.

In certain embodiments, patients are suffering from a malady selected from the group consisting of renal cell carcinoma, metastatic melanoma, hepatitis C virus (HCV), human immunodeficiency virus (HIV), acute myeloid leukemia, non-Hodgkin's lymphoma, cutaneous T-cell lymphoma, juvenile rheumatoid arthritis, atopic dermatitis, breast cancer and bladder cancer.

Advantageously, the conjugate can be administered to the patient prior to, simultaneously with, or after administration of another active agent. In some embodiments, the conjugates can be combined with anti-tumor antigen antibodies to produce synergistic innate and adaptive immune response. In some embodiments, the conjugates can be combined with anti-tumor antibodies that have their anti-tumor activities through antibody-dependent cellular cytotoxicity (ADCC) functions. The PEG-IL-2 conjugates described in this disclosure may stimulate CD8+ T-cells. Stimulation of CD8+ T-cells provides not only the benefit of direct tumor killing, but also the modulation of polymorphonuclear neutrophils (PMNs) for antibody-dependent cellular cytotoxicity (ADCC), such as through the release of cytokines like IFNγ known to promote neutrophil activity (Pelletier et al., J. Leukoc. Biol. 2010; 88:1163-1170). The combination therapy of PEG-IL-2 conjugates with anti-tumor antibodies having ADCC functions could potentially enhance the anti-tumor activities of these antibodies.

### Formulation/Administration

The conjugates and compositions disclosed herein that are administered to patients in need thereof are meant to encompass all types of formulations, in particular those that are suited for injection, e.g., powders or lyophilates that can be reconstituted as well as liquids. Examples of suitable diluents for reconstituting solid compositions prior to injection include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

The compositions of one or more embodiments of the present disclosure are typically, although not necessarily, administered via injection and are therefore generally liquid solutions or suspensions immediately prior to administration. The pharmaceutical preparation can also take other forms such as syrups, creams, ointments, tablets, powders, and the like. Other modes of administration are also included, such as pulmonary, rectal, transdernmal, transmucosal, oral, intrathecal, intratumorally, peritumorally, intraperitoneally, subcutaneous, intra-arterial, and so forth.

The disclosure also provides a method for administering a conjugate as provided herein to a patient suffering from a condition that is responsive to treatment with conjugate. The method comprises administering to a patient, generally via injection, a therapeutically effective amount of the conjugate (preferably provided as part of a pharmaceutical composition). As previously described, the conjugates can be injected (e.g., intramuscularly, subcutaneously and parenterally). Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

The method of administering the conjugate (preferably provides as part of a pharmaceutical composition) can optionally be conducted so as to localize the conjugate to a specific area. For example, the liquid, gel and solid formulations comprising the conjugate could be surgically implanted in a diseased area (such as in a tumor, near a tumor, in an inflamed area, and near an inflamed area). Conveniently, organs and tissue can also be imaged in order to ensure the desired location is better exposed to the conjugate.

The actual dose to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 100 mg, preferably in doses from 0.01 mg/day to 75 mg/day, and more preferably in doses from 0.10 mg/day to 50 mg/day. A given dose can be periodically administered up until, for example, symptoms of diseases lessen and/or are eliminated entirely.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly, once every three weekly, twice monthly, once monthly, and any combination thereof. Once the clinical endpoint has been achieved, dosing of the composition is halted.

It is to be understood that while the disclosure has been described in conjunction with the preferred specific embodiments thereof, that the foregoing description as well as the examples that follow are intended to illustrate and not limit the scope of the disclosure. Other aspects, advantages and modifications within the scope of the disclosure will be apparent to those skilled in the art to which the disclosure pertains.

All articles, books, patents and other publications referenced herein are hereby incorporated by reference in their entireties.

### Experimental

The practice of the disclosure will employ, unless otherwise indicated, conventional techniques of organic synthesis, biochemistry, protein purification and the like, which are within the skill of the art. Such techniques are fully explained in the literature. See, for example, J. March, Advanced Organic Chemistry: Reactions Mechanisms and Structure, 4th Ed. (New York: Wiley-Interscience, 1992), supra.

In the following examples, efforts have been made to ensure accuracy with respect to numbers used (e.g., amounts, temperatures, and so forth), but some experimental error and deviation should be accounted for. Unless otherwise indicated, temperature is in degrees Celsius and pressure is at or near atmospheric pressure at sea level. All reagents are obtained commercially from Sigma-Aldrich or Thermo Fisher Scientific, unless otherwise indicated. All generated NMR are obtained from a 300 or 400 MHz NMR spectrometer. All processing is carried out in glass or glass-lined vessels and contact with metal-containing vessels or equipment is avoided.

MATERIALS: Unless otherwise noted, all organic solvents and reagents (anhydrous CH₂Cl₂, 2-propanol, acetone, NMM and DBCO-amine) were purchased from Sigma Aldrich and were used as received. PyClocK was purchased from Novabiochem®. The 15 kDa, 17 kDa, and

20 kDa Y-PEG-NHS reagent was purchased from JenKem Technology USA and used as received. 5 kDa, 10 kDa and 20 kDa TheraPEG™ reagents were prepared using methods adapted from published procedures (Brocchini et a1, Nat. Protoc. 2006, 1:5, 2241-2252). DL-Dithiothreitol (DTT) was purchased from Melford and a 0.1 M solution was prepared in cell culture grade water (GE Healthcare) prior to use. Materials for buffer preparation were sourced from Thermo Fisher Scientific, Merck and Sigma-Aldrich and were used as received. PBS, pH 7.4 was prepared from DPBS (Sigma-Aldrich) by pH adjustment using 2 M NaOH (VWR). All other materials were purchased from VWR, Sigma-Aldrich, GE Healthcare, Thermo Fisher Scientific and Merck, and were used as received.

All precursor polymeric reagents referred to in these examples are commercially available unless otherwise indicated. Lyophilized powder of IL-2 (“rIL-2”) corresponding to the amino acid sequence of FIG. 1.

The mass and molar amount of the IL-2-PEG conjugates were calculated based on IL-2 amount.

SDS-PAGE analysis

Samples are analyzed by sodium dodecyl sulfate-polyacrylamide gel electrophoresis (SDS-PAGE). Samples are prepared, loaded on the gel and electrophoresis performed as described by the manufacturer.

### Size Exclusive Chromatography

A size exclusive chromatography method is used to purify the prepared PEG-rIL-2 conjugates. Details for the purification process are described below.

### R^{p}-HPLC Analysis

Samples are analyzed by reversed-phase chromatography (R^{p}-HPLC) analysis performed on an HPLC system. Analytical R^{p}-HPLC analysis was carried out on a Dionex 2 UPLC system with an ACE Excel 2superC18 column (Dimensions: 75×2.1 mm id, particle size 2 μm). The linear gradient of 0-100% Buffer B (99.95% MeCN, 0.05% TFA) in Buffer A (94.95% H₂O, 5.0% MeCN, 0.05% TFA) over 10 min was used, with a flow rate of 0.8 mL/min. Sample loading was 10 μg.

### Example 1

### 7-Azido-1-((4-fluorophenyl)sulfonyl)heptan-2-yl (2,5-dioxopyrrolidin-1-yl) carbonate (8)

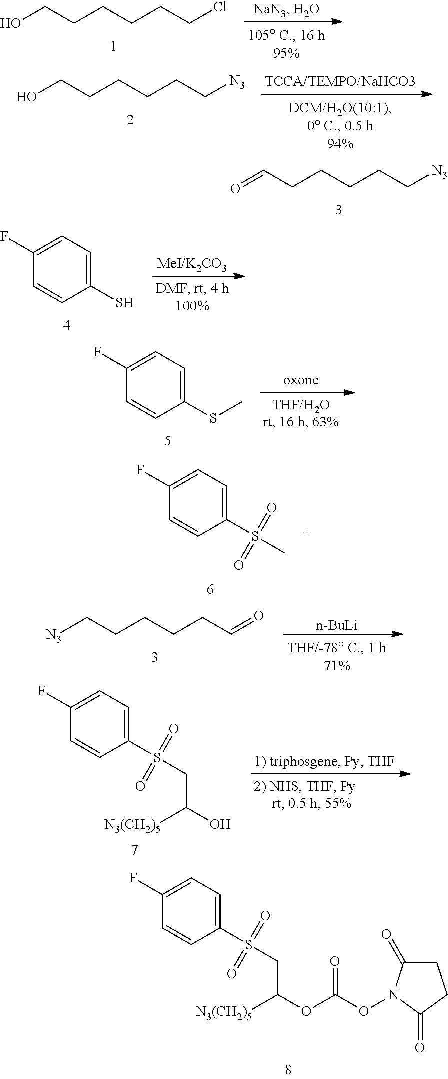

### Preparation of 6-azidohexan-1-ol (2)

To a solution of 6-chlorohexan-1-ol (75 g, 0.549 mol, 1.0 eq) in H₂O (750 mL), was added NaN₃ (97.5 g, 1.50 mol, 2.73 eq). The mixture was stirred at 105° C. for 16 h. LCMS analysis of the reaction mixture showed full conversion to the desired product. Then the mixture was extracted with ethyl acetate. The organic layer was dried over anhydrous Na₂SO₄ and concentrated under reduced pressure to afford crude compound 2 (75 g, 95%).

### Preparation of 6-azidohexanal (3)

To a solution of compound 2 (75 g, 0.523 mol, 1.0 eq), TEMPO (817 mg, 5.23 mmol, 0.01 eq) and NaHCO₃ (52.7 g, 0.628 mol, 1.2 eq) in DCM/H₂O (750 mL/75 mL), was added TCCA (45 g, 0.194 mol, 0.37 eq) in 3 portions at 0° C. The mixture was stirred at 0° C. for 0.5 h. LCMS analysis of the reaction mixture showed full conversion to the desired product. Then the mixture was filtered and diluted with water. The organic layer was dried over anhydrous Na₂SO₄ and concentrated under reduced pressure to afford crude compound 3 (70 g, 94%).

### Preparation of (4-fluorophenyl)(methyl)sulfane (5)

To a solution of compound 4 (30 g, 0.234 mol, 1.0 eq) in DMF (250 ML), was added MeI (40 g, 0.281 mol, 1.2 eq) and K₂CO₃ (97 g, 0.702 mol, 3.0 eq) at room temperature under nitrogen atmosphere. The mixture was stirred at room temperature for 4 h. TLC analysis of the reaction mixture showed full conversion to the desired product. Then the mixture was diluted with water and extracted with ethyl acetate. The organic layer was washed with 5% LiCl (aq.), dried over anhydrous Na₂SO₄ and concentrated under reduced pressure to afford crude compound 5 (45 g, 100%).

### Preparation of 1-fluoro-4-(methylsulfonyl)benzene (6)

To a solution of compound 5 (45 g, 0.317 mol, 1.0 eq) in THF/H₂O (450 mL/450 mL), was added oxone (487 g, 0.792 mol, 2.5 eq) at room temperature under nitrogen atmosphere. The mixture was stirred at room temperature for 16 h. LCMS analysis of the reaction mixture showed full conversion to the desired product. Then the mixture was filtered, diluted with water and extracted with ethyl acetate. The organic layer was washed with brine, dried over anhydrous Na₂SO₄ and concentrated under reduced pressure to afford crude compound 6 (35 g, 63%).

### Preparation of 7-azido-1-((4-fluorophenyl)sulfonyl)heptan-2-ol (7)

To a solution of compound 6 (20 g, 0.115 mol, 1.0 eq) in anhydrous THF (200 mL), was added n-BuLi (2.5 M in hexane, 60 mL, 0.149 mol, 1.3 eq) dropwise at −78° C. The cooling bath was removed and the mixture was allowed to warm to 0° C. After being stirred for 30 min, compound 3 (21 g, 0.149 mol, 1.3 eq) was added at −78° C. After being stirred for 15 min, the mixture was allowed to warm. Then the mixture was added saturated aqueous of NH₄Cl (the mixture became clear) and extracted with ethyl acetate. The organic layer was dried over anhydrous Na₂SO₄ and concentrated under reduced pressure. The residue was purified by silica gel chromatography to afford compound 7 (26 g, 71%).

### Preparation of 7-azido-1-((4-fluorophenyl)sulfonyl)heptan-2-yl (2,5-dioxopyrrolidin-1-yl) carbonate (8)

To a stirred solution of compound 7 (15 g, 47.62 mmol, 1.0 eq) and triphosgene (24 g, 80.95 mmol, 1.7 eq) in anhydrous THF (200 mL), was added pyridine (7.5 g, 95.24 mmol, 2.0 eq) dropwise at room temperature under nitrogen atmosphere. After being stirred for 10 min, the mixture was filtered and concentrated under reduced pressure. The residue was dissolved in anhydrous THF (100 mL) and treated successively with NHS (16.4 g, 0.143 mol, 3.0 eq) and pyridine (11.3 g, 0.143 mmol, 3.0 eq). After being stirred for 10 min, the mixture was concentrated under reduced pressure. The residue was dissolved in ethyl acetate (100 mL) and washed with 0.1 N HCl, water, saturated aq. NaHCO₃ and brine. The organic layer was dried over anhydrous Na₂SO₄ and concentrated under reduced pressure. The residue was purified by silica gel chromatography to afford compound 8 (12 g, 55%) as a solid. ¹H NMR (400 MHz, d₆-DMSO) δ 7.95-7.92 (m, 2H), 7.46 (t, J=8.8 Hz, 2H), 5.10-5.09 (m, 1H), 4.04-3.97 (m, 1H), 3.84 (dd, J=15.2, 2.0 Hz, 1H), 3.27-3.24 (m, 2H), 2.77 (s, 4H), 1.65-1.64 (m, 2H), 1.44-1.42 (m, 2H), 1.23-1.22 (m, 4H).

### Example 2

### 7-Azido-1-((4-(trifluoromethyl)phenyl)sulfonyl)heptan-2-yl (2,5-dioxopyrrolidin-1-yl) carbonate (13)

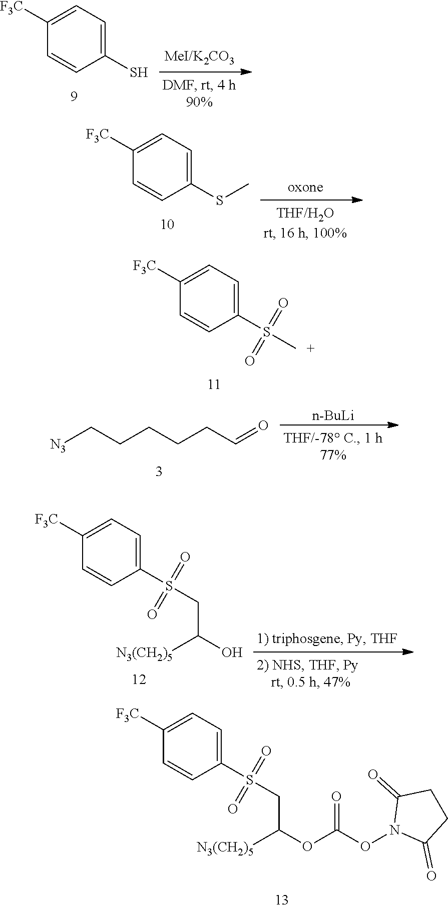

### Preparation of methyl(4-(trifluoromethyl)phenyl)sulfane (10)

To a solution of compound 9 (24.5 g, 0.138 mol, 1.0 eq) in DMF (200 mL), was added MeI (23.4 g, 0.165 mol, 1.2 eq) and K₂CO₃ (57 g, 0.413 mol, 3.0 eq) at room temperature under nitrogen atmosphere. The mixture was stirred at room temperature for 4 h. TLC analysis of the reaction mixture showed full conversion to the desired product. Then the mixture was diluted with water and extracted with ethyl acetate. The organic layer was washed with 5% LiCl (aq.), dried over anhydrous Na₂SO₄ and concentrated under reduced pressure to afford crude compound 10 (24 g, 90%).

### Preparation of 1-(methylsulfonyl)-4-(trifluoromethyl)benzene (11)

To a solution of compound 10 (24 g, 0.125 mol, 1.0 eq) in THF/H₂O (200 mL/200 mL), was added oxone (171 g, 0.264 mol, 2.1 eq) at room temperature under nitrogen atmosphere. The mixture was stirred at room temperature for 16 h. LCMS analysis of the reaction mixture showed full conversion to the desired product. Then the mixture was filtered, diluted with water and extracted with ethyl acetate. The organic layer was washed with brine, dried over anhydrous Na₂SO₄ and concentrated under reduced pressure to afford crude compound 11 (30.6 g, 100%).

### Preparation of 7-azido-1-((4-(trifluoromethyl)phenyl)sulfonyl)heptan-2-ol (12)

To a solution of compound 11 (15 g, 66.96 mmol, 1.0 eq) in anhydrous THF (150 mL), was added n-BuLi (2.5 M in hexane, 35 mL, 87.05 mmol, 1.3 eq) dropwise at −78° C. The cooling bath was removed and the mixture was allowed to warm to 0° C. After being stirred for 30 min, compound 3 (12.5 g, 87.05 mmol, 1.3 eq) was added at −78° C. After being stirred for 15 min, the mixture was allowed to warm. Then the mixture was added saturated aqueous of NH₄Cl (the mixture became clear) and extracted with ethyl acetate. The organic layer was dried over anhydrous Na₂SO₄ and concentrated under reduced pressure. The residue was purified by silica gel chromatography to afford impure compound 12 (19 g, 77%).

### Preparation of 7-azido-1-((4-(trifluoromethyl)phenyl)sulfonyl)heptan-2-yl (2,5-dioxopyrrolidin-1-yl) carbonate (13)

To a stirred solution of compound 12 (19 g, 52.05 mmol, 1.0 eq) and triphosgene (26.3 g, 88.49 mmol, 1.7 eq) in anhydrous THF (200 mL), was added pyridine (8 mL, 0.104 mol, 2.0 eq) dropwise at room temperature under nitrogen atmosphere. After being stirred for 10 min, the mixture was filtered and concentrated under reduced pressure. The residue was dissolved in anhydrous THF (100 mL) and treated successively with NHS (17.95 g, 0.156 mol, 3.0 eq) and pyridine (12.5 mL, 0.156 mmol, 3.0 eq). After being stirred for 10 min, the mixture was concentrated under reduced pressure. The residue was dissolved in ethyl acetate (100 mL) and washed with 0.1 N HCl, water, saturated aq. NaHCO₃ and brine. The organic layer was dried over anhydrous Na₂SO₄ and concentrated under reduced pressure. The residue was purified by silica gel chromatography to afford compound 13 (12.5 g, 47%) as a solid. ¹H NMR (400 MHz, d₆-DMSO) δ 8.10 (d, J=8.4 Hz, 2H), 8.01 (d, J=8.4 Hz, 2H), 5.16-5.15 (m, 1H), 4.16-4.09 (m, 1H), 3.95-3.92 (m, 1H), 3.26 (t, J=6.8 Hz, 2H), 2.77 (s, 4H), 1.66-1.65 (m, 2H), 1.44-1.42 (m, 2H), 1.24-1.23 (m, 4H).

### Example 3

### 7-Azido-1-((4-chlorophenyl)sulfonyl)heptan-2-yl (2,5-dioxopyrrolidin-1-yl) carbonate (18)

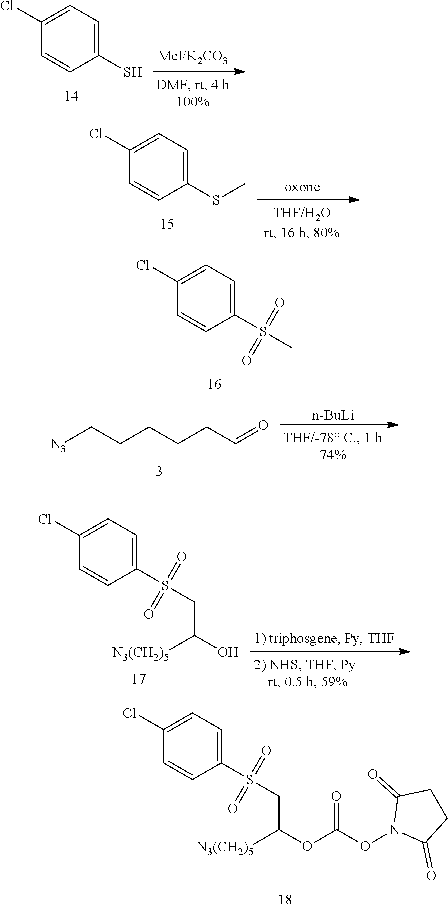

### Preparation of (4-chlorophenylmethyl)sulfane (15)

To a solution of compound 14 (30 g, 0.207 mol, 1.0 eq) in DMF (250 mL), was added MeI (35.3 g, 0.249 mol, 1.2 eq) and K₂CO₃ (85.8 g, 0.622 mol, 3.0 eq) at room temperature under nitrogen atmosphere. The mixture was stirred at room temperature for 4 h. TLC analysis of the reaction mixture showed full conversion to the desired product. Then the mixture was diluted with water and extracted with ethyl acetate. The organic layer was washed with 5% LiCl (aq.), dried over anhydrous Na₂SO₄ and concentrated under reduced pressure to afford crude compound 15 (44 g, 100%) as an orange oil. TLC: PE: EA=10:1, Rf₍₁₄₎=0.5, Rf₍₁₅₎=0.7.

### Preparation of 1-chloro-4-(methylsulfonyl)benzene (16)

To a solution of compound 15 (60 g, 0.380 mol, 1.0 eq) in THF/H₂O (400 mL/400 mL), was added oxone (583 g, 0.948 mol, 2.5 eq) at room temperature under nitrogen atmosphere. The mixture was stirred at room temperature for 16 h. LCMS analysis of the reaction mixture showed full conversion to the desired product. Then the mixture was filtered, diluted with water and extracted with ethyl acetate. The organic layer was washed with brine, dried over anhydrous Na₂SO₄ and concentrated under reduced pressure to afford crude compound 16 (57.8 g, 80%) as a white solid.

### Preparation of 7-azido-1-((4-chlorophenyl)sulfonyl)heptan-2-ol (17)

To a solution of compound 16 (20 g, 0.105 mol, 1.0 eq) in anhydrous THF (300 mL), was added n-BuLi (2.5 M in hexane, 55 mL, 0.137 mol, 1.3 eq) dropwise at −78° C. The cooling bath was removed and the mixture was allowed to warm to 0° C. After being stirred for 30 min, compound 3 (19 g, 0.137 mol, 1.3 eq) was added at −78° C. After being stirred for 15 min, the mixture was allowed to warm. Then the mixture was added saturated aqueous of NH₄Cl (the mixture became clear) and extracted with ethyl acetate. The organic layer was dried over anhydrous Na₂SO₄ and concentrated under reduced pressure. The residue was purified by silica gel chromatography to afford compound 17 (26 g, 74%) as a yellow solid.

### Preparation of 7-azido-1-((4-chlorophenyl)sulfonyl)heptan-2-yl (2,5-dioxopyrrolidin-1-yl) carbonate (18)

To a stirred solution of compound 17 (31 g, 93.42 mmol, 1.0 eq) and triphosgene (47 g, 0.159 mol, 1.7 eq) in anhydrous THF (500 mL), was added pyridine (15 mL, 0.187 mol, 2.0 eq) dropwise at room temperature under nitrogen atmosphere. After being stirred for 10 min, the mixture was filtered and concentrated under reduced pressure. The residue was dissolved in anhydrous THF (500 mL) and treated successively with NHS (32 g, 0.280 mol, 3.0 eq) and pyridine (22 mL, 0.280 mmol, 3.0 eq). After being stirred for 10 min, the mixture was concentrated under reduced pressure. The residue was dissolved in ethyl acetate (300 mL) and washed with 0.1 N HCl, water, saturated aq. NaHCO₃ and brine. The organic layer was dried over anhydrous Na₂SO₄ and concentrated under reduced pressure. The residue was purified by silica gel chromatography to afford compound 18 (26 g, 59%) as a solid. ¹H NMR (400 MHz, d₆-DMSO) δ 7.87 (d, J=8.8 Hz, 2H), 7.69 (d, J=8.8 Hz, 2H), 5.11-5.10 (m, 1H), 4.06-4.00 (m, 1H), 3.86 (dd, J=15.6, 2.4 Hz, 1H), 3.26 (t, J=6.8 Hz, 2H), 2.77 (s, 4H), 1.66-1.62 (m, 2H), 1.45-1.42 (m, 2H), 1.23-1.22 (m, 4H).

### Example 4

### 7-Azido-1-((2,4-difluorophenyl)sulfonyl)heptan-2-yl (2,5-dioxopyrrolidin-1-yl) carbonate (19)

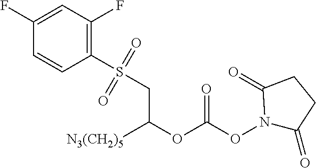

Under similar preparation procedure as example 1, example 4 was prepared using 2,4-difluorobenzenethiol. ¹H NMR (400 MHz, CDCl₃) δ 8.01-7.94 (m, 1H), 7.12-7.05 (m, 1H), 7.05-6.97 (m, 1H), 5.24 (d, J=6.6 Hz, 1H), 3.78 (dd, J=15.2, 8.4 Hz, 1H), 3.46 (dd, J=15.2, 3.4 Hz, 1H), 3.26 (t, J=6.8 Hz, 2H), 2.80 (s, 4H), 1.79 (s, 2H), 1.63-1.56 (m, 2H), 1.43-1.33 (m, 4H).

### Example 5

### 7-Azido-1-((4-fluoro-2-(trifluoromethyl)phenyl)sulfonyl)heptan-2-yl (2,5-dioxopyrrolidin-1-yl) carbonate (20)

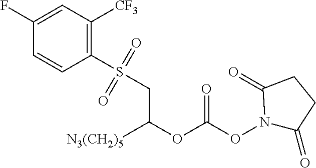

Under similar preparation procedure as example 1, example 5 was prepared using 4-fluoro-2-(trifluoromethyl)benzenethiol. ¹H NMR (400 MHz, CDCl₃) δ 8.31 (dd, J=8.8, 5.2 Hz, 1H), 7.60 (dd, J=8.8, 2.6 Hz, 1H), 7.54-7.46 (m, 1H), 5.36-5.26 (m, 1H), 3.79 (dd, J=15.2, 8.8 Hz, 1H), 3.47 (dd, J=15.2, 3.2 Hz, 1H), 3.25 (t, J=6.8 Hz, 2H), 2.81 (s, 4H), 1.83-1.70 (m, 2H), 1.61-1.52 (m, 2H), 1.45-1.34 (m, 4H).

### Example 6

### (2,7-Bis((2-(2-(2-(2-azidoethoxy)ethoxy)ethoxy)ethylcarbamoyl)-9H-fluoren-9-yl)methyl (25-dioxopyrrolidin-1-yl) carbonate (24)

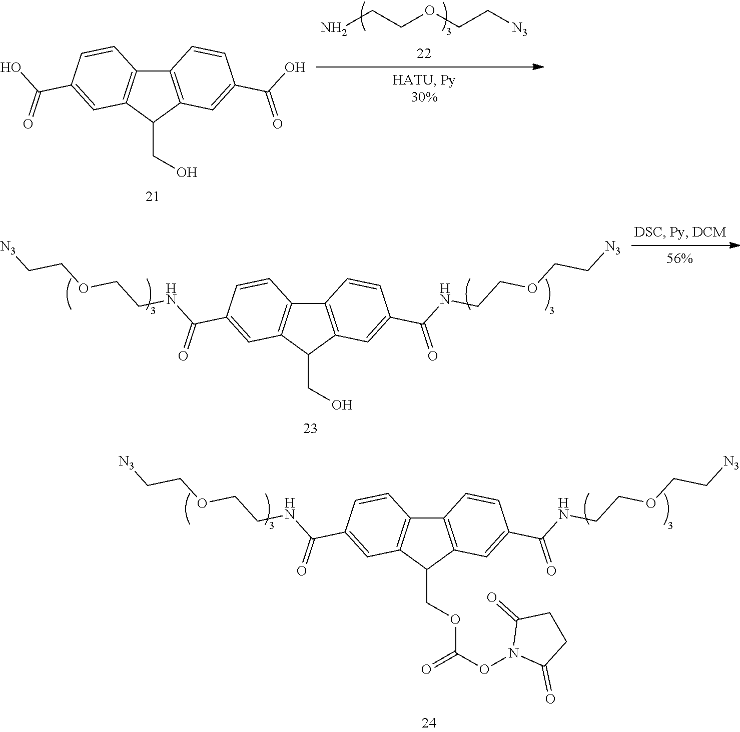

### Preparation of N₂,N₇-bis(2-(2-(2-(2-azidoethoxy)ethoxy)ethoxy)ethyl)-9-(hydroxymethyl)-9H-fluorene-2,7-dicarboxamide (23)

9-(Hydroxymethyl)-9H-fluorene-2,7-dicarboxylic acid (82.5 mg, 0.24 mmol) was dissolved in anhydrous pyridine (1.0 mL) and to the solution was added HATU (273.8 mg, 0.72 mmol) and 2-(2-(2-(2-azidoethoxy)ethoxy)ethoxy)ethan-1-amine (117.1 mg, 0.54 mmol) at rt. Then the reaction was stirred for 2 hrs. The product was purified with HPLC in 0-70% MeCN/H₂O (with 0.1% formic acid) to give compound 23 (47.4 mg, 30%). LCMS: m/z 685 (M+1)⁺.

### Preparation of (2,7-bis((2-(2-(2-(2-azidoethoxy)ethoxy)ethoxy)ethyl)carbamoyl)-9H-fluoren-9-yl)methyl (2,5-dioxopyrrolidin-1-yl) carbonate (24)

Compound 23 (47.4 mg, 0.069 mmol) was dissolved in DCM (0.2 mL) and treated with DSC (35.47 mg, 0.14 mmol) and pyridine (16.7 μL, 0.21 mmol) at rt under N₂. The reaction stirred for 1.5 hrs, and then diluted with DCM and washed with 1 N HCl and brine. The organic phase was dried over Na₂SO₄ and concentrated. The residue was purified with HPLC in MeCN/H₂O (with 0.1% TFA) to give the desired product 24 (31.7 mg, 56%, light yellow oil). LCMS: m/z 826 (M+1)⁺.

### Example 7

(2-((2-(2-(2-(2-Azidoethoxy)ethoxy)ethoxy)ethyl)carbamoyl)-9H-fluoren-9-yl)methyl (2,5-dioxopyrrolidin-1-yl) carbonate (31)

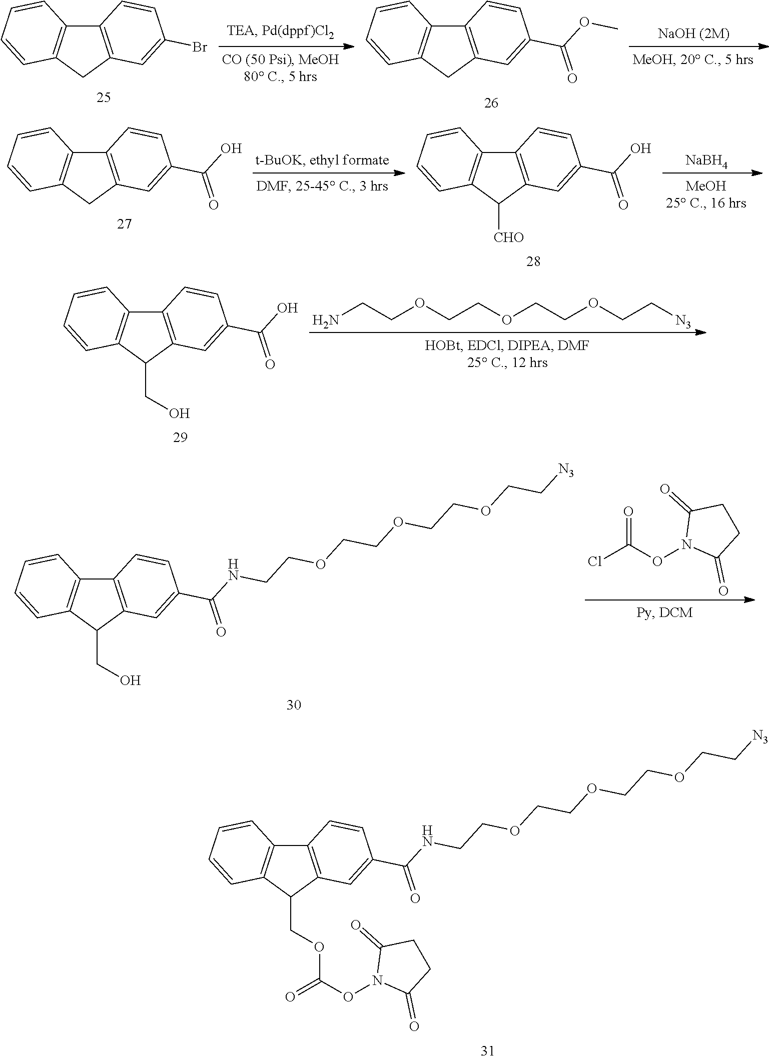

### Preparation of methyl 9H-fluorene-2-carboxylate (26)

A mixture of compound 25, 2-bromo-9H-fluorene (128 g, 522 mmol), triethylamine, TEA (106 g, 1.04 mol, 145 mL) and Pd(dppf)Cl₂ (38.2 g, 52.2 mmol) in MeOH (890 mL) was degassed and purged with CO (50 Psi) for 3 times, and then the mixture was stirred at 80° C. for 5 hrs under N₂ atmosphere. TLC (Petroleum ether/Ethyl acetate=10/1) showed the new spot (Rf=0.42) was formed. The residue was purified by column chromatography (SiO₂, Petroleum ether/Ethyl acetate=100/1 to 10/1) to give compound 26 (120 g, crude) as a white solid.

### Preparation of 9H-fluorene-2-carboxylic acid (27)

To a mixture of compound 26 (120 g, 535 mmol) in MeOH (840 mL), was added NaOH (2 M), and then the mixture was stirred at 20° C. for 5 hrs under N₂ atmosphere. TLC (Petroleum ether/Ethyl acetate=10/1) showed the starting material was consumed completely and the new spot (Rf=0.01) was formed. The solution was added water (50 mL) and then it was extracted with EtOAc (100 mL). The aqueous phase was adjusted to pH 3 with 3M HCl, then it was extracted with EtOAc (100 mL). The organic phase was concentrated under reduced pressure to give compound 27 (40.0 g, 190 mmol, 35.6% yield) as a yellow solid.

### Preparation of 9-formyl-9H-fluorene-2-carboxylic acid (28)

To a mixture of compound 27 (6.00 g, 28.5 mmol) in DMF (196 mL), was added ethyl formate (276 g, 3.73 mol) and t-BuOK (25.6 g, 228 mmol) slowly. The mixture was stirred at 45° C. for 0.5 hr, then was cooled to 25° C. for 2.5 hrs. TLC (Petroleum ether/Ethyl acetate=0/1) showed the starting material was consumed completely and the new spot (Rf=0.48) was formed. The solution was adjusted to pH 3 with 1M HCl. Then the mixture was extracted with EtOAc (50.0 mL). The organic phase was separated, dried over Na₂SO₄, filtered, concentrated under reduced pressure to give compound 28 (7.00 g, crude) as a brown solid.

### Preparation of 9-(hydroxymethyl)-9H-fluorene-2-carboxylic acid (29)

To a mixture of compound 28 (7.00 g, 29.4 mmol) in MeOH (42.0 mL), was added NaBH₄ (2.78 g, 73.5 mmol). The reaction mixture was degassed and purged with N₂ for 3 times, and then the mixture was stirred at 25° C. for 16 hrs under N₂ atmosphere. LCMS (product: RT=0.863 min) showed the desired compound MS. The solution was added water (120 mL) and then it was extracted with EtOAc (100 mL). The aqueous phase was adjusted to pH 3 with 1M HCl, then it was extracted with EtOAc (100 mL). The organic phase was separated, dried over Na₂SO₄, filtered, and concentrated under reduced pressure to give compound 29 (4.00 g, 16.7 mmol, 56.7% yield) as a yellow solid.

### Preparation of N-(2-(2-(2-(2-azidoethoxy)ethoxy)ethoxy)ethyl)-9-(hydroxymethyl)-9H-fluorene-2-carboxamide (30)

To a solution of compound 29 (1.00 g, 4.16 mmol,) and 2-(2-(2-(2-azidoethoxy)ethoxy)ethoxy)ethan-1-amine (908 mg, 4.16 mmol) in DMF (7.00 mL) was added HOBt (619 mg, 4.58 mmol), EDCl (878 mg, 4.58 mmol) and DIPEA (1.24 g, 9.57 mmol) at 25° C. The mixture was stirred at 25° C. for 12 hrs. LCMS (product: RT=1.002 min) showed the starting material was consumed completely. The reaction mixture was diluted with water (10.0 mL), extracted with EtOAc (10.0 mL×2). The combined organic phase was washed with water (10.0 mL×2) and brine (10.0 mL). The organic phase was separated, dried over Na₂SO₄, filtered and concentrated under reduced pressure to give a residue. The residue was purified by prep-HPLC (column: Welch Xtimate C18 250*50 mm*10 um; mobile phase: [water (10 mM NH₄HCO₃)-ACN]; B %: 18%-48%, 26 min) to afford compound 30 (1.40 g, 3.17 mmol, 76.2% yield, 99.8% purity) as a yellow oil. ¹H NMR (400 MHz, CDCl₃): δ 8.10 (s, 1H), 7.88-7.76 (m, 3H), 7.63 (d, J=7.2 Hz, 1H), 7.46-7.34 (m, 2H), 6.98 (s, 1H), 4.18-4.08 (m, 2H), 4.02-3.92 (m, 1H), 3.76-3.56 (m, 14H), 3.32 (t, J=5.2 Hz, 2H), 2.37 (s, 1H); LC-MS: m/z 441.1 (M+1)⁺.

### Preparation of (2-((2-(2-(2-(2-azidoethoxy)ethoxy)ethoxy)ethyl)carbamoyl)-9H-fluoren-9-yl)methyl (2,5-dioxopyrrolidin-1-yl) carbonate (31)

A solution of 1-hydroxypyrrolidine-2,5-dione (0.5 g, 1 eq) in DCM (5 mL) was cooled to −30° C. To this solution, was added dropwise trichloromethyl carbonochloridate (860 mg, 1 eq), and followed by adding dropwise DIPEA (561 mg, 1 eq) at −30° C. The mixture was warmed to 0° C. and stirred for 3 hrs. It was warmed to 25° C. and continued to stir for 6 hrs. TLC (Petroleum ether/Ethyl acetate=0/1, Rf=0.3) showed the starting material was consumed completely. The reaction mixture was filtered to give the filtrate (DCM solution of 2,5-dioxopyrrolidin-1-yl carbonochloridate) which was used directly without further purification.

To a solution of compound 30 (0.1 g, 1 eq) and Py (17.96 mg, 1 eq) in DCM (1 mL) was added 2,5-dioxopyrrolidin-1-yl carbonochloridate (10 eq, a DCM solution from the previous step) at 0° C. The mixture was stirred at 25° C. for 12 hrs. LCMS (starting material: RT=0.992 min, product: RT=1.059 min) showed 3.71% of the starting material was remained and 40.2% of the desired compound was detected. The reaction was quenched by water (2.0 mL), then adjust pH to 6 with saturated citric acid aqueous solution. The mixture was extracted with DCM (2 mL×2). The combined organic layers were washed with brine (5.0 mL), and then dried over Na₂SO₄, filtered and concentrated under reduced pressure to give a residue. The residue was purified by prep-HPLC (column: Welch Ultimate AQ-C18 150*30 mm*5 um; mobile phase: [water (0.1% TFA)-ACN]; B %: 30%-60%, 12 min). After prep-HPLC purification, the fraction was lyophilized to give compound 31 as a colorless oil. LC-MS: m/z 582.2 (M+1)⁺.

### Example 8

(2-((2-(2-(2-(2-Azidoethoxy)ethoxy)ethoxy)ethyl)carbamoyl)-7-fluoro-9H-fluoren-9-yl)methyl (25-dioxopyrrolidin-1-yl) carbonate (39)

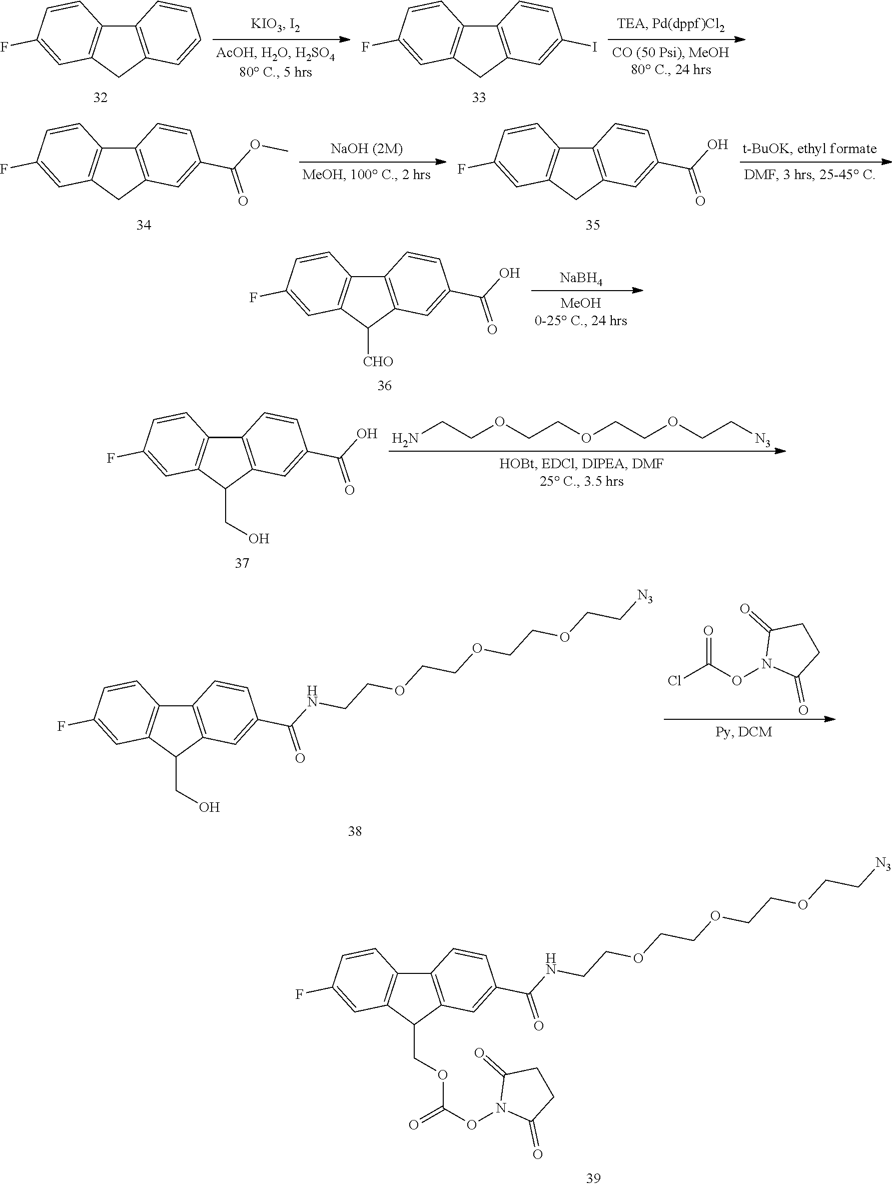

### Preparation of 2-fluoro-7-iodo-9H-fluorene (33)

A mixture of 2-fluoro-9H-fluorene 32 (24.4 g, 132 mmol), 12 (14.1 g, 55.6 mmol) and KIO₃ (7.08 g, 33.1 mmol) in CH₃COOH (408 mL), H₂SO₄ (9.60 mL) and H₂O (19.2 mL), was degassed and purged with N₂ for 3 times. The mixture was stirred at 80° C. for 5 hrs under N₂ atmosphere. HPLC (product: RT=3.515 min) showed the desired compound was detected. The aqueous solution was extracted with EtOAc (50.0 mL). The organic layer was washed with H₂O (20.0 mL), brine (10.0 mL), separated, dried over Na₂SO₄, filtered and concentrated under reduced pressure to give compound 33 (38.0 g, 123 mmol, 92.6% yield) as a brown solid. ¹H NMR (400 MHz, MeOD): 7.87 (s, 1H), 7.70-7.67 (m, 2H), 7.48-7.46 (m, 1H), 7.27-7.22 (m, 1H), 7.17-7.09 (m, 1H), 3.86 (s, 2H).

### Preparation of methyl 7-fluoro-9H-fluorene-2-carboxylate (34)

A mixture of compound 33 (38.0 g, 123 mmol), TEA (31.0 g, 306 mmol), Pd(dppf)Cl₂ (8.97 g, 12.3 mmol) in MeOH (200 mL) was degassed and purged with CO (50 Psi) for 3 times. The mixture was stirred at 80° C. for 24 hrs under CO atmosphere. TLC (Petroleum ether/Ethyl acetate=100/1) showed the starting material was consumed completely and the new spots (R_{f}=0.40) were formed. The solution was concentrated under reduced pressure to give compound 34 (40.0 g, crude) as a brown solid.

### Preparation of 7-fluoro-9H-fluorene-2-carboxylic acid (35)

To a mixture of compound 34 (40.0 g, 165 mmol) in MeOH (280 mL), was added NaOH (2 M, 206 mL, 2.5 eq) aqueous solution. The reaction mixture was stirred at 100° C. for 2 hrs under N₂ atmosphere. TLC (Petroleum ether/Ethyl acetate=0/1) showed the starting material was consumed completely and the new spot (R_{f}=0.03) was formed. The reaction solution was added H₂O (150 mL). Then it was extracted with EtOAc (250 mL). The aqueous layer was separated, and adjusted pH to 3 with 1M HCl. It was extracted with EtOAc (200 mL). The organic layer was washed with brine (20.0 mL), separated, dried over Na₂SO₄, filtered, and concentrated under reduced pressure to give compound 35 (33.0 g, 145 mmol, 87.6% yield) as a brown solid.

### Preparation of 7-fluoro-9-formyl-9H-fluorene-2-carboxylic acid (36)

To a mixture of compound 35 (33.0 g, 145 mmol) in DMF (210 mL), was added ethyl formate (507 g, 6.84 mol). Then t-BuOK (130 g, 1.16 mol) was added slowly. The mixture was stirred at 45° C. for 0.5 hr, then the mixture was cooled to 25° C. for 2.5 hrs. LCMS (product: RT=0.889) showed the desired compound was detected. The reaction solution was added water (150 mL), and extracted with EtOAc (500 mL). The aqueous phase was adjusted to pH 3 with 1M HCl, then it was extracted with EtOAc (500 mL). The organic layer was washed with brine (120 mL), separated, dried over Na₂SO₄, filtered, concentrated under reduced pressure to give compound 36 (30.0 g, crude) as a yellow solid.

### Preparation of 7-fluoro-9-(hydroxymethyl)-9H-fluorene-2-carboxylic acid (37)

To a mixture of compound 36 (30.0 g, 117 mmol) in MeOH (210 mL), was added NaBH₄ (31.0 g, 820 mmol) and then the mixture was stirred at 25° C. for 24 hrs under N₂ atmosphere. LCMS (product: RT=0.906 min) showed the desired compound was detected. The reaction solution was added water (150 mL), and extracted with EtOAc (450 mL). The aqueous phase was adjusted to pH 3 with 1M HCl. Then it was extracted with EtOAc (300 mL). The organic layer was washed with brine (120 mL), separated, dried over Na₂SO₄, filtered, concentrated under reduced pressure to give compound 37 (35.0 g, crude) as a yellow solid.

### Preparation of N-(2-(2-(2-(2-azidoethoxy)ethoxy)ethoxy)ethyl)-7-fluoro-9-(hydroxymethyl)-9H-fluorene-2-carboxamide (38)

A mixture of compound 37 (2.00 g, 7.74 mmol), HOBt (1.15 g, 8.52 mmol), EDCl (1.63 g, 8.52 mmol) and DIPEA (2.50 g, 19.4 mmol) in DMF (14.0 mL) was stirred at 25° C. for 0.5 hr. Then the mixture was added 2-[2-[2-(2-azidoethoxy)ethoxy]ethoxy]ethanamine (1.86 g, 8.52 mmol). The reaction mixture was stirred at 25° C. for 3 hrs. LCMS (product: RT=1.171 min) showed the desired compound was detected. The reaction solution was diluted with water (20 mL) and extracted with EtOAc (20 mL). The organic layer was washed with brine (20.0 mL), separated, dried over Na₂SO₄, filtered and concentrated under reduced pressure to give a residue. The crude product was purified by prep-HPLC (column: Phenomenex luna c18 250 mm*100 mm*10 um; mobile phase: [water(0.1% TFA)-ACN]; B %: 15%-53%, 25 min) to afford compound 38 (1.00 g, 2.12 mmol, 48.7% yield, 97.4% purity) as a yellow oil. ¹H NMR (400 MHz CDCl₃): δ 8.07 (s, 1H), 7.85 (d, J=7.8 Hz, 1H), 7.76-7.70 (m, 2H), 7.35 (d, J=7.2 Hz, 1H), 7.39-7.31 (m, 1H), 7.18-7.08 (m, 1H), 7.02 (s, 1H), 4.16-3.96 (m, 3H), 3.76-3.56 (m, 14H), 3.33 (t, J=4.8 Hz, 2H); LC-MS: m/z 459.1 (M+1)⁺.

### Preparation of (2-((2-(2-(2-(2-azidoethoxy)ethoxy)ethoxy)ethyl)carbamoyl)-9H-fluoren-9-yl)methyl (2,5-dioxopyrrolidin-1-yl) carbonate (39)

To a solution of compound 38 (0.1 g, 1 eq) in DCM (1 mL), was added compound 2,5-dioxopyrrolidin-1-yl carbonochloridate (10 eq, a DCM solution) at 0° C. The reaction mixture was stirred at 25° C. for 12 hrs. LCMS (starting material: RT=1.026 min, product: RT=1.084 min) showed 6.33% of the starting material was remained and 28.3% of the desired compound was detected. The reaction mixture was adjusted to pH 6 with saturated citric acid aqueous solution. The mixture was extracted with DCM (2 mL×2). The combined organic layers were washed with brine (5.0 mL), separated, dried over Na₂SO₄, filtered and concentrated under reduced pressure to give a residue. The residue was purified by prep-HPLC (column: Phenomenex Luna C18 200*40 mm*10 um; mobile phase: [water (0.1% TFA)-ACN]; B %: 25%-55%, 10 min). After prep-HPLC purification, the solution was lyophilized to give compound 39 as a colorless oil. LC-MS: m/z 600.2 (M+1)⁺.

### Example 9

### 2,5-dioxopyrrolidin-1-vi N-(2-acetoxyethyl)-N-(2-((((2,7-bis((2-(2-(2-(2-azidoethoxy)ethoxy)ethoxy)ethyl)carbamoyl)-9H-fluoren-9-yl)methoxy)carbonyl)amino)ethyl)glycinate (44)

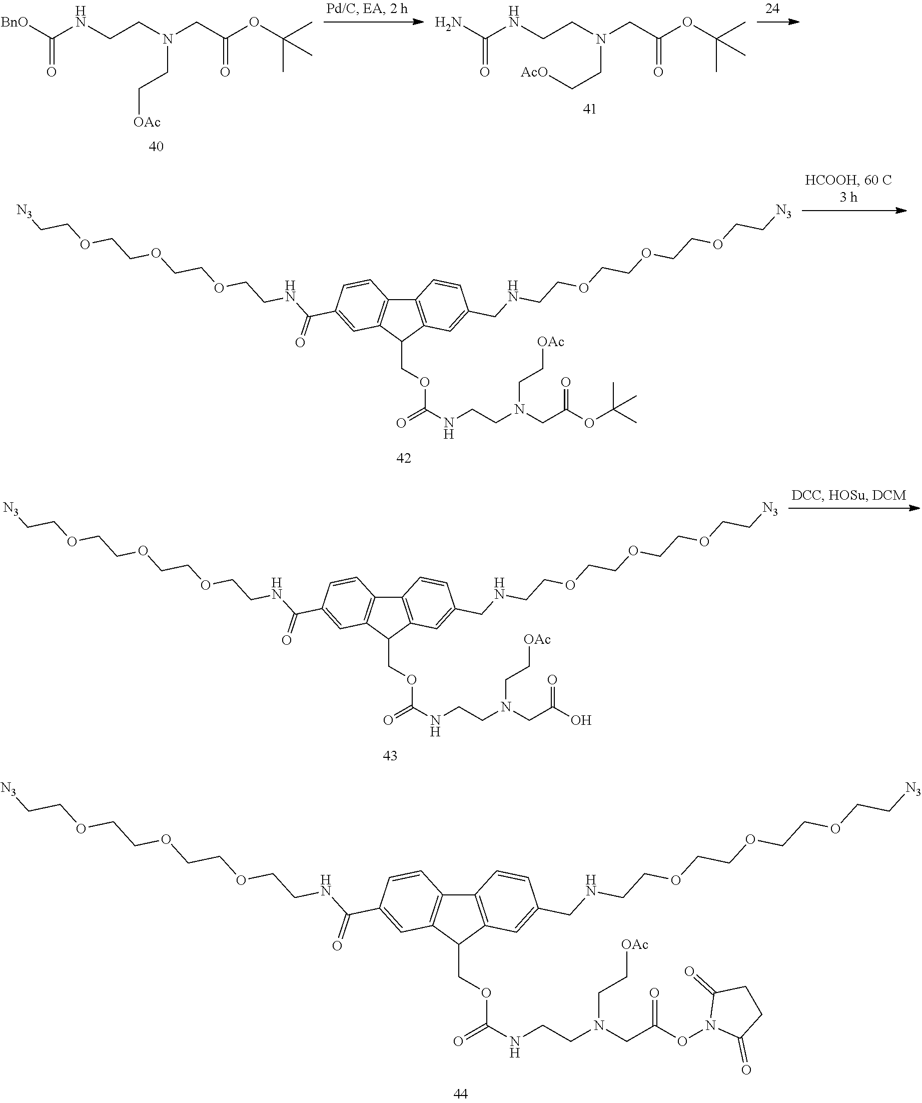 [SMILES: CC(=O)OCCN(CCNC(=O)OCc1ccccc1)CC(=O)OC(C)(C)C ; CC(=O)OCCN(CCNC(N)=O)CC(=O)OC(C)(C)C ; CC(=O)OCCN(CCNC(=O)OCC1c2cc(CNCCOCCOCCOCCN=[N+]=[N-])ccc2-c2ccc(C(=O)NCCOCCOCCOCCN=[N+]=[N-])cc21)CC(=O)OC(C)(C)C ; CC(=O)OCCN(CCNC(=O)OCC1c2cc(CNCCOCCOCCOCCN=[N+]=[N-])ccc2-c2ccc(C(=O)NCCOCCOCCOCCN=[N+]=[N-])cc21)CC(=O)O ; CC(=O)OCCN(CCNC(=O)OCC1c2cc(CNCCOCCOCCOCCN=[N+]=[N-])ccc2-c2ccc(C(=O)NCCOCCOCCOCCN=[N+]=[N-])cc21)CC(=O)ON1C(=O)CCC1=O]

### Preparation of tert-butyl N-(2-acetoxyethyl)-N-(2-aminoethyl)glycinate (41)

To a solution of tert-butyl N-(2-acetoxyethyl)-N-(2-(((benzyloxy)carbonyl)amino)ethyl)glycinate 40 (75.0 mg, 0.19 mmol, 1.0 eq) in ethyl acetate (0.6 mL), Pd/C (40 mg, 10%, dry) was added at room temperature. The reaction mixture was under replacement three times with H2. Then the mixture was stirred for 2 h under H2 at room temperature. The reaction was monitored by ¹H NMR and TLC. (PE:EA=1:1) compound 40: Rf=0.3; compound 41: Rf=0.05. The reaction solution was filtered through a pad of celite. The organic layer was concentrated to give the product 41 (48.4 mg, 98%) as light yellow oil.

### Preparation of N-(2-acetoxyethyl)-N-(2-((((2,7-bis((2-(2-(2-(2-azidoethoxy)-ethoxy)ethoxy)-ethyl)carbamoyl)-9H-fluoren-9-yl)methoxy)carbonyl)amino)ethyl)glycine (43)

The compound 41 made above was redissolved in EtOAc (0.6 mL). To which was added compound 24 (161.0 mg, 0.19 mmol) in DCM (1 mL) and followed by addition of pyridine (20 μL). The reaction was stirred at rt for 1 h, and monitored with LCMS. The reaction was taken to EtOAc (5 mL) and washed with 1 N HCl (2 mL), and the organic phase was dried over Na₂SO₄, and filtered. Then the solvent was removed in vacuo.

The crude product 42 was added HCO₂H (4 mL) and heated to 60° C. for 3 h. The product was purified with HPLC in 10-100% MeCN/H₂O (0.1% TFA) to obtain the desired compound 43 (31.4 mg, 18% for 3 steps).

### Preparation of 2,5-dioxopyrrolidin-1-yl N-(2-acetoxyethyl)-N-(2-((((2,7-bis((2-(2-(2-(2-azidoethoxy)ethoxy)ethoxy)ethyl)carbamoyl)-9H-fluoren-9-yl)methoxy)carbonyl)-amino)ethyl)glycinate (44)

Compound 43 (9.7 mg, 0.011 mmol) was dissolved in DCM (0.037 mL) and treated with HOSu (2.56 mg, 0.022 mmol) and DCC (4.54 mg, 0.022 mmol) in DCM (0.04 mL) at 0° C. The reaction was stirred for overnight at rt. The reaction was filtered and concentrated. 3-5 times volume of Et₂O was added and the solution turned to be cloudy and the cloudy solution was centrifuged. The top layer clear solution was decanted and the bottom oily solid was washed with Et₂O (2×) and dried under high vacuum to obtain compound 44 (7.2 mg, 65%). LCMS: 1012 (M+1)⁺; HPLC 96% (UV254); ¹H NMR (300 MHz, Chloroform-d) δ 8.09 (p, J=0.7 Hz, 2H), 7.84 (td, J=8.3, 1.1 Hz, 4H), 6.92 (s, 3H), 4.43 (d, J=6.9 Hz, 2H), 4.31 (m, 1H), 4.16 (t, J=5.3 Hz, 2H), 3.81 (s, 2H), 3.79-3.55 (m, 47H), 3.38-3.24 (m, 8H), 3.00-2.78 (m, 11H), 2.01 (s, 3H).

### Example 10

### 20 kDa Y-PEG-DBCO

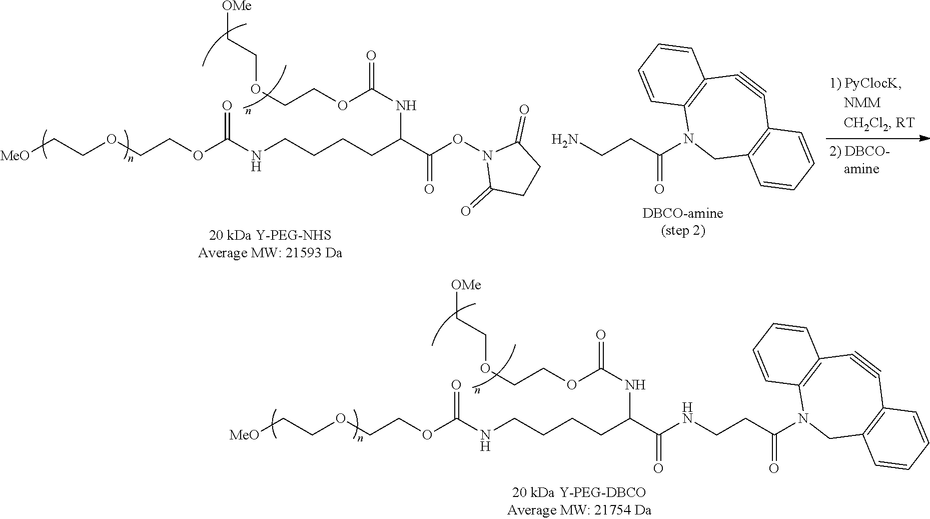

To a dried round-bottomed flask, equipped with a Teflon coated magnetic stir bar was added 20 kDa Y-PEG-NHS (1.08 g, 50.0 μmol, 1.0 equiv) and PyClocK (0.033 g, 60.0 μmol, 1.2 equiv). The flask was sealed with a rubber septum and placed under an inert atmosphere of Argon. Anhydrous CH₂Cl₂ (5.0 mL) was added, followed by N-methylmorpholine (6.10 μL, 55.0 μmol, 1.1 equiv) and the reaction solution was stirred at room temperature for 30 min. DBCO-amine (0.028 mg, 100 μmol, 2.0 equiv) was added in one portion as a solid and the reaction mixture was stirred at room temperature for a further 3 h. The crude reaction mixture was taken up into a glass pipette and added drop-wise to 2-propanol (100 mL) with vigorous stirring. A white precipitate was yielded (PEG material) and the resulting suspension was cooled to 4° C. and filtered (vacuum filtration), washing with ice-cold 2-propanol (3×50 mL). The isolated precipitate was transferred to pre-weighed falcon tubes (×2) and dissolved in warm (40° C.) acetone (90 mL). The solutions were cooled in an ice bath for 15 min to induced precipitation of the PEG material. The suspensions were pelleted by centrifugation (10500 rpm, 20 min, 4° C.) and the supernatant was carefully discarded. The pellets were re-dissolved in fresh, warm acetone (40° C.), cooled in an ice bath to induced precipitation and subjected to another round of centrifugation/decantation. This process was repeated to a total of 4 times. The pellets were dried in vacuo. Isolated white solid, mass=1.08 g (99%). R^{p}-HPLC retention time=6.9 min.

### Example 11

### mPEG2-Fmoc-Bn-20K-NHS

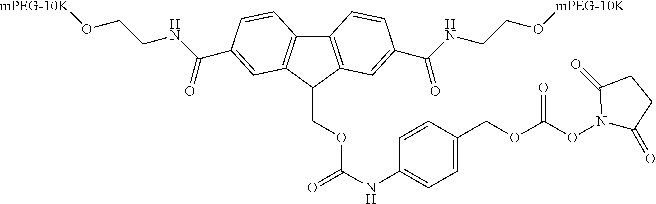

Example 11 mPEG2-Fmoc-Bn-20K-NHS was generated according to modified literature procedures from US20060293499A1 and Bioconjugate Chemistry 2003, 14, 395-403.

¹H NMR (300 MHz, d₆-DMSO) δ 9.14 (br, 1H), 8.56 (m, 2H), 8.25-8.17 (m, 2H), 8.04-7.97 (m, 4H), 7.44 (m, 2H), 7.33 (m, 2H), 5.77 (s, 2H), 4.69 (m, 2H), 4.46 (m, 1H), 3.51 (br, 1800H), 2.81 (s, 4H).

HPLC: purity 94.7%; GPC: purity 91.2%; MALDI/GPC: 21048 Da.

### Example 12

### mPEG2-Fmoc-Bi-20K-NHS

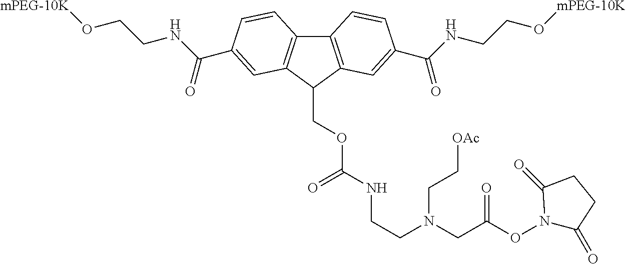

Example 12 mPEG2-Fmoc-Bi-20K-NHS is generated according to modified literature procedures from US20060293499A1 and Bioconjugate Chemistry 2006, 17, 341-351.

### Example 13

### rIL-2 Preparation

The IL-2 gene encoding the polypeptide as shown in FIG. 1 was synthesized and cloned into pET21a (+) expression vector as a Ndel/Xhol fragment. The sequences of the synthetic primers used for cloning were: forward primer: 5′-aatcatatggcacctacttcaagttctacaaa-3′ (SEQ ID NO: 4), and reverse primer: 5′-aatttatcaagttagtgttgagatgat-3′ (SEQ ID NO: 5). The positive clones were identified by restriction enzyme digestion (Ndel and Xhol) and sequenced using standard sequencing protocols.

The positive clone was selected and transformed in the E. Coli cells (BL21 DE3). Standard procedure for induction of the IL-2 protein was followed. Briefly, a single colony was inoculated in a 5 ml luria broth (LB) media containing 100 sg/ml ampicillin and grown overnight at 37° C., 200 rpm. The overnight culture was diluted 100 times in LB media containing 100 μg/ml ampicillin and grown at 37° C., 200 rpm. When absorbance at 600 nm reached around 0.8, culture was induced with 1 mM IPTG. The culture temperature was raised to 42° C. for induction period. The fermentation ended after 4 hours induction.

Following fermentation, the cells were harvested by centrifugation. The cell mass pellet was stored at −80° C. for future homogenization. The frozen cell mass pellet was re-suspended in cell wash buffer (20 mM Tris, 1 mM EDTA, pH 8.0) to a concentration of 10% (W/V) and centrifuged at 15600×g, 4° C. for 30 minutes. The supernatant was discarded. The washed pellet was re-suspended in homogenization buffer (20 mM Tris, 0.1 M NaCl, 1 mM EDTA, 1 mM PMSF, 0.5% Trition-X100, pH 8.0) and homogenized by a Sonicator (SCIENTZ-ID from SCIENTZ, Ningbo, Zhejiang, PRC) at 4-15° C. for three passes. The homogenate was centrifuged at 15600×g, 4° C. for 30 minutes. The supernatant was discarded. The inclusion body pellet was washed in buffer (20 mM Tris, 0.1 M NaCl, 2 M Urea, 1 mM EDTA, pH 8.0) and centrifuged 15600×g, 4° C. for 30 minutes. The supernatant was discarded. After centrifuging, the crude IL-2 inclusion bodies were obtained.

The crude IL-2 inclusion bodies were dissolved into buffer, 6 M guanidine, 100 mM Tris, 2 mM EDTA, 5 mM dithiothreitol (DTT), pH 8.0. The mixture was incubated at 50° C. for 30 minutes. After reduction, water was added to the mixture to reduce guanidine concentration to 4.8 M. After one hour of centrifuging at 15600×g, the resulting gel-like pellet was discarded. The guanidine concentration in the supernatant was further reduced to 3.5 M by adding water. The pH was adjusted to 5 with titration of 100% acetic acid. The mixture was incubated at room temperature for 60 minutes and centrifuged at 15600×g for one hour. The resulting pellet was suspended into 3.5 M guanidine, 20 mM acetate, 5 mM DTT, pH 5 buffer and centrifuged at 15600×g for one hour. This washing step was repeated one more time.

The clean and reduced IL-2 inclusion bodies were dissolved into 6 M guanidine, 100 mM Tris pH 8 buffer. 100 mM CuCl₂ stock was added to reach a final Cu²⁺ concentration of 0.1 mM. The mixture was incubated at 4° C. overnight.

The expressed IL-2 solution was put into dialysis bags (having a molecular weight pore size of 3 kiloDaltons). The dialysis bags were put into a reservoir containing 4.8 M guanidine, 0.1 M Tris, pH 8 buffer. After three hours equilibration, the guanidine concentration in the reservoir was first slowly reduced to 2 M by pumping water into the reservoir over a period of 15 hours, then reduced to less than 10 mM by pumping 20 mM PB pH 6.0 buffer into the reservoir over a period of 8 hours. The entire refolding process was completed at 4° C. The refolded IL-2 was checked with SEC-HPLC.

The refolded IL-2 was centrifuged at 15600×g for 60 minutes to remove precipitates. The supernatant was concentrated with Mini Pellicon TFF membrane system (Millipore Corporation, USA).

The refolded and concentrated IL-2 was loaded on a XK column (GE Healthcare Bio-Sciences AB, Uppsala Sweden) packed with SP Sepharose FF resin. The running buffer was 20 mM PB pH 6.0 and flow rate was 10 mL/min. The fractions under the IL-2 monomer peak were pooled.

The pooled SP Sepharsoe FF eluent was desalted by loading on a XK column (GE Healthcare Bio-Sciences AB, Uppsala Sweden) packed with Sephadex G25 resin. The running buffer was 20 mM PB pH 6.0 and flow rate was 25 mL/min. The fractions under the IL-2 monomer peak were pooled.

The desalted IL-2 monomer pool was loaded on a XK column (GE Healthcare Bio-Sciences AB, Uppsala Sweden) packed with Q Sepharose FF resin. The running buffer was 20 mM PB pH 6.0 and flow rate was 25 mL/min. The flow through peak was pooled. It should be noted that other suitable purification methods may also be employed, such as size exclusion chromatography and hydrophobic interaction chromatography (HIC chromatography).

The IL-2 monomer fraction pool was concentrated to about 1-2 mg/mL using Mini Pellicon TFF membrane system (Millipore Corporation, USA) at 4° C. and 10-22 psi operation pressure. The concentrated IL-2 monomer solution was dialyzed into final formulation buffer (10 mM acetate-Na, 5% trehalose, pH 4.5) at 4° C. The formulated IL-2 solution was rendered sterile by passing a 0.22 μm filter and stored in −80° C. for further use.

### Preparation of lyophilized rIL-2 for conjugation

Sixteen vials of rIL-2 (16×5 mg) were warmed to room temperature from −80° C. To each vial of lyophilized material, 0.1% aq. SDS (21 mL) was added, the contents of the vials were mixed until complete dissolution had been achieved. The rIL-2 solution was buffer exchanged into 100 mM sodium borate, pH 8 and concentrated via UF/DF (Vivaspin20, 5 kDa MWCO PES). The buffer exchanged protein solution was sterile filtered (0.22 μm PVDF) and quantified by UV-A280 using a Nanodrop 2000 spectrophotometer (3.19 mg/mL).

### Preparation of solution-based IL-2 for conjugation in pH 8.0 buffer

IL-2 (15 mg, 10 mL) was buffer exchanged into 100 mM sodium borate, pH 8, 20 mM EDTA, 0.05% SDS using a P100 column as per the manufacturer's instructions. The IL-2 solution was concentrated via UF/DF (Vivaspin20, 5 kDa MWCO PES). The buffer exchanged protein solution was sterile filtered (0.22 μm PVDF) and quantified by UV-A280 using a Nanodrop 2000 spectrophotometer (2.67 or 2.5 or 3.0 mg/mL respectively).

### Preparation of solution-based IL-2 for conjugation in pH 9.0 buffer

IL-2 (15 mg, 10 mL) was buffer exchanged into 100 mM sodium borate, pH 9, 20 mM EDTA, 0.05% SDS using a P100 column as per the manufacturer's instructions. The IL-2 solution was concentrated via UF/DF (Vivaspin20, 5 kDa MWCO PES). The buffer exchanged protein solution was sterile filtered (0.22 μm PVDF) and quantified by UV-A280 using a Nanodrop 2000 spectrophotometer (2.9 mg/mL).

### Example 14

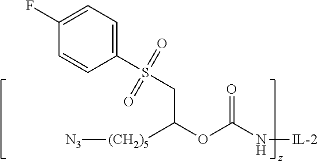

### NHS Conjuration of rIL-2 with Example 1

[rIL-2]-[F-Ph-SO₂—N₃]_{z} Production

Prior to conjugation, IL-2 was diluted to 3.09 mg/mL with 100 mM sodium borate, pH 8.

Compound 8 (4.4 mg) was dissolved in DMF (0.885 mL) to give a 4.97 mg/mL solution of the reagent. To a vial of rIL-2 (10 mg, 3.24 mL), compound 8 (1.79 mg, 360 μL, 6 eq.) was added, the reaction was mixed and incubated at 22° C. for 1 h. At 1 h, the reaction was analysed by LC-MS to determine the distribution of functionalized IL-2 species as [rIL-2]-[F-Ph-SO₂—N₃]_{z}.

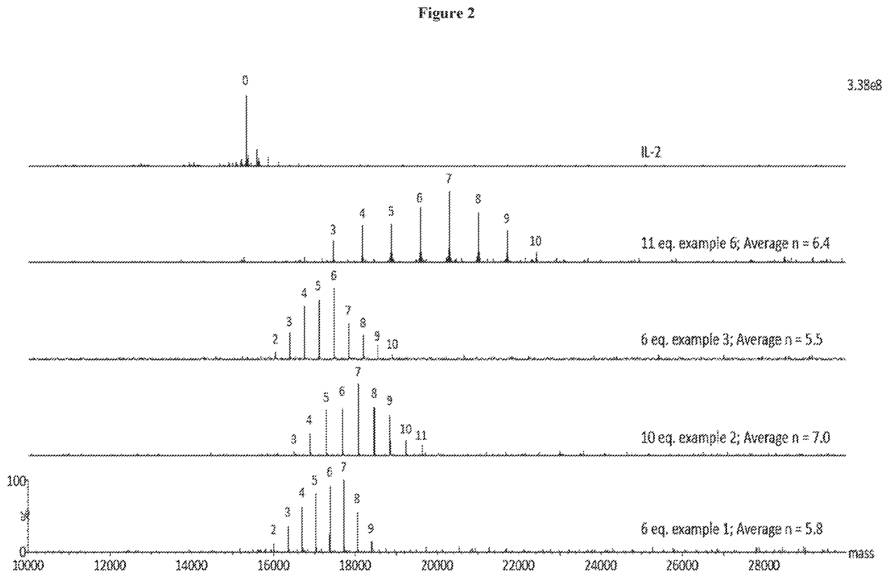

FIG. 2 showed [rIL-2]-[F-Ph-SO₂—N₃]_{z} distribution centred around 6, determined by LC-MS.

### Example 15

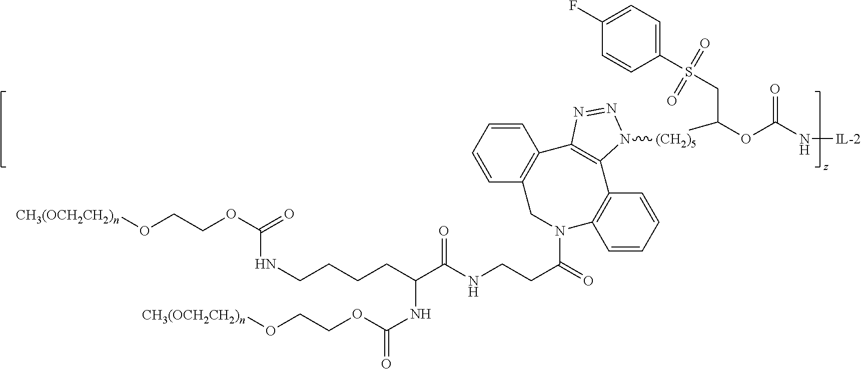

### Click-PEGylation of [rIL-2]-[F-Ph-SO₂—N₃]_{z} with 20 kDa Y-PEG-DBCO

20 kDa Y-PEG-DBCO (143.7 mg) was dissolved in 100 mM sodium borate, pH 8 (1.419 mL). To the solution of [rIL-2]-[F-Ph-SO₂—N₃]_{z} example 14 (9.5 mg, 3.42 mL), 20 kDa Y-PEG-DBCO (134 mg, 1.33 mL, 10 eq.) was added. The reaction was mixed and incubated at 22° C. The reaction mixture was analyzed by SDS-PAGE after 2 h. The crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Sample was quantified by IR using a DirectDetect instrument (8.8 mg, 92% yield). PEG:IL-2 ratio was determined by SDS-PAGE.

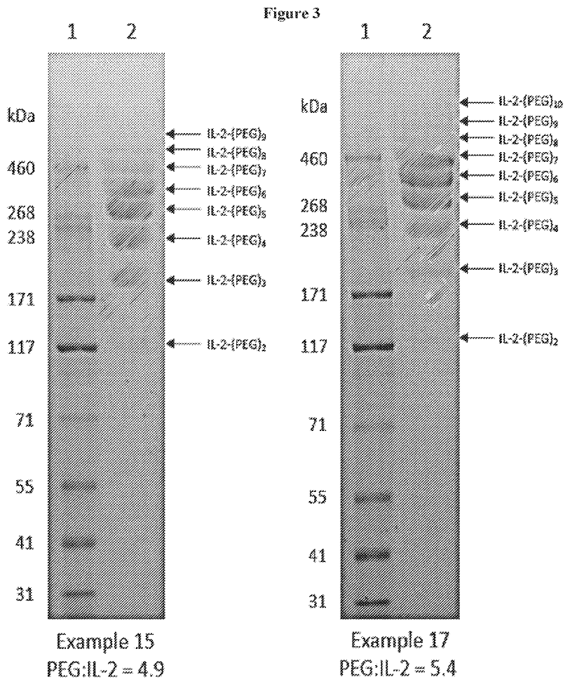

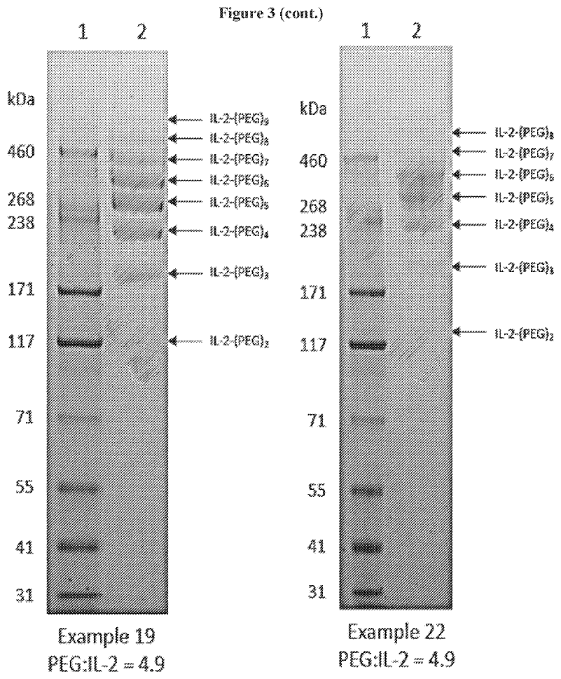

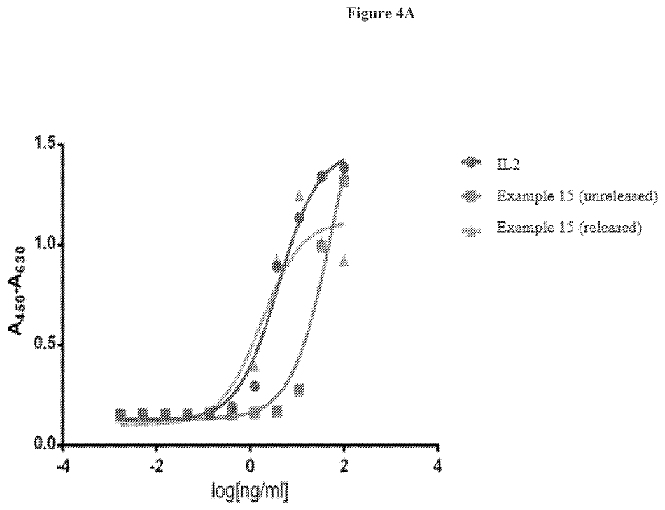

FIG. 3 shows the SDS-analysis of the [20K mPEG-(F-Ph-SO₂)]z-[rIL-2] conjugates with PEG:IL-2 ratio equaled to 4.9.

### Example 16

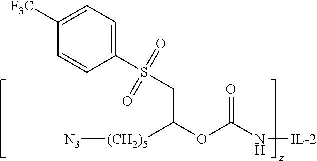

### NHS Conjugation of rIL-2 with Example 2

[rIL-2]-[CF₃-Ph-SO₂—N₃]z production:

Prior to conjugation, IL-2 was diluted to 3.09 mg/mL with 100 mM sodium borate, pH 8.

Compound 13 (7.5 mg) was dissolved in DMF (0.816 mL) to give a 9.19 mg/mL solution of the reagent. To a vial of rIL-2 (10 mg, 3.24 mL), compound 13 (3.31 mg, 360 μL, 10 eq.) was added, the reaction was mixed and incubated at 22° C. for 1 h. At 1 h, the reaction was analyzed by LC-MS to determine the distribution of functionalized IL-2 species as [rIL-2]-[CF₃-Ph-SO₂—N₃]z.

FIG. 1 showed the formation of [rIL-2]-[CF₃-Ph-SO₂—N₃]_{z} distribution centred around 6, determined by LC-MS.

### Example 17

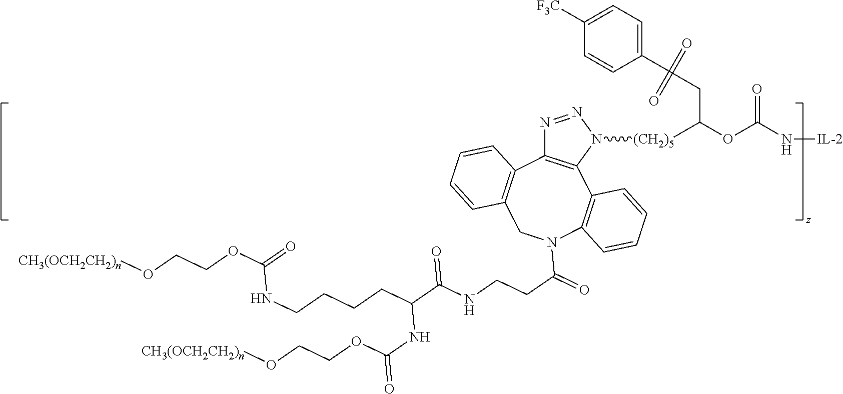

### Click-PEGylation of [rIL-2]-[CF₃-Ph-SO₂—N₃]_{z} with 20 kDa Y-PEG-DBCO

20 kDa Y-PEG-DBCO (210.9 mg) was dissolved in 100 mM sodium borate, pH 8 (1.388 mL). To the solution of [rIL-2]-[CF₃-Ph-SO₂—N₃]_{z} example 16 (9.7 mg, 3.49 mL), 20 kDa Y-PEG-DBCO (207 mg, 1.36 mL, 15 eq.) was added. The reaction was mixed and incubated at 22° C. The reaction mixture was analyzed by SDS-PAGE after 2 h. The crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Sample was quantified by IR using a DirectDetect instrument (7.9 mg, 81% yield). PEG:IL-2 ratio was determined by SDS-PAGE.

FIG. 3 shows the SDS-analysis of the [20K mPEG-(CF₃-Ph-SO₂)]z-[rIL-2] conjugates with PEG:IL-2 ratio equaled to 5.4.

### Example 18

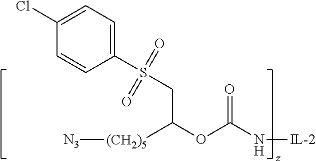

### NHS Conjugation of rIL-2 with Example 3

[rIL-2]-[Cl-Ph-SO₂—N₃]_{z} production:

Prior to conjugation, IL-2 was diluted to 3.09 mg/mL with 100 mM sodium borate, pH 8. Compound 18 (5.0 mg) was dissolved in DMF (0.971 mL) to give a 5.15 mg/mL solution of the reagent. To a vial of rIL-2 (10 mg, 3.24 mL), compound 18 (1.85 mg, 360 μL, 6 eq.) was added, the reaction was mixed and incubated at 22° C. for 1 h. At 1 h, the reaction was analyzed by LC-MS to determine the distribution of functionalized IL-2 species as [rIL-2]-[C₁-Ph-SO₂—N₃]_{z}.

FIG. 1 shows the formation of [rIL-2]-[C₁-Ph-SO₂—N₃]_{z} distribution centred around 5, determined by LC-MS.

### Example 19

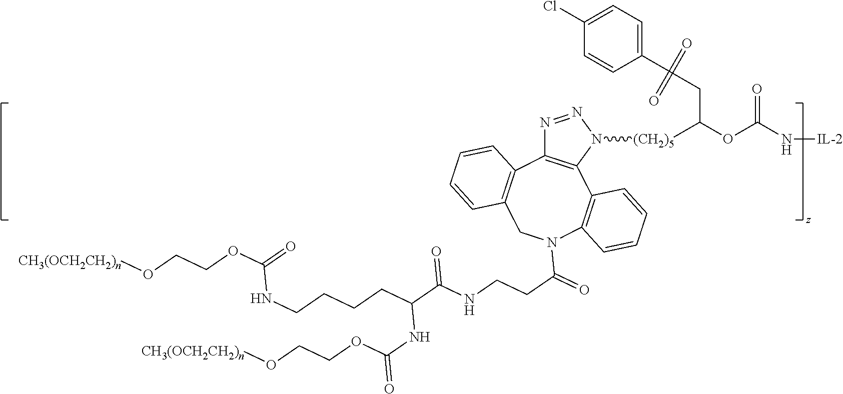

### Click-PEGylation of [rIL-2]-[Cl-Ph-SO₂—N₃]_{z} with 20 kDa Y-PEG-DBCO

20 kDa Y-PEG-DBCO (213.2 mg) was dissolved in 100 mM sodium borate, pH 8 (1.403 mL). To the solution of [rIL-2]-[Cl-Ph-SO₂—N₃]_{z} example 18 (9.7 mg, 3.49 mL), 20 kDa Y-PEG-DBCO (207 mg, 1.36 mL, 15 eq.) was added. The reaction was mixed and incubated at 22° C. The reaction mixture was analyzed by SDS-PAGE after 2 h. The crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Sample was quantified by IR using a DirectDetect instrument (8.2 mg, 84%). PEG:IL-2 ratio was determined by SDS-PAGE.

FIG. 3 shows the SDS-analysis of the [20K mPEG-(Cl-Ph-SO₂)]z-[rIL-2] conjugates with PEG:IL-2 ratio equaled to 4.9.

### Example 20

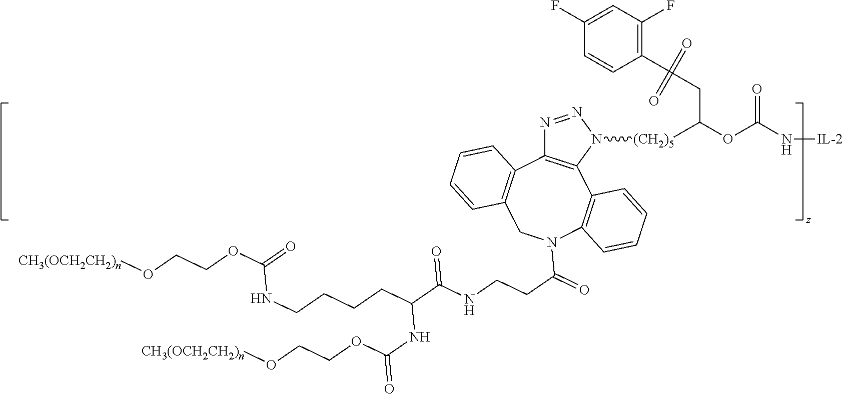

### NHS Conjugation of rIL-2 with Example 4 and Click-PEGylation with 20 kDa Y-PEG-DBCO

Example 4 (5.8 mg) was dissolved in DMF (0.677 mL) to give a 8.57 mg/mL solution of the reagent. To a vial of IL-2 (7 mg, 0.458 μmol, 2.265 mL), example 4 (2.16 mg, 4.55 μmol, 252 μL, 10 eq.) was added, the reaction was mixed and incubated at 22° C. for 1 h. After 1 h, the reaction was analysed by LC-MS to determine the average degree of IL-2 functionalisation.

20 kDa Y-PEG-DBCO (406.4 mg) was dissolved in 100 mM sodium borate, pH 8 (2.00 mL) to give a 203 mg/mL solution. To [rIL-2]-[F,F-Ph-SO₂—N₃]_{z} (7.0 mg, 0.458 μmol, 2.52 mL), 20 kDa Y-PEG-DBCO (199 mg, 9.15 μmol, 0.98 mL, 20 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Example 20 was quantified by IR using a DirectDetect instrument as [20K mPEG-(F,F-Ph-SO₂)]z-[rIL-2] (5.2 mg, 74% yield). SDS-PAGE analysis of the conjugate showed PEG:IL-2 ratio equaled to 4.8.

### Example 21

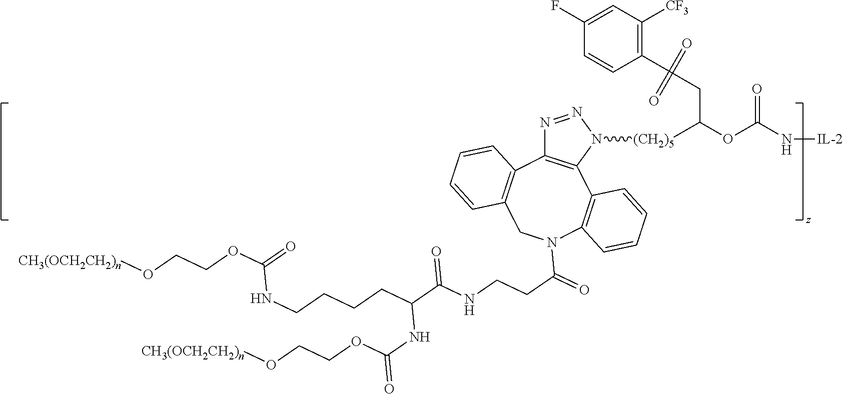

### NHS Conjugation of rIL-2 with Example 5 and Click-PEGylation with 20 kDa Y-PEG-DBCO

Example 5 (4.5 mg) was dissolved in DMF (0.528 mL) to give a 8.52 mg/mL solution of the reagent. To a vial of IL-2 (7 mg, 0.458 μmol, 2.265 mL), example 5 (2.15 mg, 4.10 μmol, 252 μL, 9 eq.) was added, the reaction was mixed and incubated at 22° C. for 1 h. After 1 h, the reaction was analysed by LC-MS to determine the average degree of IL-2 functionalisation.

20 kDa Y-PEG-DBCO (406.4 mg) was dissolved in 100 mM sodium borate, pH 8 (2.00 mL) to give a 203 mg/mL solution. To [rIL-2]-[F,CF₃-Ph-SO₂—N₃]_{z} (7.0 mg, 0.458 μmol, 2.52 mL), 20 kDa Y-PEG-DBCO (199 mg, 9.15 μmol, 0.98 mL, 20 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Example 21 was quantified by IR using a DirectDetect instrument as [20K mPEG-(F,CF₃-Ph-SO₂)]_{z}-[rIL-2] (2.9 mg, 41% yield). SDS-PAGE analysis of the conjugate showed PEG:IL-2 ratio equaled to 4.5.

### Example 22

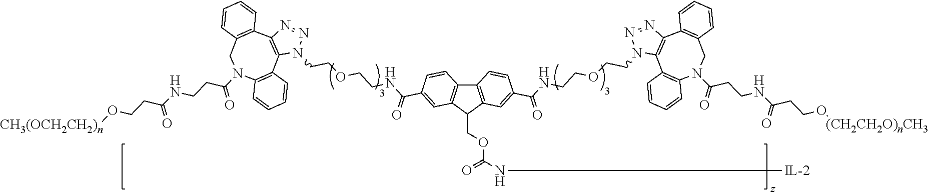

### NHS Conjugation of rIL-2 with Example 6 and Click-PEGylation with 10 kDa PEG-DBCO

Prior to conjugation, IL-2 was diluted to 3.09 mg/mL with 100 mM sodium borate, pH 8. Compound 24 (16.5 mg) was dissolved in DMF (1.107 mL) to give a 14.9 mg/mL solution of the reagent. To a vial of IL-2 (10 mg, 3.24 mL), compound 24 (5.96 mg, 400 μL, 11 eq.) was added, the reaction was mixed and incubated at 22° C. for 1 h. At 1 h, the reaction was analysed by LC-MS to determine the distribution of functionalised IL-2 species as [rIL-2]-[Fmoc-(N₃)₂]z.

10 kDa PEG-DBCO (Iris Biotech, 276.3 mg) was dissolved in 100 mM sodium borate, pH 8 (1.439 mL). To the solution of [rIL-2]-[Fmoc-(N₃)₂]z. (10 mg, 3.64 mL), 10 kDa PEG-DBCO (262 mg, 1.36 mL, 40 eq.) was added. The reaction was mixed and incubated at 22° C. The reaction mixture was analyzed by SDS-PAGE after 2 h. The crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Sample was quantified by IR using a DirectDetect instrument. PEG:IL-2 ratio was determined by SDS-PAGE.

FIG. 3 shows the SDS-analysis of the conjugates [mPEG₂-T₂-Fmoc-20K]_{z}-[rIL-2] with PEG:IL-2 ratio equaled to 4.9.

### Example 23

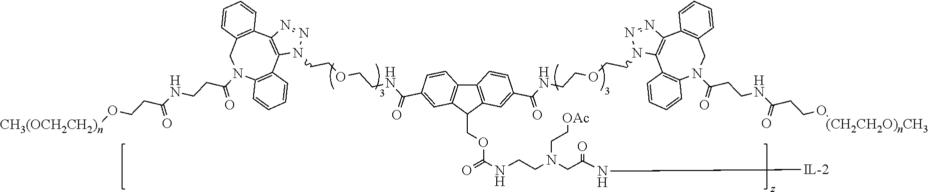

### NHS Conjugation of rIL-2 with Example 9 and Click-PEGylation with 10 kDa PEG-DBCO

Under similar preparation procedure as example 14 and 15, example 23 is prepared as [mPEG₂-T₂-Fmoc-Bi-20K]_{z}-[rIL-2] using example 9.

### Example 24

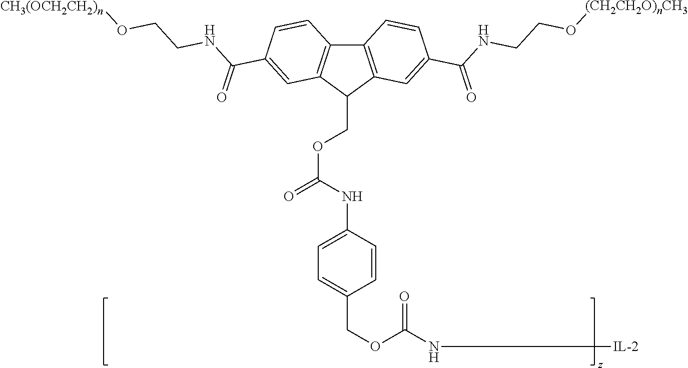

### PEGylation of rIL-2 with Example 11

Prior to conjugation, IL-2 is diluted to 1.5 mg/mL with 100 mM sodium borate, pH 8. mPEG₂-Fmoc-Bn-20K-NHS Example 11 is dissolved in 100 mM sodium borate, pH 8. and it is added to the rIL-2 (10 mg) in an amount sufficient to reach a molar ratio of mPEG₂-Fmoc-Bn-20K-NHS to rIL-2 of 100:1. The conjugation reaction is allowed to proceed for one hour at 22° C. to provide [mPEG₂-Fmoc-Bn-20K]_{z}-[rIL-2] conjugates. The crude reaction mixture is purified by SEC using a HiLoad 26/600 Superdex 200 pg. Sample is isocratically eluted with 50 mM sodium acetate, pH 4.5 (150 mM NaCl) at 3 mL/min flow rate. Fractions collected over the methods are analysed by SDS-PAGE and high purity fractions are pooled. The pooled fractions are concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

[mPEG₂-Fmoc-Bn-20K]_{z}-[rIL-2] is quantified by IR using a DirectDetect instrument PEG:IL-2 ratio is determined by SDS-PAGE.

### Example 25

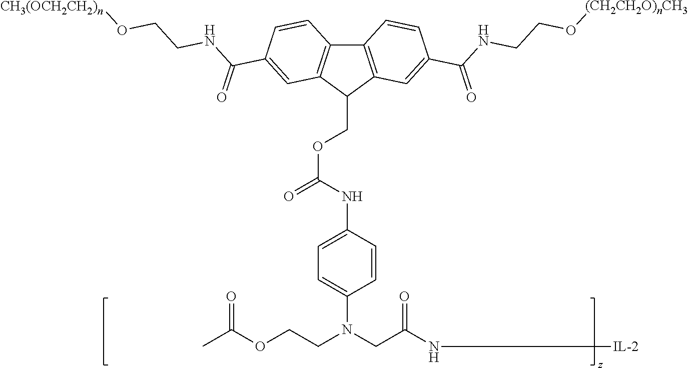

### PEGylation of rIL-2 with Example 12

Using similar PEGylation and purification conditions of example 24, PEGylation of rIL-2 with Example 12 mPEG₂-Fmoc-Bi-20K-NHS produces [mPEG₂-Fmoc-Bi-20K]_{z}-[rIL-2] conjugate.

### Example 26

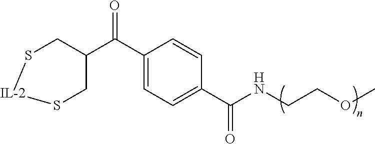

### PEGylation of rIL-2 Disulfide Bond with 10 kDa PEG bis(sulfone) 45

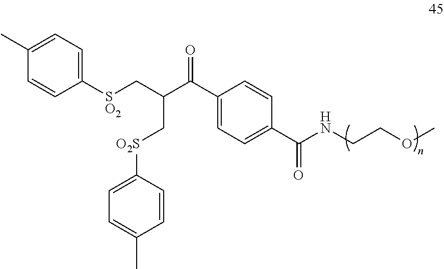

To a solution of r-IL-2 (4.2 mg, 0.25 mg/mL) in 100 mM sodium borate buffer, pH 8, was added 10 mM DTT. The solution was incubated for one hour at 22° C. The excess DTT was removed by gel filtration using 100 mM sodium borate buffer, pH 8, containing 20 mM EDTA. To the reduced protein solution was added 1.3 equiv of 10 kDa PEG bis(sulfone) 45, 0.05% w/v SDS and the solution was allowed to react for 16 h at 22° C. The reaction solution was filtered through Vivapure Q Mani H filter to remove SDS. It was then buffer-exchanged by ultrafiltration with 5 kDa MWCO spin filters into 50 mM sodium acetate, pH 4.0. The solution was then loaded onto a 5 mL MacroCapSP resin column. The conjugate eluted on washing the column with a linear gradient of 0-1 M sodium chloride in 50 mM sodium acetate buffer, pH 4. The conjugate was further isolated by size exclusion chromatography (SEC) to generate 1.4 mg product. Purity by SDS-PAGE: 97%. Purity by Analytical SEC: 87.3%

### Example 27

### PEGylation of rIL-2 with PEG Reagent 46

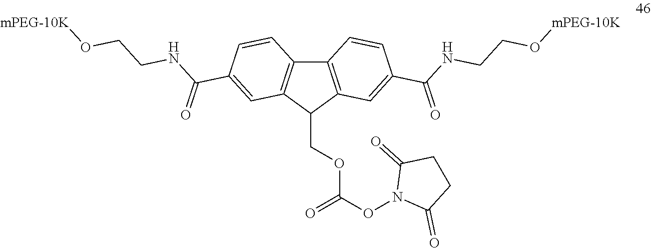

A 405 mg/mL solution of PEG reagent 46 (1.50 g) was prepared in 2 mM HCl (3.702 mL). To rIL-2 (10 mg, 3.135 mL), 405 mg/mL PEG reagent 46 (1.43 g, 3.535 mL, 100 eq.) was added. The reaction was mixed and incubated at 22° C. After 1 h the crude reaction was analysed by SDS-PAGE and was purified by SEC. Crude IL-2-(PEG)_{z} product was purified by SEC using a HiLoad 26/600 Superdex 200 pg column. The sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Protein concentration was quantified by IR using a DirectDetect instrument (6.6 mg, 66%) and the PEG:IL-2 ratio was determined by SDS-PAGE. SDS-analysis of the conjugates [mPEG₂-Fmoc-20K]_{z}-[rIL-2] showed the PEG:IL-2 ratio equaled to 5.1.

### Example 28

### 1-((3-((2-(2-(2-(2-azidoethoxy)ethoxy)ethoxy)ethyl)carbamoyl)phenyl)sulfonyl)-5-methoxynentan-2-yl (25-dioxopyrrolidin-1-yl) carbonate (52)

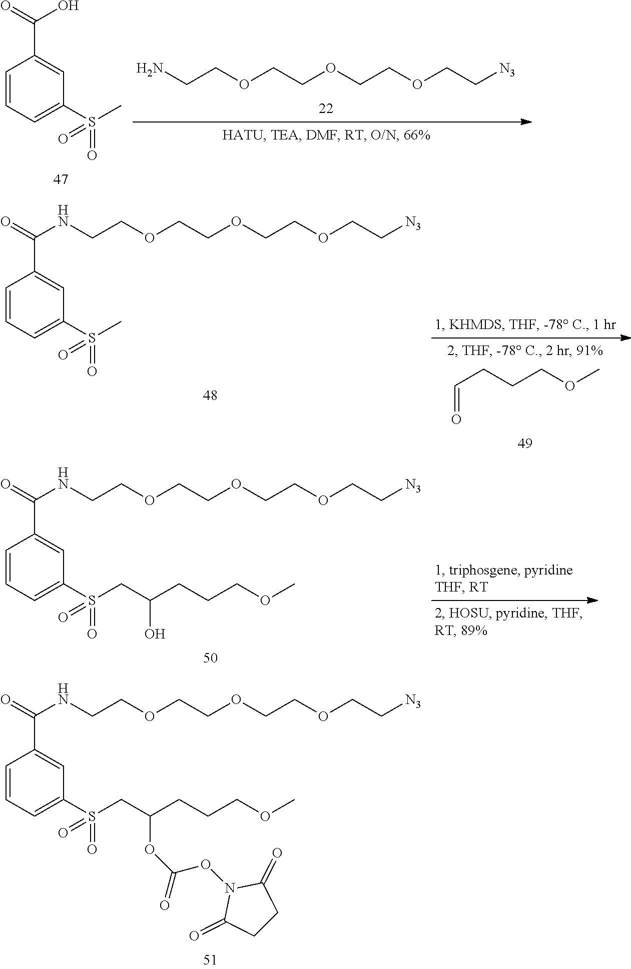

### Preparation of N-(2-(2-(2-(2-azidoethoxy)ethoxy)ethoxy)ethyl)-3-(methylsulfonyl)benzamide (48)

To a solution of compound 47 (1.0 g, 5.0 mmol) in DMF (15 mL) was added compound 22 (1.3 g, 6.0 mmol), HATU (2.47 g, 6.5 mmol) and TEA (1.01 g, 10.0 mmol). The reaction mixture was stirred at room temperature for 16 h. The reaction mixture was concentrated and dissolved with ethyl acetate and water. The mixture was extracted with ethyl acetate (3×20 mL). The combined organics were washed with brine, dried over sodium sulfate, filtered, and concentrated by rotary evaporation. The resulting residue was purified by column chromatography to give the compound 48 (900 mg) as a yellow oil.

### Preparation of N-(2-(2-(2-(2-azidoethoxy)ethoxy)ethoxy)ethyl)-3-((2-hydroxy-5-methoxypentyl)sulfonyl)benzamide (50)

To a solution of compound 48 (400 mg, 1 mmol) and compound 49 (560 mg, 5.5 mmol) in dry THF (30 mL) was added KHMDS (5.5 mL, 5.5 mmol) slowly at −78° C. under N₂. The reaction mixture was stirred at −78° C. for 2 h. The reaction mixture was quenched by saturated aqueous NH₄Cl solution. The mixture was extracted with ethyl acetate (3×20 mL). The combined organics were washed with brine, dried over sodium sulfate, filtered, and concentrated by rotary evaporation. The resulting residue was purified by column chromatography eluting by 2% CH₃OH in CH₂Cl₂ to give the compound 50 (168 mg) as a yellow oil.

### Preparation of 1-((3-((2-(2-(2-(2-azidoethoxy)ethoxy)ethoxy)ethyl)-carbamoyl)phenyl)sulfonyl)-5-methoxypentan-2-yl (2,5-dioxopyrrolidin-1-yl) carbonate (51)

To a solution of compound 50 (100 mg, 0.2 mmol) and triphosgene (89 mg, 0.3 mmol) in dry THF (5 mL) was added pyridine (64 mg, 0.8 mmol) slowly. The reaction mixture was stirred at rt. for 20 min. Then it was filtered, and concentrated by rotary evaporation. The resulting residue was used in next step.

To a solution of the resulting residue (117 mg, 0.2 mmol) and HOSu (69 mg, 0.6 mmol) in dry THF (5 mL) was added pyridine (64 mg, 0.8 mmol) slowly. The reaction mixture was stirred at rt. for 30 min. The mixture was extracted with ethyl acetate (3×10 mL). The combined organics were washed with brine, dried over sodium sulfate, filtered, and concentrated by rotary evaporation. The resulting residue was purified by prep-TLC (CH₂Cl₂:CH₃OH=30:1) to give the compound 51 (55 mg) as a colorless oil.

LCMS: m/z 644.25 [M+1].

¹H NMR (400 MHz, CDCl₃) δ 8.32 (s, 1H), 8.18 (d, J=7.6 Hz, 1H), 8.04 (d, J=7.7 Hz, 1H), 7.68 (t, J=8.0 Hz, 1H), 7.37 (br s, 1H), 5.30-5.24 (m, 1H), 3.77-3.55 (m, 15H), 3.45-3.31 (m, 5H), 3.27 (s, 3H), 2.81 (s, 4H), 1.94-1.78 (m, 2H), 1.66-1.58 (m, 2H).

### Example 29

### 1-((3-(2-(2-(2-(2-azidoethoxy)ethoxy)ethoxy)ethyl)carbamoyl)-4-(trifluoromethyl)phenyl)sulfonyl)-5-methoxynentan-2-yl (2,5-dioxopyrrolidin-1-yl) carbonate (611

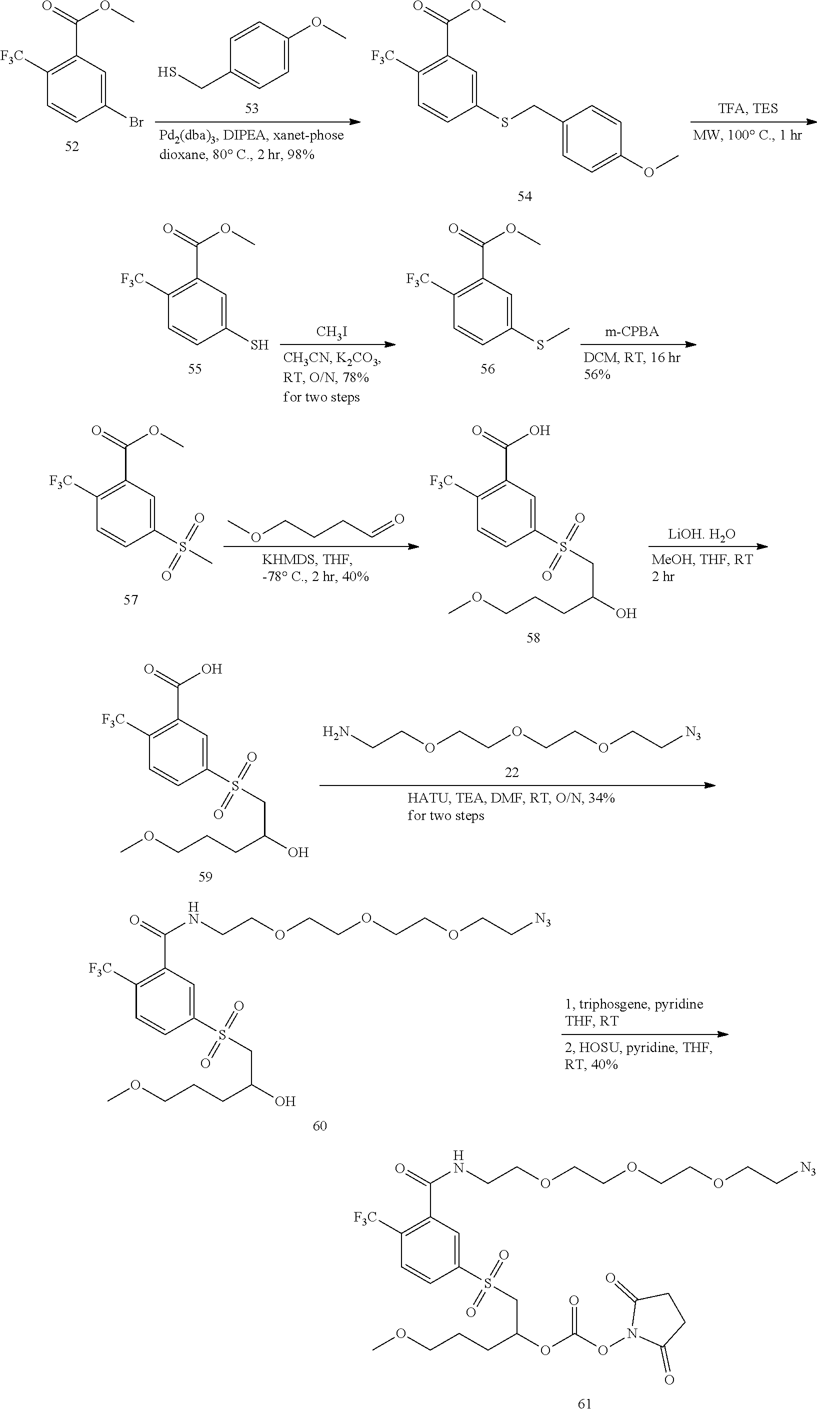

### Preparation of methyl 5-((4-methoxybenzyl)thio)-2-(trifluoromethyl)benzoate (54)

To a solution of compound 52 (5.0 g, 17.66 mmol, 1.0 eq), compound 53 (4.09 g, 26.5 mmol, 1.5 eq), Pd₂(dba)₃ (1.62 g, 1.76 mmol, 0.1 eq), Xant-phose (2.04 g, 3.52 mmol, 0.2 eq) and DIEA (6.84 g, 52.99 mol, 3.0 eq) in dioxane (50 mL) was stirred at 80° C. for 2 hrs. The result mixture was cooled to rt and filtered through a celite pad. The filtrate was concentrated and the residue was dissolved in EtOAc (100 ml). The mixture was washed with water (100 mL) and extracted with EtOAc (100 mL×3), dried over Na₂SO₄, filtered and concentrated under reduced pressure. The residue was purified by column chromatography on silica gel (PE/EA=100/1 to 80/1 to 50/1) to afford the compound 54 (6.2 g, 98%) as light-yellow oil.

TLC: PE/EA=10/1, UV, R_{f}(Compound 52)=0.80, R_{f}(Compound 54)=0.60.

LC-MS: 379.10 [M+23]⁺.

### Preparation of methyl 5-mercapto-2-(trifluoromethyl)benzoate (55)

To a solution of compound 54 (1.0 g, 2.80 mmol, 1.0 eq) and TES (0.98 g, 8.42 mmol, 3.0 eq) in TFA (15 mL) was run via microwave, 120° C. for 1 hr. The result mixture was concentrated under reduced pressure. The residue was poured into ice-water (20 ml) and the mixture was adjusted pH=7˜8 by aqueous sodium bicarbonate solution. The mixture was extracted by EtOAc (30 ml×3), dried over Na₂SO₄, filtered and concentrated under reduced pressure to afford the compound 55 (800 mg) as gray oil, which was used in next step directly without further purification.

TLC: PE/EA=5:1, UV, R_{f}(compound 54)=0.80, R_{f}(compound 55)=0.30.

### Preparation of methyl 5-(methylthio)-2-(trifluoromethyl)benzoate (56)

To a solution of compound 55 (4.8 g. 20.32 mmol, 1.0 eq) in MeCN (50 mL) was added K₂CO₃ (8.5 g, 60.96 mmol, 3.0 eq) and CH₃I (14.4 g. 101.6 mmol, 5.0 eq) dropwise at 0° C. The reaction mixture was stirred at room temperature for 16 hrs. The result mixture was added water and extracted by EtOAc (50 mL×3), dried over Na₂SO₄, filtered and concentrated under reduced pressure. The residue was purified by column chromatography on silica gel (PE) to afford the compound 56 (4.0 g, 78%) as yellow solid.

TLC: PE/EA=5:1, UV, R_{f}(compound 55)=0.30, R_{f}(compound 56)=0.85.

LC-MS: 251.00 [M+1]+.

### Preparation of methyl 5-(methylsulfonyl)-2-(trifluoromethyl)benzoate (57)

To a solution of compound 56 (4.7 g. 18.78 mmol, 1.0 eq) in DCM (50 mL) was added m-CPBA (19.5 g, 112.68 mmol, 6.0 eq) in portion at 0° C. The reaction mixture was stirred at rt for 16 hrs. The reaction mixture was quenched by solution of sodium bicarbonate. The mixture was extracted by DCM (50 mL×3), washed with NaCl solution (100 mL×3), dried over Na₂SO₄, filtered and concentrated under reduced pressure. The residue was purified by column chromatography on silica gel (PE/EA=100/1 to 50/1 to 20/1 to 10/1) to afford the compound 57 (2.97 g, 56%) as white solid.

TLC: PE/EA=5:1, UV, R_{f}(compound 56)=0.85, R_{f}(compound 57)=0.10.

### Preparation of methyl 5-((2-hydroxy-5-methoxypentyl)sulfonyl)-2-(trifluoromethyl)benzoate (58)

To a solution of compound 57 (0.9 g. 3.543 mmol, 1.0 eq) and 4-methoxybutanal (0.724 mg. 7.086 mmol, 2.0 eq) in THF (10 mL) was added KHMDS (5.4 mL, 5.315 mmol, 1.5 eq) dropwise at −78° C., the reaction mixture was stirred at −78° C. for 2 hrs. The reaction was quenched by aqueous NH₄Cl at 0° C. and extracted by EtOAc (30 mL×3). The organic phase was washed with saturated NaCl solution (100 mL×3), dried over Na₂SO₄, filtered and concentrated under reduced pressure. The residue was purified by column chromatography on silica gel (PE/EA=20/1 to 5/1 to 2/1) to afford the compound 58 (520 mg, 40%) as yellow oil.

TLC: PE/EA=2:1, UV, R_{f}(compound 57)=0.60, R_{f}(compound 58)=0.20.

LC-MS: 385.10 [M+1]+.

### Preparation of 5-((2-hydroxy-5-methoxypentyl)sulfonyl)-2-(trifluoromethyl)benzoic acid (59)

To a solution of compound 58 (510 mg. 1.327 mmol, 1.0 eq) in MeOH/THF=1/1 (6 mL), was added 5% LiOH (63.6 mg, 2.654 mmol, 2.0 eq) dropwise at 0° C. The reaction mixture was stirred at rt for 2 hrs. The reaction mixture was adjusted to pH 2 with 1N HCl. The mixture was extracted by EtOAc (20 mL×3), dried over Na₂SO₄, filtered and concentrated under reduced pressure to afford the compound 59 (505 mg, crude, 100%) as yellow oil.

LC-MS: 393.10 [M+23]⁺.

### Preparation of N-(2-(2-(2-(2-azidoethoxy)ethoxy)ethoxy)ethyl)-5-((2-hydroxy-5-methoxypentyl)sulfonyl)-2-(trifluoromethyl)benzamide (60)

To a solution of compound 59 (1.0 g, 3.24 mmol, 1.0 eq), compound 22 (0.849 g, 3.89 mmol, 1.2 eq), HATU (1.6 g, 4.21 mmol, 1.3 eq), and TEA (0.982 g, 9.72 mol, 3.0 eq) in DMF (12 mL) was stirred at rt for 16 hrs. The reaction mixture was added water (50 mL) and extracted by ethyl acetate (30 mL×3). The organic phase was washed with aqueous solution of NaCl (50 mL×3), dried over Na₂SO₄, filtered and concentrated under reduced pressure. The residue was purified by column chromatography on silica gel (PE/EA=20/1 to 10/1 to 5/1 to 2/1 to 1/1) and prep-TLC to afford the compound 60 (520 mg, 34%) as light-yellow oil.

LC-MS: 571.35 [M+1]+.

### Preparation of 1-((3-((2-(2-(2-(2-azidoethoxy)ethoxy)ethoxy)ethyl)carbamoyl)-4-(trifluoromethyl)phenyl)sulfonyl)-5-methoxypentan-2-yl (2,5-dioxopyrrolidin-1-yl) carbonate (61)

To a solution of compound 60 (0.3 g, 0.5258 mmol, 1.0 eq) in THF (3 mL) was added pyridine (0.166 g, 2.103 mmol, 4.0 eq) and triphosgene (0.39 g, 1.3145 mmol, 2.5 eq) in portion at 0° C. The mixture was stirred at rt for 30 min. The reaction mixture was filtered and concentrated under reduced pressure. The residue was dissolved in THF (3 ml). The mixture was added pyridine (0.166 g, 2.103 mmol, 4.0 eq) and HOSU (0.182 g, 1.5774 mmol, 3.0 eq) in portion at 0° C. The mixture was stirred at rt for 1 hr. The reaction mixture was quenched with water at 0° C. and extracted by EtOAc (20 mL×3). The organic phase was dried over Na₂SO₄, filtered and concentrated under reduced pressure. The residue was purified by prep-HPLC (0.1% HCOOH). The eluting solution was extracted with EtOAc. The organic phase was dried over Na₂SO₄, filtered and concentrated under reduced pressure to afford the compound 61 (150 mg, 40%) as colorless oil.

LC-MS: 712.35[M+1]⁺.

¹H NMR (400 MHz, CDCl₃) δ 8.11 (d, J=9.5 Hz, 2H), 7.94 (d, J=8.1 Hz, 1H), 6.94 (s, 1H), 5.31 (d, J=6.9 Hz, 1H), 3.65 (d, J=6.3 Hz, 8H), 3.59 (q, J=5.0 Hz, 6H), 3.36 (dt, J=18.4, 5.4 Hz, 4H), 3.29 (d, J=1.0 Hz, 3H), 2.83 (s, 4H), 1.90 (q, J=7.2 Hz, 2H), 1.65 (d, J=8.5 Hz, 2H).

### Example 30

### 1-((3-((2-(2-(2-(2-azidoethoxy)ethoxy)ethoxy)ethyl)carbamoyl)-4-chlorophenyl)sulfonyl)-5-methoxypentan-2-yl (25-dioxopyrrolidin-1-yl) carbonate (68)

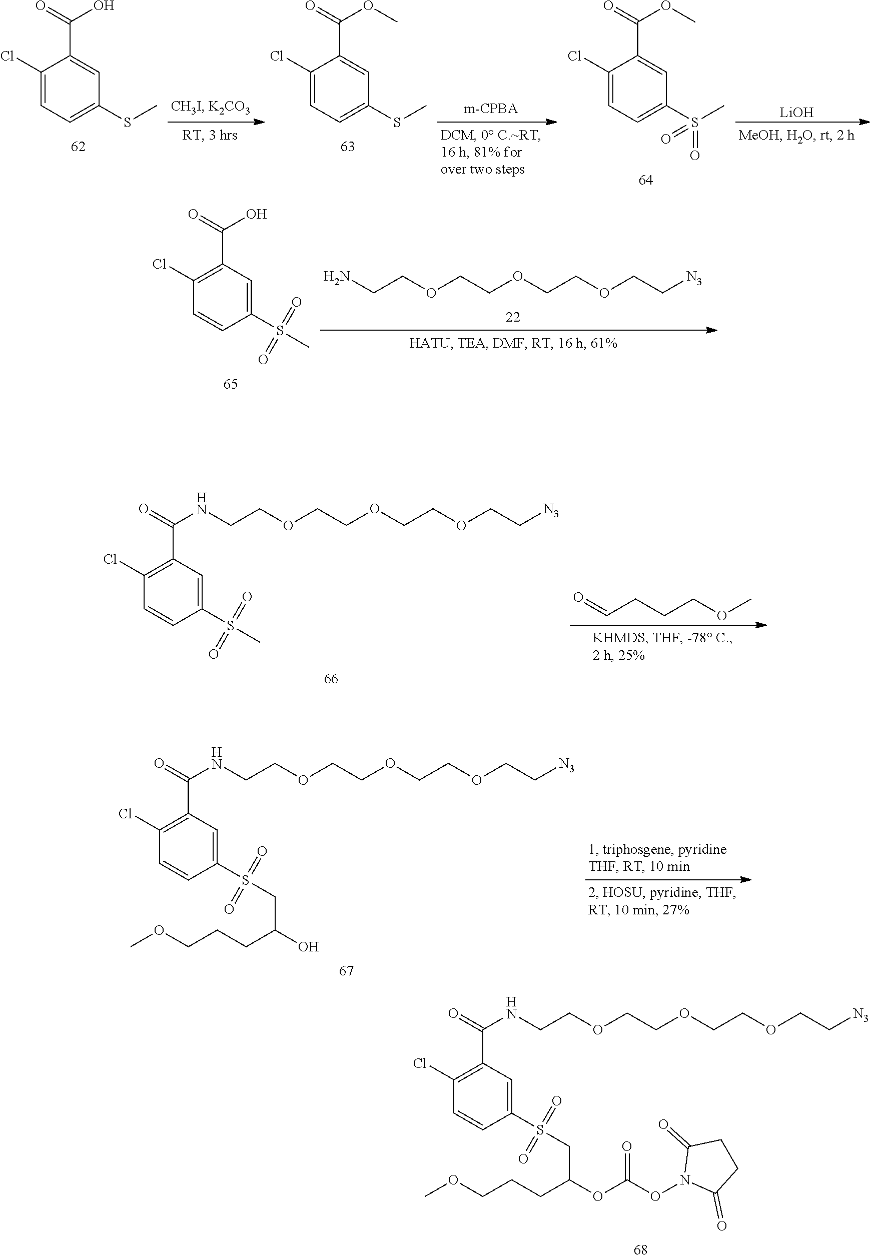

### Preparation of methyl 2-chloro-5-(methylthio)benzoate (63)

To a solution of compound 62 (10.0 g, 49.53 mmol, 1.0 eq), CH₃I (7.73 g, 54.48 mmol, 1.1 eq), was added K₂CO₃ (7.5 g, 54.48 mmol, 1.1 eq) at rt. The reaction mixture was stirred at rt for 3 hrs. The resulting mixture was added water (200 ml) and EtOAc (200 ml). The organic layer was separated, washed with 5% LiCl aqueous solution five times, dried over Na₂SO₄, filtered and concentrated under reduced pressure to afford the compound 63 (11.0 g, crude) as a yellow oil.

TLC: PE/EA=3/1, UV, R_{f}(Compound 62)=0.05, R_{f}(Compound 63)=0.85.

¹HNMR (400 MHz, CD3OD) δ 7.61 (d, J=2.3 Hz, 1H), 7.44-7.32 (m, 2H), 3.88 (s, 3H), 2.48 (s, 3H).

### Preparation of methyl 2-chloro-5-(methylsulfonyl)benzoate (64)

To a solution of compound 63 (6.0 g. 27.78 mmol, 1.0 eq) in DCM (60 ml) was added m-CPBA (28.7 g, 166.67 mmol, 6.0 eq) in portions at 0° C. The reaction mixture was stirred at rt for 16 hrs. The reaction mixture was quenched by sodium bicarbonate aqueous solution, extracted with DCM (100 mL×3), washed with NaCl aqueous solution (100 mL×3), dried over Na₂SO₄, filtered and concentrated under reduced pressure. The residue was purified by column chromatography on silica gel (PE/EA=40/1 to 20/1 to 3/1) to afford the compound 64 (5.6 g, 81%) as a white solid.

TLC: PE/EA=3/1, UV, R_{f}(Compound 63)=0.85, R_{f}(Compound 64)=0.45.

¹HNMR (CD3OD, 400 MHz) δ 8.39 (d, J=2.4 Hz, 1H), 7.96 (dd, J=8.4, 2.4 Hz, 1H), 7.66 (d, J=8.4 Hz, 1H), 3.96 (s, 3H), 3.07 (s, 3H).

### Preparation of 2-chloro-5-(methylsulfonyl)benzoic acid (65)

To a solution of compound 64 (2.5 g. 1.327 mmol, 1.0 eq) in MeOH/THF=1/1 (6 mL), was added 5% LiOH aqueous solution (63.6 mg, 2.654 mmol, 2.0 eq) dropwise at 0° C. The reaction mixture was stirred at rt for 2 hrs. The reaction was adjusted to pH=3-4 with 1N HCl, concentrated. The aqueous was extracted by EtOAc (20 mL×3), dried over Na₂SO₄, filtered and concentrated under reduced pressure to afford the compound 65 (2.1 g, crude) as light-yellow solid.

TLC: PE/EA=3:1, UV, R_{f}(compound 64)=0.45, R_{f}(compound 65)=0.05.

¹HNMR (CD3OD, 400 MHz) δ 8.36 (d, J=2.3 Hz, 1H), 8.02 (dd, J=8.4, 2.3 Hz, 1H), 7.76 (d, J=8.4 Hz, 1H), 3.15 (s, 3H).

### Preparation of N-(2-(2-(2-(2-azidoethoxy)ethoxy)ethoxy)ethyl)-2-chloro-5-(methylsulfonyl)benzamide (66)

To a suspension of compound 65 (879 mg, 3.74 mmol, 1.0 eq), compound 22 (900 mg, 4.12 mmol, 1.1 eq), HATU (1.85 g, 4.87 mmol, 1.3 eq), and TEA (1.14 g, 11.24 mol, 3.0 eq) in DMF (8 ml) was stirred at rt for 16 hrs. The reaction mixture was added water (20 ml) and extracted by ethyl acetate (30 mL×3), washed with NaCl aqueous solution (50 mL×3), dried over Na₂SO₄, filtered and concentrated under reduced pressure. The residue was purified by column chromatography on silica gel (PE/EA=100/1 to 10/1 to 5/1 to 2/1 to 1/1) to afford the compound 66 (995 mg, 61%) as a colorless oil.

TLC: PE/EA=0:1, UV, R_{f}(compound 65)=0.25, R_{f}(compound 66)=0.55.

¹HNMR (CD3OD, 400 MHz) δ 8.04-7.96 (m, 2H), 7.73 (d, J=8.3 Hz, 1H), 3.70-3.53 (m, 14H), 3.33 (s, 2H), 3.15 (s, 3H).

### Preparation of N-(2-(2-(2-(2-azidoethoxy)ethoxy)ethoxy)ethyl)-2-chloro-5-((2-hydroxy-5-methoxypentyl)sulfonyl)benzamide (67)

To a solution of compound 66 (700 mg. 1.609 mmol, 1.0 eq) and 4-methoxybutanal

(657 mg. 6.44 mmol, 4.0 eq) in THF (7 mL) was added KHMDS (5.4 mL, 5.315 mmol, 1.5 eq) dropwise at −78° C. The reaction mixture was stirred at −78° C. for 2 hrs. The reaction was quenched by NH₄Cl aqueous solution at 0° C., extracted by ethyl acetate (30 mL×3), washed with NaCl aqueous solution (100 mL×3), dried over Na₂SO₄, filtered and concentrated under reduced pressure. The residue was purified by column chromatography on silica gel (PE/EA=20/1 to 5/1 to 2/1) to afford the compound 67 (205 mg, 25%) as a light-yellow oil.

TLC: PE/EA=0:1, UV, R_{f}(compound 66)=0.55, R_{f}(compound 67)=0.50.

¹HNMR (CD3OD, 400 MHz) δ 8.01-7.92 (m, 2H), 7.71 (d, J=8.4 Hz, 1H), 4.16-4.01 (m, 2H), 3.72-3.53 (m, 12H), 3.42-3.35 (m, 3H), 3.31-3.25 (m, 5H), 1.73-1.39 (m, 4H).

### Preparation of 1-((3-((2-(2-(2-(2-azidoethoxy)ethoxy)ethoxy)ethyl)carbamoyl)-4-chlorophenyl)sulfonyl)-5-methoxypentan-2-yl (2,5-dioxopyrrolidin-1-yl) carbonate_(68)

A solution of compound 67 (200 mg, 0.372 mmol, 1.0 eq), in THF (2 mL) was added pyridine (117.5 mg, 1.49 mmol, 4.0 eq) and triphosgene (221 mg, 0.744 mmol, 2.0 eq) in portion at 0° C. The mixture was stirred at rt for 30 min. The reaction mixture was filtered and concentrated under reduced pressure. The residue was dissolved in THF (3 ml). The mixture was added pyridine (117.5 mg, 1.49 mmol, 4.0 eq) and HOSU (128 mg, 1.12 mmol, 3.0 eq) in portion at 0° C. The mixture was stirred at rt for 1 hr. The reaction mixture was quenched by water at 0° C., extracted by ethyl acetate (20 mL×3), dried over Na₂SO₄, filtered and concentrated under reduced pressure. The residue was purified by prep-HPLC (0.1% HCOOH), and extracted by ethyl acetate, dried over Na₂SO₄, filtered and concentrated under reduced pressure to afford the compound 68 (101 mg, 27%) as a light yellow oil.

TLC: PE/EA=0/1, UV, R_{f}(Compound 67)=0.50, R_{f}(Compound 68)=0.55.

LC-MS: 678.25 [M+1]⁺.

¹HNMR (400 MHz, CDCl₃) δ 8.09 (s, 1H), 7.94-7.86 (m, 1H), 7.64 (d, J=8.4 Hz, 1H), 7.00 (s, 1H), 5.29 (s, 1H), 3.74-3.52 (m, 15H), 3.44-3.31 (m, 5H), 3.28 (s, 3H), 2.82 (s, 4H), 1.87 (d, J=7.4 Hz, 2H), 1.61 (s, 2H).

### Example 31

### 7-((3-(2,7-bis((2-(2-(2-(2-azidoethoxy)ethoxy)ethoxy)ethyl)carbamoyl)-9H-carbazol-9-yl)propyl)amino)-7-oxo-1-((4-(trifluoromethyl)phenyl)sulfonyl)heptan-2-yl (2,5-dioxopyrrolidin-1-yl) carbonate (82)

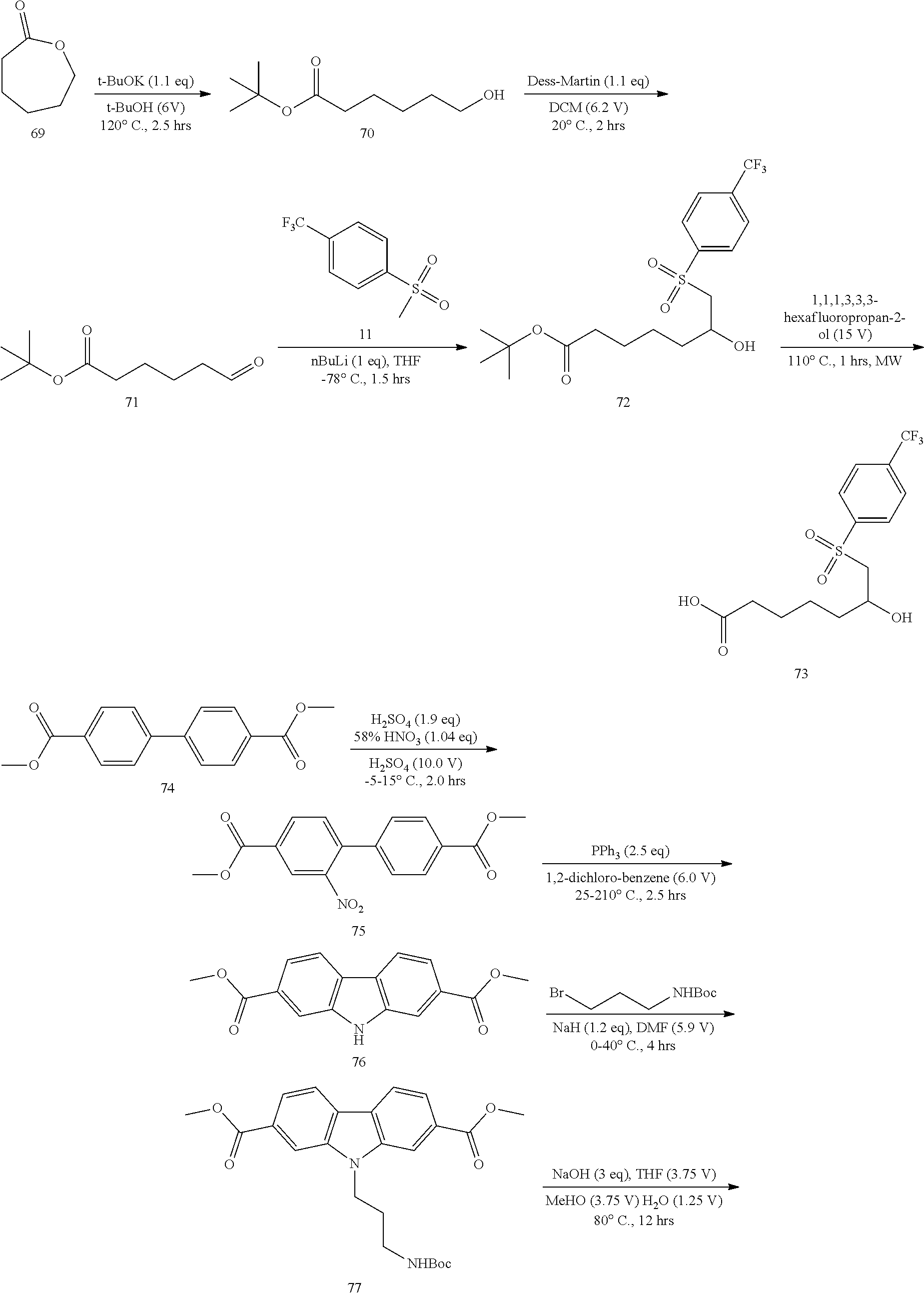

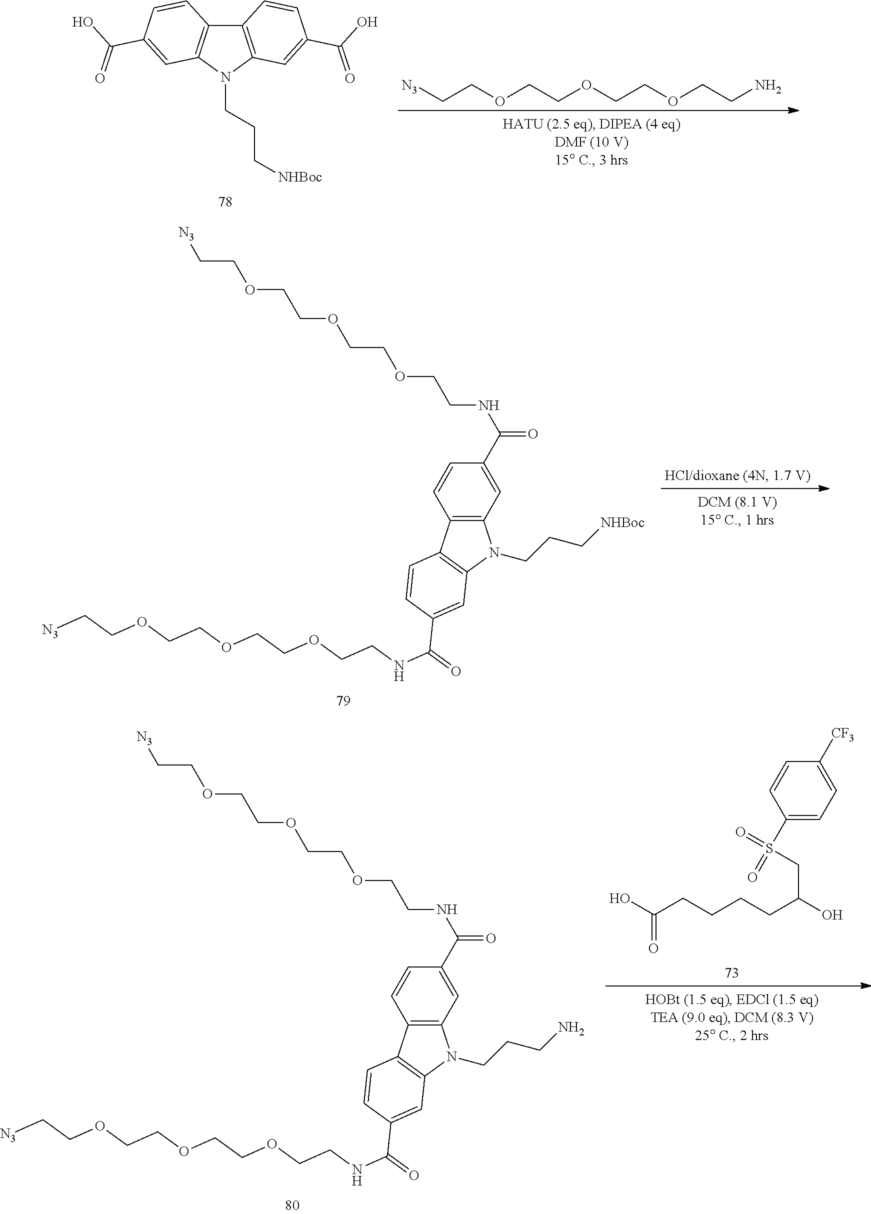 [SMILES: CC(C)(C)OC(=O)NCCCn1c2cc(C(=O)O)ccc2c2ccc(C(=O)O)cc21 ; [N-]=[N+]=NCCOCCOCCOCCN ; CC(C)(C)OC(=O)NCCCn1c2cc(C(=O)NCCOCCOCCOCCN=[N+]=[N-])ccc2c2ccc(C(=O)NCCOCCOCCOCCN=[N+]=[N-])cc21 ; [N-]=[N+]=NCCOCCOCCOCCNC(=O)c1ccc2c3ccc(C(=O)NCCOCCOCCOCCN=[N+]=[N-])cc3n(CCCN)c2c1 ; O=C(O)CCCCC(O)CS(=O)(=O)c1ccc(C(F)(F)F)cc1]

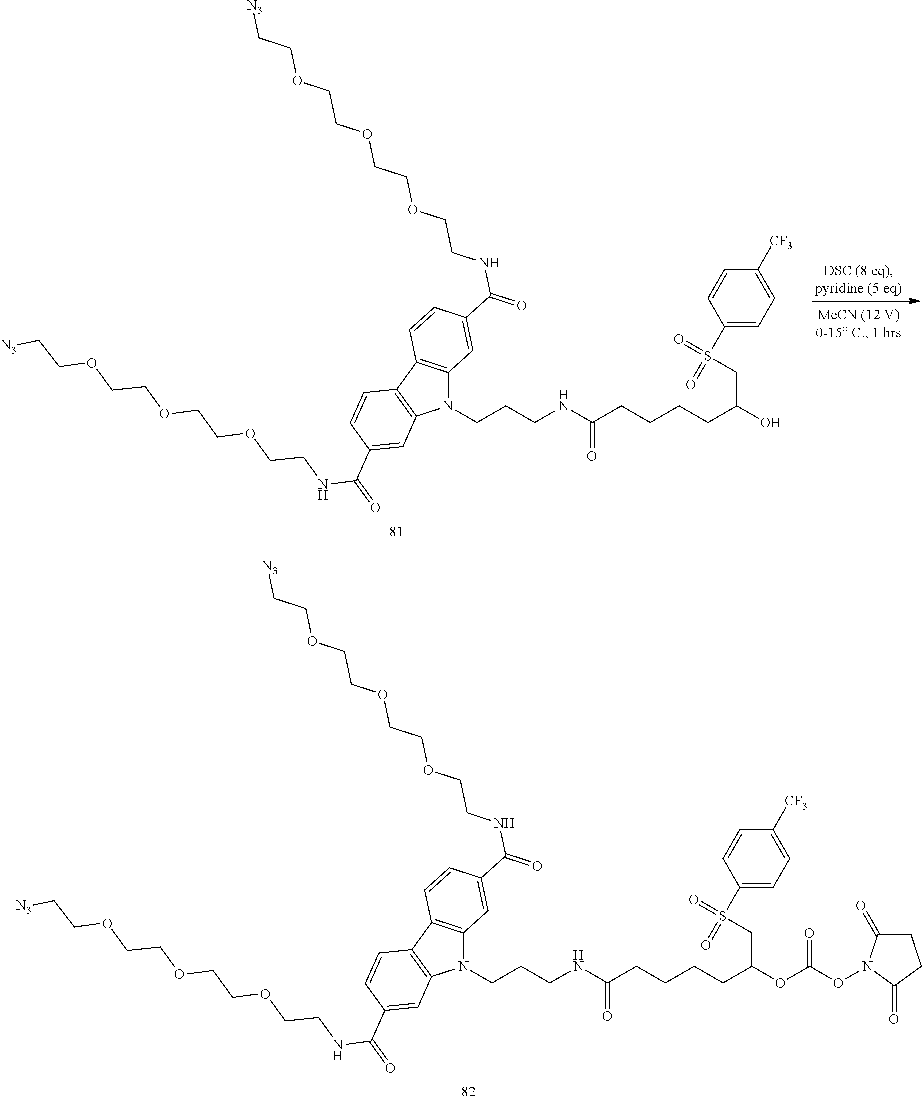 [SMILES: [N-]=[N+]=NCCOCCOCCOCCNC(=O)c1ccc2c3ccc(C(=O)NCCOCCOCCOCCN=[N+]=[N-])cc3n(CCCNC(=O)CCCCC(O)CS(=O)(=O)c3ccc(C(F)(F)F)cc3)c2c1 ; [N-]=[N+]=NCCOCCOCCOCCNC(=O)c1ccc2c3ccc(C(=O)NCCOCCOCCOCCN=[N+]=[N-])cc3n(CCCNC(=O)CCCCC(CS(=O)(=O)c3ccc(C(F)(F)F)cc3)OC(=O)ON3C(=O)CCC3=O)c2c1]

### Preparation of tert-butyl 6-hydroxyhexanoate (70)

A mixture of compound 69 (100 g, 876 mmol) and t-BuOK (108 g, 964 mmol) in t-BuOH (600 mL) was degassed and purged with N₂ for 3 times, and then the mixture was stirred at 120° C. for 2.5 hrs under N₂ atmosphere. TLC (plate 1, dichloromethane/methanol=10/1, compound 69, R_{f}=0.60, compound 70 R_{f}=0.50) indicated compound 69 was consumed completely and one new spot formed. The reaction was clean according to TLC. The reaction mixture was partitioned between dichloromethane (600 mL) and water (1.20 L). The organic phase was separated, washed with brine (300 mL), dried over anhydrous sodium sulfate, filtered and concentrated under reduced pressure to give a residue to give compound 70 (127 g, 77.2% yield) as a yellow oil and was used into the next step without further purification.

¹H NMR (400 MHz, CDCl₃) δ ppm 3.66-3.63 (m, 2H), 2.25-2.21 (m, 2H), 1.66-1.57 (m, 5H), 1.44 (s, 9H), 1.40-1.39 (m, 2H).

### Preparation of tert-butyl 6-oxohexanoate (71)

To a solution of compound 70 (64.0 g, 340 mmol) in DCM (400 mL) was added Dess-Martin reagent (159 g, 374 mmol, 116 mL). The mixture was stirred at 20° C. for 2 hrs. TLC (plate 1, petroleum ether/ethyl acetate=1/1, compound 70 R_{f}=0.40, compound 71 R_{f}=0.50) indicated compound 70 was consumed completely. The reaction mixture was quenched by addition of NaHCO₃ aqueous solution (200 mL), and extracted with DCM (100 mL×3). The combined organic layers were washed with brine (100 mL), dried over Na₂SO₄, filtered and concentrated under reduced pressure to give a residue. The residue was purified by column chromatography (SiO₂, petroleum ether/ethyl acetate=10/1 to 1/1, plate 2, petroleum ether/ethyl acetate=1/1, compound 71 R_{f}=0.50) to give compound 71 (26.8 g, 42.3% yield) as a yellow oil.

¹H NMR: (400 MHz CDCl₃) δ ppm 2.44-2.21 (m, 4H), 1.65-1.60 (m, 4H), 1.43 (s, 9H).

### Preparation of tert-butyl 6-hydroxy-7-((4-(trifluoromethyl)phenyl)sulfonyl)heptanoate (72)

To a solution of compound 11 (7.15 g, 31.9 mmol) in THF (30.0 mL) was added dropwise n-BuLi (2.5 M, 11.60 mL), the mixture was stirred at 0° C. for 30 mins. Then a solution of compound 71 (5.40 g, 29.0 mmol) in THF (5.00 mL) was added at −78° C. The mixture was stirred at −78° C. for 1.5 hrs. TLC (plate 1, petroleum ether/ethyl acetate=1/1, compound 71 R_{f}=0.70, compound 72 R_{f}=0.40) indicated compound 71 was consumed completely. The reaction mixture was quenched by addition of NH₄Cl aqueous solution (50.0 mL), and then extracted with EtOAc (20.0 mL×3). The combined organic layers were washed with brine (30.0 mL), dried over Na₂SO₄, filtered and concentrated under reduced pressure to give a residue. The residue was purified by column chromatography (SiO₂, petroleum ether/ethyl acetate=30/1 to 1/1, plate 2, petroleum ether/ethyl acetate=1/1, compound 72 R_{f}=0.40) to give compound 72 (8.57 g, 72.0% yield) as a yellow solid.

¹H NMR: (400 MHz CDCl₃) δ ppm 8.10-8.08 (d, J=8.4 Hz, 2H), 7.88-7.86 (d, J=8 Hz, 2H), 4.21-4.20 (m, 1H), 3.31-3.16 (m, 3H), 2.23-2.18 (m, 2H), 1.61-1.35 (m, 15H).

### Preparation of 6-hydroxy-7-((4-(trifluoromethyl)phenyl)sulfonyl)heptanoic acid (73)

Compound 72 (1.00 g, 2.44 mmol) was taken up into a microwave tube in 1,1,1,3,3,3-hexafluoro-2-propanol (15.0 mL). The sealed tube was heated at 110° C. for 1 hr under microwave. TLC (petroleum ether/ethyl acetate=1/1, compound 72: R_{f}=0.5, compound 73: R_{f}=0.2) indicated compound 72 was consumed completely. The reaction mixture was concentrated under reduced pressure to give a residue. The crude product was used directly for the next step without purification to give compound 73 (0.860 g, 2.43 mmol, 99.6% yield) as a yellow gum.

¹H NMR: (400 MHz DMSO) δ ppm 11.93 (s, 1H), 8.00-8.13 (m, 4H), 5.11-5.17 ((m, 1H), 4.85 (d, J=5.2 Hz, 1H), 3.90 (s, 1H), 3.43-3.48 (m, 2H), 2.16 (t, J=8.0 Hz, 2H), 1.33-1.46 (m, 6H).

### Preparation of dimethyl 2-nitro-[1,1′-biphenyl]-4,4′-dicarboxylate (75)

A solution of compound 74 (33.0 g, 122 mmol) in H₂SO₄ (330 mL) was cooled to −5° C., and a mixture of HNO₃ (13.8 g, 127 mmol, 9.85 mL, 58% purity) and H₂SO₄ (22.8 g, 232 mmol, 12.4 mL) was added drop-wise over a period of 1 hr under stirring, maintaining the temperature at −5-0° C. The mixture was then stirred for 1 hr at −5-0° C. TLC (petroleum ether/ethyl acetate=3/1, product R_{f}=0.50) showed compound 74 (R_{f}=0.60) was consumed, a main new spot with larger polarity was formed. The mixture was diluted with (300 mL) of water, and extracted with ethyl acetate (50.0 mL×2). The extract was washed with brine (50.0 mL) and a solution of sodium hydrogen carbonate (100 mL), dried over anhydrous sodium sulfate, and evaporated. The residue was purified by column chromatography (SiO₂, petroleum ether/ethyl acetate=50/1 to 0/1) to give compound 75 (16.0 g, 50.6 mmol, 41.4% yield, 99.6% purity) as a white solid.

¹H NMR: (400 MHz, CDCl₃) δ 8.57 (s, 1H), 8.31-8.29 (d, J=8.0 Hz, 1H), 8.14-8.12 (d, J=8.4 Hz, 2H), 7.56-7.54 (d, J=8.0 Hz, 1H), 7.42-7.40 (d, J=8.4 Hz, 2H), 4.01 (s, 3H), 3.96 (s, 3H).

### Preparation of dimethyl 9H-carbazole-2,7-dicarboxylate (76)

A mixture of compound 75 (20 g, 63.4 mmol), PPh₃ (41.6 g, 159 mmol) in 1,2-dichlorobenzene (112 mL) was degassed at 25° C., and purged with N₂ for 3 times, and then the mixture was stirred at 210° C. for 1.5 hrs under N₂ atmosphere. TLC (petroleum ether/ethyl acetate=1/1, compound 75: R_{f}=0.43) show compound 75 was consumed completely and one new main spot formed. The reaction was clean according to TLC. The reaction was cooled to 25° C., methanol (200 mL) was added. After 15 mins, the resulting suspension of solids was collected by filtration to give compound 76 (12.0 g, 42.4 mmol, 66.8% yield) was obtained as a gray solid.

¹H NMR: (400 MHz, DMSO) δ 11.81 (s, 1H), 8.33 (d, J=4.2 Hz, 2H), 8.17 (s, 2H), 7.82 (d, J=7.6 Hz, 2H), 3.91 (s, 6H).

### Preparation of dimethyl 9-(3-((tert-butoxycarbonyl)amino)propyl)-9H-carbazole-2,7-dicarboxylate (77)

To a solution of NaH (2.30 g, 57.6 mmol, 60% purity) in DMF (80.0 mL) was added compound 76 (13.6 g, 48.0 mmol) at 0° C. The mixture was stirred at 0° C. for 1 hr, and then tert-butyl N-(3-bromopropyl)carbamate (22.9 g, 96.0 mmol) was added, the mixture was stirred at 40° C. for 3 hrs. TLC (petroleum ether/ethyl acetate=5/1, compound 76: R_{f}=0.2, product: R_{f}=0.7) indicated compound 76 was consumed completely. The reaction mixture was diluted with aqueous NH₄Cl (100 mL) and extracted with EtOAc (150 mL×2). The combined organic layers were washed with brine (100 mL), dried over Na₂SO₄, filtered and concentrated under reduced pressure to give a residue. The residue was purified by column chromatography (SiO₂, petroleum ether/ethyl acetate=10/1 to 1/1) to give compound 77 (16.4 g, 37.2 mmol, 77.6% yield) as a yellow solid.

¹HNMR: (400 MHz, DMSO) δ 8.36 (d, J=8.4 Hz, 2H), 8.31 (s, 2H), 7.80 (d, J=8.4 Hz, 2H), 7.03 (t, J=4.8 Hz, 1H), 4.56 (t, J=6.4 Hz, 2H), 3.74 (s, 6H), 2.99-3.00 (m, 2H), 1.87-1.98 (m, 2H), 1.22-1.36 (m, 9H).

### Preparation of 9-(3-((tert-butoxycarbonyl)amino)propyl)-9H-carbazole-2,7-dicarboxylic acid (78)

A mixture of compound 77 (8.00 g, 18.2 mmol) and NaOH (2.18 g, 54.5 mmol) in THF (30.0 mL), MeOH (30.0 mL) and H₂O (10.0 mL) was degassed and purged with N₂ for 3 times, and then the mixture was stirred at 80° C. for 12 hrs under N₂ atmosphere. TLC (dichloromethane/methanol=10/1, compound 77: R_{f}=0.8) indicated compound 77 was consumed completely. The reaction mixture was poured into 100 mL of ice-water carefully and diluted with 1 N HCl to pH=4. The reaction mixture was filtered and the filter cake was washed with 20.0 mL of water, dried in vacuum. The crude product was used directly for the next step without further purification to give compound 78 (5.00 g, 12.1 mmol, 66.8% yield) as a light yellow solid.

¹HNMR: (400 MHz, CDCl₃) δ 13.01 (s, 2H), 8.34 (d, J=8.0 Hz, 2H), 8.25 (s, 2H), 7.85 (q, J=8.0 Hz, 2H), 4.56 (t, J=6.4 Hz, 2H), 2.97-3.00 (m, 2H), 1.89-1.99 (m, 2H), 1.37 (m, 8H).

### Preparation of tert-butyl (3-(2,7-bis((2-(2-(2-(2-azidoethoxy)ethoxy)ethoxy)ethyl)carbamoyl)-9H-carbazol-9-yl)propyl)carbamate (79)

To a solution of compound 78 (5.00 g, 12.1 mmol) in DMF (50.0 ML) was added HATU (11.5 g, 30.3 mmol) and DIPEA (6.27 g, 48.5 mmol) and 2-[2-[2-(2-azidoethoxy)ethoxy]ethoxy]ethanamine (5.29 g, 24.3 mmol). The mixture was stirred at 15° C. for 3 hrs. LC-MS showed one new peak (compound 79: Rt=0.752 min) with desired MS detected. The reaction mixture was diluted with water (90.0 mL) and extracted with 2-Me-THF (50.0 mL×2). The combined organic layers were washed with water (50.0 mL), dried over Na₂SO₄, filtered and concentrated under reduced pressure to give a residue. The crude product was purified by reversed-phase HPLC (0.1% NH₄HCO₃ condition) to give compound 79 (4.00 g, 4.92 mmol, 40.6% yield) as a white solid.

¹HNMR: (400 MHz, DMSO) δ 8.68 (t, J=5.2 Hz, 2H), 8.31 (d, J=8.4 Hz, 2H), 8.19 (s, 2H), 7.79 (d, J=8.4 Hz, 2H), 7.03 (t, J=4.8 Hz, 2H), 4.53 (t, J=7.2 Hz, 2H), 3.54-3.65 (m, 26H), 3.40-3.41 (m, 4H), 3.38-3.40 (m, 2H), 3.03-3.05 (m, 2H), 1.99-2.02 (m, 2H), 1.40 (s, 9H).

### Preparation of 9-(3-aminopropyl)-N2,N7-bis(2-(2-(2-(2-azidoethoxy)ethoxy)ethoxy)ethyl)-9H-carbazole-2,7-dicarboxamide (80)

To a solution of compound 79 (3.00 g, 3.69 mmol) in DCM (25.0 mL) was added HCl/MeOH (5.00 mL). The mixture was stirred at 15° C. for 1 hr. TLC (dichloromethane/methanol=10/1, compound 79: R_{f}=0.6, compound 80: R_{f}=0.05) indicated compound 79 was consumed completely. The reaction mixture was concentrated under reduced pressure to give a residue. The crude product was used directly for the next step without further purification to give compound 80 (2.70 g, 3.60 mmol, 97.7% yield, HCl salt) as a yellow solid.

¹HNMR: (400 MHz, DMSO) δ 8.78 (t, J=5.6 Hz, 2H), 8.36 (s, 2H), 8.27 (d, J=8.0 Hz, 2H), 8.05 (s, 3H), 7.77 (d, J=8.4 Hz, 2H), 4.63 (t, J=6.8 Hz, 2H), 3.65-3.60 (m, 17H), 3.50-3.56 (m, 5H), 3.36-3.37 (m, 5H), 2.88-2.91 (m, 2H), 2.14-2.18 (m, 2H).

### Preparation of N2,N7-bis(2-(2-(2-(2-azidoethoxy)ethoxy)ethoxy)ethyl)-9-(3-(6-hydroxy-7-((4-(trifluoromethyl)phenyl)sulfonyl)heptanamido)propyl)-9H-carbazole-2,7-dicarboxamide (81)

A mixture of compound 80 (1.80 g, 2.40 mmol, HCl), compound 73 (851 mg, 2.40 mmol), HOBt (487 mg, 3.60 mmol), EDCI (691 mg, 3.60 mmol) and Et₃N (2.19 g, 21.6 mmol) in DCM (15.0 mL) was degassed and purged with N₂ for 3 times, and then the mixture was stirred at 25° C. for 2 hrs under N₂ atmosphere. LC-MS showed one new peak (compound 81: Rt=1.21 min) with desired MS detected. The reaction mixture was diluted with water (30.0 mL) and extracted with EtOAc (20.0 mL×3). The combined organic layers were washed with brine (30.0 mL), dried over Na₂SO₄, filtered and concentrated under reduced pressure to give a residue. The residue was purified by prep-HPLC (column: Xtimate C18 10u 250 mm×80 mm; mobile phase: [water (10 mM NH₄HCO₃)-ACN]; B %: 35%-65%, 21 min) to give compound 81 (1.00 g, 953 umol, 39.7% yield) as a light yellow solid.

¹HNMR: (400 MHz, DMSO) δ 8.71 (t, J=5.6 Hz, 2H), 8.33 (d, J=8.4 Hz, 2H), 8.20 (s, 2H), 8.16 (d, J=8.0 Hz, 2H), 8.05 (d, J=8.4 Hz, 2H), 7.92-7.93 (m, 1H), 7.81 (d, J=8.4 Hz, 2H), 4.89 (d, J=7.0 Hz, 1H), 4.55 (t, J=7.2 Hz, 2H), 3.93 (s, 1H), 3.56-3.67 (m, 30H), 3.40-3.42 (m, 5H), 3.15-3.16 (m, 2H), 2.01-2.11 (m, 4H), 1.27-1.51 (m, 7H).

### Preparation of 7-((3-(2,7-bis((2-(2-(2-(2-azidoethoxy)ethoxy)ethoxy)ethyl)carbamoyl)-9H-carbazol-9-yl)propyl)amino)-7-oxo-1-((4-(trifluoromethyl)phenyl)sulfonyl)heptan-2-yl (2,5-dioxopyrrolidin-1-yl) carbonate (82)

To a solution of compound 81 (500 mg, 477 umol) and N,N′-disuccinimidyl carbonate (977 mg, 3.81 mmol) in ACN (6.00 mL) was added pyridine (188 mg, 2.38 mmol) at 0° C. The mixture was stirred at 15° C. for 1 hr. LC-MS showed one new peak (product: Rt=2.26 min) with desired MS detected. The reaction mixture was diluted with water (20.0 mL) and extracted with DCM (10.0 mL×5). The combined organic layers were washed with water (20.0 mL), dried over Na₂S04, filtered and concentrated under reduced pressure to give a residue. The residue was purified by prep-HPLC (column: Phenomenex luna C18 250×50 mm×10 um; mobile phase: [water (0.04% HCl)-ACN]; B %: 50%-70%, 10 min) to give 82 (0.102 g, 79.4 umol, 16.7% yield, 92.7% purity) as a yellow solid.

¹HNMR: (400 MHz, DMSO) δ 8.6 (t, J=5.6 Hz, 2H), 8.26 (d, J=8.0 Hz, 2H), 8.13-8.17 (m, 4H), 8.01-8.11 (m, 3H), 7.96 (d, J=5.6 Hz, 2H), 5.16-5.18 (m, 1H), 4.49 (t, J=6.4 Hz, 2H), 3.91-4.12 (m, 13H), 3.55-3.59 (m, 14H), 4.49-4.53 (m, 4H), 3.34-3.36 (m, 4H), 3.09-3.10 (m, 2H), 2.79 (s, 4H), 1.97-2.06 (m, 4H), 1.61-1.68 (m, 2H), 1.42-1.44 (m, 2H), 1.23-1.25 (m, 2H).

HPLC: Retention Time: 2.632 min, Area Percent: 92.0%.

LCMS: Retention Time: 2.630 min, M+H⁺=1190.4.

### Example 32

### 7-azido-1-((3-((2-(2-(2-(2-azidoethoxy)ethoxy)ethoxy)ethyl)carbamoyl)phenyl)sulfonyl)heptan-2-yl (2,5-dioxopyrrolidin-1-yl) carbonate (86)

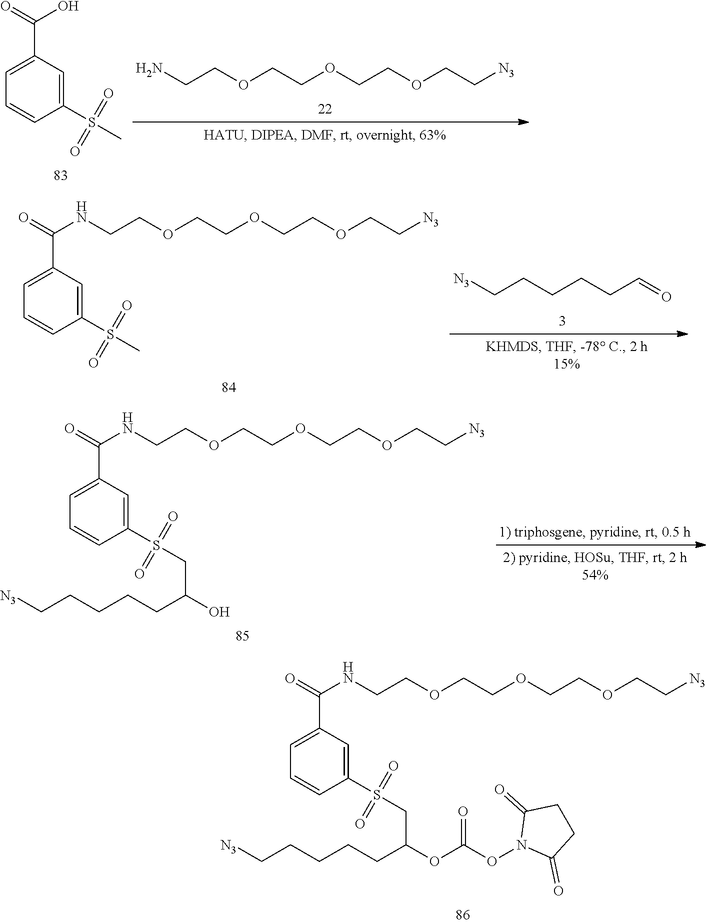 [SMILES: CS(=O)(=O)c1cccc(C(=O)O)c1 ; [N-]=[N+]=NCCOCCOCCOCCN ; CS(=O)(=O)c1cccc(C(=O)NCCOCCOCCOCCN=[N+]=[N-])c1 ; [N-]=[N+]=NCCCCCC=O ; [N-]=[N+]=NCCCCCC(O)CS(=O)(=O)c1cccc(C(=O)NCCOCCOCCOCCN=[N+]=[N-])c1 ; [N-]=[N+]=NCCCCCC(CS(=O)(=O)c1cccc(C(=O)NCCOCCOCCOCCN=[N+]=[N-])c1)OC(=O)ON1C(=O)CCC1=O]

### Preparation of N-(2-(2-(2-(2-azidoethoxy)ethoxy)ethoxy)ethyl)-3-(methylsulfonyl)benzamide (84)

To the solution of compound 83 (2.0 g, 10 mmol, 1.0 eq) and compound 22 (2.18 g, 10 mmol, 1.0 eq) in dimethylformamide (40 mL) was added 2-(7-aza-1H-benzotriazole-1-yl)-1,1,3,3-tetramethyluronium hexafluorophosphate (4.56 g, 12 mmol, 1.2 eq) and N,N-diisopropylethylamine (2.0 g, 20 mmol, 2.0 eq). The mixture was stirred at room temperature overnight. The reaction was monitored by LCMS and TLC. The mixture was diluted with water (50 mL), extracted with ethyl acetate (5×150 mL) and washed with brine (100 mL). The organic layer was dried over sodium sulfate and concentrated under reduced pressure. The residue was purified by column chromatography on a silica gel (dichloromethane: methanol, 97:3) to give compound 84 (2.5 g, 63%).

TLC: dichloromethane: methanol=10:1, UV 254 nm, by I₂, R_{f}: (Compound 83)=0.3; R_{f}: (Compound 84)=0.5.

### Preparation of 3-((7-azido-2-hydroxyheptyl)sulfonyl)-N-(2-(2-(2-(2-azidoethoxy)ethoxy)ethoxy)ethyl)benzamide (85)

To the solution of compound 84 (2.0 g, 5.0 mmol, 1.0 eq) in tetrahydrofuran (30 mL) was added a solution of potassium bis(trimethylsilyl)amide (1.0 M, 15 mL, 15 mmol, 3.0 eq) slowly at −78° C. Then compound 3 (2.1 g, 15 mmol, 3.0 eq) was added to the mixture. The reaction mixture was stirred at room temperature for 2 h. The reaction was monitored by TLC. Then the mixture was quenched with saturated ammonium chloride aqueous solution (30 mL), extracted with ethyl acetate (2×30 mL). The organic layers were washed with brine (20 mL), dried over sodium sulfate, filtered and concentrated under reduced pressure. The residue was purified by column chromatography on a silica gel (dichloromethane: methanol, 97:3) to provide compound 85 (400 mg, 15%).

TLC: dichloromethane: methanol=10:1, UV 254 nm, R_{f}: (Compound 84)=0.5; R_{f}: (Compound 85)=0.5.

### Preparation of 7-azido-1-((3-((2-(2-(2-(2-azidoethoxy)ethoxy)-ethoxy)ethyl)carbamoyl)phenyl)sulfonyl)heptan-2-yl (2,5-dioxopyrrolidin-1-yl) carbonate (86)

To the mixture of compound 85 (400 mg, 0.74 mmol, 1.0 eq) in tetrahydrofuran (4 mL) was added triphosgene (372 mg, 1.25 mmol, 1.7 eq) and pyridine (117 mg, 1.48 mmol, 2.0 eq). After stirring for 30 min, the reaction mixture was filtered. To the filtrate was added pyridine (117 mg, 1.48 mmol, 2.0 eq) and N-hydroxysuccinimide (176 mg, 0.89 mmol, 1.2 eq). The mixture was stirred at room temperature for 2 h. The reaction was monitored by LCMS. The mixture was extracted with ethyl acetate (3×5 mL) and washed with brine (5 mL). Then the organic layer was dried over sodium sulfate, filtered and concentrated under reduced pressure. The residue was purified by prep-HPLC to provide compound 86 (270 mg, 54%) as colorless oil.

LCMS: [M+1]⁺=683.

¹HNMR (400 MHz, CD₃OD): δ 8.32 (s, 1H), 8.17 (d, J=8.0 Hz, 1H), 8.04 (d, J=7.6 Hz, 1H), 7.68 (t, J=7.6 Hz, 1H), 7.29 (s, 1H), 5.25 (s, 1H), 3.59-3.66 (m, 16H), 3.37-3.32 (m, 2H), 3.25 (t, J=6.8 Hz, 2H), 2.81 (s, 4H), 1.79 (s, 2H), 1.57 (s, 2H), 1.39 (s, 4H).

### Example 33

### 1-azido-12-(2-(2-(2-(2-azidoethoxy)ethoxy)ethoxy)ethyl)-13-oxo-19-((4-(trifluoromethyl)phenyl)sulfonyl)-3,6,9-trioxa-12-azanonadecan-18-yl (2,5-dioxopyrrolidin-1-yl) carbonate (89)

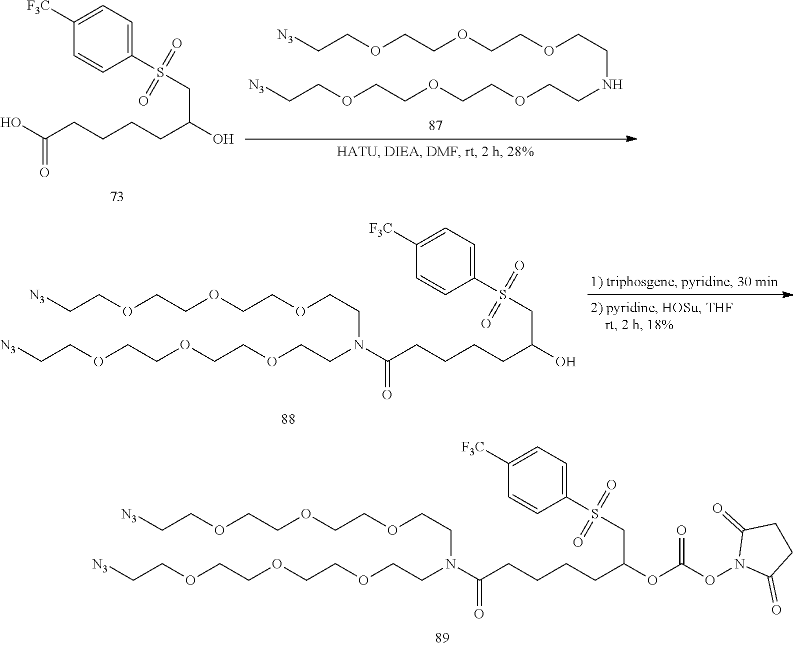 [SMILES: O=C(O)CCCCC(O)CS(=O)(=O)c1ccc(C(F)(F)F)cc1 ; [N-]=[N+]=NCCOCCOCCOCCNCCOCCOCCOCCN=[N+]=[N-] ; [N-]=[N+]=NCCOCCOCCOCCN(CCOCCOCCOCCN=[N+]=[N-])C(=O)CCCCC(O)CS(=O)(=O)c1ccc(C(F)(F)F)cc1 ; [N-]=[N+]=NCCOCCOCCOCCN(CCOCCOCCOCCN=[N+]=[N-])C(=O)CCCCC(CS(=O)(=O)c1ccc(C(F)(F)F)cc1)OC(=O)ON1C(=O)CCC1=O]

### Preparation of N,N-bis(2-(2-(2-(2-azidoethoxy)ethoxy)ethoxy)ethyl)-6-hydroxy-7-((4-(trifluoromethyl)phenyl)sulfonyl)heptanamide (88)

To the solution of compound 73 (102 mg, 0.3 mmol, 1.2 eq) in dimethylformamide (3 mL) was added 2-(7-aza-1H-benzotriazole-1-yl)-1,1,3,3-tetramethyluronium hexafluorophosphate (136 mg, 0.76 mmol, 1.5 eq) and N,N-diisopropylethylamine (124 mg, 0.96 mmol, 4.0 eq). The mixture was stirred at room temperature for 10 min. Then to the mixture was added compound 87 (100 mg, 0.24 mmol, 1.0 eq) and stirred for 2 h. The reaction was monitored by LCMS and TLC. The mixture was diluted with water (10 mL), extracted with ethyl acetate (5×10 mL) and washed with brine (10 mL). The organic layer was dried over sodium sulfate, filtered and concentrated under reduced pressure. The residue was purified by column chromatography on a silica gel (dichloromethane: methanol, 98:2) to give compound 88 (50 mg, 28%).

TLC: dichloromethane: methanol=10:1, UV 254 nm, by I₂, R_{f}: (Compound 87)=0.5; R_{f}: (Compound 88)=0.4.

### Preparation of 1-azido-12-(2-(2-(2-(2-azidoethoxy)ethoxy)ethoxy)ethyl)-13-oxo-19-((4-(trifluoromethyl)phenyl)sulfonyl)-3,6,9-trioxa-12-azanonadecan-18-yl (2,5-dioxopyrrolidin-1-yl) carbonate (89)

To the mixture of compound 88 (400 mg, 0.53 mmol, 1.0 eq) in tetrahydrofuran (4 mL) was added triphosgene (267 mg, 0.9 mmol, 1.7 eq) and pyridine (84 mg, 1.06 mmol, 2.0 eq). The reaction mixture was stirred for 30 min. The reaction mixture was filtered. To the filtrate was added pyridine (84 mg, 1.06 mmol, 2.0 eq) and N-hydroxysuccinimide (73 mg, 0.64 mmol, 1.2 eq). The mixture was stirred at room temperature for 2 h. The reaction was monitored by LCMS. The mixture was extracted with ethyl acetate (3×5 mL) and washed with brine (5 mL). Then the mixture was dried over sodium sulfate, filtered and concentrated under reduced pressure. The residue was purified by prep-HPLC to provide compound 89 (85 mg, 18%) as yellow oil.

LCMS: [M+1]⁺=897.

¹HNMR (400 MHz, CD₃OD): δ 8.15-8.13 (d, J=8.0 Hz, 2H), 7.96-7.94 (d, J=8.8 Hz, 2H), 5.27 (m, 1H), 3.89 (m, 1H), 3.73 (m, 1H), 3.59-3.61 (m, 26H), 3.35 (m, 6H), 2.81 (s, 4H), 3.46-3.42 (m, 2H), 1.79-1.77 (m, 2H), 1.58 (m, 2H) and 1.39-1.37 (m, 2H).

### Example 34

### 15 kDa Y-PEG-DBCO

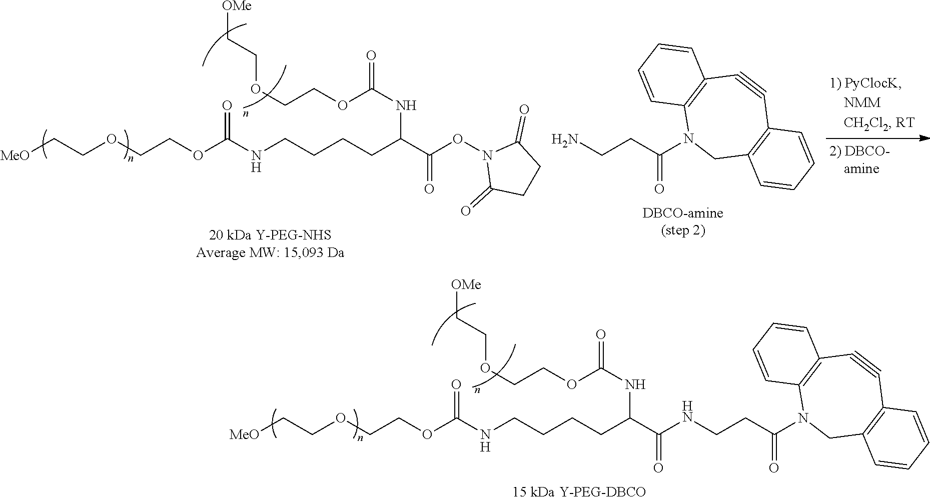

To a dried round-bottomed flask, equipped with a Teflon coated magnetic stir bar was added 15 kDa Y-PEG-NHS (1.13 g, 74.9 μmol, 1.0 equiv) and PyClocK (0.082 g, 148 μmol, 2.0 equiv). The flask was sealed with a rubber septum and placed under an inert atmosphere of Argon. Anhydrous CH₂Cl₂ (18 mL) was added, followed by N-methylmorpholine (18 μL, 164 μmol, 2.2 equiv) and the reaction was stirred at room temperature for 30 min. DBCO-amine (52 mg, 188 μmol, 2.5 equiv) was added in one portion as a solution in CH₂Cl₂ (2 mL) with N-methylmorpholine (18 μL, 164 μmol, 2.2 equiv) and the reaction mixture was stirred at room temperature for a further 5 h. The crude reaction mixture was concentrated under vacuum and then taken up hot 2-propanol (120 mL). The resulting solution was cooled in an ice-bath to form a precipitate. The isolated precipitate was transferred to pre-weighed falcon tubes (×3) and the precipitate was sedimented by centrifugation (12000 rpm, 30 min, −3° C.). The precipitation was repeated once with 2-propanol (120 mL) and three times with acetone (3×120 mL). The pellets were dried in vacuo. Isolated white solid, mass=995 mg (88%). R^{p}-HPLC retention time=6.9 min.

### Example 35

### 17 kDa Y-PEG-DBCO

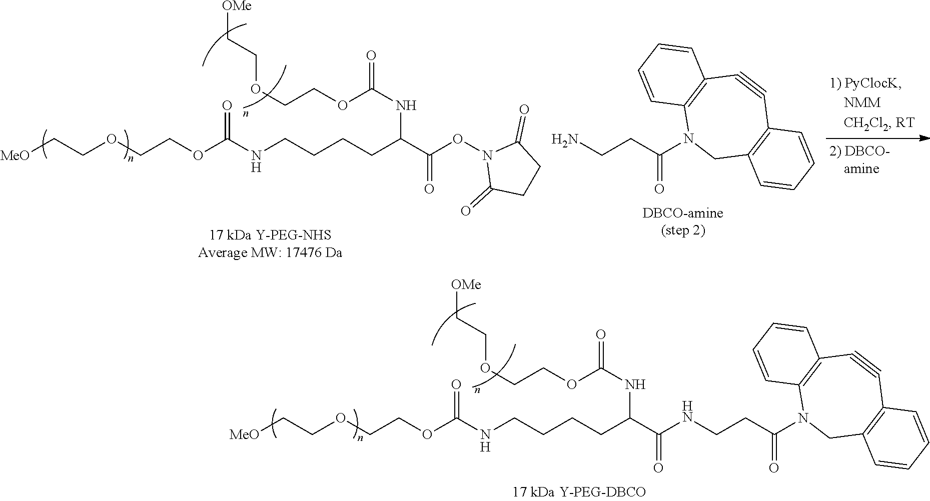

To a dried round-bottomed flask, equipped with a Teflon coated magnetic stir bar was added 17 kDa Y-PEG-NHS (1.0 g, 57.2 μmol, 1.0 equiv) and CH₂Cl₂ (18.0 mL). The flask was sealed with a rubber septum and placed under an inert atmosphere of Argon. DBCO-amine (40 mg, 145 μmol, 2.5 equiv) followed by N-methylmorpholine (19 μL, 173 μmol, 3.0 equiv) were added and the reaction was stirred at room temperature overnight. The crude reaction mixture was concentrated under vacuum and then taken up hot acetone (90 mL). The resulting solution was cooled in an ice-bath for 30 min to form a precipitate which was sedimented by centrifugation (11000 rpm, 30 min, −8° C.). The solvent was decanted and the precipitation process was repeated with once 2-propanol (90 mL) and twice with acetone (2×90 mL). The resulting solid was dried in vacuo. Isolated white solid, mass=910 mg (91%). R^{p}-HPLC retention time=6.7 min.

### Example 36

### 7.5 kDa PEG-DBCO

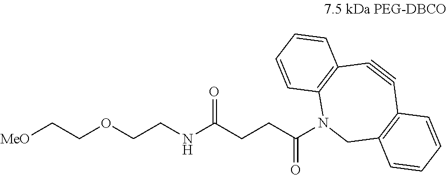 [SMILES: COCCOCCNC(=O)CCC(=O)N1Cc2ccccc2C#Cc2ccccc21]

The 7.5 kDa PEG-DBCO reagent was purchased from JenKem Technology USA. HPLC: purity 98.0%; GPC: purity 99.1%; MALDI: 7481 Da.

### Example 37

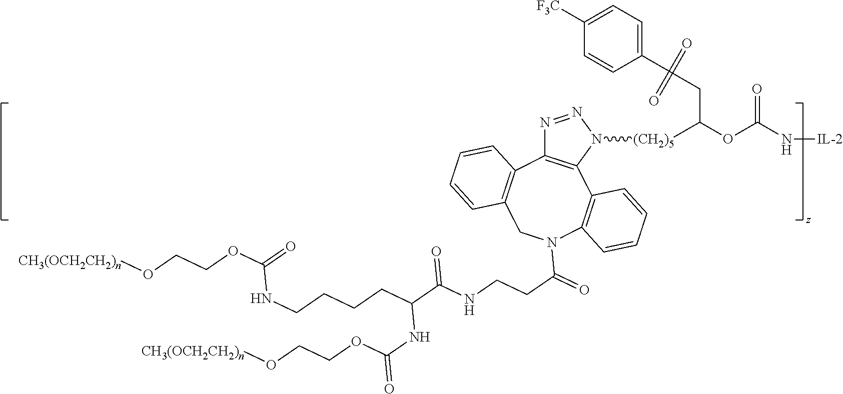

### NHS Conjugation of rIL-2 with Example 2 and Click-PEGylation with 15 kDa Y-PEG-DBCO

Example 2 (3.2 mg) was dissolved in DMF (439 μL) to give a 7.29 mg/mL solution of the reagent. IL-2 (8.0 mg, 0.523 μmol, 2.76 mL) was diluted with 100 mM sodium borate, pH 8, 20 mM EDTA, 0.05% SDS (841 μL) and example 2 (2.92 mg, 5.77 μmol, 400 μL, 11 eq.) was added. The reaction was mixed and incubated at 22° C. for 1 h. After 1 h, the reaction was analysed by LC-MS to determine the average degree of IL-2 functionalisation.

15 kDa Y-PEG-DBCO (125 mg) was dissolved in 100 mM sodium borate, pH 8.20 mM EDTA, 0.05% SDS (500 μL) to give a 250 mg/mL solution. To [rIL-2]-[CF₃-Ph-SO₂—N₃]_{z} (7.6 mg, 0.497 μmol, 3.80 mL), 15 kDa Y-PEG-DBCO (114 mg, 7.47 μmol, 455 μL, 15 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 37 was quantified by IR using a DirectDetect instrument as [15K mPEG-(CF₃-Ph-SO₂)]_{z}-[rIL-2] (5.55 mg, 73% yield). SDS-PAGE analysis of the conjugate showed PEG:IL-2 ratio equaled to 6.7.

### Example 38

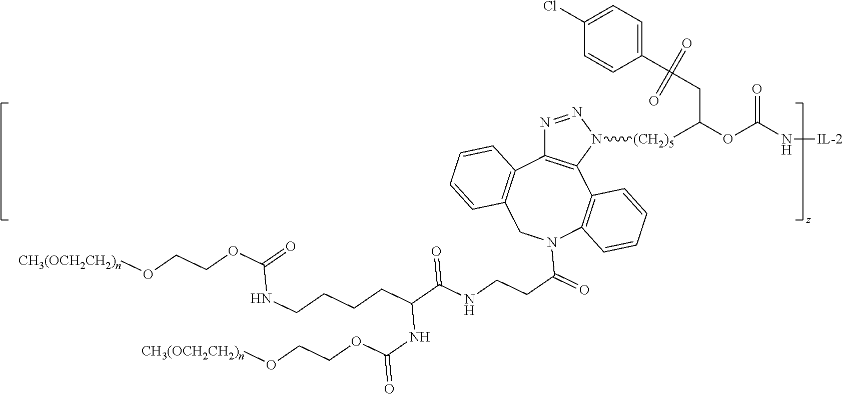

### NHS Conjugation of rIL-2 with Example 3 and Click-PEGylation with 15 kDa Y-PEG-DBCO

Example 3 (3.0 mg) was dissolved in DMF (607 μL) to give a 4.94 mg/mL solution of the reagent. IL-2 (8.0 mg, 0.523 μmol, 2.67 mL) was diluted with 100 mM sodium borate, pH 8, 20 mM EDTA, 0.05% SDS (933 μL) and example 3 (1.98 mg, 4.19 μmol, 400 μL, 8 eq.) was added. The reaction was mixed and incubated at 22° C. for 1 h. After 1 h, the reaction was analysed by LC-MS to determine the average degree of IL-2 functionalisation.

15 kDa Y-PEG-DBCO (150 mg) was dissolved in 100 mM sodium borate, pH 8.20 mM EDTA, 0.05% SDS (1.00 mL) to give a 150 mg/mL solution. To [rIL-2]-[Cl-Ph-SO₂—N₃]_{z}(7.7 mg, 0.503 μmol, 3.65 mL), 15 kDa Y-PEG-DBCO (138 mg, 9.06 μmol, 921 μL, 18 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 38 was quantified by IR using a DirectDetect instrument as [15K mPEG-(C₁-Ph-SO₂)]_{z}-[rIL-2] (6.39 mg, 83% yield). SDS-PAGE analysis of the conjugate showed PEG:IL-2 ratio equaled to 5.4.

### Example 39

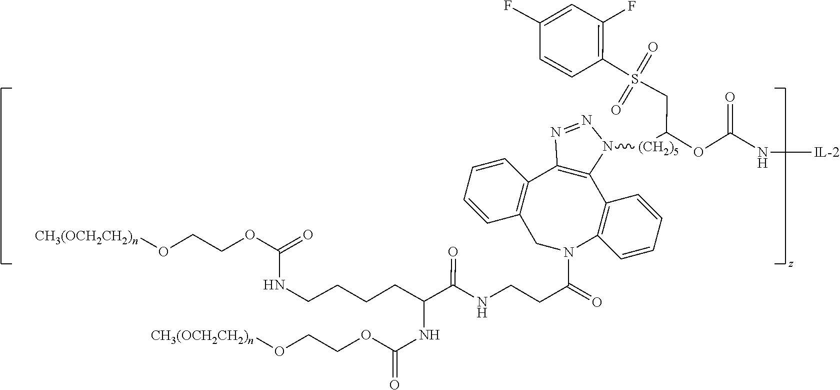

### NHS Conjugation of rIL-2 with Example 4 and Click-PEGylation with 15 kDa Y-PEG-DBCO

Example 4 (4.0 mg) was dissolved in DMF (269 μL) to give a 14.9 mg/mL solution of the reagent. To a vial of IL-2 (8 mg, 0.523 μmol, 4.00 mL), example 4 (2.98 mg, 6.28 μmol, 200 μL, 12 eq.) was added. The reaction was mixed and incubated at 22° C. for 1 h. After 1 h, the reaction was analysed by LC-MS to determine the average degree of IL-2 functionalisation.

15 kDa Y-PEG-DBCO (125 mg) was dissolved in 100 mM sodium borate, pH 8.20 mM EDTA, 0.05% SDS (833 μL) to give a 150 mg/mL solution of the reagent. To [rIL-2]-[F,F-Ph-SO₂—N₃]_{z} (8.0 mg, 0.523 μmol, 4.20 mL), 15 kDa Y-PEG-DBCO (120 mg, 7.87 μmol, 798 μL, 15 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 39 was quantified by IR using a DirectDetect instrument as [15K mPEG-(F,F-Ph-SO₂)]_{z}-[rIL-2] (7.26 mg, 91% yield). SDS-PAGE analysis of the conjugate showed PEG:IL-2 ratio equaled to 5.9.

### Example 40

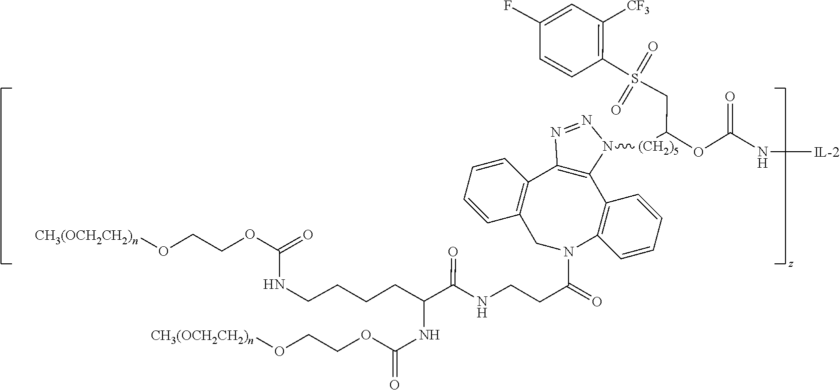

### NHS Conjugation of rIL-2 with Example 5 and Click-PEGylation with 15 kDa Y-PEG-DBCO

Example 5 (4.5 mg) was dissolved in DMF (438 μL) to give a 10.3 mg/mL solution of the reagent IL-2 (7 mg, 0.458 μmol, 2.33 mL) was diluted with 100 mM sodium borate, pH 8, 20 mM EDTA, 0.05% SDS (817 μL) and example 5 (3.61 mg, 6.88 μmol, 350 μL, 15 eq.) was added. The reaction was mixed and incubated at 22° C. for 1 h. After 1 h, the reaction was analysed by LC-MS to determine the average degree of IL-2 functionalisation.

15 kDa Y-PEG-DBCO (120 mg) was dissolved in 100 mM sodium borate, pH 8.20 mM EDTA, 0.05% SDS (800 μL) to give a 150 mg/mL solution of the reagent. To [rIL-2]-[F,CF₃-Ph-SO₂—N₃]_{z} (7.0 mg, 0.458 μmol, 3.50 mL), 15 kDa Y-PEG-DBCO (105 mg, 6.88 μmol, 698 μL, 15 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 40 was quantified by IR using a DirectDetect instrument as [15K mPEG-(F,CF₃-Ph-SO₂)]_{z}-[rIL-2] (6.73 mg, 96% yield). SDS-PAGE analysis of the conjugate showed PEG:IL-2 ratio equaled to 5.9.

### Example 41

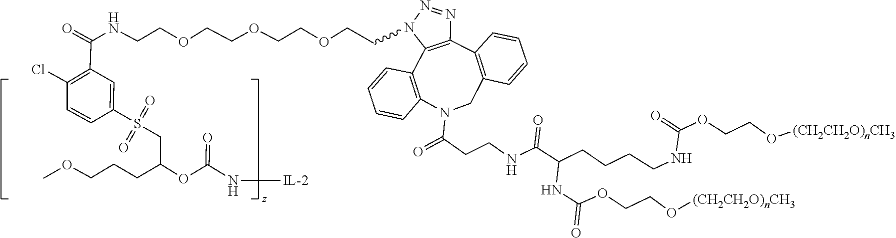

### NHS Conjugation of rIL-2 with Example 30 and Click-PEGylation with 15 kDa Y-PEG-DBCO

Example 30 (101 mg) was dissolved in DMF (2.02 mL) to give a 50 mg/mL solution of the reagent. To a vial of IL-2 (12 mg, 0.784 μmol, 4.88 mL), example 30 (21.3 mg, 31.4 μmol, 425 μL, 40 eq.) and DMF (28.3 μL) were added. The reaction was mixed and incubated at 22° C. for 1 h. After 1 h, the reaction was analysed by LC-MS to determine the average degree of IL-2 functionalisation.

15 kDa Y-PEG-DBCO (578 mg) was dissolved in 100 mM sodium borate, pH 8.20 mM EDTA, 0.05% SDS (2.09 mL) to give a 277 mg/mL solution of the reagent. To [rIL-2]-[Cl,CONH-Ph-SO₂—N₃]_{z} (11.6 mg, 0.758 μmol, 5.16 mL), 15 kDa Y-PEG-DBCO (578 mg, 37.89 μmol, 2.09 mL, 50 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 41 was quantified by IR using a DirectDetect instrument as [15K mPEG-(Cl,CONH-Ph-SO₂)]_{z}-[rIL-2] (5.37 mg, 46% yield). SDS-PAGE analysis of the conjugate showed PEG:IL-2 ratio equaled to 5.4.

### Example 42

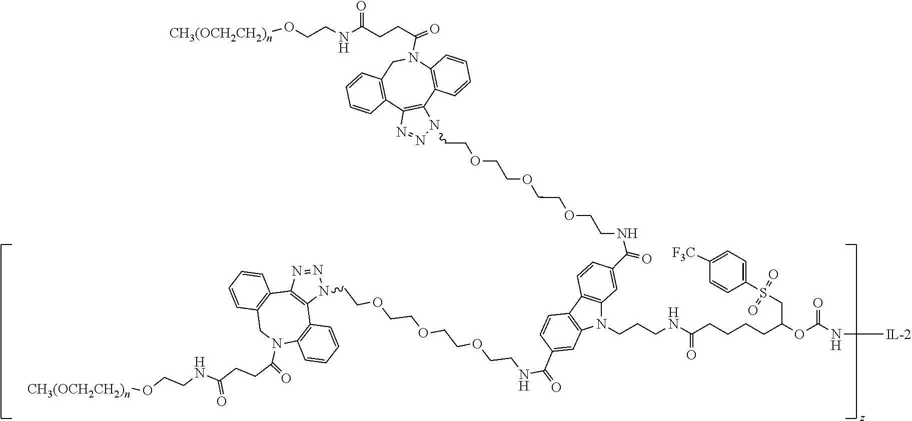

### NHS Conjugation of rIL-2 with Example 31 and Click-PEGylation with 7.5 kDa PEG-DBCO

Example 31 (50 mg) was dissolved in DMF (1.00 mL) to give a 50 mg/mL solution of the reagent IL-2 (12 mg, 0.784 μmol, 4.8 mL) was diluted with 100 mM sodium borate, pH 8, 20 mM EDTA, 0.05% SDS (0.6 mL) and example 31 (14 mg, 11.8 μmol, 280 μL, 15 eq.) and DMF (320 μL) were added. The reaction was mixed and incubated at 22° C. for 1 h. At 1 h, the reaction was analysed by LC-MS to determine the distribution of functionalised IL-2 species.

7.5 kDa PEG-DBCO (250 mg) was dissolved in 100 mM sodium borate, pH 8 (1.67 mL) to give a 150 mg/mL solution. To [rIL-2]-[CF₃-Ph-Ar—SO₂—N₃]_{z} (12 mg, 0.784 μmol, 6.0 mL), 7.5 kDa PEG-DBCO (206 mg, 27.5 μmol, 1.37 mL, 35 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Example 42 was quantified by IR using a DirectDetect instrument as [2×7.5K mPEG-(CF₃-Ph-Ar—SO₂)]_{z}-[rIL-2] (10 mg, 84% yield). SDS-PAGE analysis of the conjugate showed PEG:IL-2 ratio equaled to 5-7.

### Example 43

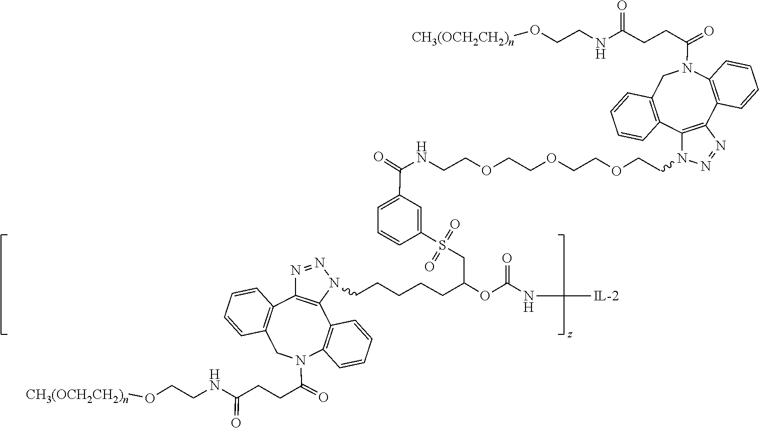

### NHS Conjugation of rIL-2 with Example 32 and Click-PEGylation with 7.5 kDa PEG-DBCO

Example 32 (99 mg) was dissolved in DMF (1.98 mL) to give a 50 mg/mL solution of the reagent IL-2 (12 mg, 0.784 μmol, 4.0 mL) was diluted with 100 mM sodium borate, pH 8, 20 mM EDTA, 0.05% SDS (1.40 mL) and example 32 (9.1 mg, 13.4 μmol, 181 μL, 17 eq.) and DMF (419 μL) were added. The reaction was mixed and incubated at 22° C. for 1 h. At 1 h, the reaction was analysed by LC-MS to determine the distribution of functionalised IL-2 species.

7.5 kDa PEG-DBCO (250 mg) was dissolved in 100 mM sodium borate, pH 8 (1.67 mL) to give a 150 mg/mL solution. To [rIL-2]-[CONH-Ph-R—SO₂—N₃]_{z} (11.8 mg, 0.771 μmol, 5.9 mL), 7.5 kDa PEG-DBCO (231 mg, 30.9 μmol, 1.54 mL, 40 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Example 43 was quantified by IR using a DirectDetect instrument as [2×7.5K mPEG-(CONH-Ph-R—SO₂)]_{z}-[rIL-2] (10.23 mg, 87% yield). SDS-PAGE analysis of the conjugate showed PEG:IL-2 ratio equaled to 5-7.

### Example 44

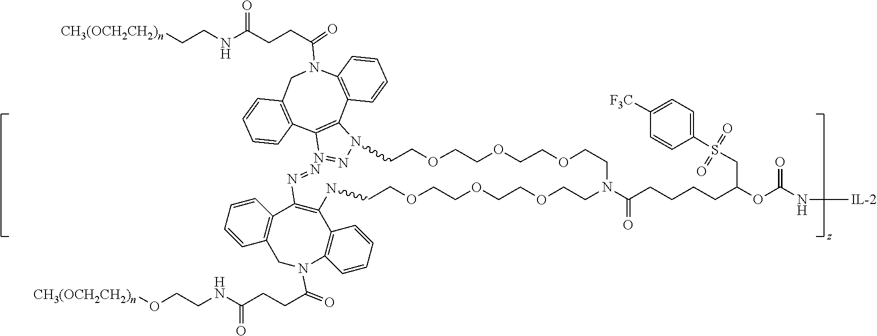

### NHS Conjugation of rIL-2 with Example 33 and Click-PEGylation with 7.5 kDa PEG-DBCO

Example 33 (11.3 mg) was dissolved in DMF (0.226 mL) to give a 50 mg/mL solution of the reagent IL-2 (10.8 mg, 0.706 μmol, 3.60 mL) was diluted with 100 mM sodium borate, pH 8, 20 mM EDTA, 0.05% SDS (1.26 mL) and example 33 (9.50 mg, 10.6 μmol, 190 μL, 15 eq.) and DMF (0.35 mL) were added. The reaction was mixed and incubated at 22° C. for 1 h. At 1 h, the reaction was analysed by LC-MS to determine the distribution of functionalised IL-2 species.

7.5 kDa PEG-DBCO (240 mg) was dissolved in 100 mM sodium borate, pH 8 (1.60 mL) to give a 150 mg/mL solution. To [rIL-2]-[CF₃-Ph-R—SO₂—N₃]_{z} (10.5 mg, 0.686 μmol, 3.85 mL), 7.5 kDa PEG-DBCO (205 mg, 27.4 μmol, 1.37 mL, 40 eq.) was added. The reaction was mixed and incubated at 22° C. The reaction mixture was analysed by SDS-PAGE after 2 h and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the method were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF). Example 44 was quantified by IR using a DirectDetect instrument as [2×7.5K mPEG-(CF₃-Ph-R—SO₂)]_{z}-[rIL-2] (10.45 mg, 99% yield). SDS-PAGE analysis of the conjugate showed PEG:IL-2 ratio equaled to 5.6.

### Example 45

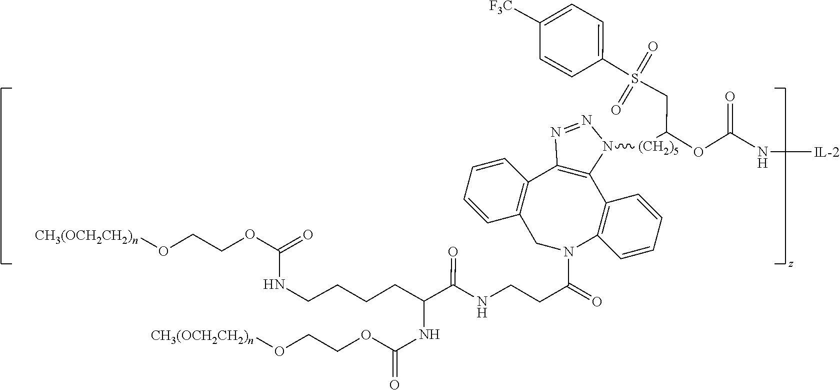

### NHS Conjugation of rIL-2 with Example 2 and Click-PEGylation with 17 kDa Y-PEG-DBCO

Example 2 (5.0 mg) was dissolved in DMF (687 μL) to give a 7.28 mg/mL solution of the reagent IL-2 (12.0 mg, 0.784 μmol, 4.14 mL) was diluted with 100 mM sodium borate, pH 9, 20 mM EDTA, 0.05% SDS (1.26 mL) and example 2 (4.37 mg, 8.63 μmol, 600 μL, 11 eq.) was added. The reaction was mixed and incubated at 22° C. for 15 min. After 15 min, the reaction was analysed by LC-MS to determine the average degree of IL-2 functionalisation. Additional example 2 (1.99 mg, 3.93 μmol, 273 μL, 5 eq.) was added to the reaction to increase the level of functionalisation. After a further 15 min incubation at 22° C., the reaction was analysed by LC-MS to determine the average degree of IL-2 functionalisation.

17 kDa Y-PEG-DBCO (302 mg) was dissolved in 100 mM sodium borate, pH 9.20 mM EDTA, 0.05% SDS (1.21 mL) to give a 250 mg/mL solution. To [rIL-2]-[CF₃-Ph-SO₂—N₃]_{z} (12.0 mg, 0.784 μmol, 6.00 mL), 17 kDa Y-PEG-DBCO (277 mg, 15.7 μmol, 1.11 ML, 20 eq.) was added. The reaction was mixed and incubated at 22° C. The reaction mixture was analysed by SDS-PAGE after 15 min and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

### Example 46

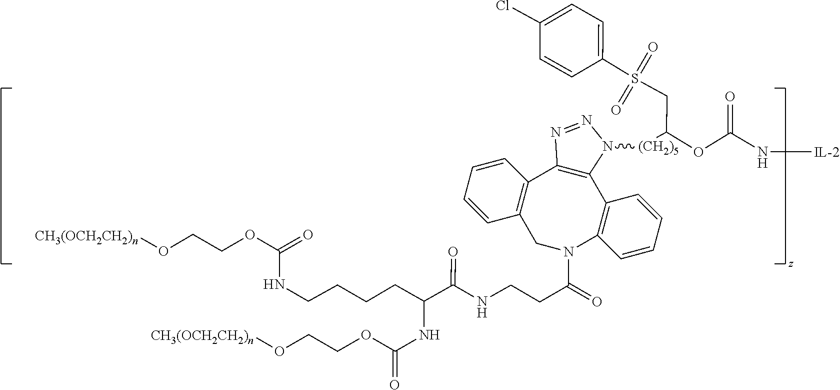

### NHS Conjugation of rIL-2 with Example 3 and Click-PEGylation with 17 kDa Y-PEG-DBCO

Example 3 (4.2 mg) was dissolved in DMF (679 μL) to give a 6.18 mg/mL solution of the reagent IL-2 (12.0 mg, 0.784 μmol, 4.14 mL) was diluted with 100 mM sodium borate, pH 9, 20 mM EDTA, 0.05% SDS (1.26 mL) and example 3 (3.71 mg, 7.84 μmol, 600 μL, 10 eq.) was added. The reaction was mixed and incubated at 22° C. for 15 min. After 15 min, the reaction was analysed by LC-MS to determine the average degree of IL-2 functionalisation.

17 kDa Y-PEG-DBCO (185 mg) was dissolved in 100 mM sodium borate, pH 9.20 mM EDTA, 0.05% SDS (740 μL) to give a 250 mg/mL solution. To [rIL-2]-[Cl-Ph-SO₂—N₃]_{z} (12.0 mg, 0.784 μmol, 6.00 mL), 17 kDa Y-PEG-DBCO (173 mg, 9.80 μmol, 692 μL, 12.5 eq.) and 100 mM sodium borate, pH 9, 20 mM EDTA, 0.05% SDS (165 μL) were added. The reaction was mixed and incubated at 22° C. The reaction mixture was analysed by SDS-PAGE after 15 min and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 46 was quantified by IR using a DirectDetect instrument as [17K mPEG-(C₁-Ph-SO₂)]_{z}-[rIL-2] (9.7 mg, 81% yield). SDS-PAGE analysis of the conjugate showed PEG:IL-2 ratio equaled to 6.2.

### Example 47

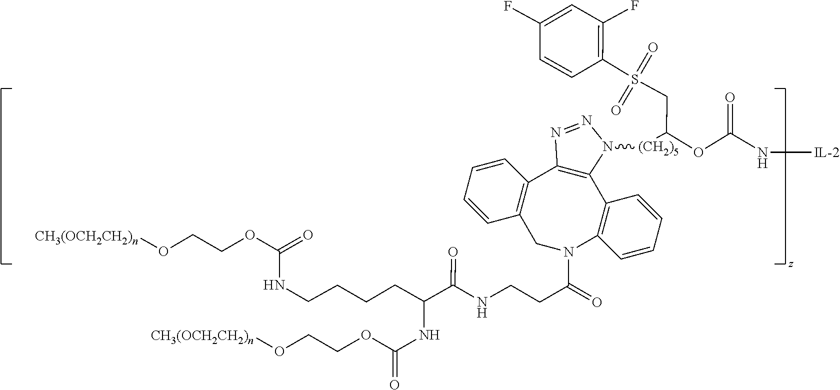

### NHS Conjugation of rIL-2 with Example 4 and Click-PEGylation with 17 kDa Y-PEG-DBCO

Example 4 (6.0 mg) was dissolved in DMF (645 μL) to give a 9.30 mg/mL solution of the reagent IL-2 (10.5 mg, 0.686 μmol, 3.62 mL) was diluted with 100 mM sodium borate, pH 9, 20 mM EDTA, 0.05% SDS (1.10 mL) and example 4 (4.88 mg, 10.3 μmol, 525 μL, 15 eq.) was added. The reaction was mixed and incubated at 22° C. for 15 min. After 15 min, the reaction was analysed by LC-MS to determine the average degree of IL-2 functionalisation.

17 kDa Y-PEG-DBCO (260 mg) was dissolved in 100 mM sodium borate, pH 9.20 mM EDTA, 0.05% SDS (1.04 mL) to give a 250 mg/mL solution of the reagent. To [rIL-2]-[F,F-Ph-SO₂—N₃]_{z} (10.5 mg, 0.686 μmol, 5.25 mL), 17 kDa Y-PEG-DBCO (242 mg, 13.7 μmol, 968 μL, 20 eq.) was added. The reaction was mixed and incubated at 22° C. The reaction mixture was analysed by SDS-PAGE after 15 min and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 47 was quantified by IR using a DirectDetect instrument as [17K mPEG-(F,F-Ph-SO₂)]_{z}-[rIL-2] (9.5 mg, 91% yield). SDS-PAGE analysis of the conjugate showed PEG:IL-2 ratio equaled to 6.5.

### Example 48

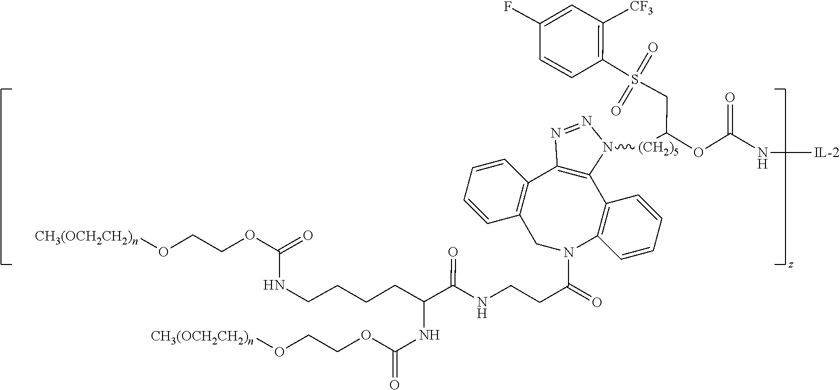

### NHS Conjugation of rIL-2 with Example 5 and Click-PEGylation with 17 kDa Y-PEG-DBCO

Example 5 (6.6 mg) was dissolved in DMF (600 μL) to give a 11.0 mg/mL solution of the reagent IL-2 (12.0 mg, 0.784 μmol, 4.49 mL) was diluted with 100 mM sodium borate, pH 9, 20 mM EDTA, 0.05% SDS (906 μL) and example 5 (6.20 mg, 11.8 μmol, 560 μL, 15 eq.) was added. The reaction was mixed and incubated at 22° C. for 15 min. After 15 min, the reaction was analysed by LC-MS to determine the average degree of IL-2 functionalisation.

17 kDa Y-PEG-DBCO (235 mg) was dissolved in 100 mM sodium borate, pH 9.20 mM EDTA, 0.05% SDS (0.94 mL) to give a 250 mg/mL solution of the reagent. To [rIL-2]-[F,CF₃-Ph-SO₂—N₃]_{z} (12.0 mg, 0.784 μmol, 6.00 mL), 17 kDa Y-PEG-DBCO (228 mg, 12.9 μmol, 912 μL, 16.5 eq.) was added. The reaction was mixed and incubated at 22° C. The reaction mixture was analysed by SDS-PAGE after 15 min and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 48 was quantified by IR using a DirectDetect instrument as [17K mPEG-(F,CF₃-Ph-SO₂)]_{z}-[rIL-2] (9.4 mg, 78% yield). SDS-PAGE analysis of the conjugate showed PEG:IL-2 ratio equaled to 7.3.

### Example 49

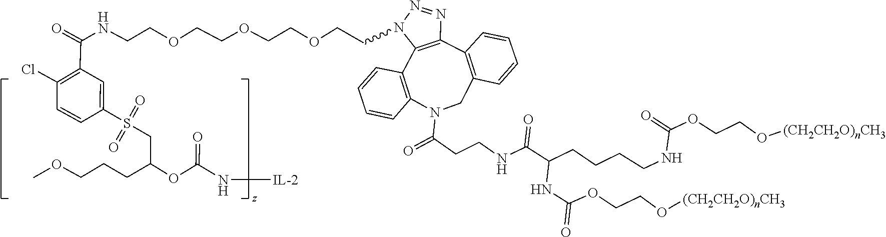

### NHS Conjugation of rIL-2 with Example 30 and Click-PEGylation with 17 kDa Y-PEG-DBCO

Example 30 (101 mg) was dissolved in DMF (2.02 mL) to give a 50 mg/mL solution of the reagent, this solution was diluted to 35.5 mg/mL with DMF prior to conjugation. IL-2 (12.0 mg, 0.784 μmol, 4.49 mL), was diluted with 100 mM sodium borate, pH 9, 20 mM EDTA, 0.05% SDS (906 μL) and example 30 (21.3 mg, 31.4 μmol, 600 μL, 40 eq.) and was added. The reaction was mixed and incubated at 22° C. for 15 min. After 15 min, the reaction was analysed by LC-MS to determine the average degree of IL-2 functionalisation.

17 kDa Y-PEG-DBCO (700 mg) was dissolved in 100 mM sodium borate, pH 9.20 mM EDTA, 0.05% SDS (2.80 mL) to give a 250 mg/mL solution of the reagent. To [rIL-2]-[Cl,CONH-Ph-SO₂—N₃]_{z} (12.0 mg, 0.784 μmol, 6.00 mL), 17 kDa Y-PEG-DBCO (692 mg, 39.2 μmol, 2.77 mL, 50 eq.) was added. The reaction was mixed and incubated at 22° C. The reaction mixture was analysed by SDS-PAGE after 15 min and the crude reaction mixture was purified by SEC using a HiLoad 26/600 Superdex 200 pg. Sample was isocratically eluted with 50 mM sodium acetate, pH 4.5 (150 mM NaCl) at 3 mL/min flow rate. Fractions collected over the methods were analysed by SDS-PAGE and high purity fractions were pooled. The pooled fractions were concentrated/buffer exchanged into 50 mM sodium acetate, pH 4.5 (150 mM NaCl) by UF/DF (Vivaspin20, 50 kDa MWCO PES) and finally sterile filtered (0.22 μm PVDF).

Example 49 was quantified by IR using a DirectDetect instrument as [17K mPEG-(Cl,CONH-Ph-SO₂)]_{z}-[rIL-2] (9.3 mg, 77% yield). SDS-PAGE analysis of the conjugate showed PEG:IL-2 ratio equaled to 7.0.

### Example 50

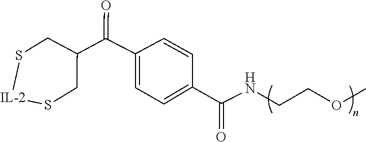

### PEGylation of rIL-2 Disulfide Bond with 20 kDa PEG bis(sulfone)

Prior to conjugation, IL-2 solution (10 mM sodium acetate, pH 4.5, 5% trehalose) was buffer exchanged by gel filtration using CentriPure P100 columns, equilibrated with 100 mM sodium borate, pH 8 (0.05% SDS) as per the manufacturer's instructions. The buffer exchanged protein solution was quantified by UV-A280 using a Nanodrop 2000 spectrophotometer (1.34 mg/mL).

IL-2 (12 mg, 8.96 mL) was diluted to 9 mL with 100 mM sodium borate, pH 8 (0.05% SDS) and to this solution 0.1 M DTT (1.0 mL, 100 μmol, 127 equiv.) was added, giving a final IL-2 concentration of 1.2 mg/mL. The resulting reduction reaction was mixed gently and incubated at 22° C. for 1 h. The reduced IL-2 was buffer exchanged into fresh 100 mM sodium borate, pH 8 (0.05% SDS) using a CentriPure P100 column as per the manufacturer's instructions and the amount of protein recovered (10.5 mg in 21.08 mL of reaction buffer) was determined by UV-A280. A solution of 20 kDa TheraPEG™ reagent (19.5 mg) was prepared in water (3.59 mL). To reduced IL-2 (10.5 mg, 0.686 μmol, 21.08 mL), 100 mM sodium borate, pH 8 (0.05% SDS) (17.71 mL) and 5.44 mg/mL solution of 20 kDa TheraPEG™ (3.37 mL, 0.892 μmol, 1.3 equiv.) were added, giving a final IL-2 concentration of 0.25 mg/mL. The conjugation reaction was mixed gently and incubated at 22° C. for 16 h. The crude reaction was analysed by SDS-PAGE and analytical SEC and then purified by preparative SEC.

Crude reaction was buffer exchanged into 50 mM sodium acetate, pH 4.5 (150 mM NaCl) and concentrated by UF/DF (Vivapsin20, 5 kDa MWCO PES). SDS was removed from PEGylated IL-2 sample using 4 mL Detergent Removal Spin Columns (Pierce®) as per the manufacturer's instructions. PEGylated IL-2 product was then purified by SEC using a HiLoad 16/600 Superdex 200 pg. Sample was isocratically eluted with 50 mM sodium acetate, pH 4.5 (150 mM NaCl) at 2 mL/min flow rate. Fractions collected over the method were analysed by SDS-PAGE and high purity fractions were pooled. The pooled fractions were sterile filtered (0.22 μm PVDF).

Sample was quantified by UV-A280 using a Nanodrop 2000 spectrophotometer and was analysed by SEC and SDS-PAGE. Example 50 was generated as 0.9 mg (8% yield) in solution. Purity by SDS-PAGE: >99%. Purity by Analytical SEC: 96.1%.

### Example 51

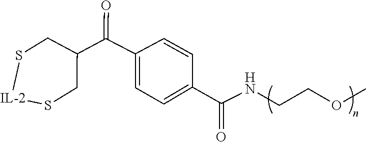

### PEGylation of rIL-2 Disulfide Bond with 5 kDa PEG bis(sulfone)

Prior to conjugation, IL-2 solution (10 mM sodium acetate, pH 4.5, 5% trehalose) was buffer exchanged by gel filtration using CentriPure P100 columns, equilibrated with 100 mM sodium borate, pH 8 (0.05% SDS) as per the manufacturer's instructions. The buffer exchanged protein solution was quantified by UV-A280 using a Nanodrop 2000 spectrophotometer (1.34 mg/mL).

IL-2 (12 mg, 8.96 mL) was diluted to 9 mL with 100 mM sodium borate, pH 8 (0.05% SDS) and to this solution 0.1 M DTT (1.0 mL, 100 μmol, 127 equiv.) was added, giving a final IL-2 concentration of 1.2 mg/mL. The resulting reduction reaction was mixed gently and incubated at 22° C. for 1 h. The reduced IL-2 was buffer exchanged into 100 mM sodium borate, pH 8 (0.05% SDS) using a CentriPure P100 column as per the manufacturer's instructions and the amount of protein recovered (11.6 mg in 14.26 mL of reaction buffer) was determined by UV-A280. A solution of 5 kDa TheraPEG™ reagent (16.1 mg) was prepared in water (11.03 mL). To reduced IL-2 (11.6 mg, 0.758 μmol, 14.26 mL), 100 mM sodium borate, pH 8 (0.05% SDS) (28.24 mL) and 1.5 mg/mL solution of 5 kDa TheraPEG™ (3.37 mL, 0.981 μmol, 1.3 equiv.) were added. The conjugation reaction was mixed gently and incubated at 22° C. for 16 h. The crude reaction was analysed by SDS-PAGE and analytical SEC and then purified by preparative SEC.

Crude reaction was buffer exchanged into 50 mM sodium acetate, pH 4.5 (150 mM NaCl) and concentrated by UF/DF (Vivapsin20, 5 kDa MWCO PES). SDS was removed from PEGylated IL-2 sample using 4 mL Detergent Removal Spin Columns (Pierce®) as per the manufacturer's instructions. PEGylated IL-2 product was then purified by SEC using a HiLoad 16/600 Superdex 200 pg. Sample was isocratically eluted with 50 mM sodium acetate, pH 4.5 (150 mM NaCl) at 2 mL/min flow rate. Fractions collected over the methods were analysed by SDS-PAGE and high purity fractions were pooled. The pooled fractions were sterile filtered (0.22 μm PVDF).

Sample was quantified by UV-A280 using a Nanodrop 2000 spectrophotometer and was analysed by SEC and SDS-PAGE. Example 51 was generated as 1.7 mg (14% yield) in solution. Purity by SDS-PAGE: >99%. Purity by Analytical SEC: 98.3%.

### Example 52

### Activity of Exemplary rIL-2-[PEG]_{z} Conjugates

The activity of aldesleukin (control), examples 15, 17, 19, 20, 22, 26, 27, 37-49 were evaluated in a cell proliferation assay using CTLL-2 cells.

CTLL-2 cells (mouse cytotoxic T lymphocyte cell line) were maintained in complete RPMI 1640 medium supplemented with 10% fetal bovine serum, and 10% IL-2 culture supplement (T-STIM™ with ConA (concanavalin-A)) at 37° C. under a 5% CO₂ atmosphere. The cells were cultured in suspension until they reach a cell density of 2-3×10⁵ cells/mL before splitting.

For the activity assay, 3-4 days after the last split, the cells were washed three times in Dulbecco's phosphate buffered saline. The cells were then re-suspended in supplemented media without T-STIM™ at a cell density of ˜5×10⁵ cells/mL and plated in 96-well white walled clear bottom microplates at 90 μl/well. Experiments were also conducted using supplemented media (without T-STIM™) adjusted to pH 6.7-7, in order to minimize the release of conjugates during the course of incubation. Then, 10 μl of 10× concentrations of test compound, diluted in supplemented media without T-STIM™, was added. The cells were incubated at 37° C. in a 5% CO₂ atmosphere for 48 hours. Following the 48 hour incubation, CCK8 reagent was added (20 μl/well) and incubated for 2 hours at 37° C., 5% CO₂. The plate was then read at 450 nM and 630 nM using the Molecular devices Spectra Max i3X.

The activity of both released IL-2 and unreleased conjugates were tested. The test compounds were stored under acidic condition (10 mM sodium acetate buffer, pH 4) to stabilize conjugation. To test the activity of conjugates, the sample was diluted from the storage buffer into supplemented media˜one hour prior to the assay. To test the activity of released IL-2, the releasable conjugates were diluted ten-fold in 100 mM (final concentration) sodium bicarbonate buffer, pH 9 and pre-incubated at 37° C. for eight hours prior to start of the assay.

The EC₅₀ values (concentration of test compound required to exhibit 50% of maximal response) for cell proliferation were obtained from non-linear regression analysis of dose-response curves, using GraphPad's Prism 5.01 software.

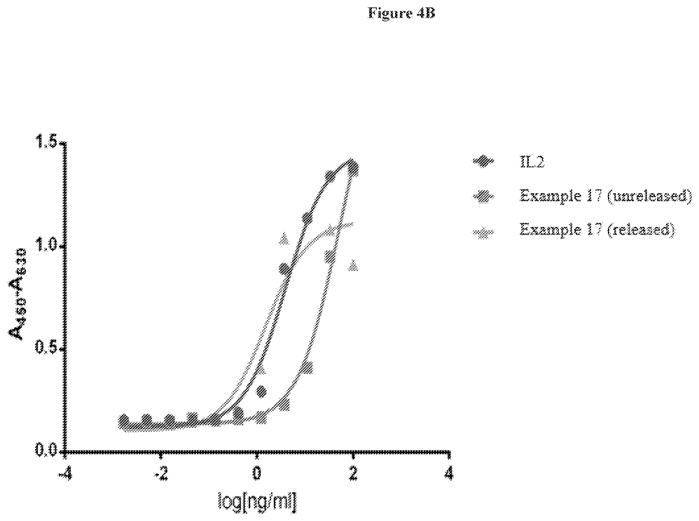

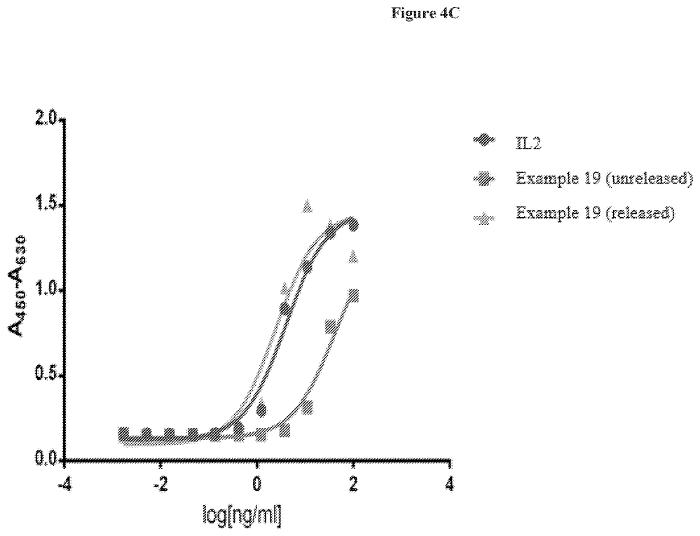

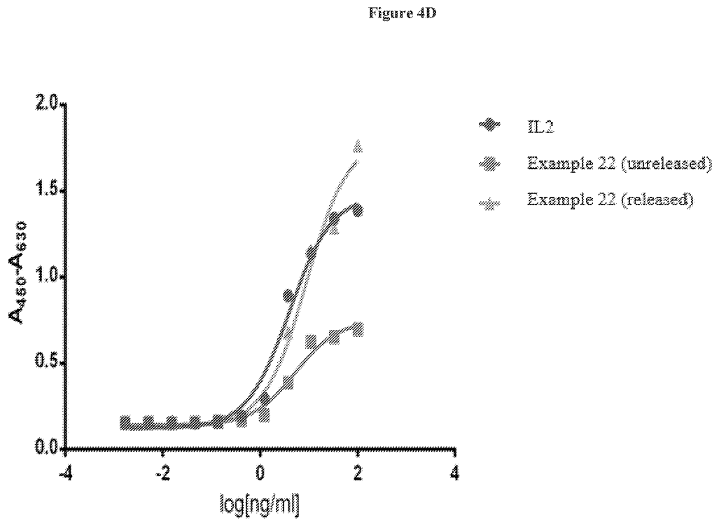

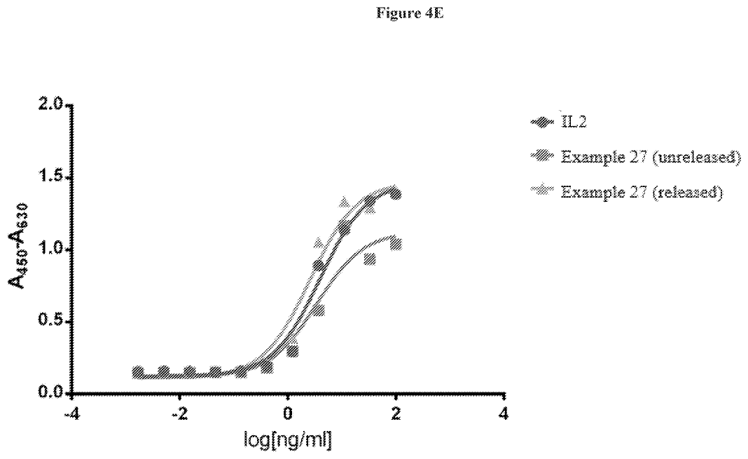

The activities of IL-2 and the conjugates were measured using a cell proliferation assay, and a summary of the results are shown in Table 3. All test articles induced growth of CTLL-2 cells in a dose-dependent manner as some examples were shown in FIG. 4A-4E. As shown in Table 3 and FIG. 4A-4E, following pre-incubation of the conjugates from Examples 15, 17, 19, 20, 22, 27, 37-49 under conditions to induce release of IL-2, activity was regained. 0.2 released from these conjugates displayed relative potency to the control IL-2.

TABLE 3
Summary of CTLL-2 Cell Proliferation in Response to IL-2 and PEG-IL-2 conjugates.

| | EC₅₀ |
|---|---|
| Test Compound | (ng/mL) |
| IL-2 control | 3.96 |
| Example 15: [20K mPEG-(F-Ph-SO₂)]_{z}-[rIL-2] (unreleased) | 58.17 |
| Example 15: [20K mPEG-(F-Ph-SO₂)]_{z}-[rIL-2] (released) | 1.71 |
| Example 17: [20K mPEG-(CF₃-Ph-SO₂)]_{z}-[rIL-2] (unreleased) | 50.80 |
| Example 17: [20K mPEG-(CF₃-Ph-SO₂)]_{z}-[rIL-2] (released) | 1.54 |
| Example 19: [20K mPEG-(Cl-Ph-SO₂)]_{z}-[rIL-2] (unreleased) | 40.87 |
| Example 19: [20K mPEG-(Cl-Ph-SO₂)]_{z}-[rIL-2] (released) | 2.45 |
| Example 22: [mPEG₂-T₂-Fmoc-20K]_{z}-[rIL-2] (unreleased) | 5.12 |
| Example 22: [mPEG₂-T₂-Fmoc-20K]_{z}-[rIL-2] (released) | 8.94 |
| Example 27: [mPEG₂-Fmoc-20K]_{z}-[rIL-2] (unreleased) | 3.40 |
| Example 27: [mPEG₂-Fmoc-20K]_{z}-[rIL-2] (released) | 2.59 |
| Example 20: [20K mPEG-(F, F-Ph-SO₂)]_{z}-[rIL-2] (unreleased) | 50.82 |
| Example 20: [20K mPEG-(F, F-Ph-SO₂)]_{z}-[rIL-2] (released) | 1.30 |
| Example 37: [15K mPEG-(CF₃-Ph-SO₂)]_{z}-[rIL-2] (unreleased) | N.A. |
| Example 37: [15K mPEG-(CF₃-Ph-SO₂)]_{z}-[rIL-2] (released) | 0.85 |
| Example 38: [15K mPEG-(Cl-Ph-SO₂)]_{z}-[rIL-2] (unreleased) | N.A. |
| Example 38: [15K mPEG-(Cl-Ph-SO₂)]_{z}-[rIL-2] (released) | 2.08 |
| Example 39: [15K mPEG-(F, F-Ph-SO₂)]_{z}-[rIL-2] (unreleased) | N.A. |
| Example 39: [15K mPEG-(F, F-Ph-SO₂)]_{z}-[rIL-2] (released) | 1.51 |
| Example 40: [15K mPEG-(F, CF₃-Ph-SO₂)]_{z}-[rIL-2] (unreleased) | N.A. |
| Example 40: [15K mPEG-(F, CF₃-Ph-SO2)]_{z}-[rIL-2] (released) | 1.28 |
| Example 41: [15K mPEG-(Cl, CONH-Ph-SO2)]_{z}-[rIL-2] (unreleased) | 116.0 |
| Example 41: [15K mPEG-(Cl, CONH-Ph-SO₂)]_{z}-[rIL-2] (released) | 0.90 |
| Example 42: [2 × 7.5K mPEG-(CF₃-Ph-Ar-SO₂)]_{z}2-[rIL-2] (unreleased) | N.A. |
| Example 42: [2 × 7.5K mPEG-(CF₃-Ph-Ar-SO₂)]_{z}-[rIL-2] (released) | 2.53 |
| Example 43: [2 × 7.5K mPEG-(CONH-Ph-R-SO₂)]_{z}-[rIL-2] (unreleased) | N.A. |
| Example 43: [2 × 7.5K mPEG-(CONH-Ph-R-SO₂)]_{z}-[rIL-2] (released) | 1.85 |
| Example 44: [2 × 7.5K mPEG-(CF₃-Ph-R-SO₂)]_{z}-[rIL-2] (unreleased) | N.A. |
| Example 44: [2 × 7.5K mPEG-(CF₃-Ph-R-SO₂)]_{z}-[rIL-2] (released) | 0.71 |
| Example 45: [17K mPEG-(CF₃-Ph-SO₂)]_{z}-[rIL-2] (unreleased) | N.A. |
| Example 45: [17K mPEG-(CF₃-Ph-SO₂)]_{z}-[rIL-2] (released) | 1.87 |
| Example 46: [17K mPEG-(Cl-Ph-SO₂)]_{z}-[rIL-2] (unreleased) | N.A. |
| Example 46: [17K mPEG-(Cl-Ph-SO₂)]_{z}-[rIL-2] (released) | 7.52 |
| Example 47: [17K mPEG-(F, F-Ph-SO₂)]_{z}-[rIL-2] (unreleased) | N.A. |
| Example 47: [17K mPEG-(F, F-Ph-SO₂)]_{z}-[rIL-2] (released) | 0.85 |
| Example 48: [17K mPEG-(F, CF₃-Ph-SO₂)]_{z}-[rIL-2] (unreleased) | N.A. |
| Example 48: [17K mPEG-(F, CF₃-Ph-SO₂)]_{z}-[rIL-2] (released) | 1.21 |
| Example 49: [17K mPEG-(Cl, CONH-Ph-SO₂)]_{z}-[rIL-2] (unreleased) | 176 |
| Example 49: [17K mPEG-(Cl, CONH-Ph-SO₂)]_{z}-[rIL-2] (released) | 1.48 |
| Example 26 (10K-Thiobridge)-IL-2 | 2.73 |

### Example 53

### pH Release Study of Exemplary rIL-2-[PEG]_{z} Conjugates

Test sample was buffer exchanged into 100 mM PBS, pH 7.4 using a P2 column. The eluate from the column was sterile filtered (0.2 μm PVDF filter) and quantified by UV-A280 using a Nanodrop spectrophotometer. The sample was diluted to 0.1 mg/mL with 100 mM PBS, pH 7.4. Fourteen vials (seven timepoints in duplicate) were loaded with test sample (100 μL). Two vials were immediately quenched with 2 M acetic acid and frozen at −80° C. (t=0 h). The remaining twelve vials were incubated at 37° C. At pre-determined timepoints (t=6, 24, 48, 72, 96 and 120 h) two vials were removed from storage at 37° C., centrifuged (1.5 min, 4000 g), quenched with 2 M acetic acid and then frozen at −80° C. Once all timepoint samples were collected, the samples were thawed and analysed by SDS-PAGE. The average PEG:IL-2 ratios were determined by densitometry analysis of the gel, these data were transformed and plotted against time using GraphPad Prism v7.04 and linker cleavage half-life was determined and summarized in Table 4. Tin was determined as the time to release half the amount of PEG from IL-2 in the conjugate.

TABLE 4
Linker cleavage half-life of PEG-IL-2 conjugates.

| Test Compound | T_{1/2} (hr) |
|---|---|
| Example 15: [20K mPEG-(F-Ph-SO₂)]_{z}-[rIL-2] | 299 |
| Example 17: [20K mPEG-(CF₃-Ph-SO₂)]_{z}-[rIL-2] | 69 |
| Example 19: [20K mPEG-(Cl-Ph-SO₂)]_{z}-[rIL-2] | 183 |
| Example 22: [mPEG₂-T₂-Fmoc-20K]_{z}-[rIL-2] | 44 |
| Example 20: [20K mPEG-(F,F-Ph-SO₂)]_{z}-[rIL-2] | 86 |
| Example 21: [20K mPEG-(F, CF₃-Ph-SO₂)]_{z}-[rIL-2] | 57 |
| Example 37: [15K mPEG-(CF₃-Ph-SO₂)]_{z}-[rIL-2] | 82 |
| Example 38: [15K mPEG-(Cl-Ph-SO₂)]_{z}-[rIL-2] | 179 |
| Example 39: [15K mPEG-(F, F-Ph-SO₂)]_{z}-[rIL-2] | 60 |
| Example 40: [15K mPEG-(F, CF₃-Ph-SO₂)]_{z}z-[rIL-2] | 53 |
| Example 41: [15K mPEG-(Cl, CONH-Ph-SO₂)]_{z}-[rIL-2] | 37 |
| Example 42: [2 × 7.5K mPEG-(CF₃-Ph-Ar-SO₂)]_{z}-[rIL-2] | 84 |
| Example 43: [2 × 7.5K mPEG-(CONH-Ph-R-SO₂)]_{z}-[rIL-2] | 257 |
| Example 44: [2 × 7.5K mPEG-(CF₃-Ph-R-SO₂)]_{z}-[rIL-2] | 55 |
| Example 45: [17K mPEG-(CF₃-Ph-SO₂)]_{z}-[rIL-2] | 89 |
| Example 46: [17K mPEG-(Cl-Ph-SO₂)]_{z}-[rIL-2] | 184 |
| Example 47: [17K mPEG-(F, F-Ph-SO₂)]_{z}-[rIL-2] | 74 |
| Example 48: [17K mPEG-(F, CF₃-Ph-SO₂)]_{z}-[rIL-2] | 58 |
| Example 49: [17K mPEG-(Cl, CONH-Ph-SO₂)]_{z}-[rIL-2] | 30 |

### Example 54

### Subcutaneous B16F10 Melanoma Efficacy Studies

1×10⁵ B16F10 cells were implanted subcutaneously for each 7-9 week old syngeneic C57BL/6 mouse at mid-dorsal region. Tumors were allowed to grow to palpable size, i.e., 70-120 cu mm before randomization and assigning groups (n=8) as designed. The mice were administered test compounds i.e., rIL-2, rIL-2-polymer conjugates or vehicle at different dose concentrations and dose regimes as indicated in Tables 5-9. The body weights and tumor volumes were measured every two or three days. The end point for this study was the time to reach median tumor volume of 2000 cu mm for a given group.

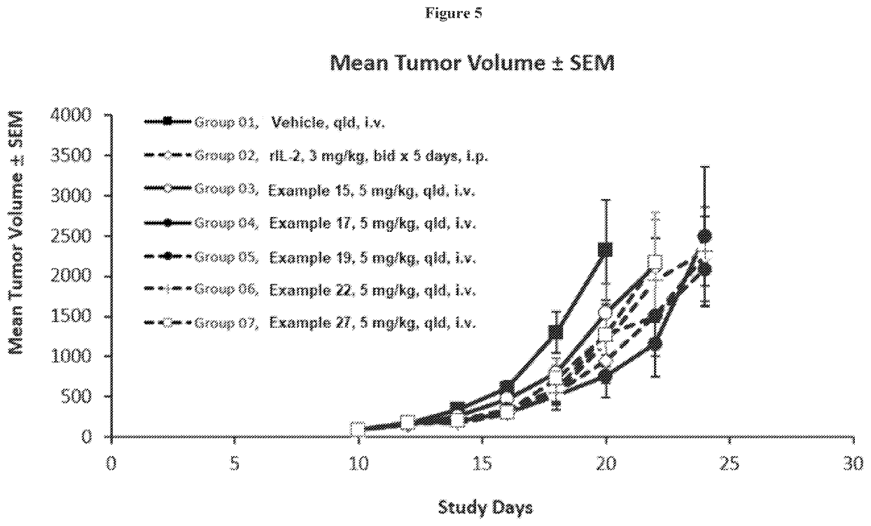

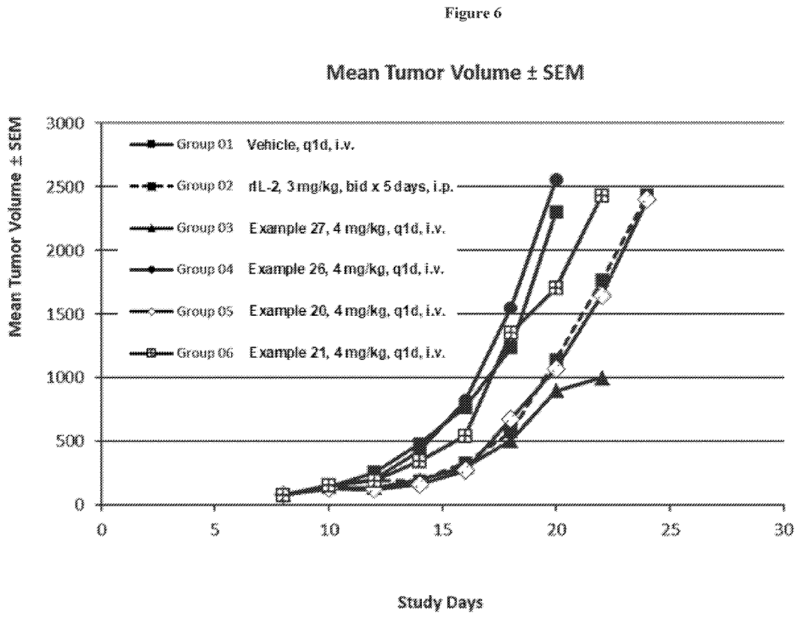

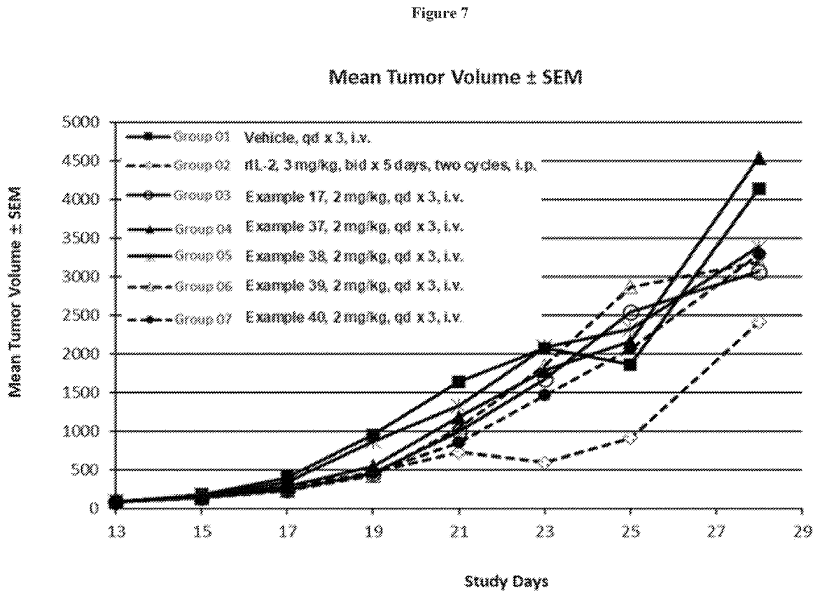

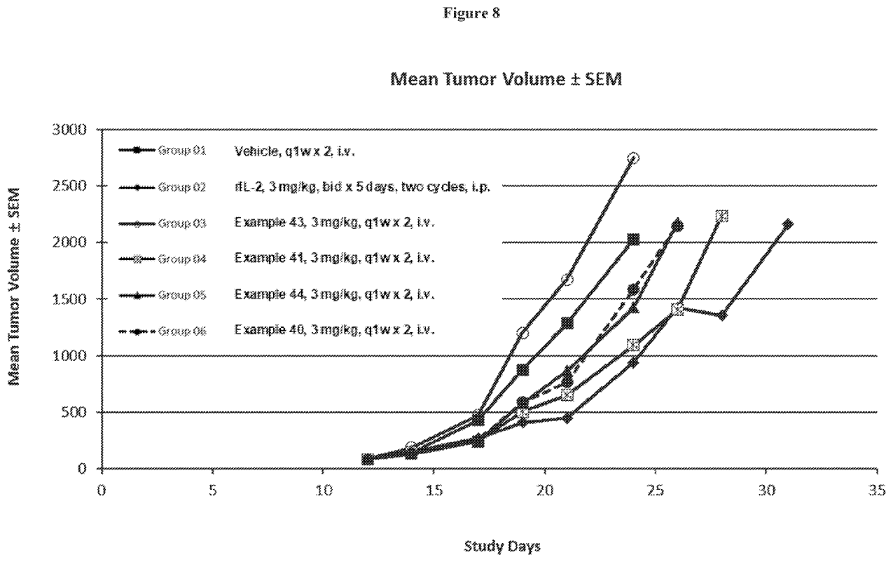

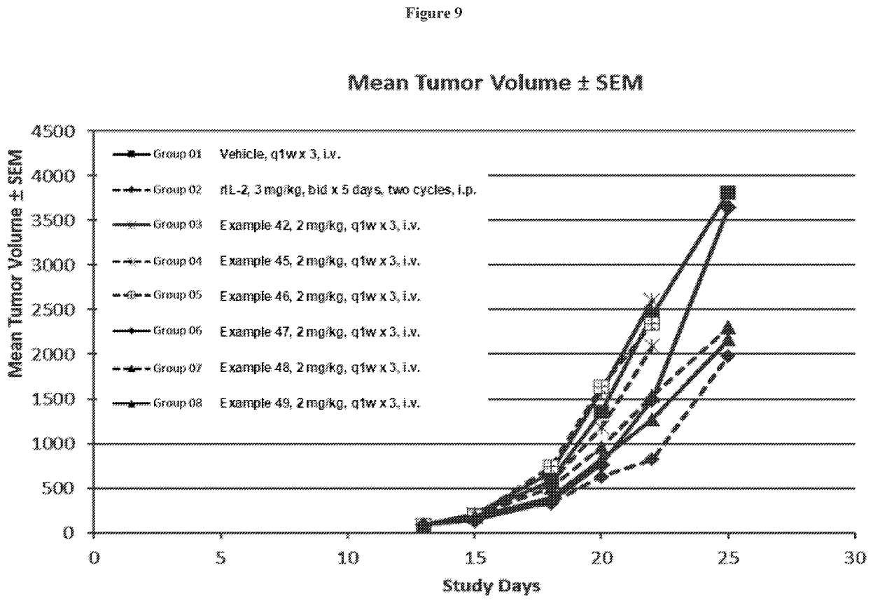

TABLE 5
Group assignments for
Example 54, FIG. 5.

| | Dose | | |
|---|---|---|---|
| | concentration | Route of | |
| Test Compound | (mg/kg) | administration | Dose |
| Vehicle: 10 mM sodium acetate, 150 mM | 0 | IV | q1d |
| NaCl, pH 4.5 | | | |
| rIL-2 control | 3 | IP | b.i.d. × 5 |
| Example 15 | 5 | IV | q1d |
| [20K mPEG-(F-Ph-SO₂)]_{z}-[rIL-2] | | | |
| Example 17 | 5 | IV | q1d |
| [20K mPEG-(CF₃-Ph-SO₂)]_{z}-[rIL-2] | | | |
| Example 19 | 5 | IV | q1d |
| [20K mPEG-(Cl-Ph-SO₂)]_{z}z-[rIL-2] | | | |
| Example 22 | 5 | IV | q1d |
| [mPEG₂-T₂-Fmoc-20K]_{z}-[rIL-2] | | | |
| Example 27 | 5 | IV | q1d |
| [mPEG₂-Fmoc-20K]_{z}-[rIL-2] | | | |

Note:
“b.i.d × 5” means twice a day for five days;
“q1d” means one dose for one day.

TABLE 6
Group assignments for Example 54, FIG. 6.

| | Dose | | |
|---|---|---|---|
| | concentration | Route of | |
| Test Compound | (mg/kg) | administration | Dose |
| Vehicle: 10 mM | 0 | IV | q1d |
| sodium acetate, 150 mM | | | |
| NaCl, pH 4.5 | | | |
| rIL-2 control | 3 | IP | b.i.d. × 5 |
| Example 27 | 4 | IV | q1d |
| [mPEG₂-Fmoc-20K]_{z}-[rIL-2] | | | |
| Example 26 | 4 | IV | q1d |
| (10K-Thiobridge)-IL-2 | | | |
| Example 20: [20K mPEG- | 4 | IV | q1d |
| (F, F-Ph-SO₂)]_{z}-[rIL-2] | | | |
| Example 21: [20K mPEG- | 4 | IV | q1d |
| (F, CF₃-Ph-SO₂)]_{z}-[rIL-2] | | | |

Note:
“b.i.d. × 5” means twice a day for five days;
“q1d” means one dose for one day.

TABLE 7
Group assignments for Example 54, FIG. 7.

| | Dose | | |
|---|---|---|---|
| | concentration | Route of | |
| Test Compound | (mg/kg) | administration | Dose |
| Vehicle: 10 mM sodium acetate, | 0 | IV | qd, dose on day 13, |
| 150 mM NaCl, pH 4.5 | | | day 21 and day 29 |
| rIL-2 control | 3 | IP | b.i.d. × 5, 2 cycles, |
| | | | dose on day 13 |
| | | | (PM), 14, 15, 16, 17, |
| | | | 18 (AM) and day 19 |
| | | | (PM), 20, 21, 22, 23, |
| | | | 24 (AM) |
| Example 17 | 2 | IV | qd, dose on day 13, |
| [20K mPEG-(CF₃-Ph-SO₂)]_{z}- | | | day 21 and day 29 |
| [rIL-2] | | | |
| Example 37: [15K mPEG-(CF₃- | 2 | IV | qd, dose on day 13, |
| Ph-SO₂)]_{z}-[rIL-2] | | | day 21 and day 29 |
| Example 38: [15K mPEG-(Cl- | 2 | IV | qd, dose on day 13, |
| Ph-SO₂)]_{z}-[rIL-2] | | | day 21 and day 29 |
| Example 39: [15K mPEG-(F, F- | 2 | IV | qd, dose on day 13, |
| Ph-SO₂)]_{z}-[rIL-2] | | | day 21 and day 29 |
| Example 40: [15KmPEG- | 2 | IV | qd, dose on day 13, |
| (F, CF₃-Ph-SO₂)]_{z}-[rIL-2] | | | day 21 and day 29 |

Note:
“b.i.d. × 5” means twice a day for five days;
“q” means one dose for one day.

TABLE 8
Group assignments for Example 54, FIG. 8.

| | Dose | | |
|---|---|---|---|
| | concentration | Route of | |
| Test Compound | (mg/kg) | administration | Dose |
| Vehicle: 10 mM | 0 | IV | Q1w × 2 |
| sodium acetate, | | | |
| 150mM NaQ, pH 4.5 | | | |
| rIL-2 control | 3 | IP | b.i.d. × 5, |
| | | | 2 cycles |
| Example 43: [2 × 7.5K | 3 | IV | Q1w × 2 |
| mPEG- | | | |
| (CONH-Ph-R-SO₂)]_{z}-[rIL-2] | | | |
| Example 41: [15K mPEG- | 3 | IV | Q1w × 2 |
| (Cl, CONH-Ph-SO₂)]_{z}-[rIL-2] | | | |
| Example 44: [2 × 7.5K | 3 | IV | Q1w × 2 |
| mPEG- | | | |
| (CF₃-Ph-R-SO₂)]_{z}-[rIL-2] | | | |
| Example 40: [15K mPEG- | 3 | IV | Q1w × 2 |
| (F, CF₃-Ph-SO₂)]z_{z}-[rIL-2] | | | |

Note
“b.i.d. × 5” means twice a day for five days;
“q1w × 2” means once a week for two cycles.

TABLE 9
Group assignments for Example 54, FIG. 9.

| | Dose | | |
|---|---|---|---|
| | concentration | Route of | |
| Test Compound | (mg/kg) | administration | Dose |
| Vehicle: 10 mM sodium acetate, | 0 | IV | Q1w × 3 |
| 150 mM NaCl, pH 4.5 | | | |
| rIL-2 control | 3 | IP | b.i.d. × 5, |
| | | | 2 cycles |
| Example 42: [2 × 7.5K mPEG- | 2 | IV | Q1w × 3 |
| (CF₃-Ph-Ar-SO₂)]_{z}-[rIL-2] | | | |
| Example 45: [17K mPEG-(CF₃- | 2 | IV | Q1w × 3 |
| Ph-SO₂)]_{z}-[rIL-2] | | | |
| Example 46: [17K mPEG-(Cl- | 2 | IV | Q1w × 3 |
| Ph-SO₂)]_{z}-[rIL-2] | | | |
| Example 47: [17K mPEG-(F, F- | 2 | IV | Q1w × 3 |
| Ph-SO₂)]-[rIL-2] | | | |
| Example 48: [17K mPEG- | 2 | IV | Q1w × 3 |
| (F, CF₃-Ph-SO₂)]_{z}-[rIL-2] | | | |
| Example 49: [17K mPEG- | 2 | IV | Q1w × 3 |
| (Cl, CONH-Ph-SO₂)]_{z}-[rIL-2] | | | |

Note:
“b.i.d. × 5” means twice a day for five days;
“qlw × 3” means once a week for three cycles.

Tumor growth inhibition following the administration of rIL-2 and rIL-2-polymer conjugates at different administration schemes are provided in FIGS. 5-9. These results indicate that the evaluated rIL-2-polymer conjugates demonstrated better efficacy at a lowered dose over rIL-2, which was dosed at 3 mg/kg twice a day for five days. No visible toxicity were observed in mouse model for IL-2-PEG conjugates, while IL-2 group mice showed lethargy and body chill symptoms. IL-2-PEG conjugates attached through different linkers possessed different anti-tumor activities. The conjugates with hydrolysis rate of 30-80 hours showed the optimal anti-tumor efficacy.

What is claimed is:

1. A releasable linker having a structure according to formula (I): 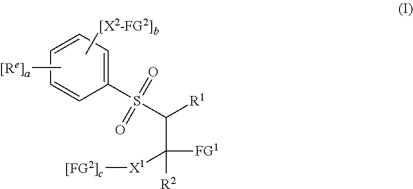
or stereoisomer, tautomer or mixture thereof, or isotopic variant thereof;
wherein:
X¹ is a first spacer moiety;
X² is a second spacer moiety;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;
a is an integer from 0 to 4;
b is an integer from 1 to 3;
c is an integer from 0 to 1;
FG¹ is a functional group capable of reacting with an amino group of an active agent to form a releasable linkage; and
FG² is a functional group capable of reacting through click chemistry.

2. The releasable linker of claim 1, wherein the releasable linker has a structure according to formula (I-B):

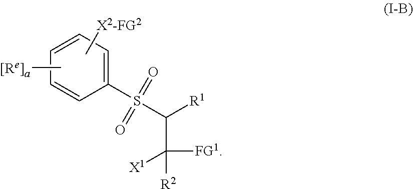

3. The releasable linker of claim 2, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHAc, —NHSO₂Me, or —OCF₃.

4. The releasable linker of claim 3, wherein the compound has following structure:

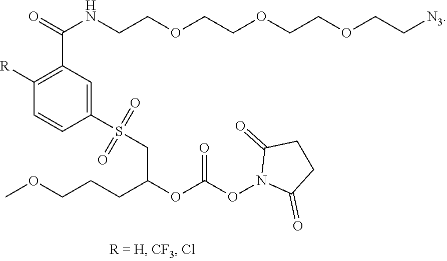

5. The releasable linker of claim 1, wherein the releasable linker has a structure according to formula (I-C):

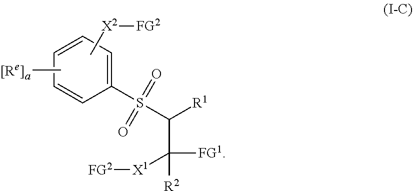

6. The releasable linker of claim 5, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHAc, —NHSO₂Me, or —OCF₃.

7. The releasable linker of claim 6, wherein the compound has following structure:

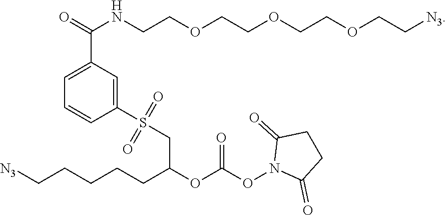 [SMILES: [N-]=[N+]=NCCCCCC(CS(=O)(=O)c1cccc(C(=O)NCCOCCOCCOCCN=[N+]=[N-])c1)OC(=O)ON1C(=O)CCC1=O]

8. A releasable linker having a structure according to formula (XVIII): 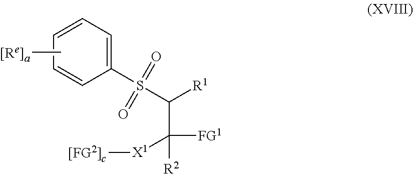
or stereoisomer, tautomer or mixture thereof, or isotopic variant thereof;
wherein:
X¹ is a spacer moiety;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;
a is an integer from 0 to 4;
c is 2;
FG¹ is a functional group capable of reacting with an amino group of an active agent to form a releasable linkage; and
FG² is a functional group capable of reacting through click chemistry.

9. The releasable linker of claim 8, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHAc, —NHSO₂Me, or —OCF₃.

10. The releasable linker of claim 9, wherein the compound has following structure:

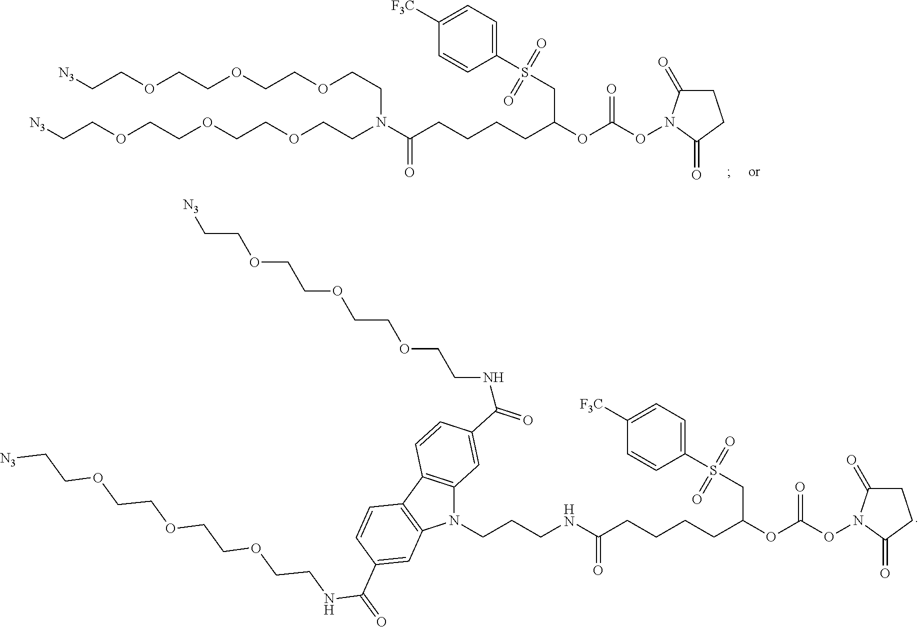 [SMILES: [N-]=[N+]=NCCOCCOCCOCCN(CCOCCOCCOCCN=[N+]=[N-])C(=O)CCCCC(CS(=O)(=O)c1ccc(C(F)(F)F)cc1)OC(=O)ON1C(=O)CCC1=O ; [N-]=[N+]=NCCOCCOCCOCCNC(=O)c1ccc2c3ccc(C(=O)NCCOCCOCCOCCN=[N+]=[N-])cc3n(CCCNC(=O)CCCCC(CS(=O)(=O)c3ccc(C(F)(F)F)cc3)OC(=O)ON3C(=O)CCC3=O)c2c1]

11. A releasable linker having a structure according to formula (II): 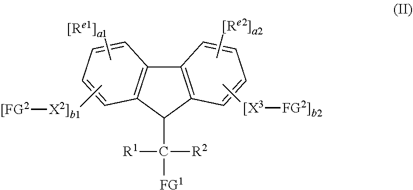
or stereoisomer, tautomer or mixture thereof, or isotopic variant thereof;
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
R^{e1}, when present, is a first electron altering group;
R^{e2}, when present, is a second electron altering group;
X², when present, is a spacer moiety;
X³, when present, is a spacer moiety;
FG¹ is a functional group capable of reacting with an amino group of an active agent to form a releasable linkage; and
FG² is a functional group capable of reacting through click chemistry.

12. The releasable linker of claim 11, wherein
a1 and a2 are each independently an integer from 0 to 2;
W and R² are each independently hydrogen, Me, or Et; and
R^{e1} and R^{e2} are each independently nitro, cyano, halogen, —CF₃, —CONHMe, —SO₂NHMe, —OMe, —NHAc, —NHSO₂Me, or —OCF₃.

13. The releasable linker of claim 11, wherein the releasable linker has a structure according to formula (II-A): 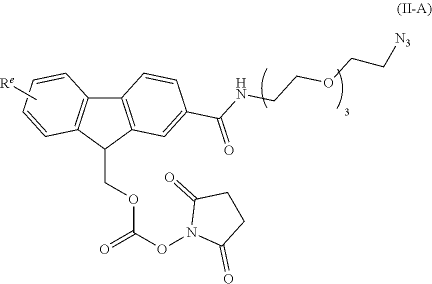
wherein R^{e} is hydrogen or an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl.

14. The releasable linker of claim 13, wherein the compound has following structure:

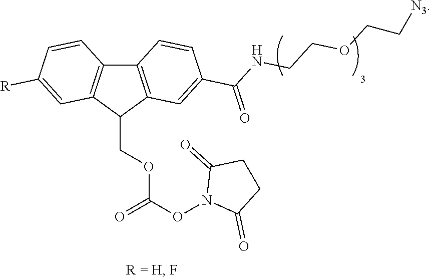

15. The releasable linker of claim 12, wherein the releasable linker has a structure according to formula (II-B):

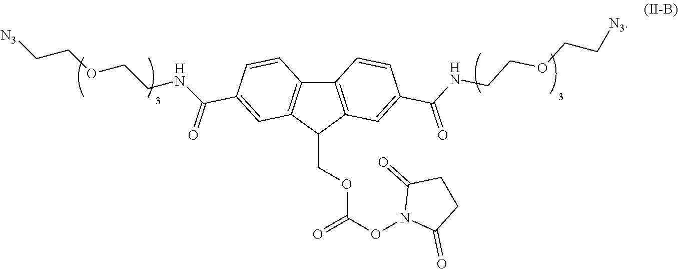

16. A releasable linker having a structure according to formula (III): 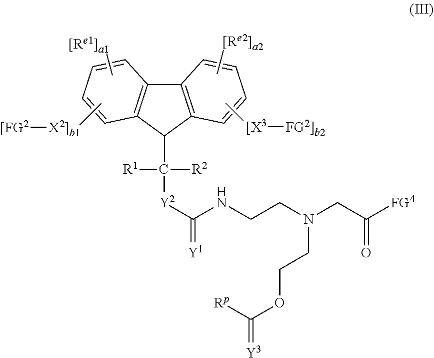
or stereoisomer, tautomer or mixture thereof, or isotopic variant thereof;
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
R^{e1}, when present, is a first electron altering group;
R^{e2}, when present, is a second electron altering group;
R^{p} is a hydrogen, alkyl, substituted alkyl, alkenyl, substituted alkenyl, alkynyl, substituted alkynyl, aryl, or substituted aryl;
X², when present, is a spacer moiety;
X³, when present, is a spacer moiety;
Y¹ is O or S;
Y² is O or S;
Y³ is O or S;
FG⁴ is a functional group capable of reacting with an amino group of an active agent to form an amide linkage; and
FG² is a functional group capable of reacting through click chemistry.

17. The releasable linker of claim 16, wherein
a1 and a2 are each independently an integer from 0 to 2;
R¹ and R² are each independently hydrogen, Me, or Et; and
R^{e1} and R^{e2} are each independently nitro, cyano, halogen, —CF₃, —CONHMe, —SO₂NHMe, —OMe, —NHAc, —NHSO₂Me, or —OCF₃.

18. The releasable linker of claim 17, wherein the releasable linker has a structure according to formula (III-A):

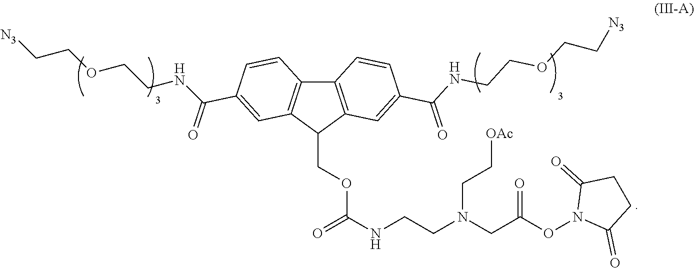

19. A releasable linker having a structure according to formula (IV): 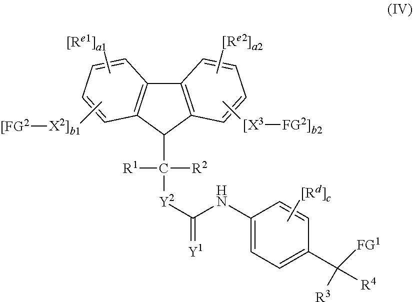
or stereoisomer, tautomer or mixture thereof, or isotopic variant thereof;
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R³ is a hydrogen, alkyl, substituted alkyl, alkenyl, substituted alkenyl, alkynyl, substituted alkynyl, aryl, or substituted aryl;
R⁴ is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
c is an integer from 0 to 4;
R^{e1}, when present, is a first electron altering group;
R^{e2}, when present, is a second electron altering group;
R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;
X², when present, is a spacer moiety;
X³, when present, is a spacer moiety;
Y¹ is O or S;
Y² is O or S;
FG¹ is a functional group capable of reacting with an amino group of an active agent to form a releasable linkage; and
FG² is a functional group capable of reacting through click chemistry.

20. The releasable linker of any one of claims 1-19, wherein FG¹ is a functional group capable of reacting with an amino group of an active agent to form a carbamate linkage.

21. The releasable linker of any one of claims 1-20, wherein FG² is an azide, an alkynyl, or a cycloalkynyl group.

22. The releasable liner of any one of claims 1-21, wherein the cycloalkynyl group is dibenzocyclooctyne (DBCO).

23. A conjugate comprising a protein covalently attached to at least one linker; wherein the conjugate comprises a structure according to formula (XIX):
Protein-(L)_{z} (XIX)
or a stereoisomer, regioisomer, tautomer or mixture thereof, or an isotopic variant thereof; or a pharmaceutically acceptable salt, solvate, hydrate, or prodrug thereof;
wherein:
z is an integer from 1 to 25;
L is a linker; and
protein is a chemokine, a chemokine antagonist, a cytokine, a cytokine antagonist, an antibody, or a therapeutic peptide.

24. The conjugate of claim 23, wherein at least one linker is a releasable linker.

25. The conjugate of claim 24, wherein the releasable linker is the releasable linker of claims 1-22.

26. The conjugate of claim 24 or 25, wherein the linker is covalently attached to an amine group of a residue within the protein.

27. The conjugate of claim 26, wherein the residue is lysine.

28. The conjugate of any one of claims 23-27, wherein one or more linkers are attached to the protein.

29. A composition comprising a mixture of the conjugates of any one of claims 23-28.

30. A conjugate comprising a protein, at least one linker, and at least one water-soluble polymer, wherein the protein is covalently attached to each water-soluble polymer via a linker, wherein the water-soluble polymer is straight or branched.

31. The conjugate of claim 30, comprising two or more linkers.

32. The conjugate of claim 31, wherein the two or more linkers comprise at least one non-releasable linker.

33. The conjugate of claim 31 or 32, wherein the two or more linkers comprise at least one releasable linker.

34. The conjugate of any one of claims 31-33, wherein the two or more linkers comprise at least one releasable linker and at least one non-releasable linker.

35. The conjugate of any one of claims 31-34, wherein the two or more linkers comprise from 1 to 8 releasable linkers and one to two non-releasable linkers.

36. The conjugate of claim 30, wherein the at least one linker is a releasable linker.

37. The conjugate of claim 30 or 36, wherein each of the linkers is a releasable linker.

38. The conjugate of claim 36 or 37, wherein the releasable linker is the releasable linker of any one of claims 1-22.

39. The conjugate of claim 30, wherein the at least one linker includes a non-releasable linker.

40. The conjugate of any one of claims 30-39, wherein the water-soluble polymer is a polymer of poly(ethylene glycol).

41. The conjugate of claim 40, wherein the poly(ethylene glycol) is terminally capped with an end-capping moiety selected from the group consisting of hydroxy, alkoxy, substituted alkoxy, alkenoxy, substituted alkenoxy, alkynoxy, substituted alkynoxy, aryloxy and substituted aryloxy.

42. The conjugate of any one of claims 30-41, wherein the water-soluble polymer has a weight-average molecular weight in a range of from about 500 Daltons to about 100,000 Daltons.

43. The conjugate of claim 42, wherein the water-soluble polymer has a weight-average molecular weight in a range of from about 500 Daltons to less than 20,000 Daltons.

44. The conjugate of claim 42, wherein the water-soluble polymer has a weight-average molecular weight in a range of from about 20,000 Daltons to less than 85,000 Daltons.

45. The conjugate of claim 42, wherein the water-soluble polymer has a weight-average molecular weight in a range of from about 85,000 Daltons to about 100,000 Daltons.

46. The conjugate of any one of claims 30-45, wherein the conjugate is covalently attached at an amine group of a residue within the protein via the linker.

47. The conjugate of claim 46, wherein the residue is lysine.

48. The conjugate of any one of claims 30-47, wherein one or more water-soluble polymers are attached to the protein via one or more linkers.

49. The conjugate of any one of claims 30-48, wherein eight or more water-soluble polymers are attached to the protein via eight or more linkers.

50. The conjugate of any one of claims 30-49, wherein the conjugate comprises a structure according to formula (XX):
Protein-(L-Macromolecule)_{z} (XX)
or a stereoisomer, regioisomer, tautomer or mixtures thereof, or an isotopic variant thereof; or a pharmaceutically acceptable salt, solvate, hydrate, or prodrug thereof;
wherein:
z is an integer from 1 to 25;
L is a linker;
protein is a chemokine, a chemokine antagonist, a cytokine, a cytokine antagonist, an antibody, or a therapeutic peptide; and
macromolecule is a water-soluble polymer, a lipid, a protein or a polypeptide.

51. The conjugate of any one of claims 30-50, wherein the conjugate comprises a structure according to the following formula: 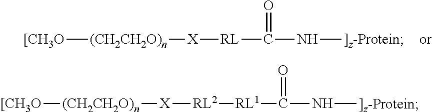
wherein:
n is an integer having a value of from 2 to 4000;
X is a spacer moiety;
RL, RL¹, and RL² are independently a releasable linker;
z is an integer from 1 to 25; and
—NH— is an amine group of a residue within the protein.

52. A composition comprising a mixture of the conjugates of any one of claims 30-51.

53. The conjugate of any one of claims 23-28, wherein the conjugate comprises a structure according to formula (VII): 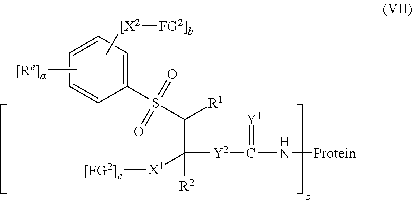
wherein:
X¹ is a first spacer moiety;
X², when present, is a second spacer moiety;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl;
a is an integer from 0 to 5;
b is an integer from 0 to 3;
c is an integer from 0 to 2;
z is an integer from 1 to 25;
Y¹ is O or S;
Y² is O or S;
FG² is a functional group capable of reacting through click chemistry selected from the group consisting of azide, alkynyl, and cycloalkynyl groups; and
—NH— is an amine group of a residue within the protein.

54. The conjugate of claim 53, wherein the conjugate comprises a structure according to formula (VII-A):

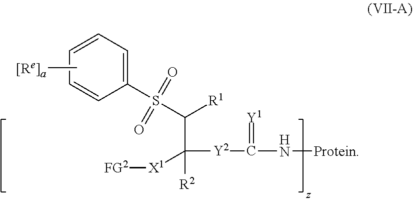

55. The conjugate of claim 54, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHAc, —NHSO₂Me, or —OCF₃.

56. The conjugate of claim 55, wherein the conjugate comprises a structure according to formula (VII-A1): 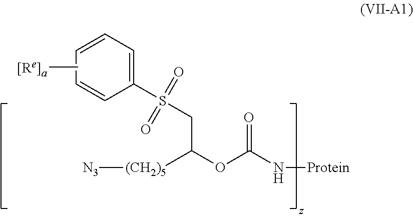
wherein a is an integer from 1 to 2; and R^{e} is 4-F, 4-Cl, 4-CF₃, 2,4-difluoro, or 2-CF₃-4-F substitution.

57. The conjugate of claim 53, wherein the conjugate comprises a structure according to formula (VII-B):

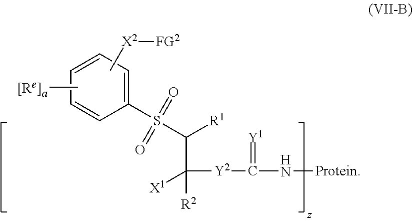

58. The conjugate of claim 57, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHAc, —NHSO₂Me, or —OCF₃.

59. The conjugate of claim 58, wherein the conjugate has following structure:

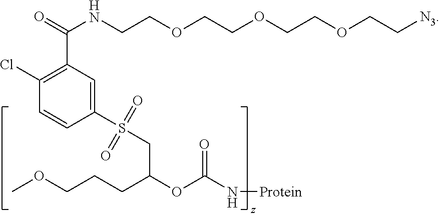

60. The conjugate of claim 53, wherein the conjugate comprises a structure according to formula (VII-C):

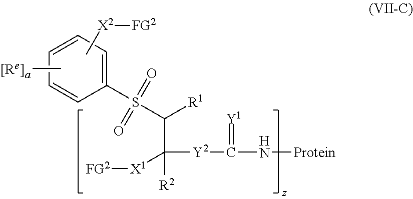

61. The conjugate of claim 60, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

62. The conjugate of claim 61, wherein the conjugate has following structure:

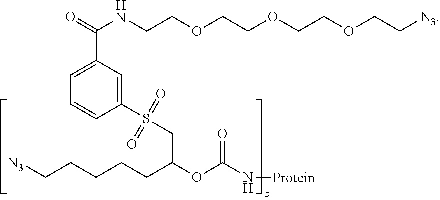

63. The conjugate of 53, wherein the conjugate comprises a structure according to formula (VII-D):

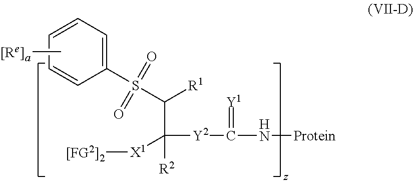

64. The conjugate of claim 63, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

65. The conjugate of claim 64, wherein the conjugate has following structure:

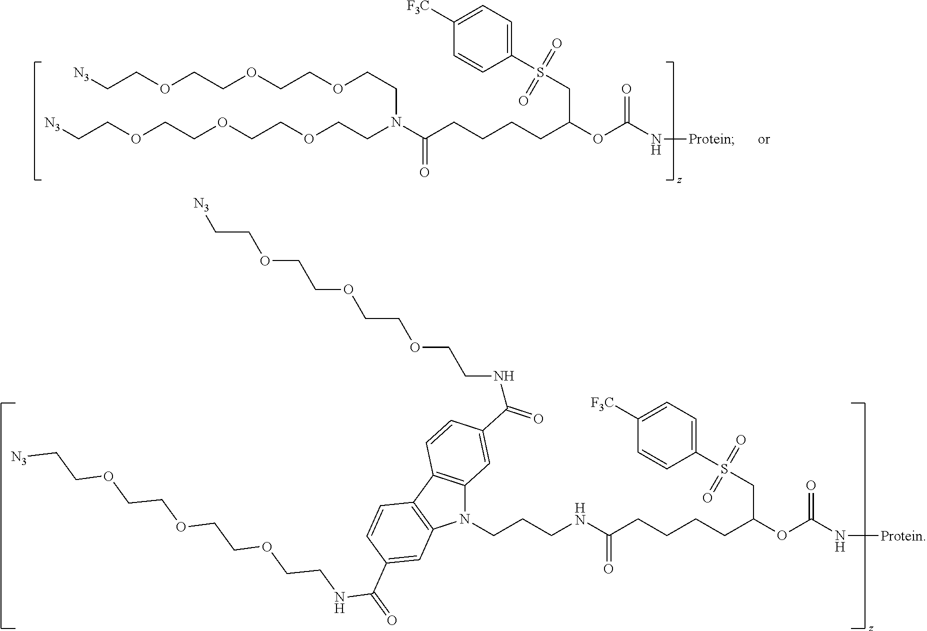

66. The conjugate of any one of claims 23-28, wherein the conjugate comprises a structure according to formula (VIII): 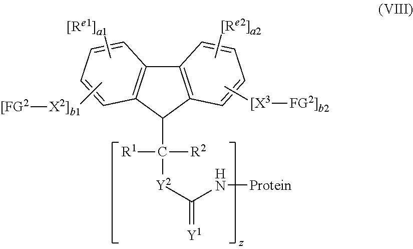
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
z is an integer from 1 to 25;
R^{e1}, when present, is a first electron altering group;
R^{e2}, when present, is a second electron altering group;
X², when present, is a spacer moiety;
X³, when present, is a spacer moiety;
Y¹ is O or S;
Y² is O or S;
FG² is a functional group capable of reacting through click chemistry selected from the group consisting of azide, alkynyl, and cycloalkynyl groups; and
—NH— is an amine group of a residue within the protein.

67. The conjugate of claim 66, wherein
a1 and a2 are each independently an integer from 0 to 2;
R¹ and R² are each independently hydrogen, Me, or Et; and
R^{e1} and R^{e2} are each independently nitro, cyano, halogen, —CF₃, —CONHMe, —SO₂NHMe, —OMe, —NHMe, —NHAc, —NHSO₂Me, or —OCF₃.

68. The conjugate of claim 67, wherein the conjugate comprises a structure according to formula (VIII-A)

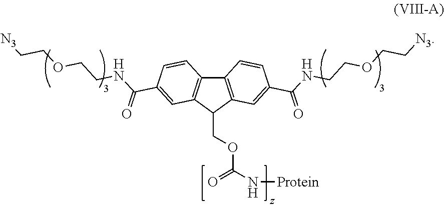

69. The conjugate of any one of claims 23-28, wherein the conjugate comprises a structure according to formula (IX): 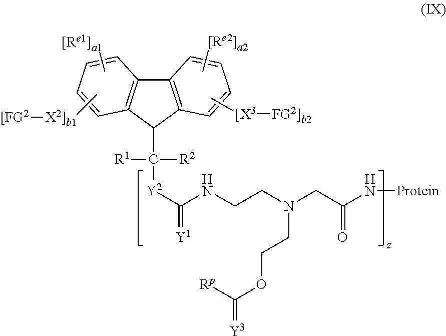
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
z is an integer from 1 to 25;
R^{e1}, when present, is a first electron altering group;
R^{e2}, when present, is a second electron altering group;
R^{p} is a hydrogen, alkyl, substituted alkyl, alkenyl, substituted alkenyl, alkynyl, substituted alkynyl, aryl, or substituted aryl;
X², when present, is a spacer moiety;
X³, when present, is a spacer moiety;
Y¹ is O or S;
Y² is O or S;
Y³ is O or S;
FG² is a functional group capable of reacting through click chemistry selected from the group consisting of azide, alkynyl, and cycloalkynyl groups; and
—NH— is an amine group of a residue within the protein.

70. The conjugate of claim 69, wherein
a1 and a2 are each independently an integer from 0 to 2;
R¹ and R² are each independently hydrogen, Me, or Et; and
R^{e1} and R^{e2} are each independently nitro, cyano, halogen, —CF₃, —CONHMe, —SO₂NHMe, —OMe, —NHMe, —NHAc, —NHSO₂Me, or —OCF₃.

71. The conjugate of claim 70, wherein the conjugate comprises a structure according to formula (IX-A):

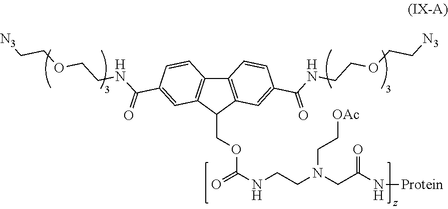

72. The conjugate of any one of claims 23-28, wherein the conjugate comprises a structure according to formula (X): 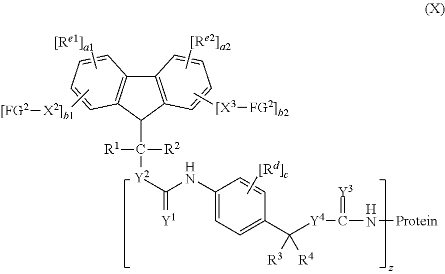
wherein:
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R³ is a hydrogen, alkyl, substituted alkyl, alkenyl, substituted alkenyl, alkynyl, substituted alkynyl, aryl, or substituted aryl;
R⁴ is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
c is an integer from 0 to 4;
z is an integer from 1 to 25;
R^{e1}, when present, is a first electron altering group;
R^{e2}, when present, is a second electron altering group;
R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;
X², when present, is a spacer moiety;
X³, when present, is a spacer moiety;
Y¹ is O or S;
Y² is O or S;
Y³ is O or S;
Y⁴ is O or S;
FG² is a functional group capable of reacting through click chemistry selected from the group consisting of azide, alkynyl, and cycloalkynyl groups; and
—NH— is an amine group of a residue within the protein.

73. The conjugate of any one of claims 30-51, wherein the conjugate comprises a structure according to formula (XI): 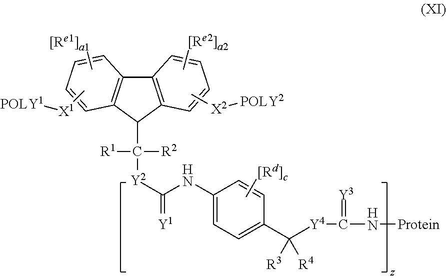
wherein:
POLY¹ is a first water-soluble polymer;
POLY² is a second water-soluble polymer;
X¹ is a first spacer moiety;
X² is a second spacer moiety;
Y¹ is O or S;
Y² is O or S;
Y³ is O or S;
Y⁴ is O or S;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R³ is a hydrogen, alkyl, substituted alkyl, alkenyl, substituted alkenyl, alkynyl, substituted alkynyl, aryl, or substituted aryl;
R⁴ is a hydrogen, alkyl, substituted alkyl, alkenyl, substituted alkenyl, alkynyl, substituted alkynyl, aryl, or substituted aryl;
a1 is an integer from 0 to 3;
a2 is an integer from 0 to 3;
c is an integer from 0 to 4;
z is an integer from 1 to 25;
R^{e1}, when present, is a first electron altering group;
R^{e2}, when present, is a second electron altering group;
R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl; and
—NH— is an amine group of a residue within the protein.

74. The conjugate of claim 73, wherein the conjugate comprises a structure according to formula (XI-A): 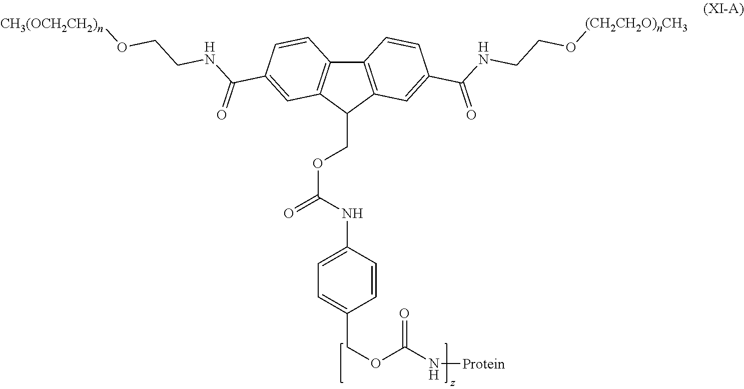
wherein n is independently an integer from 4 to 1500.

75. The conjugate of any one of claims 30-51, wherein the conjugate comprises a structure according to formula (XII): 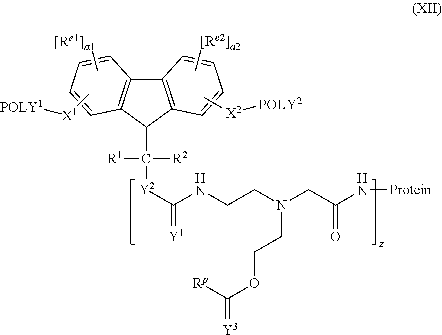
wherein:
POLY¹ is a first water-soluble polymer;
POLY² is a second water-soluble polymer;
X¹ is a first spacer moiety;
X² is a second spacer moiety;
Y¹ is O or S;
Y² is O or S;
Y³ is O or S;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 is an integer from 0-3;
a2 is an integer from 0-3;
z is an integer from 1 to 25;
R^{e1}, when present, is a first electron altering group;
R^{e2}, when present, is a second electron altering group;
R^{p} is a hydrogen, alkyl, substituted alkyl, alkenyl, substituted alkenyl, alkynyl, substituted alkynyl, aryl, or substituted aryl; and
—NH— is an amine group of a residue within the protein.

76. The conjugate of claim 75, wherein the conjugate comprises a structure according to formula (XII-A): 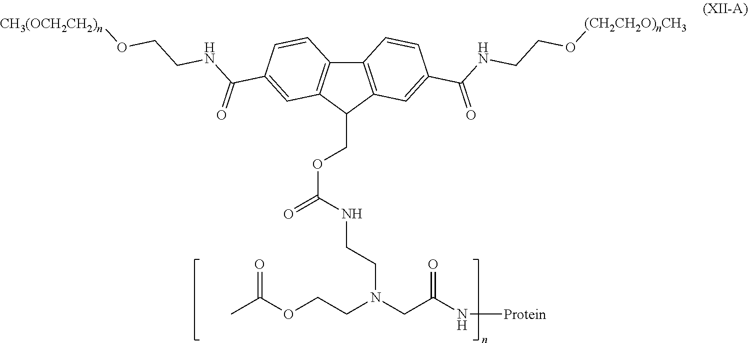
wherein n is independently an integer from 4 to 1500.

77. The conjugate of any one of claims 30-51, wherein the conjugate comprises a structure according to formula (XIII): 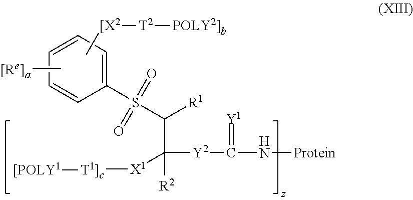
wherein:
POLY¹ is a first straight or branched water-soluble polymer;
POLY² is a second straight or branched water-soluble polymer;
X¹ is a first spacer moiety; or —X-FG²;
X², when present, is a second spacer moiety;
T¹ is a first triazole functional group;
T² is a second triazole functional group;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R^{e} is an electron altering group selected from nitro, cyano, halogen, amide, substituted amide, sulfone, substituted sulfone, sulfonamide, substituted sulfonamide, alkoxy, substituted alkoxy, alkyl, substituted alkyl, cycloalkyl, substituted cycloalkyl, aryl, substituted aryl, heteroaryl, and substituted heteroaryl; and —X-FG²;
wherein:
X is a spacer moiety; and
FG² is a functional group capable of reacting through click chemistry selecting from the group consisting of azide, alkynyl, and cycloalkynyl groups.
a is an integer from 0 to 5;
b is an integer from 0 to 3;
c is an integer from 0 to 2;
z is an integer from 1 to 25;
Y¹ is O or S;
Y² is O or S; and
—NH— is an amine group of a residue within the protein.

78. The conjugate of claim 77, wherein the conjugate comprises a structure according to (XIII-A):

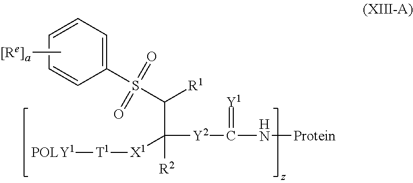

79. The conjugate of claim 78, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

80. The conjugate of claim 79, wherein the conjugate comprises a structure according to formula (XIII-A1): 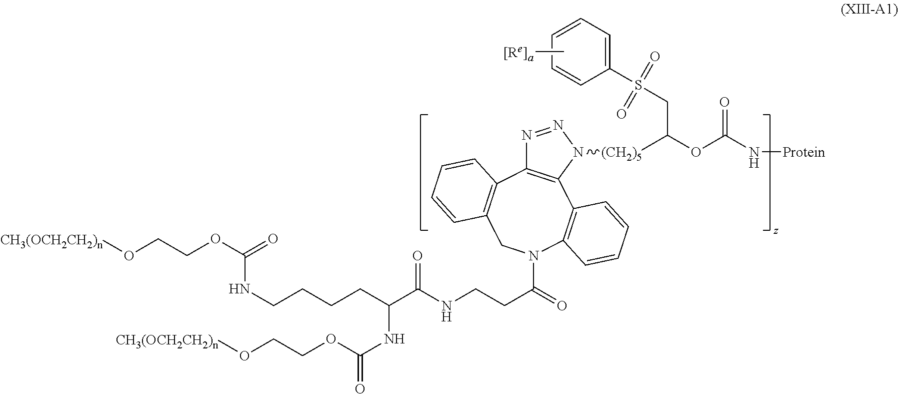
wherein:
a is an integer from 1 to 2;
R^{e} is 4-F, 4-Cl, 4-CF₃, 2,4-difluoro, or 2-CF₃₋₄-F substitution;
n is independently an integer from 4 to 1500;
z is an integer from 1 to 25; and
—NH— is an amine group of a residue within the protein.

81. The conjugate of claim 77, wherein the conjugate comprises a structure according to formula (XIII-B):

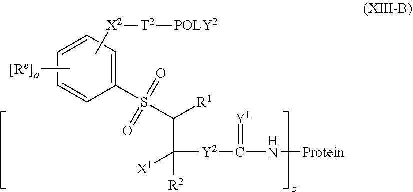

82. The conjugate of claim 81, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

83. The conjugate of claim 82, wherein the conjugate comprises a structure according to formula (XIII-B1): 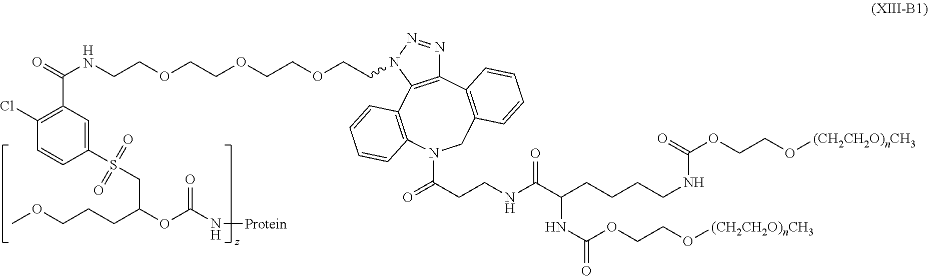
wherein:
n is independently an integer from 4 to 1500;
z is an integer from 1 to 25; and
—NH— is an amine group of a residue within the protein.

84. The conjugate of claim 77, wherein the conjugate comprises a structure according to formula (XIII-C):

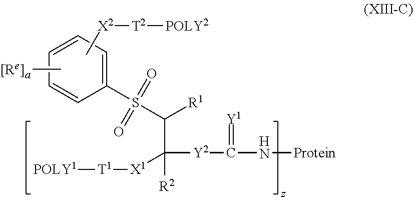

85. The conjugate of claim 84, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

86. The conjugate of claim 85, wherein the conjugate comprises a structure according to formula (XIII-C₁): 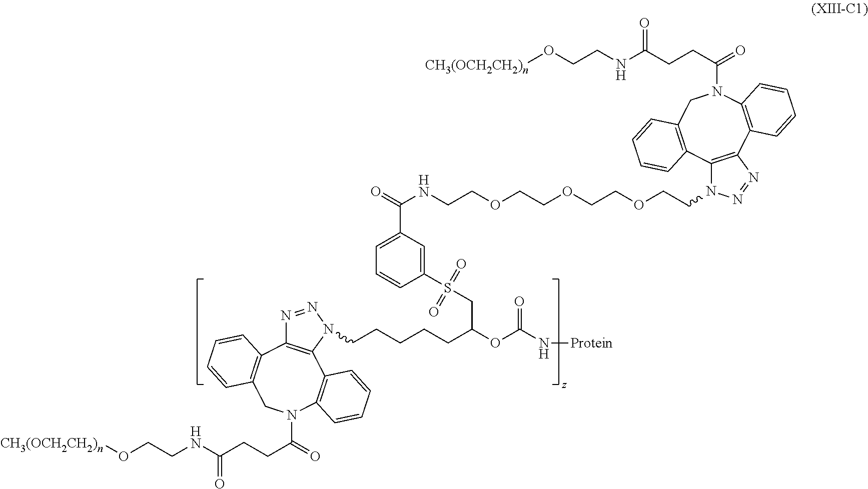
wherein:
n is independently an integer from 4 to 1500;
z is an integer from 1 to 25; and
—NH— is an amine group of a residue within the protein.

87. The conjugate of 77, wherein the conjugate comprises a structure according to formula (XIII-D):

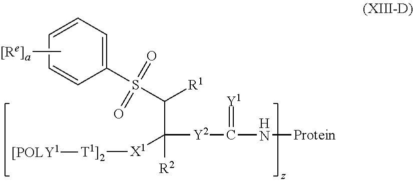

88. The conjugate of claim 87, wherein
a is an integer from 0 to 2;
R¹ and R² are each independently hydrogen, Me, or Et; and
R^{e} is nitro, cyano, halogen, —CF₃, —CONHMe, —SO₂NHMe, —OMe, —NHMe, —NHAc, —NHSO₂Me, or —OCF₃.

89. The conjugate of claim 88, wherein the conjugate comprises a structure according to formula (XIII-D1), or (XII-D2): 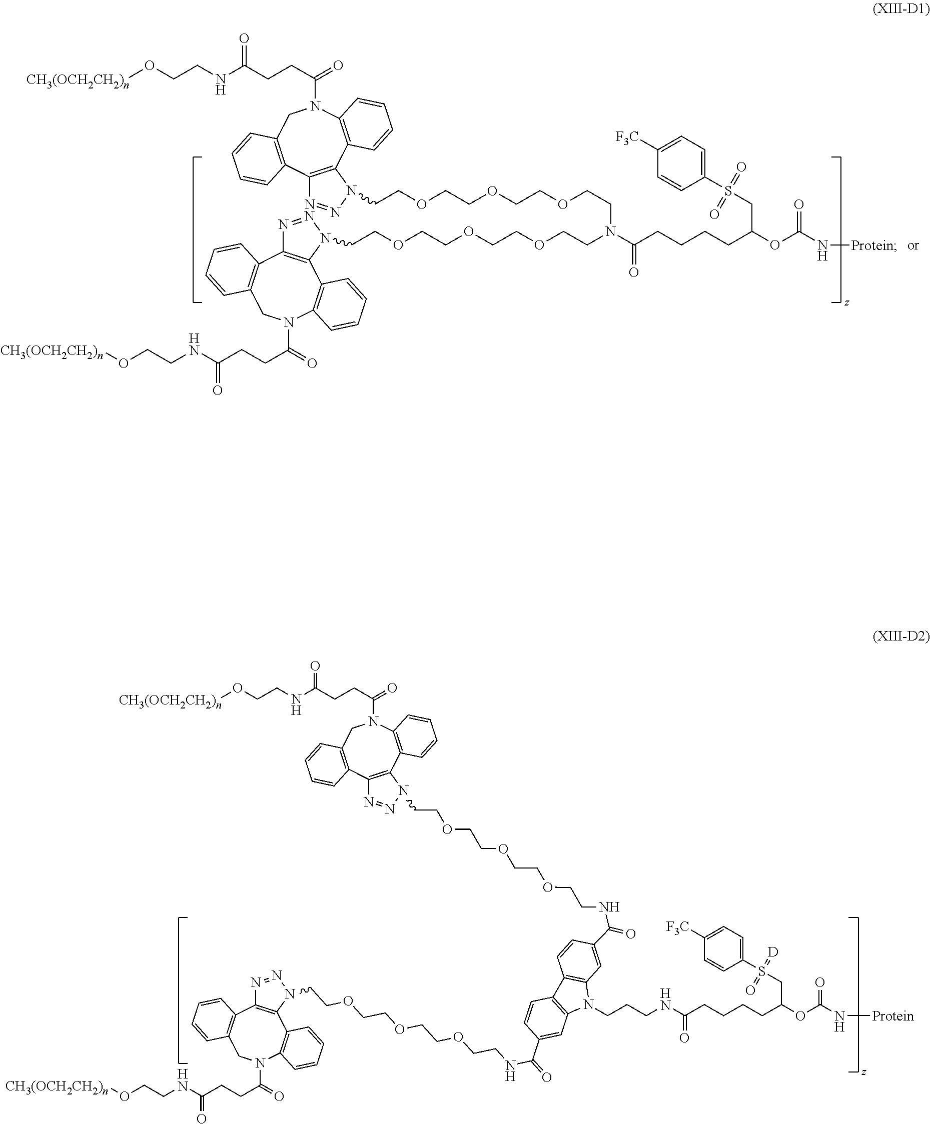
wherein:
n is independently an integer from 4 to 1500;
z is an integer from 1 to 25; and
—NH— is an amine group of a residue within the protein.

90. The conjugate of any one of claims 30-51, wherein the conjugate comprises a structure according to formula (XIV): 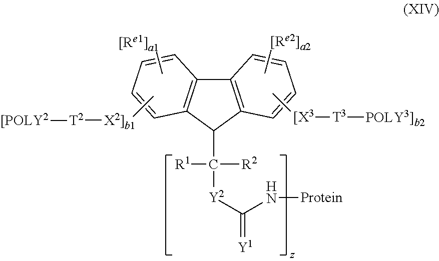
wherein:
POLY² is a straight or branched water-soluble polymer;
POLY³ is a straight or branched water-soluble polymer;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
z is an integer from 1 to 25;
R^{e1}, when present, is a first electron altering group;
R^{e2}, when present, is a second electron altering group; or —X-FG²;
wherein
X is a spacer moiety; and
FG² is a functional group capable of reacting through click chemistry selected from the group consisting of azide, alkynyl, and cycloalkynyl groups.
X², when present, is a spacer moiety;
X³, when present, is a spacer moiety;
T² is a triazole functional group;
T³ is a triazole functional group;
Y¹ is O or S;
Y² is O or S; and
—NH— is an amine group of a residue within the protein.

91. The conjugate of claim 90, wherein
a1 and a2 are each independently an integer from 0 to 2;
R¹ and R² are each independently hydrogen, Me, or Et; and
R^{e1} and R^{e2} are each independently nitro, cyano, halogen, —CF₃, —CONHMe, —SO₂NHMe, —OMe, —NHMe, —NHAc, —NHSO₂Me, or —OCF₃.

92. The conjugate of claim 91, wherein the conjugate comprises a structure according to formula (XIV-A): 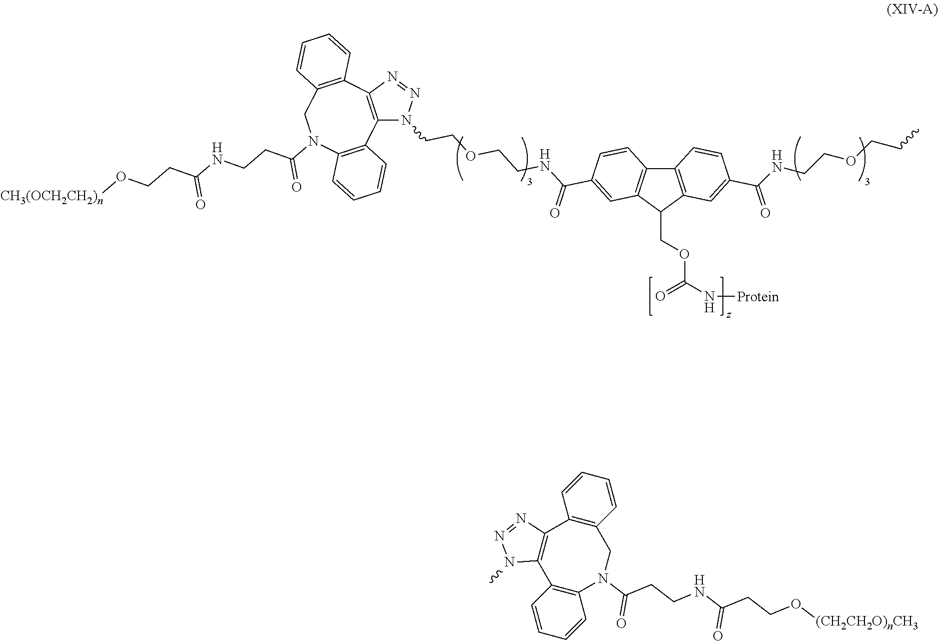
wherein:
n is independently an integer from 4 to 1500;
z is an integer from 1 to 25; and
—NH— is an amine group of a residue within the protein.

93. The conjugate of any one of claims 30-51, wherein the conjugate comprises a structure according to formula (XV): 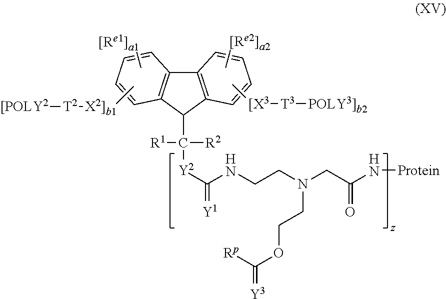
wherein:
POLY² is a straight or branched water-soluble polymer;
POLY³ is a straight or branched water-soluble polymer;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
z is an integer from 1 to 25;
R^{e1}, when present, is a first electron altering group;
R^{e2}, when present, is a second electron altering group; or —X-FG²;
wherein
X is a spacer moiety; and
FG² is a functional group capable of reacting through click chemistry selected from the group consisting of azide, alkynyl, and cycloalkynyl groups;
R^{p} is a hydrogen, alkyl, substituted alkyl, alkenyl, substituted alkenyl, alkynyl, substituted alkynyl, aryl, or substituted aryl;
X², when present, is a spacer moiety;
X³, when present, is a spacer moiety;
T² is a triazole functional group;
T³ is a triazole functional group;
Y¹ is O or S;
Y² is O or S;
Y³ is O or S; and
—NH— is an amine group of a residue within the protein.

94. The conjugate of claim 93, wherein
a1 and a2 are each independently an integer from 0 to 2;
R¹ and R² are each independently hydrogen, Me, or Et; and
R^{e1} and R^{e2} are each independently nitro, cyano, halogen, —CF₃, —CONHMe, —SO₂NHMe, —OMe, —NHMe, —NHAc, —NHSO₂Me, or —OCF₃.

95. The conjugate of claim 94, wherein the conjugate comprises a structure according to formula (XV-A): 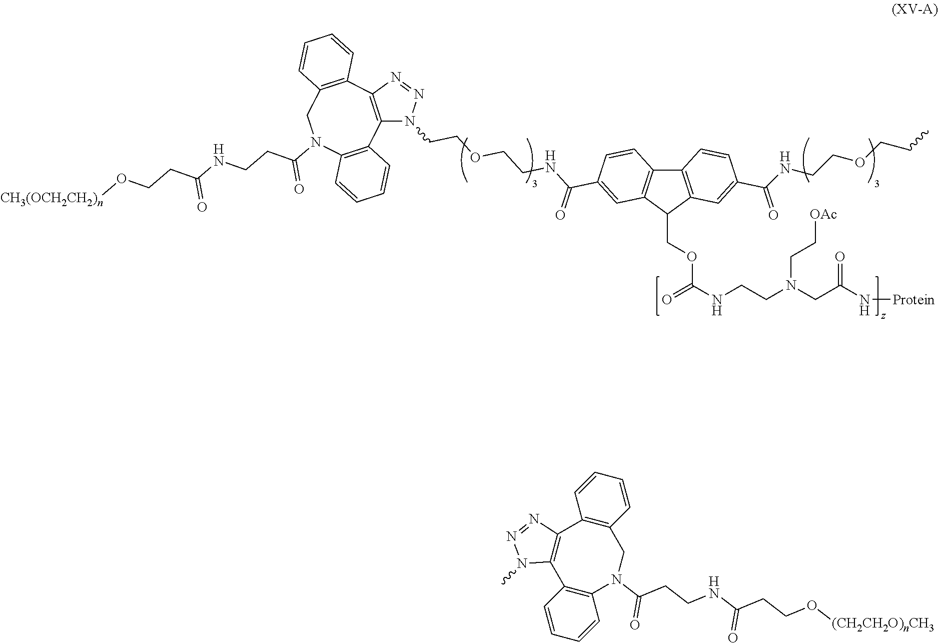
wherein:
n is independently an integer from 4 to 1500;
z is an integer from 1 to 25; and
—NH— is an amine group of a residue within the protein.

96. The conjugate of any one of claims 30-51, wherein the conjugate comprises a structure according to formula (XVI): 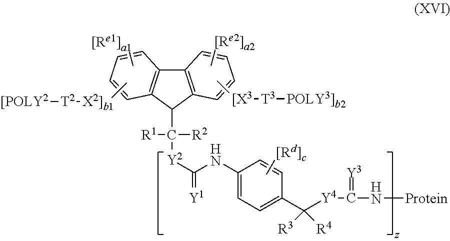
wherein:
POLY² is a straight or branched water-soluble polymer;
POLY³ is a straight or branched water-soluble polymer;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R³ is a hydrogen, alkyl, substituted alkyl, alkenyl, substituted alkenyl, alkynyl, substituted alkynyl, aryl, or substituted aryl;
R⁴ is a hydrogen, alkyl, substituted alkyl, alkenyl, substituted alkenyl, alkynyl, substituted alkynyl, aryl, or substituted aryl;
a1 and a2 are each independently an integer from 0 to 4;
b1 is 1;
b2 is an integer from 0 to 1;
c is an integer from 0 to 4;
z is an integer from 1 to 25;
R^{e1}, when present, is a first electron altering group;
R², when present, is a second electron altering group; or —X-FG²;
wherein
X is a spacer moiety; and
FG² is a functional group capable of reacting through click chemistry selected from the group consisting of azide, alkynyl, and cycloalkynyl groups;
R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl;
X², when present, is a spacer moiety;
X³, when present, is a spacer moiety;
T² is a triazole functional group;
T³ is a triazole functional group;
Y¹ is O or S;
Y² is O or S;
Y³ is O or S;
Y⁴ is O or S; and
—NH— is an amine group of a residue within the protein.

97. The conjugate of any one of claims 53-96, wherein the cycloalkynyl is dibenzocyclooctyne (DBCO).

98. The conjugate of any one of claims 23-28, 30-51, and 53-97, wherein the protein is a chemokine, a chemokine antagonist, a cytokine, a cytokine antagonist, an antibody, or a therapeutic peptide.

99. The conjugate of claim 98, wherein the cytokine is GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, or TNF-β.

100. The conjugate of claim 98 or 99, wherein the cytokine is IL-2.

101. The conjugate of claim 100, where the IL-2 comprises about 80%, 85%, 90%, 95%, 96%, 97%, 98%, or 99% sequence identity to SEQ ID NO:1.

102. The conjugate of claim 98, wherein the chemokine is MCP-1, MCP-2, MCP-3, MCP-24, MCP-5, CXCL76, I-309 (CCL1), BCA1 (CXCL13), MIG, SDF-1/PBSF, IP-10, I-TAC, MIP-1α, MIP-1β, RANTES, eotaxin-1, eotaxin-2, GCP-2, Gro-α, Gro-β, Gro-γ, LARC (CCL20), ELC (CCL19), SLC (CCL21), ENA-78, PBP, TECK(CCL25), CTACK (CCL27), MEC, XCL1, XCL2, HCC-1, HCC-2, HCC-3, or HCC-4.

103. The conjugate of claim 98, wherein the antibody targets one or more of angiopoietin 2, AXL, ACVR2B, angiopoietin 3, activin receptor-like kinase 1, amyloid A protein, β-amyloid, AOC3, BAFF, BAFF-R, B7-H3, BCMAC, A-125 (imitation), C5, CA-125, CCL11 (eotaxin-1), CEA, CSF1R, CD2, CD3, CD4, CD6, CD15, CD19, CD20, CD22, CD23, CD25, CD28, CD30, CD33, CD37, CD38, CD40, CD41, CD44, CD51, CD52, CD54, CD56, CD70, CD74, CD97B, CD125, D134, CD147, CD152, CD154, CD279, CD221, C₂₄₂ antigen, CD276, CD278, CD319, Clostridium difficile, claudin 18 isoform 2, CSF1R, CEACAM5, CSF2, carbonic anhydrase 9, CLDN18.2, cardiac myosin, CCR4, CGRP, coagulation factor III, c-Met, CTLA-4, DPP4, DR5, DLL3, DLL4, dabigatran, EpCAM, ebolavirus glycoprotein, endoglin, episialin, EPHA3, c-Met, FGFR2, fibrin II beta chain, FGF 23, folate receptor 1, GMCSF, GD2 ganglioside, GDF-8, GCGR, gelatinase B, glypican 3, GPNMB, GMCSF receptor α-chain, kallikrein, KIR2D, ICAM-1, ICOS, IGF1, IGF2, IGF-1 receptor, IL-1α, IL-1β, IL-2, IL-4Rₐ, IL-5, IL-6, IL-6 R, IL-9, IL-12, IL-13, IL17A, IL17F, IL-20, IL-22, IL-23, IL-31, IFN-α, IFN-β, IFN-γ, integrin α4β7, interferon α/β receptor, Influenza A hemagglutinin, ILGF2, HER1, HER2, HER3, HHGFR, HGF, HLA-DR, hepatitis B surface antigen, HNGF, Hsp90, HGFR, L-selectin, Lewis-Y antigen, LYPD3, LOXL2, LIV-1, MUC1, MCP-1, MSLN, mesothelin, MIF, MCAM, NCA-90, NCA-90Notch 1, nectin-4, PCDP1, PD-L1, PD-1, PCSK9, PTK7, PCDC1, phosphatidylserine, RANKL, RTN4, Rhesus factor, ROR1, SLAMF7, Staphylococcus aureus alpha toxin, Staphylococcus aureus bi-component leucocidin, SOST, selectin P, SLITRK6, SDC1, TFPI, TRAIL-R2, tumor antigen CTAA16.88, TNF-α, TWEAK receptor, TNFRSF8, TYRP1, tau protein, TAG-72, TSLP, TRAIL-R1, TRAIL-R2, TGF-0, TAG-72, TRAP, TIGIT, tenascin C, OX-40, VEGF-A, VWF, VEGFR1, or VEGFR2.

104. The conjugate of any one of claims 30-51, wherein the conjugate comprises a structure according to formula (XVII): 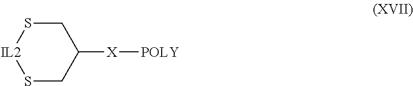
or a stereoisomer, tautomer or mixture thereof, regioisomer or mixture thereof, or an isotopic variant thereof; or a pharmaceutically acceptable salt, solvate, hydrate, or prodrug thereof;
wherein
X is a spacer moiety;
POLY is a straight or branched water-soluble polymer; and
“—S—” is a sulfur group of a residue within the IL-2 moiety.

105. The conjugate of claim 104, wherein the water-soluble polymer is a polymer poly(ethylene glycol).

106. A method for preparing Protein-Macromolecule conjugates according to scheme (I): 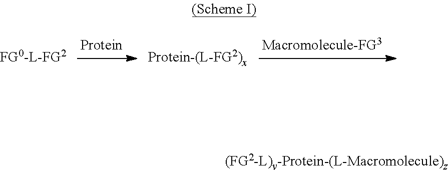
wherein x is an integer from 1 to 25;
y is an integer from 0 to 24;
z is an integer from 1 to 25, wherein x=y+z;
L is a linker;
FG⁰ is a functional group capable of reacting with a nucleophilic group of an active protein agent to form a linkage, including a carbamate linkage, a thiol bridge and the like;
FG² is a functional group capable of reacting with FG³ through click chemistry selected from the group consisting of azide, alkynyl, and cycloalkynyl groups;
FG³ is a functional group capable of reacting with FG² through click chemistry selected from the group consisting of azide, alkynyl, and cycloalkynyl groups;
protein is a chemokine, a chemokine antagonist, a cytokine, a cytokine antagonist, an antibody, or a therapeutic peptide;
macromolecule is a water-soluble polymer, a lipid, a protein or a polypeptide. It can include any of the following: a fatty acid comprises from about 6 to about 26 carbon atoms, one of the polymers selected from the group consisting of 2-methacryloyl-oxyethyl phosphoyl cholins, poly(acrylic acids), poly(acrylates), poly(acrylamides), poly(N-acryloylmorpholine), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethylene glycols), poly(ethylene oxides), poly(ethyl phosphates), poly(ethyloxazolines), poly(glycolic acids), poly(α-hydroxy acid), poly(hydroxyethyl acrylates), poly(hydroxyethyloxazolines), poly(hydroxymethacrylates), poly(hydroxyalkylmethacrylamides), poly(hydroxyalkylmethacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(oxyethylated polyol), poly(olefinic alcohol), polyphosphazene, poly(propylene glycols), poly(saccharide), poly(siloxanes), poly(urethanes), poly(vinyl alcohols), poly(vinyl amines), poly(vinylmethylethers), poly(vinylpyrrolidones), silicones, amylose, celluloses, carbomethyl celluloses, hydroxypropyl methylcelluloses, chitins, chitosans, dextrans, dextrins, gelatins, hyaluronic acids (HA) and derivatives, functionalized hyaluronic acids, mannans, pectins, heparin, heparan sulfate (HS), rhamnogalacturonans, starches, hydroxyalkyl starches, hydroxyethyl starches (HES), polysialic acid (PSA) and other carbohydrate-based polymers, xylans, and copolymers, of albumin, transferrin, transthyretin, immunoglobulin, a XTEN peptide, a glycine-rich homoamino acid polymer (HAP), a PAS polypeptide, an elastin-like polypeptide (ELP), a CTP peptide, or a gelatin-like protein (GLK) polymer.

107. The method of claim 106, wherein x or z is an integer from 2 to 25.

108. The method of claim 106, wherein x or z is an integer from 3 to 25.

109. The method of claim 106, wherein x or z is an integer from 4 to 25.

110. The method of claim 106, wherein x or z is an integer from 5 to 25.

111. The method of claim 106, wherein x or z is an integer from 6 to 25.

112. The method of any one of claims 106-111, wherein L is a releasable linker.

113. The method of claim 112, wherein the releasable linker is the releasable linker of any one of claims 1-22.

114. The method of any one of claims 106-113, wherein the protein is a chemokine, a chemokine antagonist, a cytokine, a cytokine antagonist, an antibody, or a therapeutic peptide.

115. The method of claim 114, wherein the cytokine is GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, or TNF-β.

116. The method of claim 114 or 115, wherein the cytokine is IL-2.

117. The method of claim 116, where the IL-2 comprises about 80%, 85%, 90%, 95%, 96%, 97%, 98%, or 99% sequence identity to SEQ ID NO:1.

118. The method of claim 114, wherein the chemokine is MCP-1, MCP-2, MCP-3, MCP-24, MCP-5, CXCL76, I-309 (CCL1), BCA1 (CXCL13), MIG, SDF-1/PBSF, IP-10, I-TAC, MIP-1α, MIP-1β, RANTES, eotaxin-1, eotaxin-2, GCP-2, Gro-α, Gro-β, Gro-γ, LARC (CCL20), ELC (CCL19), SLC (CCL21), ENA-78, PBP, TECK(CCL25), CTACK (CCL27), MEC, XCL1, XCL2, HCC-1, HCC-2, HCC-3, or HCC-4.

119. The method of claim 114, wherein the antibody targets one or more of angiopoietin 2, AXL, ACVR2B, angiopoietin 3, activin receptor-like kinase 1, amyloid A protein, β-amyloid, AOC3, BAFF, BAFF-R, B7-H3, BCMAC, A-125 (imitation), C5, CA-125, CCL11 (eotaxin-1), CEA, CSF1R, CD2, CD3, CD4, CD6, CD15, CD19, CD20, CD22, CD23, CD25, CD28, CD30, CD33, CD37, CD38, CD40, CD41, CD44, CD51, CD52, CD54, CD56, CD70, CD74, CD97B, CD125, D134, CD147, CD152, CD154, CD279, CD221, C242 antigen, CD276, CD278, CD319, Clostridium difficile, claudin 18 isoform 2, CSF1R, CEACAM5, CSF2, carbonic anhydrase 9, CLDN18.2, cardiac myosin, CCR4, CGRP, coagulation factor III, c-Met, CTLA-4, DPP4, DR5, DLL3, DLL4, dabigatran, EpCAM, ebolavirus glycoprotein, endoglin, episialin, EPHA3, c-Met, FGFR2, fibrin II beta chain, FGF 23, folate receptor 1, GMCSF, GD2 ganglioside, GDF-8, GCGR, gelatinase B, glypican 3, GPNMB, GMCSF receptor α-chain, kallikrein, KIR2D, ICAM-1, ICOS, IGF1, IGF2, IGF-1 receptor, IL-1α, IL-1β, IL-2, IL-4Rα, IL-5, IL-6, IL-6 R, IL-9, IL-12, IL-13, ILI7A, ILI7F, IL-20, IL-22, IL-23, IL-31, IFN-α, IFN-β, IFN-7, integrin α4β7, interferon α/β receptor, Influenza A hemagglutinin, ILGF2, HER1, HER2, HER3, HHGFR, HGF, HLA-DR, hepatitis B surface antigen, HNGF, Hsp90, HGFR, L-selectin, Lewis-Y antigen, LYPD3, LOXL2, LIV-1, MUC1, MCP-1, MSLN, mesothelin, MIF, MCAM, NCA-90, NCA-90Notch 1, nectin-4, PCDP1, PD-L1, PD-1, PCSK9, PTK7, PCDC1, phosphatidylserine, RANKL, RTN4, Rhesus factor, ROR1, SLAMF7, Staphylococcus aureus alpha toxin, Staphylococcus aureus bi-component leucocidin, SOST, selectin P, SLITRK6, SDC1, TFPI, TRAIL-R2, tumor antigen CTAA16.88, TNF-α, TWEAK receptor, TNFRSF8, TYRP1, tau protein, TAG-72, TSLP, TRAIL-R1, TRAIL-R2, TGF-β, TAG-72, TRAP, TIGIT, tenascin C, OX-40, VEGF-A, VWF, VEGFR1, or VEGFR2.

120. The method of any one of claims 106-119, wherein the cycloalkynyl is dibenzocyclooctyne (DBCO).

121. A polymeric reagent, wherein the polymeric reagent has a structure according to formula (V): 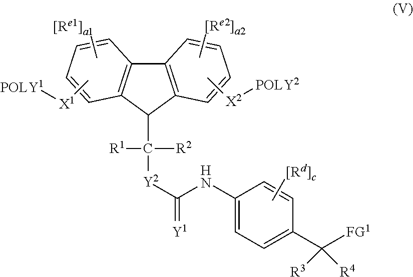
or a stereoisomer, tautomer or mixture thereof, or an isotopic variant thereof;
wherein:
POLY¹ is a first water-soluble polymer;
POLY² is a second water-soluble polymer;
X¹ is a first spacer moiety;
X² is a second spacer moiety;
Y¹ is O or S;
Y² is O or S;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
R³ is a hydrogen, alkyl, substituted alkyl, alkenyl, substituted alkenyl, alkynyl, substituted alkynyl, aryl, or substituted aryl;
R⁴ is a hydrogen, alkyl, substituted alkyl, alkenyl, substituted alkenyl, alkynyl, substituted alkynyl, aryl, or substituted aryl;
a1 is an integer from 0 to 3;
a2 is an integer from 0 to 3;
c is an integer from 0 to 4;
R^{e1}, when present, is a first electron altering group;
R^{e2}, when present, is a second electron altering group;
R^{d} is nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, aryl or heteroaryl, substituted aryl or heteroaryl; and
FG¹ is a functional group capable of reacting with an amino group of an active agent to form a releasable linkage, such as a carbamate linkage.

122. The polymeric reagent of claim 121, wherein the polymeric reagent has a structure according to formula (V-A): 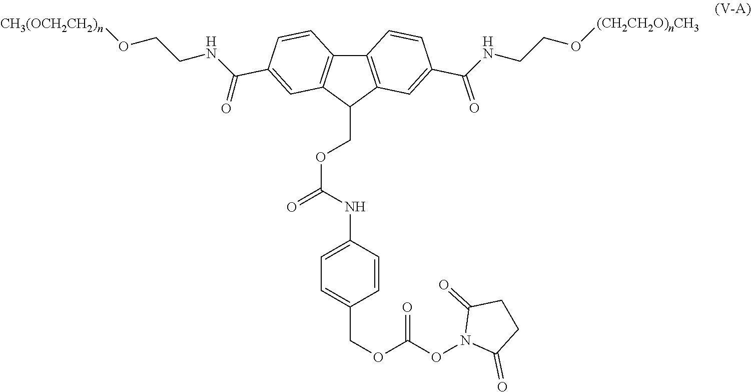
wherein n is independently an integer from 4 to 1500.

123. A polymeric reagent, wherein the polymeric reagent has a structure according to formula (VI): 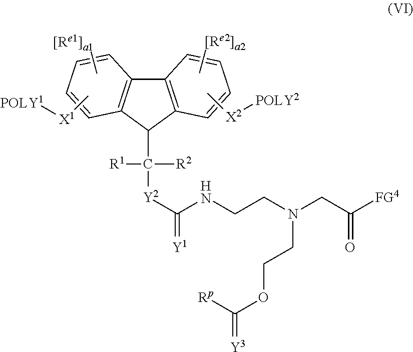
or a stereoisomer, tautomer or mixture thereof, or an isotopic variant thereof;
wherein:
POLY¹ is a first water-soluble polymer;
POLY² is a second water-soluble polymer;
X¹ is a first spacer moiety;
X² is a second spacer moiety;
Y¹ is O or S;
Y² is O or S;
Y³ is O or S;
R¹ is a hydrogen, alkyl, substituted alkyl, alkenyl, substituted alkenyl, alkynyl, substituted alkynyl, aryl, or substituted aryl;
R² is a hydrogen, alkyl, substituted alkyl, alkenyl, substituted alkenyl, alkynyl, substituted alkynyl, aryl, or substituted aryl;
a1 is an integer from 0-3;
a2 is an integer from 0-3;
R^{e1}, when present, is a first electron altering group;
R^{e2}, when present, is a second electron altering group;
R^{p} is a hydrogen, alkyl, substituted alkyl, alkenyl, substituted alkenyl, alkynyl, substituted alkynyl, aryl, or substituted aryl; and
FG⁴ is a functional group capable of reacting with an amino group of an active agent to form a releasable linkage, such as an amide linkage.

124. The polymeric reagent of claim 123, wherein the polymeric reagent has a structure according to formula (VI-A): 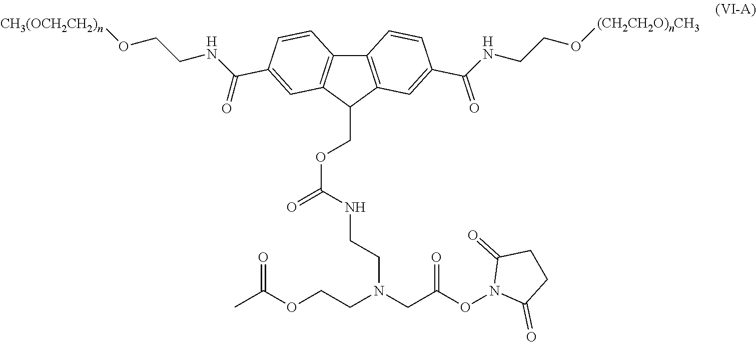
wherein n is independently an integer from 4 to 1500.

125. A pharmaceutical composition comprising the conjugate of any one of claims 23-28, 30-51, and 53-105 and one or more pharmaceutically acceptable excipients.

126. A method of treatment comprising administering to a subject in need thereof, a pharmaceutical composition of claim 125.

127. The method of claim 126, wherein the pharmaceutical composition is administered for the treatment of cancer, an infection, or an autoimmune disease.

128. A method of treatment comprising administering to a subject in need thereof, the pharmaceutical composition of claim 125 in combination with other suitable therapeutic agents.

129. The method of claim 128, wherein the therapeutic agent is an antibody.

130. The method of claim 129, wherein the antibody is an anti-tumor antigen antibody.

131. The method of claim 130, wherein the anti-tumor antigen antibody has its activity through ADCC functions.
